SEQUENCE LISTING (3 sequences)

SEQ ID NO: 1 - protein, 146 aa, Artificial Sequence
  R7 GLYAT variant polypeptide
MIEVKPINAEDTYELRHRILRPNQPIEACMFESDLLRGAFHLGGYYGGKLISIASFHQAEHSELQGQKQYQLRGMATLEGYREQKAGSSLIKHAEEILRKRGADLLWCNARTSASGYYKKLGFSEQGEVFDTPPVGPHILMYKRIT

SEQ ID NO: 2 - protein, 146 aa, Artificial Sequence
  R11 GLYAT variant polypeptide
MIEVKPINAEDTYDLRHRVLRPNQPIEACMFESDLTRSAFHLGGFYGGKLISVASFHQAEHSELQGKKQYQLRGVATLEGYREQKAGSSLVKHAEEILRKRGADMIWCNARTSASGYYRKLGFSEQGEVFDTPPVGPHILMYKRIT

SEQ ID NO: 3 - protein, 146 aa, Bacillus licheniformis
  ST401 GAT
MIEVKPINAEDTYEIRHRILRPNQPLEACMYETDLLGGAFHLGGYYRGKLISIASFHKAEHSELEGEEQYQLRGMATLEGYREQKAGSTLIRHAEELLRKKGADLLWCNARTSVSGYYEKLGFSEQGEVYDIPPIGPHILMYKKLT

(19) United States
(12) Patent Application Publication
(10) Pub. No.: US 2012/0288914 A1
(43) Pub. Date: Nov. 15, 2012

(54) CRYSTAL STRUCTURE OF GLYPHOSATE ACETYLTRANSFERASE (GLYAT) AND METHODS OF USE

(75) Inventors: Daniel Siehl, Menlo Park, CA (US); Robert J. Keenan, Chicago, IL (US); Zhenglin Hou, Ankeny, IA (US); Linda A. Castle, Mountain View, CA (US)

(73) Assignee: PIONEER HI BRED INTERNATIONAL INC., Wilmington, DE (US)

(21) Appl. No.: 13/382,780

(22) PCT Filed: Jul. 7, 2010
(86) PCT No.: PCT/US2010/041154

§ 371 Date: Jul. 20, 2012

Related U.S. Application Data
(60) Provisional application No. 61/223,613, filed on Jul. 7, 2009.

(51) Int. Cl. C12N 9/10; G06F 19/16

(52) U.S. Cl. 435/193; 703/11

(57) ABSTRACT

The presently disclosed subject matter provides compositions and methods for evaluating the potential of candidate polypeptides to associate with glyphosate with a higher binding affinity, higher binding specificity, or both or to have N-acetyltransferase activity with a higher catalytic rate when compared to a native glyphosate acetyltransferase (GLYAT) polypeptide through the provision and comparison of three-dimensional molecular structures of the candidate polypeptides and the GLYAT polypeptides provided herein. The methods further provide for identification of polypeptides with these advantageous properties using the three-dimensional molecular structures of GLYAT polypeptides.

The official copy of the sequence listing is submitted electronically via EFS-Web as an ASCII formatted sequence listing with a file named 389762SEQLIST.TXT, created on Jul. 7, 2010, and having a size of 4.14 kilobytes and is filed concurrently with the specification. The sequence listing contained in this ASCII formatted document is part of the specification and is herein incorporated by reference in its entirety.

### FIELD OF THE INVENTION

The present invention relates to the fields of molecular biology, three-dimensional structural determinations of polypeptides, and their methods of use.

### BACKGROUND OF THE INVENTION

Transgenic crops carrying herbicide resistance genes allow non-selective, broad-range herbicides such as glufosinate and glyphosate to be used as selective herbicides, effectively controlling a broader spectrum of weed species, and at the same time, minimizing injury to the crops (Castle et al. (2006) Curr. Opin. Biotechnol. 17(2):105-112). Glyphosate inhibits 5-enolpyruvylshikimate-3-phosphate (EPSP) synthase, an enzyme in the aromatic amino acid biosynthetic pathway essential for plants but absent in animals. The transgene present in most glyphosate-tolerant crops codes for a glyphosate-insensitive form of EPSPS, from Agrobacterium sp. (Padgette et al. (1996) In S. O. Duke (ed) Herbicide-Resistant Crops: Agricultural, Economic, Environmental, Regulatory, and Technological Aspects, Lewis Publishers: 53-84). An alternative glyphosate resistance strategy was recently reported (Castle et al. (2004) Science 304:1151-1154), in which glyphosate is converted to non-herbicidal N-acetylglyphosate, catalyzed by glyphosate N-acetyltransferase (GLYAT), optimized from B. licheniformis parental enzymes. In their native form, these enzymes exhibit acetylation activity to glyphosate in vitro but are unable to confer tolerance to transgenic organisms. High-efficiency variants exhibiting up to ˜5,000 fold enhancement in k_{cat}/Kₘ were obtained through multiple iterations of DNA shuffling.

Compositions and methods are needed that provide a clear understanding of how the tertiary structure of GLYAT variants impacts enzymatic activity. Such methods and compositions can be used to further develop GCN5-related N-acetyltransferases (GNATS) with improved enzymatic or substrate binding activity.

### BRIEF SUMMARY OF THE INVENTION

Compositions and methods for evaluating and identifying polypeptides that have an increased affinity or specificity for glyphosate when compared to a native glyphosate N-acetyltransferase (GLYAT) polypeptide are described. Further provided herein are methods for evaluating and identifying polypeptides having greater N-acetyltransferase activity when compared to a native N-acetyltransferase enzyme. Such methods involve the comparison of a three-dimensional molecular structure of region(s) of a GLYAT polypeptide with a three-dimensional molecular structure of a candidate polypeptide to evaluate the potential of the candidate polypeptide to bind to glyphosate with a higher binding affinity or specificity or to have higher activity than native GLYAT proteins. The methods further provide for the modification of the primary structure of the candidate polypeptide to maximize a similarity or relationship between the three-dimensional molecular structures of the GLYAT polypeptide region(s) and the candidate polypeptide in order to identify polypeptides with a higher binding affinity or activity for glyphosate.

Compositions include a computer-readable storage medium comprising the atomic coordinates of GLYAT polypeptide variants bound to glyphosate and acetyl coenzyme A (acetyl coA).

### BRIEF DESCRIPTION OF THE FIGURES

FIG. 1A and FIG. 1B provide three-dimensional representations of the liganded structures of the R7 (FIG. 1A) and R11 (FIG. 1B) variant GLYAT polypeptides with all residue substitutions of R7 compared to the wild-type and R11 compared to R7. The altered residues and ligands are shown with ball-and-stick figures. The structure of FIG. 1A is from a snapshot of a simulation of the R7 variant with AcCoA and glyphosate and the substitutions represent changes relative to the native GLYAT polypeptide. The structure of FIG. 1B is from a snapshot of a simulation of the R11 variant with AcCoA and 3PG and substitutions represent changes relative to the R7 variant.

FIG. 2A and FIG. 2B provide the molecular structure, atom names, and partial charges for glyphosate (FIG. 2A) and D-2-amino-3-phosphonopropionic acid (D-AP3; FIG. 2B). The partial charges used for the molecular modeling and MD simulations were calculated from the web server vcharge (Gilson et al (2003) J. Chem. Inf. Comput. Sci. 43(6):1982-1997). FIG. 2C and FIG. 2D show the structure conformation and atom names of 3PG (FIG. 2C) and AcCoA (FIG. 2D) in PDB:2DJJ (Siehl et al. (2007) J Biol Chem 282(15):11446-11455).

FIG. 3A and FIG. 3B provide graphs demonstrating the root mean square deviation (RMSD) and root mean square fluctuations (RMSF), respectively, for unliganded simulations. FIG. 3A graphs the heavy atom RMSD versus simulation time in picoseconds (ps). The RMSD was calculated by superimposing trajectory frames into the initial structure. All the simulations were carried out in unliganded form. The dashed line represents the R11 GLYAT variant; the solid black line represents the R7 GLYAT variant; and the gray line represents the YVII GLYAT polypeptide. FIG. 3B provides the Cα B factor profile versus residue number in the GLYAT sequence. The B factor was converted from the RMSF, B=8π<Δr²>/3 and the RMSF was calculated from the trajectory between 3 and 5 nanoseconds (ns). The dashed line represents the R11 GLYAT variant; the solid line represents the R7 GLYAT variant; and gray line represents the YVII GLYAT polypeptide. The secondary structures were assigned with DSSP based on the initial structure.

FIG. 4A provides a three-dimensional representation of the Ca trace of the open conformation of R7 GLYAT superimposed over that of the closed conformation. The gray model represents the closed conformation, which was a snapshot taken from the trajectory at ˜500 picoseconds (ps) while the black model represents the open conformation at ˜4,200 ps. The large open hole near the center of the structure is the ligand binding site. To easily monitor the openness of the active site, a distance between Q24Cα and P134Cα is marked as a dashed line. FIG. 4B shows a graph describing the openness of the glyphosate binding site as a function of simulation time. The y-axis of the graph of FIG. 4B is the distance between Q24Cα and P134Cα (as shown in FIG. 4A). A solid line represents the R7 GLYAT variant; a dashed line represents the R11 GLYAT variant; and a gray line represents the YVII GLYAT polypeptide.

FIG. 5A and FIG. 5B show a three-dimensional representation of the inter-subdomain motions of the R7 GLYAT polypeptide variant. The three superimposed structures represent the most closed, the most open, and the middle frames of trajectory projection along the first two eigenvectors. The thin black line represents the most closed form; the thick black line represents the most open form; and the gray line represents the intermediate structure. The eigenvalues and eigenvectors were calculated with principal component analysis (PCA) of the R7 trajectory ensemble before 7 nanoseconds (ns). FIG. 5A depicts the trajectory projection against the first most significant eigenvector. FIG. 5B depicts the trajectory projection against the second eigenvector.

FIG. 6A presents a three-dimensional representation of the inter-domain motions versus the wedge angles. Pseudo-dihedral angles used to measure the wedge configuration are the wedge opening angle (α+β−180°) and the wedge twisting angle (θ). FIGS. 6B-6G present graphs depicting the wedge angle population distribution of trajectory ensembles of 10 nanoseconds (ns). The x-axis of the graphs is the angle in degrees while the y-axis is the relative population. The line represents the normal distribution fitting curve with the mean (r) and standard deviation (a) provided.

FIG. 7 shows a typical β hairpin conformation taken from a snapshot of a YVII GLYAT polypeptide variant simulation at 5 ns. The β hairpin connecting β6 and β7 covers glyphosate's phosphono group and provides H138 as the catalytic base. The four tip residues (IPPI₁₃₅) forms a Vla β-turn. Proline 134 adopts a cis-peptide conformation and the dashed lines show hydrogen bond interactions.

FIG. 8 shows a stereo view of the 3PG and glyphosate binding site conformations in the crystal structure and a molecular dynamics simulation, respectively. The single black line represents the crystal structure with 3-phosphoglycerate (3PG) in the glyphosate binding site, from PDB:2JDD. The glyphosate structure was taken from a snapshot of a trajectory at 700 ps. The active site and the wedge formed by β4/5 strands in the snapshot model are represented with a double-line. Glyphosate and the acetyl part of AcCoA are shown with sticks and balls (middle). The two isolated circles are water molecules and dashed lines represent hydrogen bonds involved in glyphosate recognition.

### DETAILED DESCRIPTION OF THE INVENTION

Provided herein is the structure of the optimized R7 or R11 variant of glyphosate N-acetyltransferase (GLYAT) bound to glyphosate and acetyl coA. Table 18 provides the atomic coordinates of GLYAT R7 bound to glyphosate and acetyl coA, whereas Table 19 provides the atomic coordinates of GLYAT R11 bound to glyphosate and acetyl coA. Compositions therefore include a computer readable storage medium as well as an electronic representation of these structures.

Further provided herein are methods for evaluating the potential of a candidate polypeptide to associate with glyphosate with a higher binding affinity and/or higher binding specificity than a native GLYAT. The method comprises providing a three-dimensional molecular structure of a candidate polypeptide and comparing the candidate polypeptide molecular structure to a three-dimensional molecular structure of at least a substrate binding cavity of a GLYAT polypeptide comprising the atomic coordinates provided herein or a variant thereof to determine if the candidate polypeptide comprises the GLYAT substrate binding cavity or variant thereof. In some embodiments of the methods of the invention, the molecular structure of the GLYAT polypeptide further comprises a GNAT wedge joining region. In these embodiments, the candidate polypeptide can be a polypeptide suspected of or having N-acetyltransferase activity. The molecular structure of the candidate polypeptide is compared to the GNAT wedge joining region of the GLYAT polypeptide to determine if the candidate polypeptide comprises the wedge joining region to evaluate the potential of the candidate polypeptide to have N-acetyltransferase activity with a higher catalytic rate (K_{cat}), a higher catalytic efficiency (K_{M}/k_{cat}), or both for glyphosate when compared to a native GLYAT polypeptide. The provided molecular structures of the candidate polypeptide and GLYAT polypeptide are determined with the polypeptides bound to glyphosate and an acetyl donor (e.g., acetyl coA).

Described methods involve comparing the three-dimensional molecular structures of a GLYAT polypeptide and a candidate polypeptide to evaluate the substrate binding affinity, specificity or N-acetyl transferase activity of the candidate polypeptide. As used herein, a polypeptide having N-acetyltransferase activity refers to a polypeptide having the ability to catalyze the transfer of an acetyl group from acetyl CoA (AcCoA) or another acetyl donor to an amine (e.g., primary amine, secondary amine). For example, glyphosate N-acetyltransferase (GLYAT) can transfer an acetyl group from acetyl CoA to the nitrogen of glyphosate. As used herein, a GLYAT polypeptide or enzyme comprises a polypeptide which has glyphosate-N-acetyltransferase activity (“GLYAT” activity), i.e., the ability to catalyze the acetylation of glyphosate. In specific embodiments, a polypeptide having glyphosate-N-acetyltransferase activity can transfer the acetyl group from acetyl CoA to the N of glyphosate. Some GLYAT polypeptides are also capable of catalyzing the acetylation of glyphosate analogs and/or glyphosate metabolites, e.g., aminomethylphosphonic acid. Methods to assay for this activity are disclosed, for example, in U.S. Application Publication Nos. 2003/0083480 and 2004/0082770, and U.S. Pat. No. 7,405,074, International Application Publication Nos. WO2005/012515, WO2002/36782, and WO2003/092360, each of which is herein incorporated by reference in its entirety.

The term “GLYAT polypeptide” can refer to native GLYAT polypeptides as well as variants thereof. As used herein, a “native” GLYAT polynucleotide or polypeptide comprises a naturally occurring nucleotide sequence or amino acid sequence, respectively, that encodes or comprises a polypeptide having GLYAT activity. It should be noted, however, that the term “native GLYAT polypeptide” can be used to refer to GLYAT sequences found in nature that have been expressed recombinantly or used in other molecular biological methods. Non-limiting examples of native GLYAT polypeptides include GLYAT polypeptides from Bacillus licheniformis, including the 401, B6, and DS3 polypeptides that are encoded by the genes found in GenBank under the accession numbers AX543338, AX543339, and AX543340, respectively (Castle et al. (2004) Science 304:1151-1154, which is herein incorporated by reference in its entirety). Non-limiting variants of GLYAT polypeptides are set forth in U.S. Application Publication No. 2004/0082770 and U.S. Application. Publication No. 2005/0246798, both of which are herein incorporated by reference in their entirety.

In embodiments, a recombinant GNAT polypeptide is described having an array of amino acid side chains which together comprise a glyphosate acetyltransferase active site, said active site being composed of: (i) at least the atomic coordinates of Table 1 or Table 2; or (ii) a structural variant of the substrate binding cavity of part (i), wherein said structural variant comprises a root mean square deviation from the back-bone atoms of the amino acids of Table 1 or Table 2 of not more than 2 Å, wherein said GNAT polypeptide has less than about 60% sequence identity to the native GLYAT as set forth in SEQ ID NO: 3. In embodiments, the recombinant GNAT polypeptide has less than about 55%, 50%, 45%, 40%, 35%, 30%, 25% or 20% sequence identity to SEQ ID NO: 3.

In embodiments, a recombinant GNAT polypeptide is described having an array of amino acid side chains which together comprise a glyphosate acetyltransferase active site, said active site being composed of: (i) at least the atomic coordinates of Table 7 or Table 8; or (ii) a structural variant of the GNAT wedge joining region of part (i), wherein said structural variant comprises a root mean square deviation from the back-bone atoms of the amino acids of Table 7 or Table 8 of not more than 2 Å, wherein said GNAT polypeptide has less than about 60% sequence identity to the native GLYAT as set forth in SEQ ID NO: 3. In embodiments, the recombinant GNAT polypeptide has less than about 55%, 50%, 45%, 40%, 35%, 30%, 25% or 20% sequence identity to SEQ ID NO: 3.

The active sites described herein can be combined with any polypeptide scaffold. Thus, a de novo polypeptide or protein can be designed having the active site described herein.

The methods of the invention also encompass the use of three-dimensional molecular structures of fragments and variants of GLYAT and candidate polypeptides. By “fragment” is intended a portion of the polynucleotide or a portion of the amino acid sequence and hence polypeptide encoded thereby. In general, three-dimensional molecular structures of polypeptides are determined with the entire polypeptide sequence because tertiary structures of the polypeptide can comprise interactions between amino acid residues that are distantly located within the primary structure of the polypeptide. In some embodiments, however, a molecular structure of a fragment of a polypeptide (candidate polypeptide or GLYAT polypeptide) is provided. Fragments of a polynucleotide may encode biologically active portions of GLYAT polypeptides. A biologically active fragment of a GLYAT polypeptide is one that retains glyphosate N-acetyltransferase activity or retains the ability to bind to glyphosate, acetyl CoA, or both.

A fragment of a GLYAT polynucleotide that encodes a biologically active portion of a GLYAT polypeptide will encode at least 15, 25, 30, 50, 100, 150, 200, or 250 contiguous amino acids, or up to the total number of amino acids present in a full-length GLYAT polypeptide. A biologically active portion of a GLYAT polypeptide can be prepared by isolating a portion of one of the native or variant GLYAT polynucleotides, expressing the encoded portion of the GLYAT polypeptide (e.g., by recombinant expression in vitro), and assessing the activity of the encoded portion of the GLYAT. Polynucleotides that are fragments of a GLYAT nucleotide sequence comprise at least 15, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length GLYAT polynucleotide.

Molecular structures of variant GLYAT polypeptides are provided. As used herein, a variant GLYAT polypeptide is a polypeptide having GLYAT activity that is not found in nature without human intervention. A variant can be encoded by a variant polynucleotide that comprises a deletion and/or addition of one or more nucleotides at one or more internal sites within the native GLYAT polynucleotide and/or a substitution of one or more nucleotides at one or more sites in the native polynucleotide. For polynucleotides, conservative variants include those sequences that, because of the degeneracy of the genetic code, encode the amino acid sequence of one of the native GLYAT polypeptides. Variant polynucleotides include synthetically derived polynucleotides, such as those generated, for example, by using site-directed mutagenesis but which still encode a polypeptide having GLYAT activity. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein. The mutations that will be made in the polynucleotide encoding the variant must not place the sequence out of reading frame and optimally will not create complementary regions that could produce secondary mRNA structure.

Variants of a particular native. GLYAT polynucleotide (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the reference protein (i.e., native GLYAT polypeptide) by deletion or addition of one or more amino acids at one or more internal sites in the reference protein and/or substitution of one or more amino acids at one or more sites in the reference protein. Variant proteins encompassed by the present invention are biologically active, that is they continue to possess the desired biological activity of the reference protein, that is, glyphosate N-acetyl transferase activity or the ability to bind to glyphosate and/or acetyl coA as described herein. Biologically active variants of a GLYAT protein of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein may differ from that protein by as few as 1-15 amino acid residues, as few as 1-10, such as 6-10, as few as 5, as few as 4, 3, 2, or even 1 amino acid residue.

The proteins may be altered in various ways including amino acid substitutions, deletions, truncations, and insertions. Methods for such manipulations are generally known in the art. For example, amino acid sequence variants and fragments of the GLYAT proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The deletions, insertions, and substitutions of the protein sequence encompassed herein are not expected to produce radical negative changes in the characteristics of the protein. However, to confirm the effect of the substitution, deletion, or insertion in advance of doing so, one skilled in the art will appreciate that the effect may be evaluated by routine screening assays. Assays for measuring the acetylation of glyphosate are disclosed, for example, in U.S. Application Publication Nos. 2003/0083480 and 2004/0082770, and U.S. Pat. No. 7,405,074, and International Application Publication Nos. WO2005/012515 and WO2002/36782, each of which are herein incorporated by reference in its entirety.

Variant polynucleotides and proteins also encompass sequences and proteins derived from a mutagenic and recombinogenic procedure such as DNA shuffling. With such a procedure, one or more different GLYAT coding sequences can be manipulated to create a new GLYAT possessing the desired properties (having GLYAT activity). In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a first GLYAT gene and other known GLYAT genes to obtain a new gene coding for a protein with an improved property of interest, such as a decreased K_{M}. Strategies for such DNA shuffling are known in the art. See, for example, Stemmer (1994) Proc. Natl. Acad. Sci. USA 91:10747-10751; Stemmer (1994) Nature 370:389-391; Crameri et al. (I 997) Nature Biotech. 15:436-438; Moore et al. (1997) J. Mol. Biol. 272:336-347; Zhang et al. 0997) Proc. Natl. Acad. Sci. USA 94:4504-4509; Crameri et al. (1998) Nature 391:288-291; and U.S. Pat. Nos. 5,605,793 and 5,837,458.

Such gene shuffling procedures were used to identify optimized variants of GLYAT polypeptides with enhanced binding, specificity, or catalytic activities (Castle et al. (2004) Science 304:1151-1154). These optimized GLYAT polypeptides and the polynucleotides encoding them are known in the art and particularly disclosed, for example, in U.S. Application Publication Nos. 2003/0083480, 2004/0082770, and 2008/0234130 and U.S. Pat. No. 7,405,074, each of which is herein incorporated by reference in its entirety.

The GLYAT polypeptide used to generate the atomic coordinates provided in herein is a GLYAT R7 variant resulting from seven rounds of DNA shuffling of a native GLYAT polypeptide (Keenan et al. (2005) Proc Natl Acad Sci USA 102:8887-8892, which is herein incorporated by reference in its entirety) for which a crystal structure was determined (Siehl et al. (2007) J Biol Chem 282:11446-11455; Protein Databank (PDB):2JDC; PDB:2JDD; each of which is herein incorporated by reference in its entirety). In some embodiments, the R7 GLYAT variant polypeptide comprises the sequence set forth in SEQ JD NO: 1. The R7 GLYAT variant exhibits an improved catalytic efficiency for glyphosate in comparison to native GLYAT polypeptides (Siehl et al. (2007) J Biol Chem 282:11446-11455, which is herein incorporated by reference in its entirety). Thus, in some embodiments, the GLYAT polypeptide for which a molecular structure is provided for comparison to the structure of a candidate polypeptide has the sequence set forth in SEQ ID NO: 1. In other embodiments, the molecular structure represents an R11 GLYAT variant from the eleventh round of DNA shuffling (Keenan et al. (2005) Proc Natl Acad Sci USA 102:8887-8892) referred to by Siehl et al. (2007) J Biol Chem 282:11446-11455. In some embodiments, the R 11 GLYAT variant polypeptide has the sequence set forth in SEQ ID NO: 2.

Described methods are used to evaluate candidate polypeptides to determine if the polypeptides bind glyphosate with a higher binding affinity or greater specificity or if they exhibit greater catalytic activity than a native GLYAT polypeptide. As used herein, a “candidate polypeptide” refers to polypeptides that are being evaluated in the methods of the invention. The candidate polypeptide can be a naturally-occurring polypeptide or one that is not found in nature. Naturally-occurring candidate polypeptides may be from any organism, including but not limited to, a bacterium, fungus, animal, or human. The non-naturally occurring candidate polypeptide may have resulted from the mutagenesis or gene shuffling of a naturally-occurring sequence and may have been produced through recombinant or synthetic means.

In some embodiments, the candidate polypeptide has been shown to exhibit N-acetyltransferase activity or has sequence similarity to an N-acetyltransferase enzyme known in the art. Several families of N-acetyltransferase polypeptides are known. Such families include the GCN5 family, the p300/CBP family, the TAF250 family, the SRC) family, the MOZ family, and the N-terminal acetyltransferases (NAT) family. See, for example, Kouzarides et al., (2002) The EMBO J. 19:1176-1179; Kouzarides (1999) Current Opinions in Genetics Development 79:40-48, and Polevoda et al. (2003) J. Mol. Biol. 325:595-622, each of which are herein incorporated by reference in its entirety. Another family of N-acetyltransferases includes the GCN5-related N-acetyltransferases. See, INTERPRO Acc. No. IPRO00182, PFAM Accession No. PF00583 and Prosite profile PS51186. The GNAT superfamily includes aminoglycoside N-acetyltransferases, serotonin N-acetyltransferase (also known as aryl alkylamine N-acetyltransferase or AANAT), phosphinothricin acetyltransferase (PAT); glucosamine-6-phosphate N-acetyltransferase, glyphosate-N-acetyltransferase, the histone acetyltransferases, mycothiol synthase, protein N-myristoyltransferase, and the Fern family of amino acyl transferases (see Dyda et al, (2000) Annu. Rev. Biophys. Biomol. Struct. 29:81-103, which is herein incorporated in its entirety).

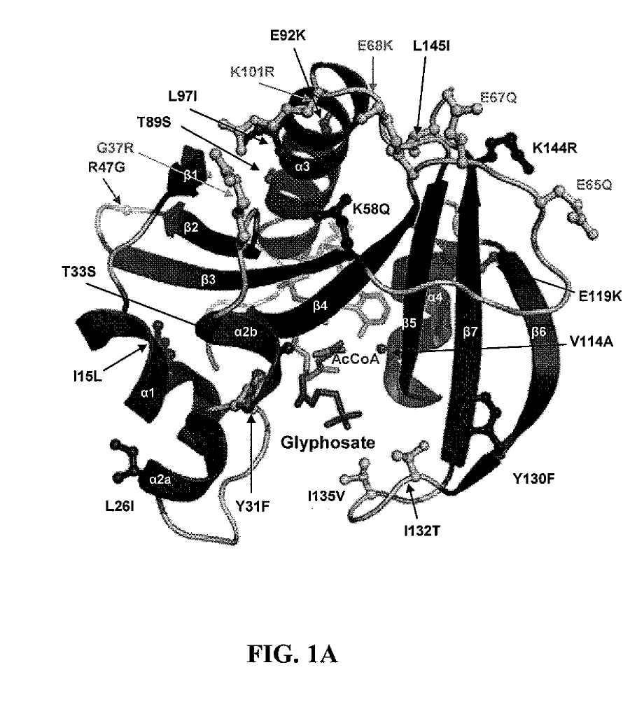

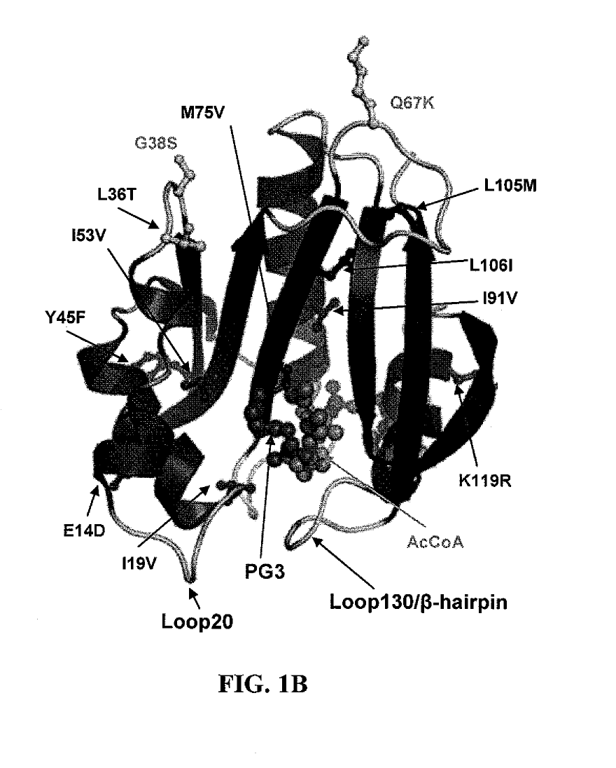

In some of these embodiments, the candidate polypeptide shares at least 50%, 55%, 60%, 70%, 75%, 80%, 85%, 90%, 95%, 96%, 97%, 98%, 99%, or greater sequence identity with a known N-acetyltransferase enzyme over the full-length of the polypeptide or with a fragment of the polypeptide. The candidate polypeptide and known N-acetyltransferase enzyme may share sequence similarity over at least about 20, 30, 40, 50, 100, 200, 300, 400, 500, 600, 700, 800, 900, 1000, 2000 or more contiguous amino acids. The candidate polypeptide or the N-acetyltransferase with which a candidate polypeptide shares sequence identity may be a known member of the GCN5-related N-acetyltransferase (GNAT) superfamily of enzymes. In some embodiments, the three-dimensional molecular structure of the candidate polypeptide comprises a GNAT wedge. As used herein, a GNAT wedge comprises a V-shaped wedge formed by two central parallel beta strands splaying apart at the middle point (see β4 and β5 in FIG. 1).

In some embodiments, the candidate polypeptide exhibits a similar primary structure to a native or variant GLYAT polypeptide. For example, the candidate polypeptide may share at least 50%, 55%, 60%, 70%, 75%, 80%, 85%, 90%, 95%, 96%, 97%, 98%, 99%, or greater sequence identity with a native GLYAT polypeptide or an optimized variant GLYAT polypeptide.

In some embodiments, the candidate polypeptide exhibits a similar primary structure to a native or variant phosphinothricin acetyltransferase (PAT) polypeptide, another enzyme capable of herbicide detoxification (De Block et al. (1987) EMBO J. 6:2513-2518). PAT polypeptides acetylate and detoxify phosphinothricin herbicides, such as glufosinate. Interestingly, GLYAT and PAT not only carry out the same acetylation reaction, but also share similar three-dimensional structures. Despite sequence divergence, the structural alignment between GLYAT PDB:2bsw (Keenan et al. (2005) Proc. Natl. Acad. Sci. USA 102(25):8887-8892) and PAT PDB:1 yr0 (Berman et al. (2000) Nucleic Acids Research 28:235-242) shows the two structures possessing the same fold with a Dali Z-score of 14.7 and an RMSD of 2.2 Å (Holm & Sander (1996) Science 273(5275):595-603). Furthermore, both glyphosate and glufosinate are similar in their chemical composition and structure.

Three-dimensional molecular structures of a GLYAT polypeptide and a candidate polypeptide are described herein. As used herein, the terms “molecular structure” refer to the arrangement of atoms within a particular object (e.g., polypeptide). Polypeptides can comprise a primary, secondary, and a tertiary molecular structure. A primary structure of a polypeptide consists of the linear arrangement of its amino acid residues, which is described by the amino acid sequence of the polypeptide. The secondary structure of a polypeptide consists of local inter-residue interactions by hydrogen bonds between backbone amide and carbonyl groups. The most common secondary structures are alpha helices and beta sheets. The tertiary structure represents the folding of the polypeptide chain, combining the elements of secondary structure, linked by turns and loops imparted by non-bond interactions and disulfide bonds. A three-dimensional molecular structure refers to the three-dimensional arrangement of atoms within a particular object (e.g., the three-dimensional structure of the atoms that comprise a polypeptide, and, optionally, the atoms that comprise a substrate that interacts with the polypeptide). In reference to a polypeptide, a three-dimensional molecular structure of a polypeptide is a representation of the tertiary structure of the polypeptide.

As used herein, a “beta-sheet” refers to two or more polypeptide chains (or beta-strands) that run alongside each other and are linked in a regular manner by hydrogen bonds between the main chain C═O and N—H groups. Therefore all hydrogen bonds in a beta-sheet are between different segments of a polypeptide. Hydrogen bonds in anti-parallel sheets are perpendicular to the chain direction and spaced evenly as pairs between strands. Hydrogen bonds in parallel sheets are slanted with respect to the chain direction and spaced evenly between strands.

As used herein, an “alpha helix” refers to the most abundant helical conformation found in globular proteins and the term is used in accordance with the standard meaning of the art. In an alpha helix, all amide protons point toward the N-terminus and all carbonyl oxygens point toward the C-terminus. Hydrogen bonds within an alpha helix also display a repeating pattern in which the backbone C═O of residue X (wherein X refers to ally amino acid) hydrogen bonds to the backbone H—N of residue X+4. The alpha helix is a coiled structure characterized by 3.6 residues per turn, and translating along its axis 1.5 Å per amino acid. Thus the pitch is 3.6×1.5 or 5.4 Å. The screw sense of alpha helices is always right-handed.

As used herein, a “loop” refers to any other conformation of amino acids (i.e. not a helix, strand or sheet). Additionally, a loop may contain hydrogen bond interactions between amino acids, including the side chains of the amino acids, but not in a repetitive, regular fashion.

A three-dimensional molecular structure of a polypeptide or a fragment thereof is most often provided through a solved structure based on X-ray diffraction data from a crystal of the polypeptide. One of skill in the art will also appreciate that, along with X-ray crystallography, three-dimensional molecular structures can also be generated using nuclear magnetic resonance (NMR) spectroscopy. Although NMR spectroscopy advantageously allows for the structure of a particular polypeptide to be determined in solution, the utility of NMR for structure determination is limited to very small proteins. Methods for structure determination using NMR can be found, for example, in Wüthrich (1986) NMR of proteins and nucleic acids, Wiley New York; Wüthrich (1990) J Biol Chem 265:22059-22062; Cavanagh et al, (1996) Protein NMR Spectroscopy, Academic Press; San Diego), each of which is herein incorporated by reference in its entirety.

In some embodiments, the three-dimensional molecular structures of a GLYAT polypeptide, a candidate polypeptide, or both are determined using X-ray crystallography, wherein the polypeptides are purified, crystallized, and exposed to an X-ray beam to generate diffraction data from which a three-dimensional molecular structure can be determined.

As used herein, the term “crystal” refers to any three-dimensional ordered array of molecules that diffracts X-rays. In order to generate crystals of a polypeptide or for structure determination via NMR spectroscopy, the polypeptide must be purified and concentrated. The polypeptide can be naturally or synthetically derived or produced by recombinant means. For example, a bacterial host, such as E. coli, can be used to express large quantities of the GLYAT or candidate polypeptide. The polypeptide can be purified by methods known in the art, including, but not limited to, selective precipitation, dialysis, chromatography, and/or electrophoresis. In some embodiments, the GLYAT polypeptide is purified using CoA-agarose affinity chromatography and gel filtration. Purification may be monitored by SDS-PAGE or by measuring the ability of a fraction to perform the catalytic activity. Any standard method of measuring acetyltransferase activity may be used.

For certain embodiments, it may be desirable to express the polypeptide as a fusion protein. In specific non-limiting embodiments, the fusion protein comprises a tag which facilitates purification of the GLYAT or candidate polypeptide. As referred to herein, a “tag” is any added series of amino acids which are provided in a protein at either the C-terminus, the N-terminus, or internally that contributes to the identification or purification of the protein. Suitable tags include but are not limited to tags known to those skilled in the art to be useful in purification including but not limited to a His tag, flag tag, glutathione-s-transferase, and maltose binding protein. Such tagged proteins may also be engineered to comprise a cleavage site, such as a thrombin, enterokinase or factor X cleavage site, for ease of removal, of the tag before, during or after purification. Vector systems which provide a tag and a cleavage site for removal of the tag are particularly useful to make expression constructs for expression and purification of the polypeptide. A tagged polypeptide may be purified by immuno-affinity or conventional chromatography, including but not limited to, chromatography employing the following:

glutathione-sepharose (Amersham-Pharmacia, Piscataway, N.J.) or an equivalent resin, nickel or cobalt-purification resins, nickel-agarose resin, anion exchange chromatography, cation exchange chromatography, hydrophobic resins, gel filtration, antibody-conjugated resin, and reverse phase chromatography. In some embodiments, after purification, at least about 50%, 60%, 70%, 80%, 90%, 95%, 96%, 97%, 98%, 99%, or greater of total protein is the GLYAT or candidate polypeptide or a mixture of the polypeptide and one or more substrates or modulators thereof (e.g., glyphosate, acetyl coA). The polypeptide or complexed polypeptide may be concentrated to achieve a concentration equal to or greater than about 1 mg/ml for crystallization purposes, including but not limited to about 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, or greater. In one embodiment, the concentration is greater than about 5 mg/ml. In some embodiments, the concentration is about 10 mg/ml.

Crystals can be grown from an aqueous solution containing the purified and concentrated GLYAT or candidate polypeptide by a variety of techniques. These techniques include batch, liquid, bridge, dialysis, vapor diffusion, and hanging drop methods (McPherson (1982) John Wiley, New York; McPherson (1990) Eur. J. Biochem. 189:1-23; Webber (1991) Adv. Protein Chem. 41:1-36, each of which is herein incorporated by reference in its entirety). Seeding of the crystals in some instances may be required to obtain X-ray quality crystals. Standard micro and/or macro seeding of crystals may therefore be used. In general, crystals are grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

In some embodiments, the GLYAT or candidate polypeptide is crystallized via hanging drop vapor diffusion against a crystallization solution. In some embodiments, the crystallization solution comprises sodium acetate, ammonium sulfate, and polyethylene glycol. In some of these embodiments, the concentration of sodium acetate within the crystallization solution ranges from about 50 mM to about 200 mM, including but not limited to about 50 mM, 60 mM, 70 mM, 80 mM, 90 mM, 100 mM, 125 mM, 150 mM, 175 mM, and 200 mM. In these embodiments, the pH of the sodium acetate can range from about 3.5 to about 6.0, including but not limited to about 3.5, 3.6, 3.7, 3.8, 3.9, 4.0, 4.1, 4.2, 4.3, 4.5, 4.6, 4.7, 4.8, 4.9, 5.0, 5.1, 5.2, 5.3, 5.4, 5.5, 5.6, 5.7, 5.8, 5.9, and 6.0. In particular embodiments, the crystallization solution comprises 100 mM sodium acetate at a pH of about 4.6. In certain embodiments, the concentration of ammonium sulfate within the crystallization solution ranges from about 150 mM to about 300 mM, including but not limited to, about 150 mM, 175 mM, 200 mM, 225 mM, 250 mM, 275 mM, and 300 mM. In some embodiments, the crystallization solution comprises PEG4000 at a concentration ranging from about 15% to about 40%, including but not limited to about 15%, 20%, 25%, 30%, 35%, and 40%. In certain embodiments, the concentration of PEG4000 in the crystallization solution ranges from about 20% to about 25%. In particular embodiments, the crystallization solution comprises about 100 mM sodium acetate at a pH of about 4.6, 150 mM to about 300 mM ammonium sulfate, and about 20% to about 25% PEG4000.

To collect diffraction data From the crystals of the GLYAT polypeptide or candidate polypeptide, the crystals may be flash-frozen in the crystallization solution employed for the growth of said crystals. In some embodiments, the crystals are flash frozen in a buffer wherein the precipitant concentration is higher than the crystallization buffer. If the precipitant is not a sufficient cryoprotectant (i.e. a glass is not formed upon flash-freezing), cryoprotectants (e.g. glycerol, ethylene glycol, low molecular weight PEGs, alcohols, etc.) may be added to the solution in order to achieve glass formation upon flash-freezing, providing the cryoprotectant is compatible with preserving the integrity of the crystals. In some embodiments, the cryoprotectant solution comprises sodium acetate, glycerol, and polyethylene glycol. In some of these embodiments, the concentration of sodium acetate within the cryoprotectant solution ranges from about 50 mM to about 200 mM, including but not limited to about 50 mM, 60 mM, 70 mM, 80 mM, 90 mM, 100 mM, 125 mM, 150 mM, 175 mM, and 200 mM. In these embodiments, the pH of the sodium acetate can range from about 3.5 to about 6.0, including but not limited to about 3.5, 3.6, 3.7, 3.8, 3.9, 4.0, 4.1, 4.2, 4.3, 4.5, 4.6, 4.7, 4.8, 4.9, 5.0, 5.1, 5.2, 5.3, 5.4, 5.5, 5.6, 5.7, 5.8, 5.9, and 6.0. In particular embodiments, the cryoprotectant solution comprises about 100 mM sodium acetate at a pH of about 4.6. In some embodiments, the cryoprotectant solution comprises PEG4000 at a concentration ranging from about 15% to about 40%, including but not limited to about 15%, 20%, 25%, 30%, 35%, and 40%. In certain embodiments, the concentration of PEG4000 in the cryoprotectant solution is about 20%. The cryoprotectant solution can comprise glycerol at a concentration ranging from about 10% to about 30%, including but not limited to about 10%, 15%, 20%, 25%, and 30%. In particular embodiments, the cryoprotectant solution comprises about 100 mM sodium acetate at a pH of about 4.6, about 20% PEG4000, and about 20% glycerol.

In those embodiments wherein a molecular structure of the GLYAT or candidate polypeptide in complex with substrate(s) is desired, the substrate(s) can be added to the crystallization solution and the cryoprotectant solution. One of skill in the art will appreciate that the substrate(s) should be included at a concentration that is at, near or above the concentration required for saturation of the substrate binding site of the enzyme. As used herein, a “substrate” refers to a molecule that is capable of binding to the enzyme and being acted upon by the enzyme. The term substrate comprises metabolites, cofactors, coenzymes, and prosthetic groups (e.g., heme) that are required for enzymatic catalysis. Thus, in some embodiments, acetyl CoA is added to the crystallization and cryoprotectant solution. In some of these embodiments, the concentration of acetyl CoA in the crystallization and cryoprotectant solution ranges from about 0.1 mM to about 20 mM, including but not limited to about 0.1 mM, 0.2 mM, 0.3 mM, 0.4 mM, 0.5 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In certain embodiments, the concentration of acetyl CoA in the crystallization and cryoprotectant solutions is about 2 mM.

In some embodiments, glyphosate is added to the crystallization and cryoprotectant solution. In some of these embodiments, the concentration of glyphosate in the crystallization and cryoprotectant solution ranges from about 2 mM to about 50 mM, including, but not limited to about 2 mM, 5 mM, 10 mM, 15 mM, 20 mM, 25 mM, 30 mM, 35 mM, 40 mM, 45 mM and 50 mM. In certain embodiments, the concentration of glyphosate in the crystallization and cryoprotectant solution is about 20 mM.

In particular embodiments, both glyphosate and acetyl CoA are added to the crystallization and cryoprotectant solutions and the three-dimensional molecular structures of the GLYAT polypeptide and candidate polypeptide are determined in complex with both glyphosate and acetyl CoA. In some of these embodiments, the concentration of glyphosate is about 20 mM and the concentration of acetyl coA is about 2 mM in the crystallization and cryoprotectant solutions.

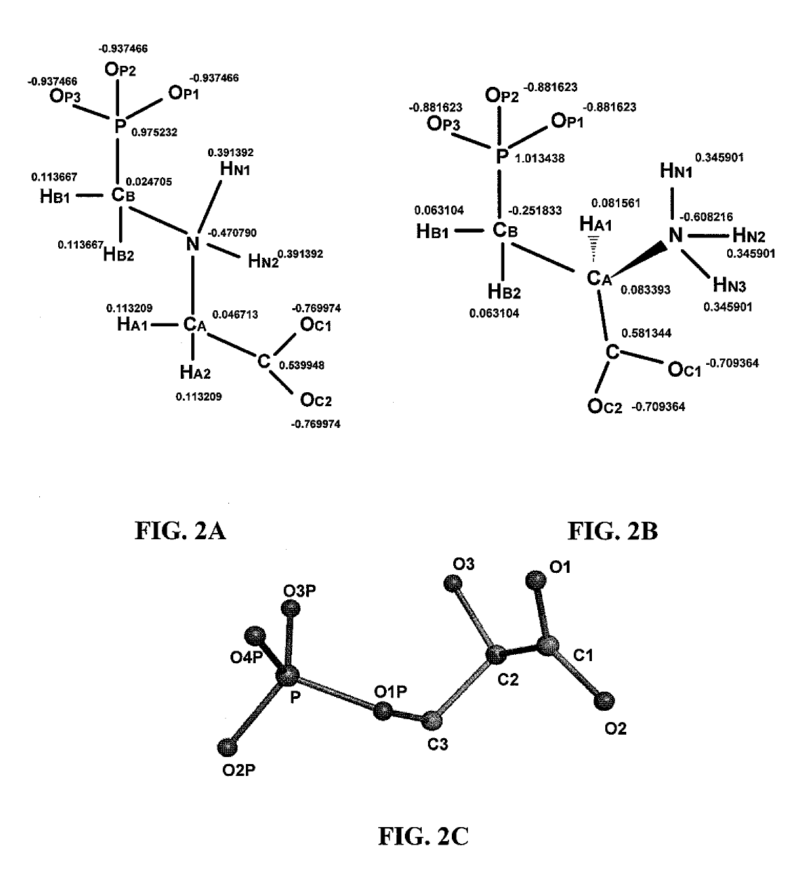

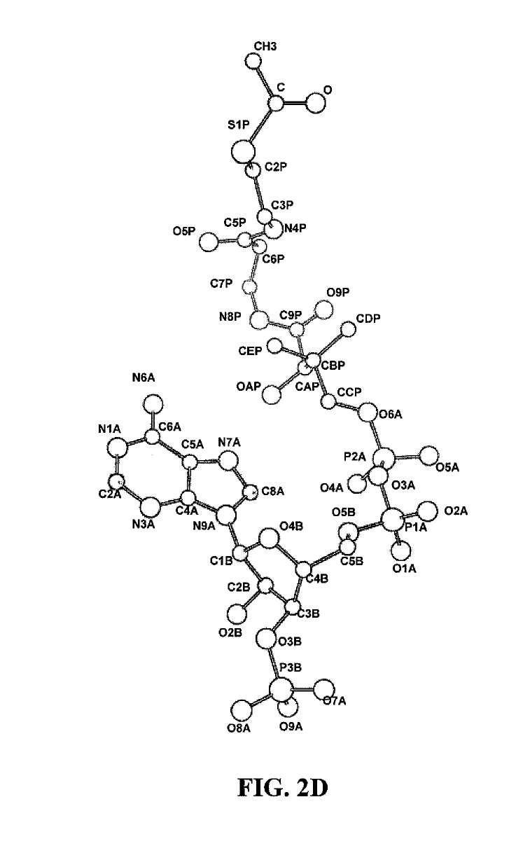

As used herein, the term “glyphosate” refers to the molecule whose chemical structure is depicted in FIG. 2A and any active metabolite, or salt thereof. An “active” metabolite or salt of glyphosate is one that is capable of inhibiting a 5-enolpyruvylshikimate-3-phosphate (EPSP) synthase or of otherwise injuring a plant. Non-limiting examples of active metabolites or salts of glyphosate include N-(phosphonomethyl)glycine (C3H8NO2P), glyphosate ammonium salt (C3H11N2O5P), glyphosate isopropylamine salt (C6H 17N2O5P), glyphosate potassium salt (C3H7KNO5P), and aminomethylphosphonate (CH6NO3P). One of skill in the art will also appreciate that the GLYAT polypeptide and/or candidate polypeptide can be crystallized in the presence of an analog of glyphosate (e.g., D-2-amino-3-phosphonopropionic acid, 3-phosphoglycerate) and the structural model derived therefrom can be modified using any of the computational methods known in the art and described elsewhere herein to replace the glyphosate analog with glyphosate in the molecular model of the polypeptide.

The flash-frozen crystals are maintained at a temperature of less than about −110° C. in some embodiments and in other embodiments, less than about −150° C. during the collection of the crystallographic data by X-ray diffraction. The diffraction data is generally obtained by placing a crystal in an X-ray beam. The incident X-rays interact with the electron cloud of the molecules that make up the crystal, resulting in X-ray scatter. The combination of X-ray scatter with the lattice of the crystal gives rise to non-uniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the l index gives the number of parts into which the c edge of the unit cell is cut by the set of hkl planes.

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections are recorded on the detector, resulting in a diffraction pattern.

Sources of X-rays include, but are not limited to, a rotating anode X-ray generator such as a Rigaku RU-200 or a beamline at a synchrotron light source. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

The unit cell dimensions and space group of a crystal can be determined from its diffraction pattern. The “unit cell” is the crystal's repeating unit. The spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, 85%, 90%, 95%, 96%, 97%, 98%, 99%, or greater of unique reflections are recorded. In some embodiments, a complete dataset is collected using one crystal. In another embodiment, a complete dataset is collected using more than one crystal of the same type.

Once a dataset of intensities for the reflections is collected, the information is used to determine the three-dimensional structure of the molecule in the crystal. However, in the absence of a suitable molecular model, this cannot be done from a single measurement of reflection intensities because certain information, known as phase information, is lost between the three-dimensional shape of the molecule and its Fourier transform, the diffraction pattern. This phase information must be acquired by methods described below in order to perform a Fourier transform on the diffraction pattern to obtain the three-dimensional structure of the molecule in the crystal. It is the determination of phase information that in effect refocuses X-rays to produce the image of the molecule.

In one approach, if the polypeptide for which the structure is to be solved forms crystals that are isomorphous, i.e., that have the same unit cell dimensions and space group as a related molecule whose structure has been determined, then the phases and/or co-ordinates for the related molecule can be combined directly with newly observed amplitudes to obtain electron density maps and, consequently, atomic co-ordinates of the polypeptide with unknown structure.

In another approach, if the polypeptide of unknown structure is related to another molecule of known three-dimensional structure, but crystallizes in a different unit cell with different symmetry, the skilled artisan may use a technique known as molecular replacement to obtain useful phases from the co-ordinates of the molecule whose structure is known (M. G. Rossmann, ed. “The Molecular Replacement Methods,” Sci. Rev. J. No. 13, Gordon & Breach, New York, N.Y. (1972); Eaton Lattman, “Use of Rotation and Translation Functions,” H. W Wyckoff C. H. W. Hist. (S, N. Timasheff, ed.) Methods in Enzmmology, 115: 55-77 (1985)). For an example of the application of molecular replacement, see, for example, Rice & Steitz (1994) EMBO J. 13:1514-24). Specifically, molecular replacement is a method of calculating initial phases for a new crystal of a polypeptide or polypeptide co-complex whose structure coordinates are unknown by orienting and positioning a related polypeptide whose structure coordinates are known within the unit cell of the new crystal so as to best account for the observed diffraction pattern of the new crystal. To enable this, the related molecule must have a similar three dimensional structure. Briefly, the principle behind the method of molecular replacement is as follows. The three-dimensional structure of the known molecule is positioned within the unit cell of the new crystal by finding the orientation and position that provides the best agreement between observed diffraction amplitudes and those calculated from the co-ordinates of the positioned polypeptide. From this modeling, approximate phases for the unknown crystal can be derived. Once the orientation of a test molecule is known, the position of the molecule must be found using a translational search. X-PLOR (Brunger et al. (1987) Science 235:458-460; CNS (Crystallography & NMR System), Brunger et al., (1998) Acta Cryst. Sect. D 54: 905-921), and AMORE: an Automatic Package for Molecular Replacement (Navaza, J. (1994) Acta Cryst. Sect. A, 50: 157-163) are computer programs that can execute rotation and translation function searches. Once the known structure has been positioned in the unit cell of the unknown molecules, phases for the observed diffraction data can be calculated from the atomic co-ordinates of the structurally related atoms of the known molecules. By using the calculated phases and X-ray diffraction data for the unknown molecule, the skilled artisan can generate an electron density map and/or atomic co-ordinates of the GLYAT polypeptide of candidate polypeptide.

In general, the success of molecular replacement for solving structures depends on the fraction of the structures that are related and their degree of identity. For example, if about 50% or more of the structure shows a root mean square (RMS) deviation between corresponding atoms in the range of about 2 Å or less, the known structure can be successfully used to solve the unknown structure.

The term “root mean square deviation” means the square root of the arithmetic mean of the squares of the deviations from the mean. It is a way to express the deviation or variation from a trend or object. For example, the “root mean square deviation” can define the variation in the backbone of a polypeptide from the relevant portion of the backbone of a GLYAT polypeptide or a portion thereof as defined by the structure coordinates described herein.

A third method of phase determination is multi-wavelength anomalous dispersion or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson (1985) Trans. Am. Crystallogr. Assoc., 21:11; Hendrickson et al. (1990) EMBO J. 9:1665; and Hendrickson (1991) Science 4:91.

A fourth method of determining phase information is single wavelength anomalous dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. A detailed discussion of SAD analysis can be found in Brodersen et al. (2000) Acta Cryst. D56:431-441.

A fifth method of determining phase information is single isomorphous replacement with anomalous scattering or SIRAS. This technique combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North (1965) Acta Cryst. 18:212-216; Matthews (1966) Acta Cryst. 20:82-86.

To generate a heavy atom derivative of a polypeptide, the crystals of the polypeptide may be soaked in heavy-atoms. As used herein, heavy atom derivative or derivatization refers to the method of producing a chemically modified form of a protein or protein complex crystal wherein said protein is specifically bound to a heavy atom within the crystal. In practice, a crystal is soaked in a solution containing heavy metal atoms or salts, or organometallic compounds (e.g., lead chloride, gold cyanide, thimerosal, lead acetate, uranyl acetate, mercury chloride, gold chloride) which can diffuse through the crystal and bind specifically to the protein. The location(s) of the bound heavy metal atom(s) or salts can be determined by X-ray diffraction analysis of the soaked crystal. This information is used to generate phase information which is used to construct the three-dimensional structure of the crystallized polypeptide.

In another approach, if no crystals are available for the candidate polypeptide, but it is homologous to another molecule whose three-dimensional structure is known, the skilled artisan may use a process known as homology modeling to produce a three-dimensional model of the candidate polypeptide. Accordingly, information concerning the crystals and/or atomic co-ordinates of one molecule can greatly facilitate the determination of the structures of related molecules.

As used herein, the term “homology modeling” refers to the practice of deriving models for three-dimensional structures of macromolecules from existing three-dimensional structures for their homologues. In general, the procedure may comprise one or more of the following steps: aligning the amino acid sequence of an unknown molecule against the amino acid sequence of a molecule whose structure has previously been determined; identifying structurally conserved and structurally variable regions; generating atomic co-ordinates for core (structurally conserved) residues of the unknown structure from those of the known structure(s); generating conformations for the other (structurally variable) residues in the unknown structure; building side chain conformations; and refining structure through energy minimization and molecular dynamics, and/or evaluating the unknown structure. Homology models are obtained using computer programs that make it possible to alter the identity of residues at positions where the sequence of the molecule of interest is not the same as that of the molecule of known structure. For example, homology modeling was used to generate the R11 and YVII revertant mutant described elsewhere herein (see Experimental section).

Once phase information is obtained, it is combined with the diffraction data to produce an electron density map, an image of the electron clouds that surround the molecules in the unit cell. For basic concepts and procedures of collecting, analyzing, and utilizing X-ray diffraction data for the construction of electron densities see, for example, Campbell et al. (1984) Biological Spectroscopy, The Benjamin/Cummings Publishing Co., Inc., (Menlo Park, Calif.); Cantor et al. (1980) Biophysical Chemistry, Part II: Techniques for the study of biological structure and function, W. H. Freeman and Co., San Francisco, Calif.; A. T. Brunger (1993) X-PLOR Version 3.1: A system for X-ray crystallography and NMR, Yale Univ. Pr., (New Haven, Conn.); M. M. Woolfson (1997) An Introduction to X-ray Crystallography, Cambridge Univ. Pr., (Cambridge, UK); J. Drenth (1999) Principles of Protein X-ray Crystallography (Springer Advanced Texts in Chemistry), Springer Verlag; Berlin; Tsirelson et al. (1996) Electron Density and Bonding in Crystals: Principles, Theory and X-ray Diffraction Experiments in Solid State Physics and Chemistry, Inst. of Physics Pub.; U.S. Pat. No. 5,942,428; U.S. Pat. No. 6,037,117; U.S. Pat. No. 5,200,910 and U.S. Pat. No. 5,365,456 (“Method for Modeling the Electron Density of a Crystal”).

The higher the resolution of the data, the more distinguishable are the features of the electron density map, e.g., amino acid side chains and the positions of carbonyl oxygen atoms in the peptide backbones, because atoms that are closer together are resolvable. In certain embodiments, the protein crystals and protein-substrate complex crystals of the GLYAT polypeptide or candidate polypeptide diffract to a high resolution limit. As used herein, the term “resolution” in relation to electron density is a measure of the resolvability in the electron density map of a molecule. In X-ray crystallography, resolution is the highest resolvable peak in the diffraction pattern. Resolution is expressed in terms of the lowest resolvable distance between two atoms, measured in angstroms (Å). In some embodiments, the maximal resolution of crystals of the GLYAT polypeptide or candidate polypeptide, alone or complexed with one or more substrate (e.g., glyphosate) is less than or equal to about 3.5 Å, including, but not limited to about 3.5 Å, 3.4 Å, 3.3 Å, 3.2 Å, 3.1 Å, 3.0 Å, 2.9 Å, 2.8 Å, 2.7 Å, 2.6 Å, 2.5 Å, 2.4 Å, 2.3 Å, 2.2 Å, 2.1 Å, 2.0 Å, 1.9 Å, 1.8 Å, 1.7 Å, 1.6 Å, 1.5 Å, 1.4 Å, 1.3 Å, 1.2, Å, 1.1 Å, 1.0 Å, or less than 1.0 Å. In particular embodiments, the polypeptide or polypeptide-substrate complex crystal have a resolution limit of about 1.6 Å.

The electron density maps generated from the diffraction and phase data are used to establish the positions of the individual atoms within a single polypeptide, which are expressed as atomic coordinates. As used herein, the term “atomic coordinates” refers to mathematical co-ordinates (represented as “X,” “Y” and “Z” values) that describe the positions of atoms in a crystal of a polypeptide with respect to a chosen crystallographic origin. As used herein, the term “crystallographic origin” refers to a reference point in the crystal unit cell with respect to the crystallographic symmetry operation. These atomic coordinates can be used to generate a three-dimensional representation of the molecular structure of the polypeptide.

A model of the macromolecule is then built into the electron density map with the aid of a computer, using as a guide all available information, such as the polypeptide sequence and the established rules of molecular structure and stereochemistry. Interpreting the electron density map is a process of finding the chemically realistic conformation that fits the map precisely. The atomic co-ordinates are entered into one or more computer programs for molecular modeling, as known in the art. By way of illustration, a list of computer programs useful for viewing or manipulating three-dimensional structures include: Midas (University of California, San Francisco); MidasPlus (University of California, San Francisco); MOIL (University of Illinois); Yumrnie (Yale University); Sybyl (Tripos, Inc.); Insight/Discover (Biosym Technologies); MacroModel (Columbia University); Quanta (Molecular Simulations, Inc.); Cerius (Molecular Simulations, Inc.); Alchemy (Tripos, Inc.); LabVision (Tripos, Inc,); Rasmol (Glaxo Research and Development); Ribbon (University of Alabama); NAOMI (Oxford University); Explorer Eyecbem (Silicon Graphics, Inc.); Univision (Cray Research); Molscript (Uppsala University); Chem-3D (Cambridge Scientific); Chain (Baylor College of Medicine); 0 (Uppsala University); GRASP (Columbia University); X-Plor (Molecular Simulations, Inc.; Yale University); Spartan (Wavefunction, Inc.); Catalyst (Molecular Simulations, Inc.); Molcadd (Tripos, Inc.); VMD (University of Illinois/Beckman Institute); Sculpt (Interactive Simulations, Inc.); Procheck (Brookhaven National Library); DGEOM (QCPE); REVIEW (Brunell University); Modeller (Birbeck College, University of London); Xmol (Minnesota Supercomputing Center); Protein Expert (Cambridge Scientific); HyperChcm (Hypercube); MD Display (University of Washington); PKB (National Center for Biotechnology Information, NIH); ChemX (Chemical Design, Ltd.); Cameleon (Oxford Molecular, Inc.); and Iditis (Oxford Molecular, Inc.).

After a model is generated, the structure is refined. Refinement is the process of minimizing the function Φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function Φ converges on a minimum wherein the model fits the electron density map and is stereochemically and conformationally reasonable. During refinement, ordered solvent molecules are added to the structure.

While Cartesian coordinates are important and convenient representations of the three-dimensional molecular structure of a polypeptide, those of skill in the art will readily recognize that other representations of the structure are also useful. Therefore, the three-dimensional molecular structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein or protein co-complex in which an atom is located, and connectivity information, which indicates to which atoms a particular atom is bonded, is also useful for representing a three-dimensional molecular structure.

The three-dimensional molecular structures for the GLYAT R7 variant polypeptide was determined with the GLYAT variant in complex with oxidized coA (a binary complex) and in complex with acetyl coA and 3PG (ternary complex) (Siehl et al. (2007) J Biol Chem 282:I′1446-11455). The atomic coordinates and structural information for the binary and ternary complexes can be found in the Protein Data Bank (Berman et al. (2000) Nucleic Acids Research 28, 235-242; see also, the web page at the URL resb.org/pdb/) with the accession numbers PDB ID: 2JDC and PDB ID: 2JDD, respectively, which are herein incorporated by reference in their entireties (Siehl et al. (2007) J Biol Chem 282:11446-11455). The GLYAT R7 variant exhibits enhanced catalytic activity for glyphosate over the native GLYAT polypeptide. The optimized GLYAT polypeptide was generated through iterative DNA shuffling of a native GLYAT polypeptide.

As will be apparent to those of ordinary skill in the art, the atomic structures presented herein are independent of their orientation, and the atomic co-ordinates identified herein merely represent one possible orientation of a particular GLYAT polypeptide. The atomic coordinates are a relative set of points that define a shape in three dimensions. Thus, it is possible that a different set of coordinates could define a similar or identical shape. Therefore, slight variations in the individual coordinates will have little effect on overall shape. It is apparent, therefore, that the atomic co-ordinates identified herein may be mathematically rotated, translated, scaled, or a combination thereof, without changing the relative positions of atoms or features of the respective structure. The variations in coordinates discussed may bc generated because of mathematical manipulations of the structure coordinates. For example, the structure coordinates could bc manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions and/or deletions of amino acids, or other changes in any of the components that make up the crystal could also account for variations in the structure coordinates. If such variations are within an acceptable standard of error as compared to the original coordinates, the resulting three-dimensional shape is considered to be the same. Thus, in one aspect of the present invention, any molecule or molecular complex that has a RMSD of conserved residue backbone atoms (N, Calpha, C, O) of less than about 4 Å, 2 Å, 1.5 Å, 1 Å, or 0.5 Å when superimposed on the relevant backbone atoms described by the coordinates listed in any one of Tables 1-10 are considered identical.

Using the methods of the invention, candidate polypeptides are evaluated for the potential of having an improved enzymatic activity in comparison to native GLYAT enzymes based on three-dimensional structural similarities with an optimized GLYAT. Enzymatic activity can be characterized using the conventional kinetic parameters k_{cat}, K_{M}, and k_{cat}/K_{M}. The catalytic constant, k_{cat}, can be thought of as a measure of the maximum rate of acetylation, particularly at high substrate concentrations; K_{M} is a measure of the affinity of an enzyme for its substrate (e.g., glyphosate) and cofactor (e.g., acetyl CoA); and k_{cat}/K_{M} is a measure of catalytic efficiency that takes both substrate affinity and catalytic rate into account. k_{cat}/Kₘ is particularly important in the situation where the concentration of a substrate is at least partially rate-limiting. In general, an enzyme with a higher k_{cat} or k_{cat}/K_{M} is a more efficient catalyst than another enzyme with a lower k_{cat}, or k_{cat}/K_{M}. An enzyme with a lower K_{M} binds its substrate with a higher affinity and is a more efficient catalyst than another enzyme with a higher K_{M}. Thus, to determine whether one GLYAT is more effective than another, one can compare kinetic parameters for the two enzymes. The relative importance of k_{cat}, k_{cat}/K_{M} and K_{M} will vary depending upon the context in which the GLYAT will be expected to function, e.g., the anticipated effective concentration of glyphosate relative to the K_{M} for glyphosate.

Thus, the GLYAT polypeptide used to evaluate the candidate polypeptide or the candidate polypeptide itself may have a higher affinity, and thus, a lower K_{M}, for glyphosate than native GLYAT enzymes. For example, in some embodiments, the K_{M} of the GLYAT polypeptide or candidate polypeptide is less than about 1 mM, including but not limited to, about 0.9 mM, 0.8 mM, 0.7 mM, 0.6 mM, 0.5 mM, 0.4 mM, 0.3 mM, 0.2 mM, 0.1 mM, 0.05 mM, or less.

The GLYAT polypeptide or candidate polypeptide may have a higher k_{ca}, for a substrate (e.g., glyphosate) than native GLYAT polypeptides. For example, in some embodiments, the GLYAT polypeptide or candidate polypeptide has a k_{cat} of at least about 20 min⁻¹, including but not limited to, about 50 min⁻¹, 100 min⁻¹, 200 min⁻¹, 500 min⁻¹, 1000 min⁻¹, 1100 min⁻¹, 1200 min⁻¹, 1250 min⁻¹, 1300 min⁻¹, 1400 min⁻¹, 1500 min⁻¹, 1600 min⁻¹, 1700 min⁻¹, 1800 min⁻¹, 1900 min⁻¹, 2000 min⁻¹ or higher. GLYAT polypeptides or the candidate polypeptides may have a higher k_{cat}/K_{M} for a substrate (e.g., glyphosate) than native GLYAT enzymes. In some embodiments, the GLYAT polypeptide or candidate polypeptide has a k_{cat}/K_{M} of at least about 100 mM⁻¹ min⁻¹, 500 mM⁻¹ min⁻¹, 1000 mM⁻¹ min⁻¹, 2000 mM⁻¹ min⁻¹, 3000 mM⁻¹ min⁻¹, 4000 mM⁻¹ min⁻¹, 5000 mM⁻¹ min⁻¹, 6000 mM⁻¹ min⁻¹, 7000 mM⁻¹ min⁻¹, or 8000 mM⁻¹ min⁻¹, or higher. The activity of GLYAT enzymes is affected by, for example, pH and salt concentration; appropriate assay methods and conditions are known in the art (see, e.g., WO2005012515, which is herein incorporated by reference in its entirety). Such improved enzymes identified using the presently disclosed methods may find particular use in methods of growing a crop in a field where the use of a particular herbicide or combination of herbicides and/or other agricultural chemicals would result in damage to the plant if the enzymatic activity (i.e., k_{cat}, K_{M}, or k_{cat}/K_{M}) were lower.

In some embodiments, the GLYAT polypeptide for which a molecular structure is provided for comparison to a candidate polypeptide or the candidate polypeptide itself exhibits a greater specificity for glyphosate than native GLYAT polypeptides., As used herein, “specificity” refers to the preference of a polypeptide to bind and/or catalyze one substrate over another. For example, a polypeptide with a greater specificity for glyphosate over other potential GLYAT substrates binds to glyphosate with an affinity that is at least two times greater than its affinity for another substrate (e.g., D-AP3). In some embodiments, the affinity, k_{cat}, and/or k_{cat}/K_{M} is about 2 times, about 3, about 4, about 5, about 6, about 7, about 8, about 9, about 10, about 11, about 12, about 13, about 14, about 15, about 16, about 17, about 18, about 19, about 20, about 25, about 30, about 40, about 50, about 100, about 200, about 500, about 1000, or greater times that of the native GLYAT polypeptide for glyphosate over another substrate (e.g, D-AP3). In those embodiments wherein the affinity is greater, the K_{M} of the GLYAT polypeptide or candidate polypeptide for glyphosate is equivalently lower than the K_{M} of the polypeptide for the other substrate.

In some embodiments, the specificity of the GLYAT polypeptide for which the molecular structure is constructed and/or the candidate polypeptide exhibit a greater specificity for glyphosate than native GLYAT polypeptides. In certain embodiments, the GLYAT polypeptide or candidate polypeptide is able to bind compounds with at least five main chain atoms with a higher affinity than native GLYAT polypeptides. Kinetic data has demonstrated that optimizing GLYAT for activity with glyphosate shifted the binding preference to ligands with a main-chain length of 5-atoms from those of 4-atoms in the wild-type enzyme (Siehl et al. (2007) J Biol Chem 282:11446-11455). For example, the R7 and R11 variants of GLYAT have a higher binding affinity and higher catalytic activity on compounds with five main chain atoms (e.g., glyphosate) than native GLYAT polypeptides, which exhibit a preference for smaller compounds with three to four main chain atoms (e.g., D-AP3). Thus, in some embodiments, the GLYAT polypeptide or candidate polypeptide bind compounds with at least five main chain atoms with an affinity that is at least about 2 fold greater than native GLYAT polypeptides, including but not limited to at least about 2-fold, 3-fold, 4-fold, 5-fold, 10-fold, 20-fold, 30-fold, 40-fold, 50-fold, 100-fold, or greater.

The analysis of the molecular structure of the GLYAT R7 variant polypeptide complexed with acetyl CoA and glyphosate provided herein has provided the identity and location of the residues important for the binding of substrates to GLYAT polypeptides. Importantly, the analysis has provided a molecular basis for the enhanced affinity and specificity exhibited by the GLYAT variant polypeptides over that of the native GLYAT polypeptide.

The atomic coordinates of the GLYAT R7 variant polypeptide that comprise the substrate binding cavity are presented in Table 1, wherein the GLYAT R7 variant polypeptide is bound to glyphosate and acetyl coA. Table 2 provides the atomic coordinates of the substrate binding cavity of GLYAT R11 variant polypeptide when bound to glyphosate and acetyl coA. As used herein, a “substrate binding cavity” refers to the atoms of a polypeptide that directly contact (e.g., through hydrogen bonds, van der Waals interactions) the substrate (e.g., glyphosate) or are within about 4 Å of the substrate (e.g., glyphosate). A “substrate binding cavity” can also include residues that contribute to the structure or flexibility of the residues directly contacting or within 4 Å of the substrate. In some embodiments, the substrate binding cavity comprises at least the atomic coordinates of Table 1.

TABLE 1
Contacts between the R7 GLYAT variant polypeptide
and AcCoA and glyphosate when the polypeptide
is bound to AcCoA and glyphosate.

| | | | | | | Glyph- | |
|---|---|---|---|---|---|---|---|
| Residue | Amino | GLYAT | | | | osate | Distance |
| ID | Acid | Atom^{a} | X^{b} | Y^{b} | Z^{b} | Atom^{c} | (A) |
| 20 | LEU | CD1 | 24.54 | 6.61 | 10.65 | C1 | 3.96 |
| | | | | | | N | 3.65 |
| | | CD2 | 22.35 | 7.82 | 11.09 | N | 3.76 |
| 21 | ARG | CZ | 26.49 | 8.65 | 16.97 | C1 | 3.95 |
| | | | | | | OC2 | 3.65 |
| | | | | | | OP2 | 3.60 |
| | | | | | | OP3 | 3.63 |
| | | NE | 27.54 | 8.59 | 16.10 | OC2 | 3.65 |
| | | NH1 | 25.95 | 9.81 | 17.39 | OP2 | 3.60 |
| | | | | | | OP3 | 3.85 |
| | | NH2 | 25.95 | 7.49 | 17.41 | C | 3.29 |
| | | | | | | C1 | 3.41 |
| | | | | | | OC2 | 2.76 |
| | | | | | | OP2 | 3.52 |
| | | | | | | OP3 | 2.73 |
| | | | | | | P | 3.61 |
| 31 | PHE | CE1 | 29.68 | 5.11 | 14.89 | OC2 | 3.44 |
| | | CE2 | 28.57 | 3.11 | 14.11 | C | 3.87 |
| | | | | | | OC2 | 3.54 |
| | | CZ | 28.66 | 4.50 | 14.15 | C | 3.54 |
| | | | | | | C1 | 3.99 |
| | | | | | | OC2 | 3.02 |
| 73 | ARG | CA | 24.84 | 0.27 | 13.99 | OC1 | 3.90 |
| | | CD | 25.39 | 0.54 | 17.17 | OC1 | 3.64 |
| | | CZ | 27.24 | 2.14 | 17.85 | OC1 | 3.70 |
| | | | | | | OC2 | 3.50 |
| | | NE | 26.07 | 1.88 | 17.20 | C | 3.48 |
| | | | | | | OC1 | 2.79 |
| | | | | | | OC2 | 3.41 |
| | | NH2 | 27.81 | 3.35 | 17.70 | C | 3.58 |
| | | | | | | OC1 | 3.70 |
| | | | | | | OC2 | 2.69 |
| 74 | GLY | N | 25.02 | 2.30 | 12.66 | C | 3.85 |
| | | | | | | OC1 | 3.37 |
| 111 | ARG | CD | 18.36 | 8.96 | 14.19 | OP1 | 3.66 |
| | | CG | 17.90 | 7.82 | 15.11 | OP1 | 3.43 |
| | | CZ | 20.53 | 10.16 | 13.77 | OP1 | 3.52 |
| | | | | | | OP2 | 3.57 |
| | | NE | 19.83 | 9.19 | 14.41 | OP1 | 2.69 |
| | | | | | | OP2 | 3.64 |
| | | | | | | P | 3.64 |
| | | NH2 | 21.85 | 10.31 | 14.04 | OP1 | 3.48 |
| | | | | | | OP2 | 2.64 |
| | | | | | | P | 3.57 |
| 135 | VAL | CB | 21.83 | 11.15 | 17.99 | OP1 | 3.76 |
| | | | | | | OP2 | 3.99 |
| | | CG1 | 23.02 | 11.69 | 17.18 | OP2 | 3.66 |
| | | CG2 | 22.29 | 10.19 | 19.11 | OP1 | 3.82 |
| | | | | | | OP3 | 3.67 |
| | | | | | | P | 3.91 |
| 138 | HIS | CD2 | 18.74 | 6.23 | 18.66 | OP1 | 3.66 |
| | | CE1 | 19.31 | 8.24 | 19.29 | OP1 | 3.45 |
| | | NE2 | 19.49 | 7.33 | 18.35 | OP1 | 2.61 |
| | | | | | | OP3 | 3.88 |
| | | | | | | P | 3.73 |

^{a}The data are derived from a modeled structure based on PDB: 2JDD, in which 3PG was replaced by glyphoshate (FIG. 1). The structural model underwent a series of energy minimization with CHARMm, on newly added hydrogen (CONJ, 500 cycles), on hydrogen and glyphosate (500 cycles), on non-backbone atoms (200 cycles), and on whole system (200 cycles). The amino acid atom is the specific atom of the amino acid, as identified in Protein Data Bank file 2JDD;
^{b}X, Y, and Z are the three-dimensional coordinates specifying the distance in Angstroms of the amino acid atom relative to the center of mass of the crystal defined by the PDB file 2JDD;
^{c}Atoms of glyphosate are defined in FIG. 2A.

TABLE 2
Contacts between the R11 GLYAT variant polypeptide
and AcCoA and glyphosate when the polypeptide is bound
to AcCoA and glyphosate^{a}.

| | | | | | | Glyph- | |
|---|---|---|---|---|---|---|---|
| Residue | Amino | GLYAT | | | | osate | Distance |
| ID | Acid | Atom | X | Y | Z | Atom | (Å) |
| 20 | LEU | CB | 24.59 | 8.95 | 11.21 | N | 3.95 |
| | | CD1 | 24.62 | 6.63 | 10.14 | N | 3.62 |
| | | CD2 | 22.41 | 7.72 | 10.69 | N | 3.59 |
| | | CG | 23.87 | 7.96 | 10.27 | N | 3.87 |
| 21 | ARG | CZ | 26.11 | 7.85 | 17.58 | C1 | 3.95 |
| | | | | | | OC2 | 3.38 |
| | | | | | | OP3 | 3.84 |
| | | NE | 26.95 | 7.84 | 16.51 | C | 3.78 |
| | | | | | | C1 | 3.53 |
| | | | | | | OC2 | 3.09 |
| | | NH2 | 25.52 | 6.69 | 17.93 | C | 3.41 |
| | | | | | | C1 | 3.73 |
| | | | | | | OC2 | 2.76 |
| | | | | | | OP3 | 3.09 |
| 31 | PHE | CD2 | 29.42 | 2.91 | 14.73 | OC2 | 3.97 |
| | | CE1 | 29.7 | 5.66 | 15.1 | OC2 | 3.67 |
| | | CE2 | 28.58 | 3.79 | 14.04 | C | 3.63 |
| | | | | | | OC2 | 3.24 |
| | | CZ | 28.73 | 5.17 | 14.21 | C | 3.63 |
| | | | | | | C1 | 3.92 |
| | | | | | | OC2 | 3.08 |
| 73 | ARG | C | 24.34 | 0.9 | 12.48 | OC1 | 3.77 |
| | | CA | 24.13 | 0.2 | 13.79 | OC1 | 3.71 |
| | | CB | 25.52 | 0.11 | 14.48 | OC1 | 3.7 |
| | | CD | 24.95 | 0.65 | 16.96 | OC1 | 3.58 |
| | | CZ | 26.86 | 2.12 | 17.76 | OC1 | 3.84 |
| | | CZ | 26.86 | 2.12 | 17.76 | OC2 | 3.37 |
| | | NE | 25.8 | 1.88 | 16.94 | C | 3.41 |
| | | | | | | OC1 | 2.85 |
| | | | | | | OC2 | 3.19 |
| | | NH2 | 27.53 | 3.28 | 17.62 | C | 3.66 |
| | | | | | | OC1 | 3.91 |
| | | | | | | OC2 | 2.67 |
| 74 | GLY | CA | 25.02 | 3.04 | 11.45 | N | 3.82 |
| | | | | | | OC1 | 3.67 |
| | | N | 24.48 | 2.25 | 12.53 | C | 3.7 |
| | | | | | | N | 3.9 |
| | | | | | | OC1 | 2.88 |
| 111 | ARG | CD | 17.91 | 8.99 | 14.2 | OP1 | 3.57 |
| | | CG | 17.52 | 7.93 | 15.23 | OP1 | 3.19 |
| | | CZ | 20.1 | 10.14 | 13.76 | OP1 | 3.62 |
| | | | | | | OP2 | 3.54 |
| | | NE | 19.4 | 9.08 | 14.21 | OP1 | 2.71 |
| | | | | | | OP2 | 3.56 |
| | | | | | | P | 3.64 |
| | | NH2 | 21.44 | 10.2 | 13.97 | OP1 | 3.64 |
| | | | | | | OP2 | 2.64 |
| | | | | | | P | 3.64 |
| 135 | VAL | CB | 21.65 | 10.66 | 17.73 | OP2 | 3.74 |
| | | CG1 | 22.96 | 11.05 | 17.01 | OP2 | 3.42 |
| 138 | HSP | CD2 | 18.66 | 5.91 | 18.62 | OP1 | 3.72 |
| | | CE1 | 19.17 | 8.03 | 18.68 | OP1 | 3.47 |
| | | NE2 | 19.35 | 6.92 | 18 | OP1 | 2.65 |
| | | | | | | OP3 | 3.52 |
| | | | | | | P | 3.56 |

^{a}The atom naming convention is the same as in Table 1.

According to the methods of the invention, a candidate polypeptide is evaluated for its potential to associate with glyphosate with a higher binding affinity, higher binding specificity, or both when compared to a native GLYAT polypeptide. In these embodiments, a three-dimensional molecular structure of at least a substrate binding cavity of a GLYAT polypeptide is provided. The three-dimensional molecular structure is determined with the GLYAT polypeptide bound to glyphosate and an acetyl donor, such as acetyl coA. As used herein the terms “bind,” “binding,” “bound,” “bond,” or “bonded,” when used in reference to the association of atoms, molecules, or chemical groups, refer to any physical contact or association of two or more atoms, molecules, or chemical groups. Such contacts and associations include covalent and non-covalent types of interactions.

The three-dimensional molecular structure of the substrate binding cavity can comprise at least the atomic coordinates of Table 1. In other embodiments, the substrate binding cavity comprises at least the atomic coordinates of Table 2. Alternatively, the substrate binding cavity can comprise a structural variant of the substrate binding cavity defined by the atomic coordinates of Table 1 or Table 2. As used herein, a “structural variant” comprises a three-dimensional molecular structure that is similar to another three-dimensional molecular structure. In some embodiments, the structural variant comprises a root mean square deviation from the back-bone atoms of the amino acids of Table 1 or Table 2 of not more than about 4 Å, including but not limited to about 3.5 Å, 3 Å, 2.5 Å, 2 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å, and 0.1 Å. In some of these embodiments, the structural variant substrate binding cavity comprises a root mean square deviation from the back-bone atoms of the amino acids of Table 1 or Table 2 of not more than about 2.0 Å.

Two loops (loop20 and loop130, which is more specifically described as a β-hairpin) cover the bound substrate from opposite sides and join together at their tip points, creating the substrate binding cavity (FIG. 1B). Loop20 (residues 20-25) and its adjacent residues interact with the substrate's carboxyl group and main-chain atoms. Leu20's side-chain directly contacts the glyphosate/3PG's main-chain atoms, forming the back wall of the binding cavity. The Arg21 guanidinium group forms a salt bridge with the substrate's carboxyl group. Phe31 makes direct contact with glyphosate. In a homology model of wild type GLYAT (not shown), the phenol of the tyrosine residue at position 31 in the wild type GLYAT polypeptide hydrogen bonds with the carboxyl of glyphosate or D-AP3, which maintains the local conformation of the polypeptide. Without being bound by any theory or mechanism of action, it is believed that the abolishment of this hydrogen bond due to the mutation of Y31F of the R7 GLYAT variant polypeptide increased the local flexibility, allowing the polypeptide to adapt to binding a larger substrate (e.g., glyphosate). In some embodiments, the substrate binding cavity further comprises the atomic coordinates of loop 20 provided in Table 3 in addition to the atomic coordinates provided in Table 1 or a structural variant thereof. In other embodiments, the substrate binding cavity further comprises the atomic coordinates of loop 20 provided in Table 4 in addition to the atomic coordinates provided in Table 2 or a structural variant thereof. The minimum distances between loop20 residues and glyphosate are also shown in Tables 3 and 4.

TABLE 3
The minimum distance between the R7 GLYAT
variant loop20 residues and glyphosate^{a}.

| | | Amino | | | | Glyph- | Minimum |
|---|---|---|---|---|---|---|---|
| Residue | Amino | Acid | | | | osate | Distance |
| ID | Acid | Atom | X | Y | Z | Atom | (Å)^{b} |
| 16 | ARG | CG | 28.91 | 5.75 | 9.92 | C1 | 6.08 |
| 17 | HIS | N | 29.37 | 10.24 | 9.01 | C1 | 8.27 |
| 18 | ARG | C | 26.05 | 13.16 | 6.97 | OP2 | 9.98 |
| 19 | ILE | O | 23.11 | 12.57 | 9.59 | OP2 | 7.05 |
| 20 | LEU | CD1 | 24.54 | 6.61 | 10.65 | N | 3.65 |
| 21 | ARG | NH2 | 25.95 | 7.49 | 17.41 | OP3 | 2.73 |
| 22 | PRO | CD | 25.3 | 14.83 | 11.68 | OP2 | 7.58 |
| 23 | ASN | N | 27.54 | 16.84 | 13.46 | OP2 | 9.54 |
| 24 | GLN | OE1 | 26.93 | 12.63 | 17.1 | OP2 | 5.77 |
| 25 | PRO | O | 33.23 | 12.71 | 14.76 | OC2 | 10.28 |
| 26 | ILE | O | 34.98 | 9.77 | 13.04 | OC2 | 10.19 |
| 27 | GLU | O | 35.17 | 8.21 | 16.66 | OC2 | 9.25 |
| 28 | ALA | O | 31.2 | 7.9 | 16.13 | OC2 | 5.5 |
| 29 | CYS | CA | 32.06 | 7.68 | 13.45 | OC2 | 6.72 |
| 30 | MET | N | 33.89 | 6.31 | 14.28 | OC2 | 7.74 |
| 31 | PHE | CZ | 28.66 | 4.5 | 14.15 | OC2 | 3.02 |

^{a}The atom naming convention is the same as in the Table 1.
^{b}The minimum distance in Angstroms between the listed pairs of atoms in loop20 and glyphosate.

TABLE 4
The minimum distance between the R11 GLYAT variant loop20
residues and glyphosate^{a}.

| Resi- | | Amino | | | | | Minimum |
|---|---|---|---|---|---|---|---|
| due | Amino | Acid | | | | Glyphosate | Distance |
| ID | Acid | Atom | X | Y | Z | Atom | (Å)^{b} |
| 16 | ARG | CG | 28.96 | 5.86 | 10.04 | C1 | 5.91 |
| 17 | HIS | N | 29.39 | 10.43 | 9.22 | C1 | 8.12 |
| 18 | ARG | C | 25.79 | 13.41 | 7.5 | OP2 | 9.72 |
| 19 | VAL | O | 22.13 | 11.15 | 9.25 | OP2 | 6.59 |
| 20 | LEU | CD2 | 22.41 | 7.72 | 10.69 | N | 3.59 |
| 21 | ARG | NH2 | 25.52 | 6.69 | 17.93 | OC2 | 2.76 |
| 22 | PRO | CD | 25.27 | 14.48 | 12.31 | OP2 | 7.32 |
| 23 | ASN | OD1 | 24 | 16.65 | 15.29 | OP2 | 8.53 |
| 24 | GLN | OE1 | 27.22 | 11.43 | 18.3 | OP2 | 6.37 |
| 25 | PRO | O | 33.11 | 12.43 | 14.5 | OC2 | 10.32 |
| 26 | ILE | O | 35.28 | 10.04 | 12.63 | OC2 | 10.94 |
| 27 | GLU | O | 36.02 | 8.4 | 16.25 | OC2 | 10.42 |
| 28 | ALA | O | 31.54 | 8.43 | 16.16 | OC2 | 6.4 |
| 29 | CYS | O | 32.43 | 5.63 | 12.88 | OC2 | 6.97 |
| 30 | MET | C | 34.17 | 4.54 | 15.71 | OC2 | 7.97 |
| 31 | PHE | CZ | 28.73 | 5.17 | 14.21 | OC2 | 3.08 |

^{a}The atom naming convention is the same as in the Table 1.
^{b}The minimum distance in Angstroms between the listed pairs of atoms in loop20 and glyphosate.

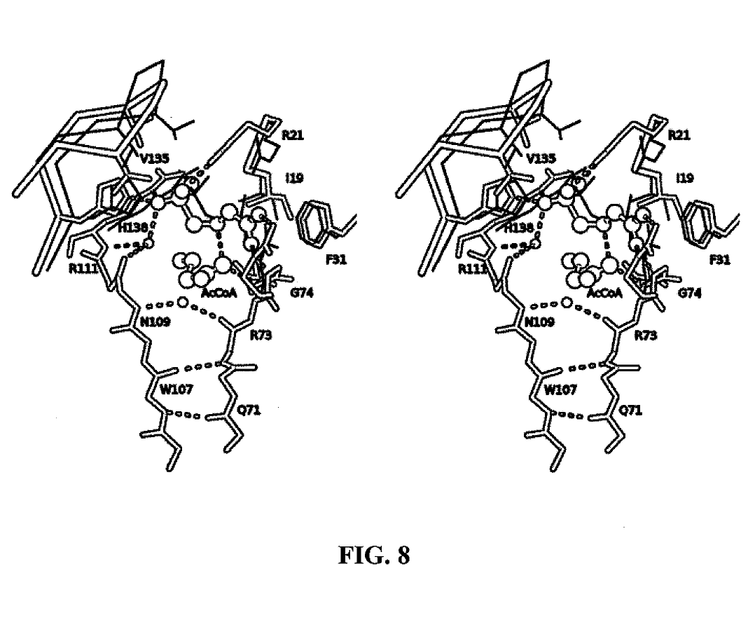

The substrate-binding β-hairpin comprises residues 130-138 (FDTPPVGPH of the GLYAT R7 variant). The substrate-binding β-hairpin connects strands 6 and 7, with the four middle residues (TPPV) forming a typical Via β-turn (Richardson (1981) Adv Protein Chem. 1981; 34:167-339). As described elsewhere herein, the two consecutive prolines Pro133 and Pro 134 reduce the flexibility of the β-turn with Pro 133 adopting a trans- and Pro 134 a cis-conformation. The β-hairpin covers glyphosates phosphono group and harbors the putative catalytic base H is)₃₈ (see FIG. 8). This β-turn is one of the least conserved motifs in the GLYAT family and thus it is exquisitely evolved to recognize the phosphono group of glyphosate or D-AP3. Val135 directly contacts either substrate's phosphono group through van der Waals interaction while Thr132's OG1 is ˜4.5 Å from the phosphono oxygen, a suitable distance for forming a water-bridged hydrogen bond, H is 138's NE2 strongly hydrogen bonds to 3PG's O2P with a short distance of ˜2.4 Å. The binding of substrate's phosphono group is also reinforced by a double salt-bridge to the side-chain of Arg111 at β5.

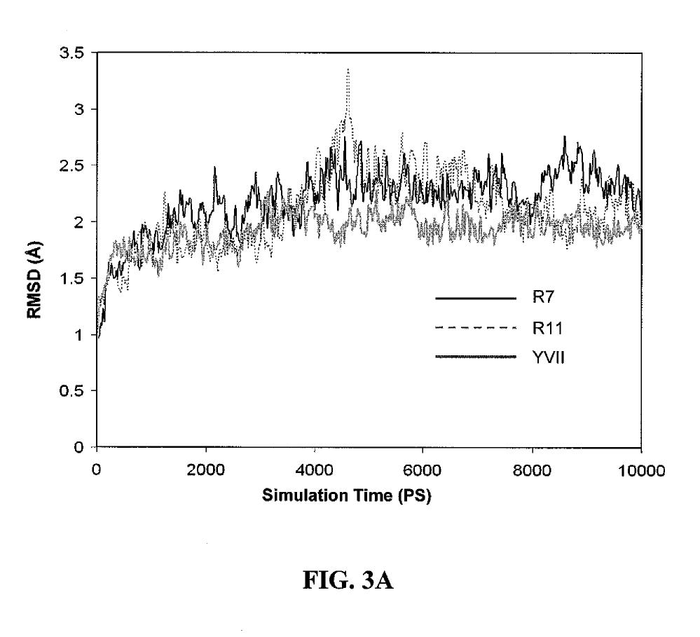

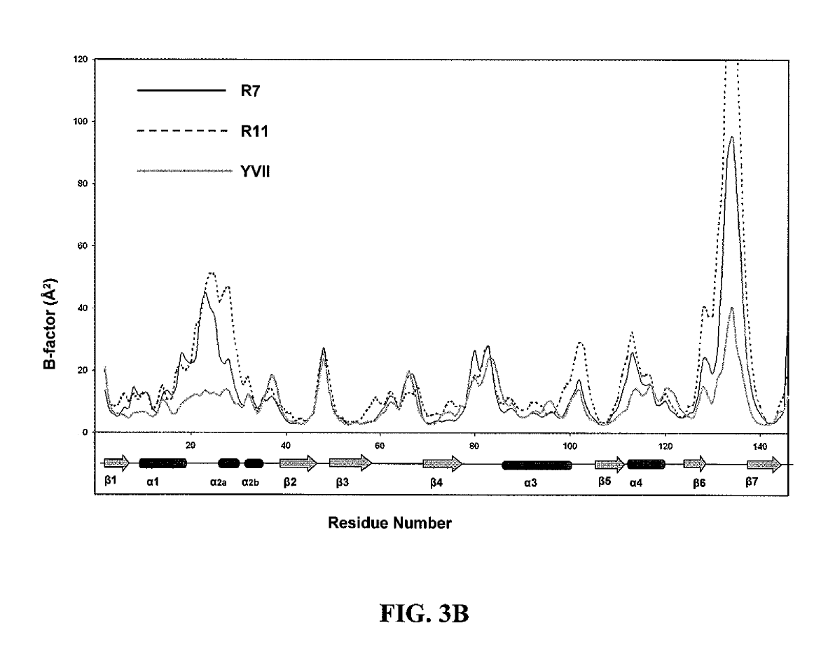

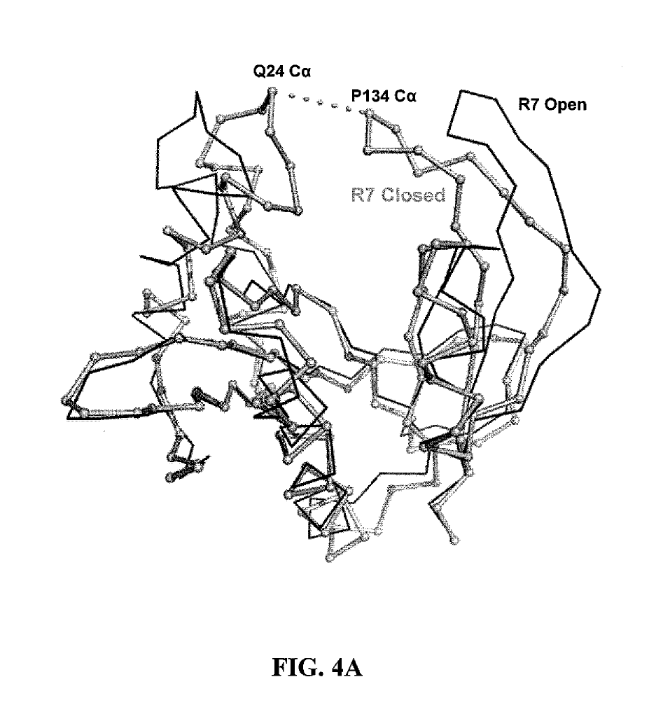

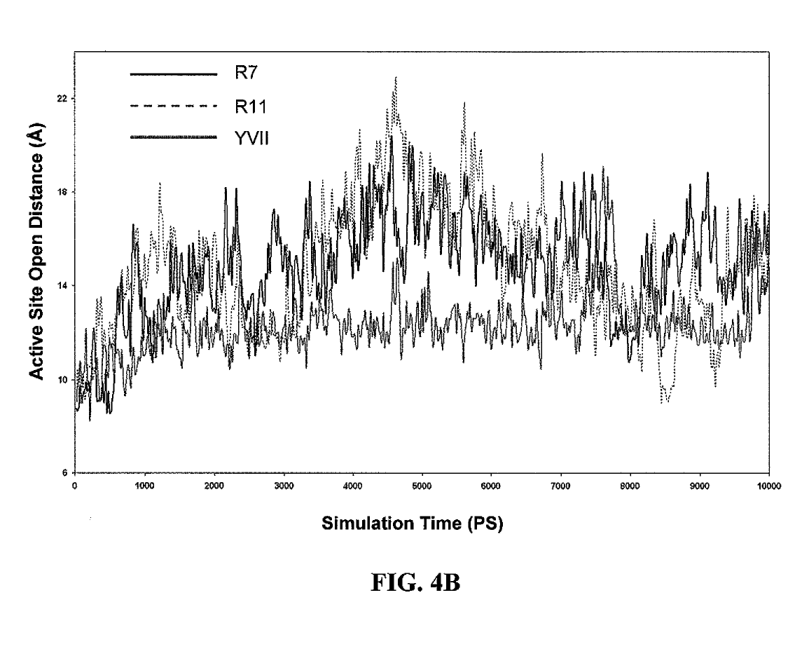

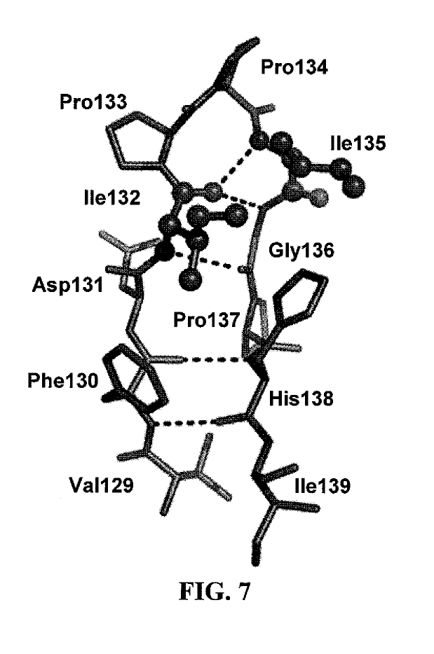

As described elsewhere herein, amino acid substitutions I132T and I135V, introduced by gene shuffling, had a significant impact on β-hairpin stability by reducing hydrophobic packing strength among the paired side chains (see FIG. 8). In the YVII or native enzyme, the side chains of I132, P133, cis-Pro 134, and I135 (and possibly H138 as well) form a hydrophobic cluster, stabilizing the type Vla β-turn and hairpin (FIG. 7). In optimized GLYATs, however, two strong hydrophobic isoleucines are replaced by a weaker valine at 135 and even a hydrophilic threonine at 132. As a consequence, the β-hairpin in the optimized GLYAT exhibits greater flexibility (FIG. 3A, FIG. 3B, and FIG. 4B) during the molecular dynamics (MD) simulation described elsewhere herein (see Experimental Example 1).

In some embodiments, the substrate binding cavity further comprises the full atomic coordinates of the substrate-binding β-hairpin (residues 130-138) defined by the atomic coordinates provided in Table 5 in addition to the atomic coordinates provided in Table 1, Table 3, or both or a structural variant thereof. In other embodiments, the substrate binding cavity further comprises the full atomic coordinates of the substrate-binding β-hairpin defined by the atomic coordinates provided in Table 6 in addition to the atomic coordinates provided in Table 2, Table 4, or both or a structural variant thereof. The minimum distances between β-hairpin residues and glyphosate are also shown in Tables 5 and 6.

TABLE 5
The minimum contact distance between the R7 GLYAT
variant beta-hairpin residues and glyphosate^{a}.

| | | | | | | Gly- | |
|---|---|---|---|---|---|---|---|
| Resi- | | Amino | | | | phos- | Minimum |
| due | Amino | Acid | | | | ate | Distance |
| ID | Acid | Atom | X | Y | Z | Atom | (Å)^{b} |
| 131 | ASP | C | 18.609 | 9.61 | 24.855 | OP1 | 9.0278 |
| 132 | THR | CG2 | 22.798 | 8.423 | 22.815 | OP3 | 5.5732 |
| 133 | PRO | O | 24.323 | 12.02 | 22.781 | OP3 | 7.368 |
| 134 | PRO | C | 22.024 | 14.283 | 19.783 | OP1 | 7.359 |
| 135 | VAL | CG1 | 23.022 | 11.685 | 17.176 | OP2 | 3.6598 |
| 136 | GLY | N | 19.115 | 12.611 | 20.034 | OP1 | 6.3429 |
| 137 | PRO | O | 15.28 | 8.747 | 19.112 | OP1 | 6.4985 |
| 138 | HSP | NE2 | 19.486 | 7.328 | 18.349 | OP1 | 2.6087 |

^{a}The atom naming convention is the same as in Table 1.
^{b}The minimum distance in Angstroms between the listed pairs of atoms in beta-hairpin and glyphosate.

TABLE 6
The minimum contact distance between the R11 GLYAT variant
beta-hairpin residues and glyphosate^{a}.

| Resi- | | Amino | | | | | Minimum |
|---|---|---|---|---|---|---|---|
| due | Amino | Acid | | | | Glyphosate | Distance |
| ID | Acid | Atom | X | Y | Z | Atom | (Å)^{b} |
| 131 | ASP | C | 18.59 | 9.66 | 24.82 | OP3 | 9.52 |
| 132 | THR | CG2 | 22.66 | 8.39 | 22.57 | OP3 | 6.06 |
| 133 | PRO | O | 24.23 | 11.93 | 22.45 | OP3 | 8.04 |
| 134 | PRO | C | 22.05 | 13.92 | 19.13 | OP2 | 7.01 |
| 135 | VAL | CG1 | 22.96 | 11.05 | 17.01 | OP2 | 3.42 |
| 136 | GLY | O | 17.82 | 10.22 | 20.91 | OP1 | 6.71 |
| 137 | PRO | O | 15.2 | 8.8 | 19.25 | OP1 | 6.68 |
| 138 | HIS | NE2 | 19.35 | 6.92 | 18 | OP1 | 2.65 |

^{a}The atom naming convention is the same as in Table 1.
^{b}The minimum distance in Angstroms between the listed pairs of atoms in beta-hairpin and glyphosate.

Without being bound by any theory or mechanism of action, the mutated residues of the β-hairpin of the optimized GLYAT variants contribute to its reduced stability and greater flexibility, which might contribute to an acceleration of the opening of the active site and determine substrate specificity. In addition, the phenol of wild-type GLYAT residue Y130 hydrogen bonds with the side chain of Asn 109. The R7 GLYAT variant polypeptide has a Y130F mutation and without being bound by any theory or mechanism of action, we believe that the absence of this hydrogen bond might allow the optimized GLYAT variant to more easily adjust then β-hairpin conformation to accommodate new substrate (e.g., glyphosate).

In any of these embodiments, a structural variant of the substrate binding cavity can be used for comparison to a three-dimensional molecular structure of a candidate polypeptide comprising the provided atomic coordinates in Table 1, Table 1 and Table 3, Table 1 and Table 5, Tables 1, 3, and 5, Table 2, Table 2 and 4, Table 2 and 6, or Tables 2, 4, and 6, wherein the structural variant comprises a root mean square deviation from the back-bone atoms of the amino acids for which the atomic coordinates are provided of not more than about 4 Å, and in some embodiments, not more than about 2 Å, including but not limited to about 4 Å, 3.5 Å, 3 Å, 2.5 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å, and 0.1 Å.

The three-dimensional molecular structures of the GLYAT polypeptide and the candidate polypeptide are compared to determine if the candidate polypeptide comprises the substrate binding cavity of the GLYAT polypeptide (comprising the atomic coordinates of Table 1, Table 1 and Table 3, Table 1 and Table 5, Tables 1, 3, and 5, Table 2, Table 2 and 4, Table 2 and 6, or Tables 2, 4, and 6). A candidate polypeptide is considered to comprise the substrate binding cavity of the GLYAT polypeptide if the candidate polypeptide comprises a region wherein the back-bone atoms of the amino acids of this region have no more than about 4 Å root mean square deviation from the backbone atoms of the amino acids provided in Table 1, and optionally Table 3, and Table 5, including but not limited to about 4 Å, 3.5 Å, 3 Å, 2.5 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å, and 0.1 Å. In other embodiments, a candidate polypeptide is considered to comprise the substrate binding cavity oldie GLYAT polypeptide if the candidate polypeptide comprises a region wherein the back-bone atoms of the amino acids of this region have no more than about 4 Å root mean square deviation from the backbone atoms of the amino acids provided in Table 2, and optionally Table 4, and Table 6, including but not limited to about 4 Å, 3.5 Å, 3 Å, 2.5 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å, and 0.1 Å. In some embodiments, the two molecular structures are considered the same if the root mean square deviation between the back-bone atoms of the amino acids of this region are not more than about 2 Å. Any method known in the art can be used to compare the two three-dimensional molecular structures to determine if the candidate polypeptide comprises the optimized substrate binding cavity. Such analyses may be carried out in current software applications, such as the Molecular Similarity application of QUANTA (Molecular Simulations Inc., San Diego, Calif.) and as described in the accompanying User's Guide. The Molecular Similarity application permits comparisons between different structures, different conformations of the same structure, and different parts of the same structure. The procedure used in Molecular Similarity to compare structures is divided into four steps: 1) load the structures to be compared; 2) define the atom equivalences in these structures; 3) perform a fitting operation; and 4) analyze the results. Each structure is identified by a name. One structure is identified as the target (i.e., the fixed structure); all remaining structures are working structures (i.e., moving structures). Since atom equivalency within QUANTA is defined by user input, for the purpose of this invention we will define equivalent atoms as protein backbone atoms (N, C.alpha., C and O) for all conserved residues between the two structures being compared. Many other structural comparison tools automatically identify equivalent atoms (usually the alpha carbons of equivalent residues). Since the geometrical distance between the alpha carbons of any two residues in a 3D structure does not directly reflect the position of the residues in the corresponding primary ID sequence, the identified equivalent residues of two proteins can be non-consecutive, not the same residue number, or even not in the same sequential order. The widely available software packages include, but are not limited to, Dali (Holm & Sander (1993) J Mol Biol. 233(1):123-138), SSM (Krissinel & Henrick (2004) Acta Cryst. D60:2256-2268), VAST (Gibrat et al. (1996) Curr Opin Struct Biol 6(3):377-385), and CE (Shindyalov & Bourne (1998) Protein Engineering 11(9):739-747). We will also consider only rigid fitting operations. When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA and others.

In embodiments, the present subject matter is directed to an electronic representation comprising the atomic coordinates of any glyphosate N-acetyltransferase (GLYAT) or variant thereof described herein. In a preferred embodiment, an electronic representation comprises the atomic coordinates of a glyphosate N-acetyltransferase (GLYAT) polypeptide crystal. In another preferred embodiment, an electronic representation comprises the atomic coordinates found in Tables 18 or 19.

In another embodiment, the present subject matter is directed to a data array comprising the atomic coordinates of a glyphosate N-acetyltransferase (GLYAT) polypeptide crystal said atomic coordinates comprising, a) a three-dimensional representation of at least one of a substrate binding cavity comprising atomic coordinates described herein; and b) a variant of the three-dimensional representation of part (a), wherein said variant comprises a root mean square deviation from the back-bone atoms of said amino acids of not more than 1.9 Å.

In another embodiment, the present subject matter is directed to an electronic representation comprising the atomic coordinates of a glyphosate N-acetyltransferase (GLYAT) polypeptide crystal said atomic coordinates comprising, a) a three-dimensional representation of at least one of a substrate binding cavity comprising atomic coordinates described herein; and b) a variant of the three-dimensional representation of part (a), wherein said variant comprises a root mean square deviation from the back-bone atoms of said amino acids of not more than 1.9 Å.

It is to be noted that the candidate polypeptide can be considered to comprise the GLYAT substrate binding cavity of Table 1, and in some embodiments, Table 3, Table 5, or both, or the GLYAT substrate binding cavity of Table 2, and in some embodiments, Table 4, Table 6, or both, even if the particular residue number between the GLYAT polypeptide and candidate polypeptide are dissimilar, so long as the atomic coordinates of the amino acid atoms that contact glyphosate are the same (or wherein the back-bone atoms of the amino acids of this region have no more than about 4 Å root mean square deviation from the backbone atoms of the amino acids provided in Table 1, Table 1 and Table 3, Table 1 and Table 5, Tables 1, 3, and 5, Table 2, Table 2 and 4, Table 2 and 6, or Tables 2, 4, and 6, as discussed above). For example, the leucine residue at position 20 in the substrate binding cavity of the GLYAT R7 variant polypeptide listed in Table 1 can correspond to a leucine residue in the substrate binding cavity of the candidate polypeptide that is not at the 20 position in the amino acid sequence of the candidate polypeptide. One of skill in the art will appreciate that the two molecular structures can still be considered the same or similar so long as the three-dimensional molecular structure of the candidate polypeptide comprises the atomic coordinates within Table 1, Table 1 and Table 3, Table 1 and Table 5, Tables 1, 3, and 5, Table 2, Table 2 and 4, Table 2 and 6, or Tables 2, 4, and 6 (or a variation thereof), regardless of the positioning of a given residue within the polypeptide chain.

In some embodiments, the methods of the invention further comprise altering the primary structure of the candidate polypeptide to maximize a similarity or relationship between the three-dimensional molecular structures of the candidate polypeptide and the substrate binding cavity of the GLYAT polypeptide (comprising the atomic coordinates of Table 1, Table 1 and Table 3, Table 1 and Table 5, Tables 1, 3, and 5, Table 2, Table 2 and 4, Table 2 and 6, or Tables 2, 4, and 6). Any method known in the art can be used to alter the primary structure of the candidate polypeptide, including any mutagenic or recombinogenic methods described elsewhere herein. One of skill in the art will appreciate that mutations introduced outside of the substrate binding cavity may influence the secondary or tertiary structure of the polypeptide and indirectly alter the three-dimensional structure of the substrate binding cavity. Candidate polypeptides, particularly those whose primary structure have been modified to provide a better fit with the substrate binding cavity of the GLYAT polypeptide, can be produced and assayed for the ability to bind to glyphosate with a higher binding affinity or specificity when compared to a native GLYAT polypeptide using any method known in the art. In this way, the methods of the invention provide for the identification of additional optimized GLYAT polypeptides that exhibit enhanced affinity or specificity for glyphosate over native GLYAT polypeptides.

As used herein, the term “maximize” includes enhance, increase, improve and the like. Thus, the term is not limited to a highest measure but is meant to also describe incremental enhancements, improvements and the like.

In some embodiments of the methods of the invention, the candidate polypeptide is evaluated for its potential to have N-acetyltransferase activity with a higher catalytic rate (k_{cat}) for a substrate when compared to a native GLYAT polypeptide. In these embodiments, a three-dimensional molecular structure of at least a GNAT wedge joining region of a GLYAT polypeptide is provided and the three-dimensional molecular structure of a candidate polypeptide are compared to determine if the candidate polypeptide has the potential to have N-acetyltransferase activity with a higher k_{cat} for a substrate when compared to a native GLYAT polypeptide. The molecular structure is determined from a GLYAT polypeptide bound to glyphosate and an acetyl donor (e.g., AcCoA). GLYAT polypeptides comprise the classic GNAT wedge shape that comprises a V-shaped wedge formed by two central parallel beta strands splaying apart at the middle point (for example, see beta strands β4 and β5 of GLYAT in FIG. 1). The GNAT wedge of GLYAT essentially separates the polypeptide into two subdomains, with β1-β4 in subdomain I and strands β5-β7 in subdomain II. As used herein, a “GNAT wedge joining region” refers to the region of the GNAT wedge where the two central parallel beta strands meet. For example, the wedge joining region of the R7 GLYAT variant polypeptide comprises the area where beta strands β4 and β5 meet. The unique wedge topology of GNAT proteins is responsible for the highly conserved AcCoA binding mode. The parting of the two parallel β4 and β5 allows the bound AcCoA to place its acetyl group in the wedge joining region, forming the reaction center. The acetyl and pantetheine moieties of AcCoA, mimicking a pseudo peptide β-strand, projects carbonyl and amide groups to both sides and hydrogen bonds to the backbone of the adjacent β4, allowing the main β sheet to extend to some degree.

Beyond substrate binding, two other residues, Try118 and Met75, are essential to catalysis. Try118 is about 3.6 Å from AcCoA SIP and is in position to serve as the general base protonating the thiolate anion of CoA (Sichl et al. (2007) J Biol Chem 282:11446-11455). A characteristic feature of GLYAT, the β-bulge at strand 4, formed by residues Gly74 and Met75, orients the amide of Met75 to the reaction center, forming a hydrogen bond to the carbonyl of the AcCoA's thioester (FIG. 8). This hydrogen bond both positions the thioester properly for the acylation reaction and further polarizes the carbonyl making the carbon atom more susceptible to nucleophilic attack by the glyphosate amine. In the GLYAT R11, Met75 was replaced by a valine. The side chain alteration fine-tunes this amide group to better fit glyphosate.

The wedge also contributes two residues that recognize glyphosate through their side-chains (Arg73 and Arg111). Atomic coordinates found within about 4 Å of the bound AcCoA, where the two beta strands meet are considered part of the wedge joining region. In some embodiments, the GNAT wedge joining region comprises the atomic coordinates provided in Table 7 or Table 8.

TABLE 7
Contacts between AcCoA and the R7 GLYAT variant polypeptide^{a}
when the polypeptide is bound to AcCoA and glyphosate^{a}.

| Residue | Amino | GLYAT | | | | AcCoA^{b} | Distance |
|---|---|---|---|---|---|---|---|
| ID | Acid | Atom | X | Y | Z | Atom) | (A) |
| 20 | LEU | CD2 | 22.35 | 7.82 | 11.09 | C5P | 3.91 |
| | | | | | | C6P | 3.62 |
| | | | | | | N4P | 3.64 |
| 72 | LEU | O | 22.80 | −1.08 | 15.38 | CH3 | 3.58 |
| 73 | ARG | C | 25.13 | 0.95 | 12.68 | O | 3.82 |
| 74 | GLY | C | 24.61 | 2.93 | 10.29 | O | 3.84 |
| | | CA | 25.45 | 3.10 | 11.53 | O | 3.68 |
| | | N | 25.02 | 2.30 | 12.66 | O | 3.06 |
| 75 | MET | CB | 21.10 | 1.81 | 9.75 | O | 3.99 |
| | | N | 23.28 | 2.75 | 10.44 | O | 3.07 |
| | | O | 21.43 | 4.75 | 9.44 | C2P | 3.26 |
| | | | | | | C3P | 3.60 |
| | | | | | | N4P | 2.99 |
| 76 | ALA | C | 21.81 | 5.16 | 5.32 | O9P | 3.82 |
| | | CA | 21.83 | 5.35 | 6.81 | CDP | 3.90 |
| | | | | | | O9P | 3.72 |
| 77 | THR | C | 20.99 | 7.49 | 2.84 | O9P | 3.80 |
| | | CA | 20.94 | 6.02 | 3.18 | O9P | 3.82 |
| | | CB | 19.68 | 5.40 | 2.60 | O5A | 3.41 |
| | | | | | | O6A | 3.55 |
| | | | | | | P2A | 3.90 |
| | | CG2 | 19.63 | 5.55 | 1.07 | O5A | 3.47 |
| | | N | 21.01 | 5.98 | 4.63 | C9P | 3.96 |
| | | | | | | CDP | 3.84 |
| | | | | | | O9P | 2.97 |
| | | O | 20.31 | 8.31 | 3.44 | C9P | 4.00 |
| | | | | | | O9P | 3.01 |
| | | OG1 | 19.64 | 4.01 | 2.89 | O5A | 2.85 |
| | | | | | | O6A | 3.17 |
| | | | | | | P2A | 3.58 |
| 82 | ARG | C | 14.96 | 6.74 | −1.09 | O4A | 3.69 |
| | | CA | 16.21 | 7.16 | −0.38 | O4A | 3.35 |
| | | CB | 15.84 | 8.18 | 0.72 | O4A | 3.36 |
| | | CD | 16.63 | 9.38 | 2.83 | CAP | 3.97 |
| | | | | | | OAP | 3.63 |
| | | CG | 17.01 | 8.47 | 1.67 | O4A | 3.65 |
| | | CZ | 18.00 | 10.62 | 4.55 | C9P | 3.68 |
| | | | | | | O9P | 3.50 |
| | | NE | 17.86 | 9.60 | 3.66 | C9P | 3.52 |
| | | | | | | CAP | 3.75 |
| | | | | | | O9P | 3.31 |
| | | | | | | OAP | 3.78 |
| | | NH2 | 19.23 | 10.89 | 5.04 | C7P | 3.96 |
| | | | | | | C9P | 3.95 |
| | | | | | | O9P | 3.44 |
| 83 | GLU | C | 13.11 | 4.42 | −2.20 | O1A | 3.59 |
| | | CA | 12.95 | 5.26 | −0.95 | O1A | 3.35 |
| | | CD | 10.95 | 6.33 | 1.21 | C2B | 3.73 |
| | | | | | | O2B | 3.40 |
| | | N | 14.21 | 5.79 | −0.47 | O1A | 3.51 |
| | | | | | | O4A | 3.03 |
| | | OE1 | 9.85 | 5.77 | 0.98 | C2B | 3.27 |
| | | | | | | O2B | 2.64 |
| | | OE2 | 11.59 | 6.21 | 2.29 | C2B | 3.43 |
| | | | | | | C8A | 3.21 |
| | | | | | | N7A | 3.74 |
| | | | | | | N9A | 3.78 |
| | | | | | | O2B | 3.40 |
| | | | | | | O4A | 3.99 |
| 84 | GLN | N | 14.29 | 3.78 | −2.36 | O1A | 3.39 |
| 85 | LYS | C | 16.41 | −0.29 | −0.94 | O2A | 3.74 |
| | | CA | 14.94 | −0.07 | −1.21 | O1A | 3.43 |
| | | | | | | O2A | 3.49 |
| | | | | | | P1A | 4.00 |
| | | CE | 10.60 | −1.73 | −0.71 | O7A | 3.37 |
| | | N | 14.70 | 1.23 | −1.81 | O1A | 3.00 |
| | | NZ | 10.00 | −0.41 | −0.99 | O7A | 2.72 |
| 86 | ALA | C | 18.92 | 0.13 | 0.79 | O5A | 3.83 |
| | | CA | 18.64 | 0.67 | −0.59 | O5A | 3.92 |
| | | CB | 19.39 | 2.00 | −0.77 | O5A | 3.73 |
| | | N | 17.22 | 0.79 | −0.83 | O5A | 3.68 |
| 87 | GLY | C | 17.80 | −1.14 | 3.38 | O2A | 3.84 |
| | | CA | 18.39 | 0.22 | 3.18 | O2A | 3.95 |
| | | | | | | O5A | 3.63 |
| | | N | 18.23 | 0.67 | 1.82 | O2A | 3.67 |
| | | | | | | O5A | 2.89 |
| 88 | SER | CA | 15.94 | −2.69 | 2.84 | O2A | 3.60 |
| | | CB | 14.62 | −2.71 | 2.03 | O2A | 3.30 |
| | | N | 16.62 | −1.40 | 2.77 | O2A | 2.91 |
| | | OG | 13.66 | −1.85 | 2.62 | C5B | 3.27 |
| | | | | | | O2A | 2.63 |
| | | | | | | P1A | 3.85 |
| 108 | CYS | SG | 18.71 | 0.07 | 14.80 | CH3 | 3.60 |
| | | | | | | S1P | 3.86 |
| 109 | ASN | O | 18.88 | 3.13 | 17.87 | CH3 | 3.82 |
| 111 | ARG | CD | 18.36 | 8.96 | 14.19 | O5P | 3.61 |
| 113 | SER | C | 12.36 | 8.02 | 12.16 | N6A | 4.00 |
| | | O | 12.12 | 8.30 | 10.99 | C6A | 3.61 |
| | | | | | | N1A | 3.54 |
| | | | | | | N6A | 2.92 |
| 114 | ALA | CA | 13.31 | 5.81 | 11.60 | N6A | 3.89 |
| | | CB | 14.84 | 5.62 | 11.54 | C3P | 3.86 |
| | | | | | | O5P | 3.54 |
| 116 | GLY | C | 9.33 | 2.36 | 9.53 | C2A | 3.63 |
| | | | | | | N3A | 3.45 |
| | | CA | 8.65 | 3.11 | 10.64 | C2A | 3.64 |
| | | | | | | N3A | 3.94 |
| | | O | 8.71 | 1.52 | 8.88 | N3A | 3.79 |
| 117 | TYR | CA | 11.47 | 1.90 | 8.36 | C4A | 3.73 |
| | | | | | | N3A | 3.91 |
| | | | | | | N9A | 3.84 |
| | | | | | | O4B | 3.60 |
| | | CB | 12.82 | 2.67 | 8.20 | C4A | 3.75 |
| | | | | | | C5A | 3.69 |
| | | | | | | C8A | 3.80 |
| | | | | | | CEP | 3.97 |
| | | | | | | N7A | 3.75 |
| | | | | | | N9A | 3.82 |
| | | CD1 | 14.03 | 1.41 | 6.35 | CCP | 3.67 |
| | | | | | | O3A | 3.77 |
| | | | | | | O4B | 3.79 |
| | | | | | | O5B | 3.79 |
| | | CD2 | 15.07 | 1.63 | 8.53 | CEP | 3.80 |
| | | CE1 | 15.16 | 0.71 | 5.89 | CCP | 3.79 |
| | | | | | | O2A | 3.73 |
| | | | | | | O3A | 3.64 |
| | | CG | 13.99 | 1.88 | 7.67 | CCP | 3.95 |
| | | | | | | CEP | 3.81 |
| | | N | 10.64 | 2.62 | 9.30 | C2A | 3.58 |
| | | | | | | C4A | 3.66 |
| | | | | | | N3A | 3.41 |
| 118 | TYR | OH | 18.16 | 1.23 | 11.55 | S1P | 3.58 |
| 120 | LYS | CB | 8.38 | −1.09 | 6.26 | O4B | 3.94 |
| | | CD | 5.94 | −0.45 | 5.76 | O8A | 3.57 |
| | | CE | 4.89 | 0.66 | 5.64 | O8A | 3.57 |
| | | CG | 7.32 | 0.03 | 6.25 | O3B | 3.79 |
| | | | | | | O4B | 3.98 |
| | | NZ | 5.33 | 1.70 | 4.68 | O3B | 3.34 |
| | | | | | | O8A | 2.82 |
| | | | | | | P3B | 3.66 |

^{a}The naming convention of amino acid atoms and all the atomic coordinates is the same as Table 1 and the structure model used here is the same as that in Table 1.

TABLE 8
Contacts between AcCoA and the R11 GLYAT variant polypeptide^{a}
when the polypeptide is bound to AcCoA and glyphosate^{a}.

| Resi- | | | | | | | |
|---|---|---|---|---|---|---|---|
| due | Amino | GLYAT | | | | AcCoA | Distance |
| ID | Acid | Atom | X | Y | Z | Atom | (Å) |
| 151 | Bound | C2 | 22.35 | 5.74 | 14.33 | C | 3.4 |
| | Glyphosate | | | | | CH3 | 3.71 |
| | | | | | | O | 3.56 |
| | | | | | | S1P | 3.9 |
| 19 | VAL | O | 22.13 | 11.15 | 9.25 | C6P | 3.65 |
| 20 | LEU | CD2 | 22.41 | 7.72 | 10.69 | C5P | 3.7 |
| | | | | | | C6P | 3.55 |
| | | | | | | N4P | 3.45 |
| 72 | LEU | O | 21.94 | −1.07 | 15.05 | CH3 | 3.87 |
| 73 | ARG | C | 24.34 | 0.9 | 12.48 | O | 3.67 |
| 74 | GLY | C | 24.27 | 3.04 | 10.14 | O | 3.99 |
| | | N | 24.48 | 2.25 | 12.53 | O | 3.32 |
| 75 | VAL | CA | 22.18 | 2.69 | 8.91 | O | 3.9 |
| | | CB | 20.9 | 1.88 | 9.06 | O | 3.75 |
| | | CG2 | 21.21 | 0.6 | 9.86 | O | 3.58 |
| | | N | 22.93 | 2.85 | 10.14 | O | 3.07 |
| | | O | 21.24 | 4.89 | 8.94 | C2P | 3.28 |
| | | | | | | C3P | 3.72 |
| | | | | | | C5P | 3.98 |
| | | | | | | N4P | 3 |
| 76 | ALA | C | 21.99 | 5.21 | 4.86 | CDP | 3.86 |
| | | | | | | O9P | 3.79 |
| | | CA | 22.01 | 5.47 | 6.34 | CDP | 3.73 |
| | | | | | | O9P | 3.72 |
| 77 | THR | C | 21.41 | 7.44 | 2.27 | O9P | 3.77 |
| | | CA | 21.19 | 6.01 | 2.67 | O9P | 3.75 |
| | | CB | 19.88 | 5.47 | 2.1 | O5A | 3.49 |
| | | | | | | O6A | 3.54 |
| | | | | | | P2A | 3.93 |
| | | CG2 | 19.81 | 5.6 | 0.57 | O5A | 3.55 |
| | | N | 21.23 | 6.04 | 4.11 | C9P | 3.89 |
| | | | | | | CDP | 3.72 |
| | | | | | | O9P | 2.91 |
| | | O | 20.88 | 8.37 | 2.87 | O9P | 3 |
| | | OG1 | 19.77 | 4.08 | 2.41 | CDP | 3.99 |
| | | | | | | O5A | 2.88 |
| | | | | | | O6A | 3.11 |
| | | | | | | P2A | 3.57 |
| 82 | ARG | C | 15.1 | 7.11 | −1.42 | O4A | 3.74 |
| | | CA | 16.4 | 7.47 | −0.75 | O4A | 3.42 |
| | | CB | 16.13 | 8.5 | 0.37 | O4A | 3.48 |
| | | CD | 17.14 | 9.74 | 2.37 | OAP | 3.77 |
| | | CG | 17.37 | 8.73 | 1.24 | O4A | 3.81 |
| | | CZ | 18.78 | 10.95 | 3.86 | C9P | 3.87 |
| | | | | | | O9P | 3.51 |
| | | NE | 18.44 | 9.88 | 3.1 | C9P | 3.53 |
| | | | | | | CAP | 3.93 |
| | | | | | | O9P | 3.18 |
| | | NH2 | 20.02 | 11 | 4.39 | C7P | 3.92 |
| | | | | | | O9P | 3.41 |
| 83 | GLU | C | 13.25 | 4.81 | −2.54 | O1A | 3.8 |
| | | CA | 13.12 | 5.58 | −1.25 | O1A | 3.46 |
| | | CD | 11.08 | 6.2 | 0.99 | C2B | 3.69 |
| | | | | | | O2B | 3.46 |
| | | N | 14.38 | 6.15 | −0.8 | O1A | 3.75 |
| | | | | | | O4A | 3.04 |
| | | OE1 | 11.69 | 6.12 | 2.09 | C2B | 3.3 |
| | | | | | | C8A | 3.16 |
| | | | | | | N7A | 3.74 |
| | | | | | | N9A | 3.74 |
| | | | | | | O2B | 3.35 |
| | | OE2 | 10.1 | 5.46 | 0.68 | C2B | 3.27 |
| | | | | | | O2B | 2.86 |
| 84 | GLN | N | 14.41 | 4.16 | −2.75 | O1A | 3.7 |
| 85 | LYS | C | 16.29 | −0.04 | −1.44 | O2A | 3.88 |
| | | CA | 14.84 | 0.36 | −1.56 | O1A | 3.38 |
| | | | | | | O2A | 3.57 |
| | | CD | 11.97 | −0.5 | −0.65 | O1A | 3.89 |
| | | | | | | O2A | 3.95 |
| | | CE | 10.45 | −0.49 | −0.48 | O7A | 3.26 |
| | | | | | | O9A | 3.59 |
| | | N | 14.66 | 1.64 | −2.2 | O1A | 3.13 |
| | | NZ | 9.84 | 0.7 | −1.1 | O7A | 3.61 |
| | | | | | | O9A | 2.87 |
| | | | | | | P3B | 3.88 |
| 86 | ALA | C | 18.83 | 0.17 | 0.33 | O5A | 3.81 |
| | | CA | 18.59 | 0.72 | −1.05 | O5A | 3.92 |
| | | CB | 19.43 | 2.01 | −1.22 | O5A | 3.76 |
| | | N | 17.19 | 0.96 | −1.28 | O5A | 3.64 |
| 87 | GLY | C | 17.84 | −0.88 | 3.09 | O2A | 3.79 |
| | | CA | 18.46 | 0.45 | 2.76 | O2A | 3.93 |
| | | | | | | O5A | 3.51 |
| | | N | 18.25 | 0.82 | 1.37 | O2A | 3.68 |
| | | | | | | O5A | 2.82 |
| | | | | | | P2A | 3.98 |
| 88 | SER | CA | 15.98 | −2.45 | 2.59 | O2A | 3.58 |
| | | CB | 14.64 | −2.49 | 1.81 | O2A | 3.3 |
| | | N | 16.66 | −1.17 | 2.48 | O2A | 2.88 |
| | | OG | 13.69 | −1.64 | 2.41 | C5B | 3.24 |
| | | | | | | O2A | 2.64 |
| | | | | | | O5B | 4 |
| | | | | | | P1A | 3.85 |
| 108 | CYS | SG | 18.39 | −0.26 | 14.64 | CH3 | 3.56 |
| 109 | ASN | C | 18.03 | 2.04 | 17.94 | CH3 | 3.95 |
| | | O | 18.85 | 2.91 | 17.67 | CH3 | 3.23 |
| 111 | ARG | CD | 17.91 | 8.99 | 14.2 | O5P | 3.86 |
| 113 | SER | O | 11.71 | 8.62 | 11.64 | N1A | 3.6 |
| 114 | ALA | CB | 14.77 | 6.2 | 11.8 | C3P | 3.89 |
| 116 | GLY | C | 9.46 | 2.68 | 9.84 | C2A | 3.8 |
| | | | | | | N3A | 3.6 |
| | | O | 8.84 | 1.89 | 9.13 | N3A | 3.92 |
| 117 | TYR | CA | 11.62 | 2.3 | 8.65 | C4A | 3.69 |
| | | | | | | N3A | 3.67 |
| | | | | | | N9A | 3.93 |
| | | | | | | O4B | 3.76 |
| | | CB | 13.01 | 3.01 | 8.6 | C4A | 3.71 |
| | | | | | | C5A | 3.73 |
| | | | | | | N9A | 4 |
| | | CD1 | 13.98 | 1.8 | 6.56 | C8A | 3.8 |
| | | | | | | CCP | 3.89 |
| | | | | | | O4B | 3.58 |
| | | | | | | O5B | 3.96 |
| | | CD2 | 15.33 | 2.06 | 8.54 | CEP | 3.8 |
| | | CE1 | 15.01 | 1.07 | 5.95 | CCP | 3.8 |
| | | | | | | O3A | 3.84 |
| | | CG | 14.12 | 2.28 | 7.87 | CEP | 3.88 |
| | | N | 10.77 | 2.95 | 9.63 | C2A | 3.51 |
| | | | | | | C4A | 3.87 |
| | | | | | | N3A | 3.37 |
| | | O | 11.61 | −0.04 | 8.15 | O4B | 3.9 |
| 118 | TYR | OH | 18.18 | 1.37 | 11.23 | C | 3.64 |
| | | | | | | C2P | 3.66 |
| | | | | | | O | 3.65 |
| | | | | | | S1P | 3.52 |
| 120 | LYS | CB | 8.72 | −0.78 | 6.24 | C4B | 3.78 |
| | | | | | | O4B | 3.75 |
| | | CD | 6.44 | 0.33 | 5.78 | O3B | 3.57 |
| | | | | | | O8A | 3.5 |
| | | | | | | P3B | 3.93 |
| | | CE | 5.61 | 1.63 | 5.81 | O3B | 3.83 |
| | | | | | | O8A | 3.61 |
| | | CG | 7.86 | 0.49 | 6.33 | C4B | 3.87 |
| | | | | | | O3B | 3.51 |
| | | | | | | O4B | 3.59 |
| | | NZ | 6.17 | 2.66 | 4.9 | O2B | 3.77 |
| | | | | | | O3B | 2.93 |
| | | | | | | O8A | 3.15 |
| | | | | | | P3B | 3.66 |

^{a}The naming convention of amino acid atoms and all the atomic coordinates is the same as Table 1 and the structure model used here is the same as that in Table 1.

In some embodiments, the three-dimensional molecular structure of the GNAT wedge joining region can be described as comprising the backbone atomic coordinates and the inter-strand C-alpha atom distance of Table 9, which are found in the GLYAT R7 variant polypeptide, and the GNAT wedge joining region further comprises the atomic coordinates of Table 9, in addition to those of Table 7. In other embodiments, the three-dimensional molecular structure of the GNAT wedge joining region can be described as comprising the backbone atomic coordinates and the inter-strand C-alpha atom distance of Table 10, which are found in the GLYAT R11 variant polypeptide, and the GNAT wedge joining region further comprises the atomic coordinates of Table 10, in addition to those of Table 8.

TABLE 9
The wedge of GLYAT R7 variant polypeptide defined by backbone atoms of beta 4 and beta 5.

| Beta 4 Strand | | | | | | Beta 5 Strand | | | | | | |
|---|---|---|---|---|---|---|---|---|---|---|---|---|
| Residue | Amino | Atom | | | | Residue | Amino | Atom | | | | Distance |
| ID | Acid | name^{a} | X^{b} | Y | Z | ID | Acid | name | X | Y | Z | (Å)^{c} |
| 69 | GLN | N | 22.87 | −13.02 | 19.2 | 105 | LEU | N | 18.38 | −13.28 | 16.3 | |
| | | CA | 22.11 | −11.86 | 18.78 | | | CA | 17.69 | −12.03 | 16.05 | 5.2 |
| | | C | 23.05 | −10.9 | 18.12 | | | C | 18.68 | −10.95 | 15.72 | |
| | | O | 24.23 | −10.82 | 18.48 | | | O | 19.87 | −11.05 | 16.01 | |
| 70 | TYR | N | 22.57 | −10.13 | 17.11 | 106 | LEU | N | 18.16 | −9.88 | 15.09 | |
| | | CA | 23.39 | −9.19 | 16.38 | | | CA | 18.87 | −8.68 | 14.75 | 4.83 |
| | | C | 22.78 | −7.83 | 16.5 | | | C | 18.15 | −7.59 | 15.49 | |
| | | O | 21.56 | −7.68 | 16.52 | | | O | 16.92 | −7.53 | 15.45 | |
| 71 | GLN | N | 23.64 | −6.79 | 16.57 | 107 | TRP | N | 18.89 | −6.71 | 16.2 | |
| | | CA | 23.22 | −5.42 | 16.72 | | | CA | 18.29 | −5.68 | 17.01 | 4.94 |
| | | C | 23.59 | −4.66 | 15.47 | | | C | 18.97 | −4.37 | 16.75 | |
| | | O | 24.69 | −4.8 | 14.94 | | | O | 20.06 | −4.3 | 16.2 | |
| 72 | LEU | N | 22.64 | −3.83 | 14.98 | 108 | CYS | N | 18.27 | −3.27 | 17.12 | |
| | | CA | 22.8 | −2.94 | 13.87 | | | CA | 18.7 | −1.94 | 16.82 | 5.14 |
| | | C | 23.29 | −1.6 | 14.37 | | | C | 18.17 | −1.04 | 17.9 | |
| | | O | 22.8 | −1.08 | 15.38 | | | O | 17.03 | −1.2 | 18.34 | |
| 73 | ARG | N | 24.27 | −1.01 | 13.67 | 109 | ASN | N | 18.96 | −0.03 | 18.32 | |
| | | CA | 24.84 | 0.27 | 13.98 | | | CA | 18.48 | 1.1 | 19.09 | 8.2 |
| | | C | 25.12 | 0.95 | 12.68 | | | C | 18.14 | 2.16 | 18.07 | |
| | | O | 25.45 | 0.3 | 11.68 | | | O | 18.88 | 3.12 | 17.87 | |
| 74 | GLY | N | 25.02 | 2.3 | 12.65 | 110 | ALA | N | 17 | 1.96 | 17.38 | |
| | | CA | 25.45 | 3.1 | 11.53 | | | CA | 16.52 | 2.79 | 16.3 | 10.13 |
| | | C | 24.61 | 2.93 | 10.29 | | | C | 16.22 | 4.19 | 16.74 | |
| | | O | 25.14 | 2.95 | 9.18 | | | O | 15.73 | 4.41 | 17.85 | |
| 75 | MET | N | 23.28 | 2.75 | 10.44 | 111 | ARG | N | 16.45 | 5.19 | 15.86 | |
| | | CA | 22.38 | 2.56 | 9.32 | | | CA | 15.88 | 6.51 | 16.02 | 10.13 |
| | | C | 21.98 | 3.9 | 8.75 | | | C | 14.37 | 6.42 | 15.9 | |
| | | O | 21.43 | 4.75 | 9.44 | | | O | 13.85 | 5.6 | 15.15 | |
| 76 | ALA | N | 22.25 | 4.13 | 7.45 | 112 | THR | N | 13.63 | 7.28 | 16.63 | |
| | | CA | 21.83 | 5.35 | 6.81 | | | CA | 12.17 | 7.24 | 16.66 | 14.92 |
| | | C | 21.81 | 5.16 | 5.32 | | | C | 11.57 | 7.77 | 15.38 | |
| | | O | 22.51 | 4.29 | 4.79 | | | O | 10.4 | 7.54 | 15.09 | |

^{a}The amino acid atom is the specific atom of the amino acid, as identified in Protein Data Bank file 2JDD;
^{b}X, Y, and Z are the three-dimensional coordinates specifying the distance in Angstroms of the amino acid atom relative to the center of mass of the crystal.
^{c}The distance is the interstrand (β4/β5) distance of the two corresponding C-alpha atoms.

TABLE 10
The wedge of GLYAT R11 variant polypeptide defined by backbone atoms of beta 4 and beta 5.

| Beta 4 Strand | | | | | | Beta 5 Strand | | | | | | |
|---|---|---|---|---|---|---|---|---|---|---|---|---|
| Residue | Amino | Atom | | | | Residue | Amino | Atom | | | | Distance |
| ID | Acid | name^{a} | X^{b} | Y | Z | ID | Acid | name | X | Y | Z | (Å) |
| 69 | GLN | N | 23.05 | −13.02 | 19.14 | 105 | MET | N | 18.66 | −13.46 | 16.04 | |
| | | CA | 22.27 | −11.82 | 18.98 | | | CA | 17.96 | −12.21 | 15.92 | 5.3 |
| | | C | 23.16 | −10.84 | 18.28 | | | C | 18.88 | −11.1 | 15.5 | |
| | | O | 24.33 | −10.69 | 18.64 | | | O | 20.1 | −11.17 | 15.66 | |
| 70 | TYR | N | 22.63 | −10.15 | 17.25 | 106 | ILE | N | 18.26 | −10.03 | 14.96 | |
| | | CA | 23.39 | −9.19 | 16.48 | | | CA | 18.88 | −8.77 | 14.65 | 4.88 |
| | | C | 22.64 | −7.89 | 16.57 | | | C | 18.06 | −7.76 | 15.41 | |
| | | O | 21.42 | −7.87 | 16.65 | | | O | 16.84 | −7.8 | 15.41 | |
| 71 | GLN | N | 23.38 | −6.76 | 16.57 | 107 | TRP | N | 18.72 | −6.84 | 16.15 | |
| | | CA | 22.82 | −5.44 | 16.69 | | | CA | 18.02 | −5.88 | 16.97 | 4.83 |
| | | C | 23.17 | −4.65 | 15.45 | | | C | 18.63 | −4.53 | 16.75 | |
| | | O | 24.28 | −4.71 | 14.92 | | | O | 19.7 | −4.41 | 16.17 | |
| 72 | LEU | N | 22.16 | −3.9 | 14.96 | 108 | CYS | N | 17.92 | −3.46 | 17.15 | |
| | | CA | 22.18 | −3.08 | 13.78 | | | CA | 18.33 | −2.13 | 16.79 | 4.98 |
| | | C | 22.55 | −1.67 | 14.16 | | | C | 17.89 | −1.18 | 17.87 | |
| | | O | 21.94 | −1.07 | 15.05 | | | O | 16.78 | −1.3 | 18.39 | |
| 73 | ARG | N | 23.58 | −1.1 | 13.5 | 109 | ASN | N | 18.73 | −0.18 | 18.2 | |
| | | CA | 24.13 | 0.2 | 13.79 | | | CA | 18.32 | 0.98 | 18.97 | 7.82 |
| | | C | 24.34 | 0.9 | 12.48 | | | C | 18.03 | 2.04 | 17.94 | |
| | | O | 24.4 | 0.28 | 11.42 | | | O | 18.85 | 2.91 | 17.67 | |
| 74 | GLY | N | 24.48 | 2.25 | 12.53 | 110 | ALA | N | 16.84 | 1.95 | 17.31 | |
| | | CA | 25.02 | 3.04 | 11.45 | | | CA | 16.36 | 2.86 | 16.31 | 9.93 |
| | | C | 24.27 | 3.04 | 10.14 | | | C | 16.08 | 4.23 | 16.88 | |
| | | O | 24.89 | 3.19 | 9.09 | | | O | 15.73 | 4.36 | 18.05 | |
| 75 | VAL | N | 22.93 | 2.85 | 10.14 | 111 | ARG | N | 16.22 | 5.3 | 16.06 | |
| | | CA | 22.18 | 2.69 | 8.91 | | | CA | 15.63 | 6.59 | 16.35 | 10.65 |
| | | C | 21.85 | 4.03 | 8.3 | | | C | 14.13 | 6.48 | 16.35 | |
| | | O | 21.24 | 4.89 | 8.93 | | | O | 13.57 | 5.66 | 15.62 | |
| 76 | ALA | N | 22.26 | 4.24 | 7.03 | 112 | THR | N | 13.41 | 7.31 | 17.14 | |
| | | CA | 22.01 | 5.47 | 6.34 | | | CA | 11.95 | 7.31 | 17.12 | 14.86 |
| | | C | 21.99 | 5.2 | 4.86 | | | C | 11.42 | 7.96 | 15.86 | |
| | | O | 22.64 | 4.28 | 4.38 | | | O | 10.28 | 7.73 | 15.47 | |

^{a}The amino acid atom is the specific atom of the amino acid, as identified in Protein Data Bank file 2JDD;
^{b}X, Y, and Z are the three-dimensional coordinates specifying the distance in Angstroms of the amino acid atom relative to the center of mass of the crystal.
^{c}The distance is the interstrand (β4/β5) distance of the two corresponding C-alpha atoms.

Alternatively, the GNAT wedge joining region can comprise a structural variant of the GNAT wedge joining region defined by the atomic coordinates of Table 7, Tables 7 and 9, Table 8, or Tables 8 and 10, wherein the structural variant comprises a root mean square deviation from the back-bone atoms of the amino acids of Table 7, Tables 7 and 9, Table 8, or Tables 8 and 10 of not more than about 4 Å, including but not limited to about 3.5 Å, 3 Å, 2.5 Å, 2 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å, and 0.1 Å. In some of these embodiments, the variant GNAT wedge joining region comprises a root mean square deviation from the back-bone atoms of the amino acids of the structure defined by the atomic coordinates of Table 7, Tables 7 and 9, Table 8, or Tables 8 and 10 of not more than about 2.0 Å.

The analysis described elsewhere herein (see Experimental Example 1) describes two independent structural inter-subdomain motion modes within the GLYAT polypeptide involving the GNAT wedge, wherein the wedge joining region serves as a hinge for both the observed wedge opening and wedge twisting motions. Without being bound by any theory or mechanism of action, it is believed that these motions play a role in controlling the access of AcCoA, determining bound AcCoA's conformation, facilitating the egress of CoA, and facilitating the binding of glyphosate and that the mutations in the wedge joining region found in the optimized GLYAT variants contribute to the enhanced catalytic activity (and perhaps the enhanced glyphosate binding affinity and specificity) associated with these optimized variants.

The three-dimensional molecular structure of the GLYAT wedge joining region is compared to the provided three-dimensional molecular structure of a candidate polypeptide to determine if the structure of the candidate polypeptide comprises the wedge joining region of the GLYAT polypeptide (comprising the atomic coordinates of Table 7, Tables 7 and 9, Table 8, or Tables 8 and 10). En some of these embodiments, the candidate polypeptide is known to comprise a GNAT wedge or is suspected of comprising a GNAT wedge based on sequence similarity to protein members of the GNAT superfamily (see Dyda et al. (2000) Annu Rev. Biophys. Biomol. Struct. 29:81-103, which is herein incorporated by reference in its entirety). A candidate polypeptide can be suspected of comprising a GNAT wedge if the candidate polypeptide exhibits at least 30%, 40%, 50%, 60%, 70%, 80%, 85%, 90%, 95%, 96%, 97%, 98%, 99%, or higher sequence similarity to a member of the GNAT superfamily of N-acetyltransferases. In some of these embodiments, the candidate polypeptide has been shown to exhibit N-acetyltransferase activity or is suspected of having N-acetyltransferase activity (based on sequence similarity with other N-acetyltransferases). The candidate polypeptide can be suspected of having N-acetyltransferase activity if the candidate polypeptide exhibits at least 30%, 40%, 50%, 60%, 70%, 80%, 85%, 90%, 95%, 96%, 97%, 98%, 99%, or higher sequence similarity to a known N-acetyltransferase. In certain embodiments, the candidate polypeptide comprises a GLYAT polypeptide and the substrate comprises glyphosate.

A candidate polypeptide is considered to comprise the GNAT wedge joining region of the GLYAT polypeptide if the candidate polypeptide comprises a region wherein the back-bone atoms of the amino acids of this region have no more than about 4 Å root mean square deviation from the backbone atoms of the amino acids provided in Table 7, Tables 7 and 9, Table 8, or Tables 8 and 10, including but not limited to about 4 Å, 3.5 Å, 3 Å, 2.5 Å, 2.0 Å, 1.9 Å, 1.8 Å, 1.7 Å, 1.6 Å, 1.5 Å, 1.4 Å, 1.3 Å, 1.2 Å, 1.1 Å, 1.0 Å, 0.9 Å, 0.8 Å, 0.7 Å, 0.6 Å, 0.5 Å, 0.4 Å, 0.3 Å, 0.2 Å, and 0.1 Å. In some embodiments, the two molecular structures are considered the same if the root mean square deviation between the back-bone atoms of the amino acids of this region are no more than about 2 Å. Any method known in the art can be used to compare the two three-dimensional molecular structures to determine if the candidate polypeptide comprises the GNAT wedge joining region, including those described elsewhere herein.

It is to be noted that the candidate polypeptide can be considered to comprise the GNAT wedge joining region of the GLYAT polypeptide (comprising the atomic coordinates of Table 7, Tables 7 and 9, Table 8, or Tables 8 and 10) even lithe particular residue number between the GLYAT polypeptide and candidate polypeptide are dissimilar as long as the atomic coordinates of the amino acid atoms are the same (or wherein the back-bone atoms of the amino acids of this region have no more than about 4 Å root mean square deviation from the backbone atoms of the amino acids provided in Table 7, Tables 7 and 9, Table 8, or Tables 8 and 10, as discussed above). For example, the arginine residue at position 73 in the GNAT wedge joining region of the GLYAT R7 variant polypeptide listed in Table 9 can correspond to an arginine residue in the substrate binding cavity of the candidate polypeptide that is not at the 73^{rd} position in the amino acid sequence of the candidate polypeptide. One of skill in the art will appreciate that the two molecular structures can still be considered the same or similar as long as the three-dimensional molecular structure of the candidate polypeptide comprises the atomic coordinates within Table 9 (or a variation thereof), regardless of the positioning of a given residue with the polypeptide chain.

In some embodiments, the methods of the invention further comprise altering the primary structure of the candidate polypeptide to maximize a similarity or relationship between the three-dimensional molecular structures of the candidate polypeptide and the GNAT wedge joining region of the GLYAT polypeptide (comprising the atomic coordinates of Table 7, Tables 7 and 9, Table 8, or Tables 8 and 10). Any method known in the art can be used to alter the primary structure of the candidate polypeptide, including those described elsewhere herein. Candidate polypeptides whose primary structure have been modified to provide a better fit with the GNAT wedge joining region of the GLYAT polypeptide can be tested for the ability to acetylate its substrate at a higher catalytic rate when compared to a native GLYAT polypeptide using any method known in the art. In these embodiments, the catalytic rate will be determined under optimal conditions (e.g., non-limiting substrate). In this way, the methods of the invention provide for the identification of N-acetyltransferases that exhibit enhanced catalytic activity over native GLYAT polypeptides.

The methods can further comprise producing the candidate polypeptide having the GNAT wedge joining region described herein (comprising the atomic coordinates of Table 7, Tables 7 and 9, Table 8, or Tables 8 and 10). The candidate polypeptide can be synthesized using any method known in the art. The catalytic rate of the candidate polypeptide against a substrate (e.g., glyphosate) can then be assayed to determine if the candidate polypeptide has improved catalytic activity when compared to native GLYAT.

The presently disclosed subject matter further provides methods for evaluating the potential of a variant GLYAT polypeptide to associate with glyphosate with a higher binding affinity when compared to a native GLYAT polypeptide, higher binding specificity when compared to a native GLYAT polypeptide, or a combination thereof through the provision of a three-dimensional molecular structure of a variant GLYAT polypeptide. As described elsewhere herein, structural analysis of the altered amino acid residues between the optimized R11 and R7 variants compared with the native GLYAT identified three residue substitution trends associated with improved functionality; (1) increased positive charge through surface residue substitution, (2) expansion of the substrate binding cavity and (3) relaxation of the protein's interior packing density through downsizing amino acid substitution.

There are a total of 21 amino acid substitutions from the native GLYAT to the R7 variant, and 12 more from the R7 to R11 (FIG. 1, Tables 13-16). Based on structural location, the substitutions are divided into two groups, at the protein surface and in the interior. There are 10 surface substitutions from the native to R7 (G37R, R47G, K58Q, E65Q, E67Q, E68K, E92K, K 101R, E119K and K 144R) (Table 13) and 4 more from the R7 to R 11 (E14D, G38S, Q67K and K119R) (Table 15). The surface substitutions increase the protein's net positive charge by 7 from the native to R7 and by 1 more from the R7 to R11. Both the cofactor AcCoA and glyphosate are heavily negatively charged species, and therefore the enhanced positive charge in the optimized GLYAT variants may increase the attraction to its substrates, which in turn may accelerate catalysis. The surface substitutions might also result in part from pressure during shuffling to select variants with improved expression in E. coli and solubility in buffer.

Of the interior substitutions, only 4, Y31F-V114A-I132T-I135V, are at the active site and they are all downsizing changes, i.e. residues with larger side-chain are replaced by relatively smaller ones (Table 14). V114A makes a direct contact with the pantetheine motif of AcCoA. I132T and I135V are located at the β-hairpin and interact with glyphosate's phosphono group. Y31F directly contacts the substrate carboxyl group through a van der Waals attraction in R7 and/or a hydrogen bond in the native GLYAT. These four substitutions effectively increase the size of the substrate binding-site. As described earlier (Siehl et al. (2007) J Biol Chem 282:11446-11455), the substrate most active with native GLYAT is D-AP3 (FIG. 2B). Considering that glyphosate is longer than D-AP3, the resulting larger active site in the optimized GLYATs better accommodate glyphosate, thus increasing catalytic efficiency and specificity to glyphosate.

Besides the four substitutions at the active site, other interior substitutions show the same downsizing trend, totaling 7 from the native to R7 (Y31F, T33S, T89S, V 114A, Y130F, I132T and I135V, Table 14) and 6 more substitutions from the R7 to R11 (119V, L36T, Y45F, 153V, M75V and 191V, Table 16). As a consequence, the overall molecular weight of R7 was 90 units smaller, 16,600 Da (R7) vs. 16,690 Da (native). These downsizing substitutions systematically created numerous small cavities, as with T33S and M75V, or abolished some internal hydrogen bonds, such as Y45F and Y130F, in the protein core, relaxing the protein's packing density. It is well documented that structural flexibility is inversely related to packing density (Halle (2002) Proc. Natl. Acad. Sci. USA 99:1274-1279). Mutagenesis and theoretical approaches have shown that introducing new interior cavities in some instances may decrease a protein's thermal stability (Matsumura et al. (1988) Nature, 334, 406-410; Eriksson et al (1992) Science, 255, 17K-183; Xu et al. (1998) Protein Sci. 7(1):158-177). On the other hand, in some instances, filling cavities can inhibit the motion of functionally important regions of a protein, thereby diminishing its catalytic activity (Ogata et al., (1996). Nat. Struct. Biol., 3, 178-187). Thus, the greater flexibility of optimized GLYATs is important for its improved functionality.

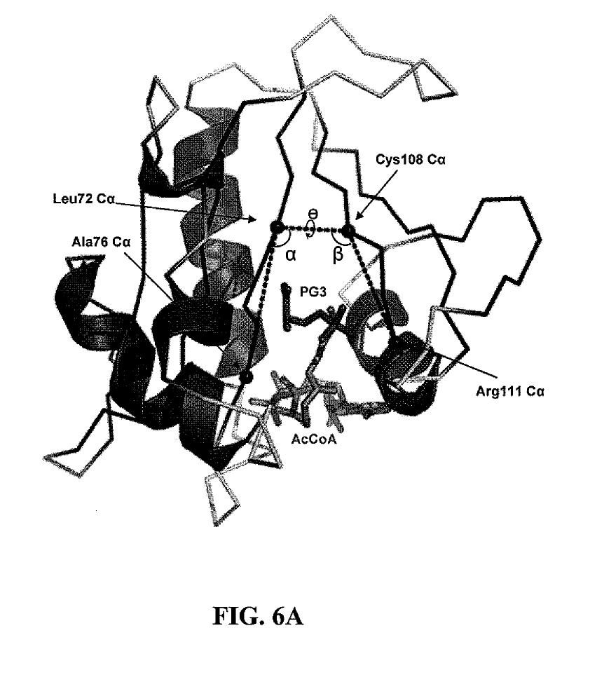

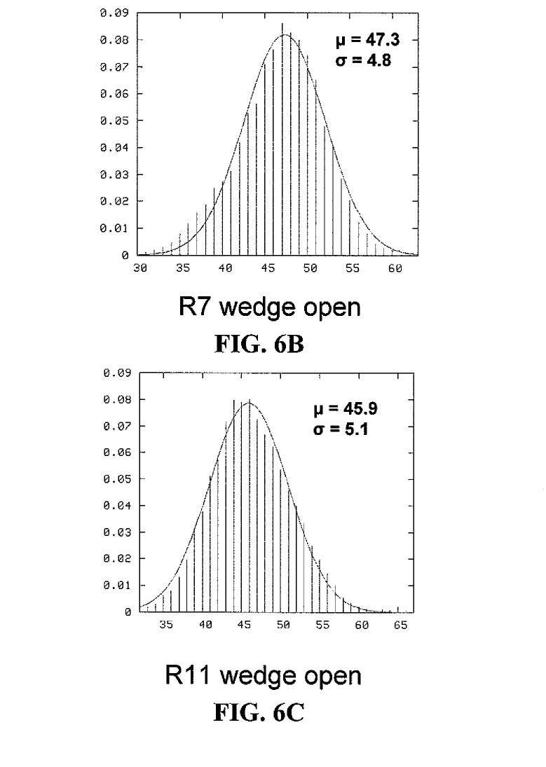

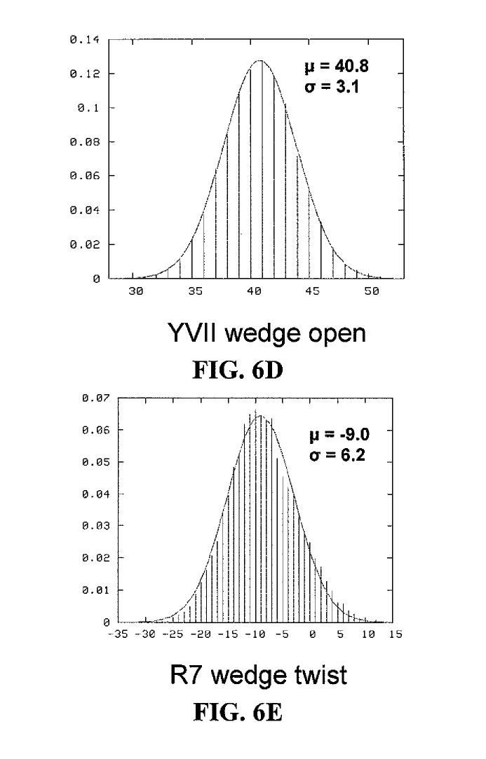

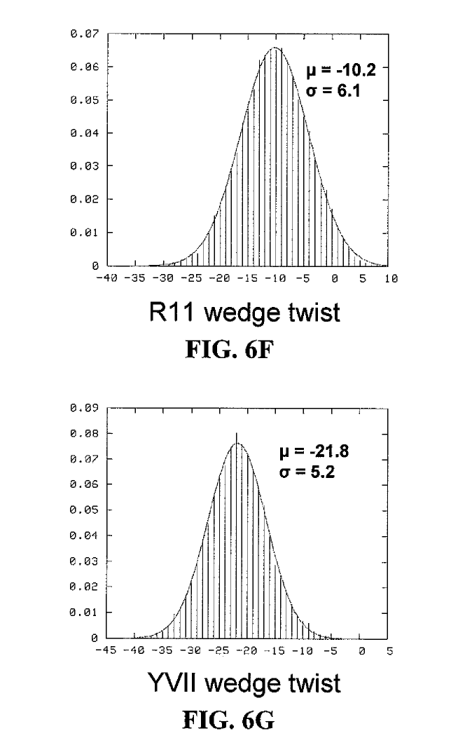

The GLYANT variant's structural characteristics in the absence of both substrate and cofactor AcCoA can be studied by a molecular dynamics simulation of an unliganded apo-enzyme. Without the bound ligands, the protein undergoes a large and hinge-like subdomain motion along the V-shaped wedge, and consequently the binding cavities for both substrate and cofactor are wide open. The binding site openness can be measured by calculating the average wedge angle and by measuring an inter-loop distance of the substrate binding loops, the β-hairpin and loop20. As used herein, a “wedge angle” is defined by the formula α+β−180°, wherein a comprises the angle formed by the Cα carbons in the following amino acid residues: alanine at position 76, leucine at position 72 and cysteine at position 108; and wherein β comprises the angle formed by the Cα carbons in the following amino acid residues: leucine at position 72, cysteine at position 108, and arginine at position 111 (see FIG. 6A). In some embodiments, an average wedge angle of at least about 41″, including but not limited to about 42°, 43°, 44°, 45°, 46°, 47°, 48°, 49°, 50°, 51°, 52°, 53°, 54°, 55° or greater indicates the variant GLYAT polypeptide associates with glyphosate with a higher binding affinity, higher binding specificity or both when compared to a native GLYAT polypeptide. The distance between the substrate-binding beta hairpin and loop20 is determined by two alpha carbons of Gln24 and Pro134 (FIG. 4). A distance between the alpha carbons of Gln24 and Pro 134 of greater than about 14 Å indicates that the active site of the polypeptide is in an open state. Compared to D-AP3 with 4 main-chain atoms, glyphosate has 5 main-chain atoms and thus is a larger and longer molecule. Therefore, a variant GLYAT polypeptide capable of opening its substrate binding site wider is associated with a higher binding affinity or higher binding specificity to glyphosate when compared to a native GLYAT polypeptide (FIG. 4B). In some embodiments, an average interloop distance of about 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, or greater indicates the variant GLYAT polypeptide associates with glyphosate with a higher binding affinity, specificity, or both when compared to a native GLYAT polypeptide.

As used herein, a “molecular dynamics simulation” refers to a simulation method devoted to the calculation of the time dependent behavior of a molecular system in order to investigate the structure, dynamics and thermodynamics of molecular systems by solving the equation of motion for a molecule. This equation of motion provides information about the time dependence and magnitude of fluctuations in both positions and velocities of a given molecule. The direct output of molecular dynamics simulations is a set of “snapshots” (coordinates and velocities) taken at equal time intervals, or sampling intervals. Depending on the desired level of accuracy, the equation of motion to be solved may be the classical (Newtonian) equation of motion, a stochastic equation of motion, a Brownian equation of motion, or even a combination (Becker et al. (2001) cds. Computational Biochemistry and Biophysics New York). There are a number of ways to implement molecular dynamics simulations and examples of suitable simulation packages include, but are not limited to, CHARMM 983) J Comp. Chem. 4:187-217), AMBER ((2005) J. Computat. Chem. 26:1668-1688), GROMACS (van der Spoel et al. (2005) J Comp. Chem. 26:1701-1718, TINKER (Ponder et al. (1987) J. Comput. Chem. 8:1016-1024), NAMD (Phillips et al. (2005) J. Comput. Chem. 26:1781-1802) and LAMMPS (Plimpton (1995) J. Comp. Phys. 117:1-19). Any method known in the art for performing a molecular dynamics simulation can be used, including the methods described elsewhere herein (see Experimental section). For example, CHARMM 27 (MacKerell et al. (2004) Journal of Computational Chemistry 25:1400-1415) or GROMACS simulations, OPLS-AA/L (Jorgensen et al. (1996) J. Am. Chem. Soc. 118: 11225-11236; Kaminski et al. (2001) J. Phys. Chem. 105:6474-6487) can be performed.

The sampling interval (that is, the duration of the molecular dynamics trajectory) is determined according to the time scale of the protein motion to be sampled. In some embodiments of the presently disclosed methods, the sampling interval of the molecular dynamics simulation is about 0.1, 1, 2, 4, 6, 8, 10, 15, 20, 30, 40, 50, 60, 70, 80, 90, 100, 500 nanoseconds or greater. In some of these embodiments, the molecular dynamics simulation occurs over an interval of about 10 nanoseconds. The average wedge angle of the GNAT wedge of the variant GLYAT polypeptide is determined over the specified sampling interval. In certain embodiments, the maximal wedge angle over an entire sampling interval of a molecular simulation of at least about 41°, including but not limited to about 42′, 43°, 44°, 45°, 46°, 47°, 48°, 49°, 50°, 51″, 52°, 53″, 54°, 55° or greater indicates the variant GLYAT polypeptide associates with glyphosate with a higher binding affinity, higher binding specificity or both when compared to a native GLYAT polypeptide.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4: 11-17; the local alignment algorithm of Smith et al., (1981) Adv. Appl. Math. 2:482; the global alignment algorithm of Needleman and Wunsch (1970) J. Mol. Biol. 48: 443-453; the search-for-local alignment method of Pearson and Lipman (1988) Proc. Natl. Acad. Sci. 85: 2444-2448; the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877.

Computer implementations of these mathematical algorithms can be utilized for comparison of sequences to determine sequence identity. Such implementations include, but are not limited to: CLUSTAL in the PC/Gene program (available from Intelligenctics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73: 237-244 (1988); Higgins et al. (1989) CABIOS 5: 151-153; Corpct et al. (1988) Nucleic Acids Res. 16:10881-90; Huang et al. (1992) CABIOS 8: 155-65; and Pearson et al. (1994) Meth. Mol. Biol. 24: 307-331. The ALIGN program is based on the algorithm of Myers and Miller (1988) supra. A PAM 120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences. BLASTX for proteins) can be used. BLAST software is publicly available on the NCBI website. Alignment may also be performed manually by inspection.

In some embodiments in the present methods, some steps, preferably the determining step can be implemented by a machine whereas the evaluation or evaluating step is conducted by a person. Computer programs disclosed herein or known in the art for comparing three-dimensional molecular structures are suitable for the present methods. More specifically, the one or more steps are implemented by a machine-readable program code on a machine readable medium and configured for execution by a machine such as a computer. General purpose machines may be used with the programs described herein or other suitable programs for executing one or more steps of the presently described methods. However, preferably embodiments are implemented in one or more computer programs executing on programmable systems each comprising at least one processor, at least one data storage system (including volatile and non-volatile memory and/or storage elements), at least one input device, and at least one output device. The program is executed on the processor to perform the functions described herein.

Each such program may be implemented in any desired computer language (including machine, assembly, high level procedural, object oriented programming languages, or the like) to communicate with a computer system. In any case, the language may be a compiled or interpreted language. The computer program will typically be stored on a storage media or device (e.g., ROM, CD-ROM, or magnetic or optical media) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

As used herein, the phrase “computer-readable storage medium” refers to any medium or mechanism for storing or transmitting information in a form readable by a machine (e.g., a computer). For example, a machine readable medium includes machine readable storage media (read only memory (“ROM”); random access memory (“RAM”); magnetic disk storage media; optical storage media; flash memory devices); machine readable transmission media (electrical, optical, acoustical or other form of propagated signals, e.g., carrier waves, infrared signals, digital signals, etc.); floppy disks, optical disks, CD-ROMs, and magnetic-optical disks, EPROMs, EEPROMs, magnetic or optical cards, or any type of media suitable for storing electronic instructions.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89: 10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

It is to be noted that the term “a” or “an” entity refers to one or more of that entity; for example, “a polypeptide” is understood to represent one or more polypeptides. As such the terms “a” (or “an”),“one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### Example 1

### Structural Analysis and Molecular Dynamics Simulation of glyphosate N-acetyltransferase

Optimized variants of glyphosate N-accetyltransferase (GLYAT) from B. licheniformis efficiently catalyze the acetylation of glyphosate, a broad-spectrum and non-selective herbicide, and confer resistance in transgenic plants. Structural modeling and molecular dynamics (MD) simulations were performed on the native enzyme, 7^{th} (R7) and 11^{th} (R11) round variants from DNA shuffling experiments (Keenan et al. (2005) Proc Natl Acad Sci USA 102(25):8887-8892), and a revertant form of R7 in which all four active site substitutions were changed back to the wild type form (YVII). Structural analysis revealed that the efficiency enhancement of the shuffling variants coincided with interior bulky residues being mutated to smaller ones. Substitutions that exemplify that trend in evolving native GLYAT to R7 include Y31F, T33S, T89S, V114A, I132T, Y130F and I135V; and from R7 to R11, 119V, L36T, Y45F, 153V, M75V, 191V. MD simulations showed that the more optimized GLYAT roughly had a larger amplitude of fluctuation and inter-subdomain motion, supporting the hypothesis that the interior downsizing mutations reduced the enzyme's core packing strength, resulting in more flexibility. Two major substrate binding elements, loop20 connecting the α1 and α2 helices and the β-hairpin connecting the β6 and β7 strands, were the most flexible. In the absence of ligand, loop20 and the β-hairpin drift more than 16 Å apart from their closed form when bound to ligand. The β-hairpin, containing a type Via β turn and two downsizing mutations I132V and I135T, apparently plays a role in regulating the active site conformation and determining substrate specificity. The Principal Component Analysis of a MD trajectory identified two novel, independent inter-subdomain motion modes involving the signature v-shaped wedge: wedge opening and wedge twisting. These long range motions might be a unique feature of the GCN5-related N-acetyltransferase (GNAT) superfamily fold and could be useful in understanding GNAT's structure-function relationship.

X-ray crystal structures of R7 GLYAT (from the 7^{th} round of gene shuffling) complexed with AcCoA and 3-phosphoglycerate (3PG), a competitive inhibitor with respect to glyphosate, revealed the active site architecture. See PDB:2JDD for the atomic coordinates and structure factors of the X-ray crystal structure of the ternary complex of R7 GLYAT with AcCoA and 3PG and PDB:2JDC for the atomic coordinates and structure factors of the X-ray crystal structure of the binary complex of R7 GLYAT with oxidized CoA and sulfate bound in the glyphosate binding pocket. See Tables 11 and 12 for the atoms of the R7 GLYAT variant polypeptide and of AcCoA that contact 3PG (i.e., the substrate binding cavity) and the residues of R7 that contact AcCoA, respectively.

TABLE 11
Contacts between the R7 GLYAT variant polypeptide and 3PG and
AcCoA when the polypeptide is bound to AcCoA and 3PG.

| | | GLYAT | | | | | |
|---|---|---|---|---|---|---|---|
| | | or | | | | | |
| Residue | Amino | AcCoA | | | | 3PG^{c} | Distance |
| ID | Acid | Atom^{a} | X^{b} | Y^{b} | Z^{b} | Atom | (A) |
| 20 | LEU | CB | 24.62 | 9.03 | 11.645 | O3 | 3.69 |
| | | CD1 | 24.57 | 6.61 | 10.99 | C2 | 3.46 |
| | | | | | | C3 | 3.93 |
| | | | | | | O3 | 3.86 |
| 21 | ARG | CD | 28.10 | 9.68 | 15.24 | O3 | 3.85 |
| | | CG | 27.02 | 10.61 | 14.71 | O3 | 3.90 |
| | | CZ | 27.41 | 8.40 | 17.27 | O1 | 3.84 |
| | | | | | | O3 | 3.54 |
| | | NE | 27.59 | 8.51 | 15.95 | O1 | 3.88 |
| | | | | | | O3 | 2.96 |
| | | NH2 | 26.96 | 7.25 | 17.77 | C1 | 3.97 |
| | | | | | | O1 | 2.90 |
| | | | | | | O3 | 3.46 |
| 31 | PHE | CE1 | 29.29 | 5.50 | 15.09 | O1 | 3.64 |
| | | CE2 | 28.33 | 3.62 | 13.99 | C1 | 3.58 |
| | | | | | | O1 | 3.62 |
| | | | | | | O2 | 3.56 |
| | | CZ | 28.33 | 4.97 | 14.26 | C1 | 3.31 |
| | | | | | | C2 | 3.80 |
| | | | | | | O1 | 3.15 |
| | | | | | | O2 | 3.76 |
| | | | | | | O3 | 3.79 |
| 73 | ARG | C | 24.93 | 0.91 | 12.48 | O2 | 3.66 |
| | | CA | 24.71 | 0.21 | 13.81 | O2 | 3.57 |
| | | CB | 26.01 | 0.22 | 14.60 | O2 | 3.55 |
| | | CZ | 27.35 | 2.30 | 17.81 | O1 | 3.49 |
| | | | | | | O2 | 3.95 |
| | | NE | 26.50 | 1.62 | 17.06 | C1 | 3.86 |
| | | | | | | O1 | 3.57 |
| | | | | | | O2 | 3.26 |
| | | NH2 | 27.36 | 3.61 | 17.80 | C1 | 3.55 |
| | | | | | | O1 | 2.58 |
| | | | | | | O2 | 3.73 |
| 74 | GLY | CA | 25.28 | 3.01 | 11.31 | O2 | 3.74 |
| | | N | 24.87 | 2.24 | 12.48 | C1 | 3.76 |
| | | | | | | O2 | 2.84 |
| 111 | ARG | CD | 18.73 | 8.66 | 14.12 | O2P | 3.59 |
| | | CG | 18.18 | 7.71 | 15.15 | O2P | 3.23 |
| | | CZ | 20.84 | 9.92 | 13.92 | O2P | 3.61 |
| | | | | | | O3P | 3.77 |
| | | NE | 20.13 | 8.92 | 14.42 | O2P | 2.76 |
| | | | | | | O3P | 3.79 |
| | | | | | | P | 3.80 |
| | | NH2 | 22.10 | 10.05 | 14.28 | O2P | 3.60 |
| | | NH2 | 22.10 | 10.05 | 14.28 | O3P | 2.86 |
| | | NH2 | 22.10 | 10.05 | 14.28 | P | 3.80 |
| 135 | VAL | CB | 22.07 | 11.45 | 17.93 | O3P | 3.75 |
| | | CG1 | 23.25 | 12.06 | 17.19 | O3P | 3.78 |
| | | CG2 | 22.54 | 10.38 | 18.90 | O2P | 3.97 |
| | | | | | | O3P | 3.46 |
| | | | | | | O4P | 3.37 |
| | | | | | | P | 3.78 |
| 138 | HIS | CD2 | 18.83 | 6.13 | 18.81 | O2P | 3.43 |
| | | CE1 | 19.22 | 8.25 | 19.22 | O2P | 3.28 |
| | | | | | | O4P | 3.98 |
| | | NE2 | 19.56 | 7.23 | 18.44 | O2P | 2.39 |
| | | | | | | O4P | 3.35 |
| | | | | | | P | 3.43 |
| * | AcCoA | CH3 | 20.65 | 3.43 | 15.29 | O1P | 3.47 |
| | | | | | | C3 | 3.65 |
| | | C | 20.37 | 3.37 | 13.84 | C3 | 3.90 |
| | | O | 21.28 | 2.95 | 13.05 | C3 | 3.91 |

^{a}The amino acid atom is the specific atom of the amino acid, as identified in Protein Data Bank file 2JDD;
^{b}X, Y, and Z are the three-dimensional coordinates specifying the distance in Angstroms of the amino acid atom relative to the center of mass of the crystal;
^{c}Atoms of 3PG or AcCoA are defined in PDB:2JDD and FIG. 2.

TABLE 12
Contacts between the R7 GLYAT variant polypeptide and AcCoA
when the polypeptide is bound to AcCoA and 3PG.

| Residue | Amino | GLYAT | | | | AcCoA | Distance |
|---|---|---|---|---|---|---|---|
| ID | Acid | Atom | X | Y | Z | Atom | (A) |
| 19 | ILE | CG2 | 22.17 | 10.01 | 7.13 | C6P | 3.82 |
| | | | | | | C7P | 3.97 |
| 20 | LEU | CD2 | 22.43 | 7.90 | 11.10 | C6P | 3.71 |
| | | | | | | N4P | 3.95 |
| 74 | GLY | N | 24.87 | 2.24 | 12.48 | O | 3.70 |
| 75 | MET | C | 21.88 | 3.82 | 8.56 | N4P | 3.99 |
| | | CB | 20.92 | 1.81 | 9.66 | O | 3.59 |
| | | CG | 19.81 | 1.60 | 8.65 | CDP | 3.97 |
| | | N | 23.15 | 2.62 | 10.25 | O | 3.38 |
| | | O | 21.33 | 4.67 | 9.24 | C2P | 3.68 |
| | | | | | | C3P | 3.27 |
| | | | | | | C5P | 4.00 |
| | | | | | | N4P | 2.86 |
| 76 | ALA | C | 21.95 | 5.23 | 5.15 | O9P | 3.61 |
| | | CA | 22.02 | 5.34 | 6.66 | O9P | 3.41 |
| 77 | THR | CA | 21.01 | 6.18 | 3.10 | O9P | 3.84 |
| | | CB | 19.73 | 5.56 | 2.52 | O5A | 3.53 |
| | | | | | | O6A | 3.69 |
| | | CG2 | 19.68 | 5.75 | 1.02 | O5A | 3.61 |
| | | N | 21.07 | 6.03 | 4.55 | C9P | 3.88 |
| | | | | | | CDP | 3.89 |
| | | | | | | O9P | 2.85 |
| | | O | 20.43 | 8.45 | 3.43 | O9P | 3.35 |
| | | OG1 | 19.70 | 4.16 | 2.82 | CDP | 3.96 |
| | | | | | | O5A | 2.94 |
| | | | | | | O6A | 3.25 |
| | | | | | | P2A | 3.71 |
| 82 | ARG | C | 14.99 | 6.70 | −1.05 | O4A | 3.58 |
| | | CA | 16.21 | 7.12 | −0.26 | O4A | 3.28 |
| | | CB | 15.80 | 8.04 | 0.89 | O4A | 3.23 |
| | | CD | 16.66 | 9.38 | 2.84 | OAP | 3.59 |
| | | CG | 16.98 | 8.42 | 1.74 | O4A | 3.74 |
| | | CZ | 18.06 | 10.62 | 4.46 | C7P | 3.98 |
| | | | | | | C9P | 3.84 |
| | | | | | | N8P | 3.79 |
| | | NE | 17.90 | 9.67 | 3.55 | C9P | 3.74 |
| | | | | | | CAP | 3.87 |
| | | | | | | O9P | 3.91 |
| | | | | | | OAP | 3.79 |
| | | NH2 | 19.23 | 10.77 | 5.06 | C7P | 3.57 |
| | | | | | | C9P | 3.88 |
| | | | | | | N8P | 3.81 |
| | | | | | | O9P | 3.73 |
| 83 | GLU | C | 13.15 | 4.46 | −2.27 | O1A | 3.60 |
| | | CA | 12.95 | 5.32 | −1.03 | O1A | 3.38 |
| | | | | | | O4A | 3.94 |
| | | CD | 11.34 | 7.10 | 1.00 | O2B | 3.81 |
| | | N | 14.20 | 5.81 | −0.44 | O1A | 3.45 |
| | | | | | | O4A | 2.88 |
| | | OE1 | 10.53 | 6.16 | 1.02 | C2B | 3.26 |
| | | OE1 | 10.53 | 6.16 | 1.02 | O2B | 2.72 |
| 84 | GLN | N | 14.33 | 3.85 | −2.38 | O1A | 3.38 |
| 85 | LYS | C | 16.43 | −0.32 | −0.95 | O2A | 3.68 |
| | | CA | 14.92 | −0.07 | −1.18 | O1A | 3.41 |
| | | | | | | O2A | 3.32 |
| | | | | | | P1A | 3.85 |
| | | CD | 12.16 | −1.38 | −0.55 | O2A | 3.94 |
| | | CE | 10.64 | −1.33 | −0.46 | O7A | 3.32 |
| | | N | 14.62 | 1.24 | −1.80 | O1A | 2.94 |
| | | | | | | O2A | 3.96 |
| | | | | | | P1A | 3.92 |
| | | NZ | 10.04 | −0.22 | −1.23 | O7A | 3.07 |
| | | NZ | 10.04 | −0.22 | −1.23 | O9A | 3.91 |
| 86 | ALA | C | 18.81 | −0.004 | 0.80 | O5A | 3.90 |
| | | CB | 19.40 | 1.83 | −0.78 | O5A | 3.76 |
| | | N | 17.23 | 0.74 | −0.94 | O5A | 3.76 |
| 87 | GLY | C | 17.79 | −1.18 | 3.35 | O2A | 3.92 |
| | | CA | 18.38 | 0.20 | 3.16 | O5A | 3.69 |
| | | N | 18.24 | 0.68 | 1.78 | O2A | 3.80 |
| | | | | | | O5A | 2.92 |
| 88 | SER | CA | 15.92 | −2.72 | 2.95 | O2A | 3.58 |
| | | CB | 14.56 | −2.71 | 2.24 | O2A | 3.15 |
| | | N | 16.59 | −1.41 | 2.82 | O2A | 2.91 |
| | | OG | 13.62 | −1.90 | 2.92 | C5B | 3.31 |
| | | | | | | O2A | 2.62 |
| | | | | | | P1A | 3.89 |
| 109 | ASN | C | 18.24 | 2.21 | 18.08 | CH3 | 3.88 |
| | | O | 19.06 | 3.04 | 17.65 | CH3 | 2.87 |
| 110 | ALA | CA | 16.52 | 2.87 | 16.46 | S1P | 3.98 |
| 111 | ARG | CD | 18.73 | 8.66 | 14.12 | C2P | 3.93 |
| | | | | | | O5P | 3.62 |
| | | CG | 18.18 | 7.71 | 15.15 | C2P | 3.97 |
| | | N | 16.68 | 5.25 | 16.01 | S1P | 3.63 |
| 113 | SER | C | 12.37 | 7.99 | 12.27 | N6A | 3.85 |
| | | O | 11.81 | 8.18 | 11.20 | C6A | 3.59 |
| | | | | | | N1A | 3.49 |
| | | | | | | N6A | 2.90 |
| 114 | ALA | CA | 13.18 | 5.76 | 11.62 | N6A | 3.63 |
| | | CB | 14.67 | 5.37 | 11.48 | C3P | 3.93 |
| | | | | | | O5P | 3.96 |
| 116 | GLY | C | 9.20 | 2.31 | 9.59 | C2A | 3.62 |
| | | | | | | N3A | 3.59 |
| | | CA | 8.48 | 3.11 | 10.65 | C2A | 3.58 |
| | | O | 8.62 | 1.42 | 8.96 | N3A | 3.99 |
| 117 | TYR | CA | 11.32 | 1.88 | 8.44 | C4A | 3.81 |
| | | | | | | N3A | 3.94 |
| | | | | | | O4B | 3.82 |
| | | CB | 12.63 | 2.63 | 8.24 | C4A | 3.76 |
| | | | | | | C5A | 3.66 |
| | | | | | | C8A | 3.93 |
| | | | | | | N7A | 3.80 |
| | | | | | | N9A | 3.93 |
| | | CD1 | 13.82 | 1.53 | 6.28 | C5B | 3.79 |
| | | | | | | C8A | 3.93 |
| | | | | | | CCP | 3.73 |
| | | | | | | O3A | 3.70 |
| | | | | | | O4B | 3.72 |
| | | | | | | O5B | 3.69 |
| | | CD2 | 14.90 | 1.52 | 8.41 | CEP | 3.91 |
| | | CE1 | 14.90 | 0.87 | 5.73 | CCP | 3.72 |
| | | | | | | O2A | 3.71 |
| | | | | | | O3A | 3.33 |
| | | | | | | O5B | 3.87 |
| | | | | | | P1A | 3.94 |
| | | N | 10.47 | 2.64 | 9.36 | C2A | 3.50 |
| | | | | | | C4A | 3.76 |
| | | | | | | N1A | 3.87 |
| | | | | | | N3A | 3.44 |
| 118 | TYR | CE2 | 15.86 | 1.53 | 12.30 | S1P | 3.87 |
| | | OH | 18.10 | 1.23 | 11.51 | C | 3.89 |
| | | | | | | O | 3.93 |
| | | | | | | S1P | 3.31 |
| 120 | LYS | CD | 6.24 | −0.19 | 5.34 | O8A | 3.67 |
| | | CE | 5.18 | 0.92 | 5.37 | O8A | 3.50 |
| | | NZ | 5.57 | 2.18 | 4.66 | O3B | 3.42 |
| | | | | | | O8A | 2.82 |
| | | | | | | P3B | 3.83 |

^{a}The name convention and structure are the same as in Table 11.

In the ternary complex, 3PG sits on a platform defined by the pseudo-β sheet of the two splaying β4 and β5 strands and the pantetheine moiety of the cofactor, with the main-chain of 3PG perpendicular to the β-strands. The inhibitor is covered by two tip-joining loops, loop20 connecting α1/α2 and loop130 (or n-hairpin) spanning β6/β7. Surprisingly, the 21 amino-acid differences between the R7 and wild-type GLYAT are almost evenly distributed across the entire structure; none of the 3PG ligation residues—L20, Arg21, Gly74, Arg73, Arg111, and His138—are altered; and only four amino acid differences are in the perimeter of the active site, with Y31F, I132T, and I135V near 3PG and V114A close to AcCoA (Siehl et al. (2007) J Biol Chem 282:11446-11455). On the other hand, it has been documented that mutations distal to the active site can affect protein functions such as drug resistance (Perryman et al. (2004) Protein Sci 13:1108-1123), allosteric regulation (Taly et al. (2006) Proc. Natl. Acad. Sci. USA 103(45):16965-16970; Berendsen & Hayward (2000) Curr Opin Struct Biol 10(2):165-169), and ligand binding specificity (Ma et al. (2005) Biophysical Journal 89:1183-1193), often through long range correlated motion or conformational changes (Ma et al. (2002) Protein Sci 11:184-197). Thus, investigating GLYAT's dynamic characteristics and conformational flexibility is crucial to understanding the mechanism of its functional evolution and to further facilitate new herbicide tolerant gene development. Provided herein is a structural modeling and/or molecular dynamics (MD) study on the 7^{th} round (R7), the 11^{th} round (R11), YVII, and wild type GLYAT in various ligation states. YVII is a revertant mutant in which the four substitutions near the active site of R7 (Y31, V114, I132 and I135) were mutated back to wild-type. In fully liganded complex MD simulations, glyphosate, 3PG, or D-AP3 were modeled separately to examine the intimate details of the interaction between ligand and the enzymatic active site. To verify the findings, some simulations were carried out on two independent platforms, CHARMm 31b1 with CHARMM 27 force field and Gromacs with OPL-AA. All the simulations were performed in explicit solvent for multiple nanoseconds. This study characterized a novel open conformation, a transition mechanism between an open and closed active site, and inter-subdomain hinge motions around the wedge, and showed that the activity enhancement resulting from shuffling correlated with decreased protein core packing density or increased structural fluctuation. This is the first major simulation study applied to a member of the GNAT superfamily.

Analysis of Shuffling Changes through Structure Modeling:

Structure models of R11 and native GLYAT with bound ligands were built based on the crystal structure of R7 GLYAT complexed with AcCoA and 3PG (Siehl et al. (2007) J Biol Chem 282:11446-11455). After a series of energy minimizations under various constraints, the resulting models were similar to the R7 structure with RMSDs of <0.9 Å over all Cα atoms. MD simulations in explicit solvent were applied to further relax any outstanding strains. Harmonic constraints on heavy atoms in the protein were applied for the first 300 ps, followed by free simulation for the next >500 ps. In the presence of ligands, the models remained stable over the course of the simulations and the trajectory RMSDs of heavy atoms over the initial structures were comparable to those observed in R7 GLYAT, suggesting that the models were reasonably accurate.

The complete atomic coordinates of the GLYAT R7 variant bound to acetyl coA and glyphosate can be found in Table 18, whereas the complete atomic coordinates of the GLYAT R11 variant bound to acetyl coA and glyphosate are provided in Table 19.

Between the native GLYAT and the R7 variant, there are a total of 21 amino acid substitutions (FIG. 1A, Tables 13 and 14). Based on the solvent accessibility, hydrophobicity, and interactions with other residues, these amino acid changes were divided into two categories: ten surface mutations: G37R, R47G, K58Q, E65Q, E67Q, E68K, E92K, K101R, E119K and K144R (Table 13); and 11 interior mutations: 115L, L261, Y31F, T33S, T89S, L971, V114A, Y130F, I132T, I135V and L14.51 (Table 14). All ten surface mutations were hydrophilic substitutions including 3 R/K, 3 E/K, 2 E/Q, and 2 G/R switches. None of these mutations were close to the active site and seven of them were clustered at the vertex of the V-shaped wedge, the farthest location from bound glyphosate in the structure. These cluster mutations mainly occurred in loops, including G37R at the α2/β2 loop, K58Q, E65Q, E67Q and E68K at the β3/β4 loop, E92K at the α3/β4 loop, and K144R near the C-terminus. These localized mutations increased the cluster's net positive charge by four and therefore altered the protein's electric dipole. In total, R7 GLYAT gained 7 net positive charges compared to the native GLYAT. Considering that both the cofactor AcCoA and glyphosate are heavily negatively charged species, the enhanced positive charge of R7 GLYAT may increase the attraction to its substrates. Overall, the mutations improved the protein's surface physical characteristics and allowed the R7 GLYAT in the presence of ligands to be easily crystallized to diffraction-quality, which was difficult to achieve with native protein (Keenan et al. (2005) Proc. Natl. Acad. Sci. USA 102(25):8887-8892). Thus, the surface substitutions might result in part from pressure during shuffling to select variants with improved expression in E. coli and solubility in buffer.

TABLE 13
Substitution of surface residues from native GLYAT to the R7 GLYAT variant polypeptide.

| | Substitution | | | | | | | | | | |
|---|---|---|---|---|---|---|---|---|---|---|---|
| | G37R | R47G | K58Q | E65Q | E67Q | E68K | E92K | K101R | E119K | K144R | Total |
| Δcharge | +1 | −1 | −1 | +1 | +1 | +2 | +2 | 0 | +2 | 0 | +7 |

Regarding the 11 interior mutations, four of them were simply isomer switches between Leu and Ile (I15L, L261, L97I, and L145I) that are unlikely to alter catalytic efficiency in a significant way. Strikingly, the other 7 buried or partially buried substitutions all showed a clear trend that the larger residues of the native protein were replaced by smaller ones in R7: Y31F, T33S, T89S, V114A, Y130F, I132T, and I135V (Table 14). As a consequence, the overall molecular weight of R7 was 90 units smaller, 16,600 Da (R7) vs. 16,690 Da (native). Of these downsizing substitutions, Y31F, V114A, I132T and I135V are at the active site. V114A makes direct contact with the pantetheine motif of AcCoA. I132T and I135V are located at the glyphosate binding β-hairpin while Y31F directly contacts the substrate through either a hydrogen bond in the native or a van der Waals attraction in R7. These four substitutions effectively increase the size of the enzyme's substrate binding site. As described earlier (Siehl et al. (2007) J Biol Chem 282: 11446-11455), the substrate most active with native GLYAT is D-AP3 (FIG. 2B). Considering that glyphosate is longer than D-AP3, the resulting larger active site of R7 GLYAT could better accommodate glyphosate. Indeed, in vitro assays demonstrated that YVII GLYAT has substrate specificity similar to that of the native enzyme, preferring D-AP3 over glyphosate (data not shown). T33S, in helix 2a near F32(R7), hydrogen bonds to the side chain of Arg73 which, in turn, directly interacts with glyphosate. Based on the model, the methyl group of T33 in the native enzyme stacks against the imidazole ring of H57, and the lack of this methyl group in R7 attenuated the contact strength, presumably fine tuning the active site conformation. The T89S substitution occurred in the helix α3 and the methyl group in native GLYAT was well buried, making hydrophobic interactions with the side chains of L90, V4, and L2. Residue Y130V is part of the substrate binding β-hairpin and its phenol in the native enzyme hydrogen bonds with the side chain of Asn 109. Loss of it in optimized GLYAT variants allows the β-hairpin to easier adjust its conformation to accommodate glyphosate. Interestingly, other homologous sequences all have phenylanine at this position, suggesting that native GLYAT might be uniquely selected for its native substrate (Siehl et al. (2007) J Biol Chem 282:11446-11455).

TABLE 14
Substitution of interior residues from native GLYAT to the R7 GLYAT polypeptide.

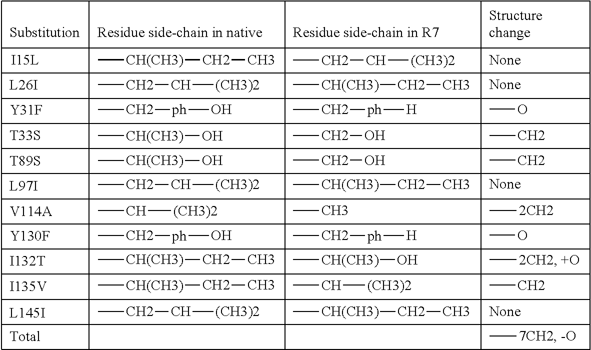

* The shaded rows are residues in the active site;
ph: phenol

TABLE 15
Substitution of surface residues from the R7 GLYAT
variant polypeptide to the R11 variant polypeptide.

| Substitution | E14D | G38S | Q67K | K119R | Total |
|---|---|---|---|---|---|
| Δcharge | 0 | 0 | + 1 | 0 | +1 |

TABLE 16
Substitution of interior residues from the R7 GLYAT
variant polypeptide to the R11 variant polypeptide.

| | | | Structure |
|---|---|---|---|
| Substitution | Residue side-chain in R7 | Residue side-chain in R11 | change |
| I19V | —CH(CH3)—CH2—CH3 | —CH—(CH3)2 | —CH2 |
| L36T | —CH2—CH—(CH3)2 | —CH(CH3)—OH | —2CH2, +O |
| Y35F | —CH2—ph—OH | —CH2—ph—H | —O |
| I53V | —CH(CH3)—CH2—CH3 | —CH—(CH3)2 | —CH2 |
| M75V | —(CH2)2—S—CH3 | —CH—(CH3)2 | —CH2, −S |
| I91V | —CH(CH3)—CH2—CH3 | —CH—(CH3)2 | —CH2 |
| L105M | —CH2—CH—(CH3)2 | —(CH2)2—S—CH3 | —CH2, +S |
| L106I | —CH2—CH—(CH3)2 | — CH(CH3)—CH2—CH3 | None |
| Total | | | —7CH2 |

A total of 12 more substitutions were observed between R7 and R11 with only four mutations (E14D, G38S, Q67K and K₁₁₉R) on the surface and eight mutations (I119V, L36T, Y45F, 153V, M75V, I191V, L105M and L1061) being fully or partially buried in the liganded structure (FIG. 1B, Tables 15 and 16). The relatively few changes on the surface might indicate that by the 7^{th} round of shuffling, a plateau had been reached in terms of optimization of the surface structure. Two of the surface mutations (G38S and Q67K) again occurred in the cluster identified above and deposited one more extra positive charge on the area (Table 15). The same downsizing trend was also clear from the interior mutations between R7 and R11. In addition to preserving all the size-reduction substitutions observed in R7, R11 had 6 more substitutions, 119V, L36T, Y45F, I53V, M75V, and I91V, wherein larger residues are replaced with smaller ones (Table 16). The only exception of interior substitution increasing the molecular weight was L105M, where the branched Leu was replaced with a linear Met. This residue, at the N-terminus of β4, packs against the folded-over loop β3/β4. The L105M mutation reduces the hydrophobicity of the side chain at this position from 97 to 74 (hydrophobic indices, Monera et al. (1995) J Pept Sci 1(5):319-329), thereby reducing structural stiffness. I19V is located in the substrate binding loop20 and its side chain hydrophobically interacts with L15, L20, L78, and AcCoA's pantetheine moiety. L20 defines one wall of the substrate binding site, holding the substrate in a favorable position for acetylation. The I19V mutation presumably allowed the secondary amine of glyphosate to align better with the acetyl group. L36T, at the C-terminal end of helix 2b and near the substitution T33S observed in R7, seemed to further loosen this helix. G38S, at the N-terminal end of β2, apparently increases the protein rigidity though exposed to solvent. The effect of the loss of the phenol group in the Y45F mutation is less clear, but Keenan et al. (2005) Proc. Natl. Acad. Sci. USA 102(25):8887-8892 showed that this mutation might alter protein-protein interaction in the crystal packing. I53V was at the packing interface between the core 13 sheet and helix al. The M75V at the β-bulge orients its amide to the reaction center, hydrogen bonding to the carbonyl of the AcCoA's thioester (FIG. 8). This hydrogen bond both positions the thioester properly for the acylation reaction and also further polarizes the carbonyl, making the carbon atom more susceptible to nucleophilic attack by the glyphosate amine. The replacement of Met75 by a valine might fine-tune this amide group to better fit glyphosate. Similarly, I91V was also at the protein core, sandwiched by the packing interface of the β sheet and helix α3.

Gene shuffling has reshaped the protein surface properties such as increasing the net positive charge and altering the dipole. It also directly increased the volume of the substrate binding site to accommodate the larger glyphosate. Other systematically downsizing substitutions created numerous small cavities and/or abolished some internal hydrogen bonds in the protein core. Structural flexibility is inversely related to protein packing density (Halle (2002) Proc. Natl. Acad. Sci. USA 99:1274-1279). On the other hand, filling cavities can inhibit the motion of functionally important regions of a protein, thereby diminishing its catalytic activity (Ogata et al., (1996). Nat. Struct. Biol., 3, 178-187). Thus, the greater flexibility of optimized GLYATs may be needed for its functional improvement.

### Unliganded Protein MD Simulations:

The improvement of GLYAT catalytic efficiency by gene shuffling was contributed in part through an enhancement of substrate recognition, as the glyphosate K_{M} decreased from 1.27 mM for native GLYAT, to 0.24 mM for R7, and to 0.055 mM for R11 (Siehl et al. (2007) J Biol Chem 282:11446-11455). The crystal structures in complex with ligands showed that the glyphosate binding site is located near the center of the enzyme and buried by the two binding loops, loop20 and loop130, or β-hairpin (FIG. 1A and FIG. 1B). Because of the requirement for ammonium sulfate for crystal formation, an apoenzyme structure was not obtained. Instead, part of the glyphosate binding site was occupied by sulfate, resulting in an even more closed active site than observed with 3PG (Keenan et al. (2005) Proc. Natl. Acad. Sci. USA 102(25):8887-8892; Siehl et al. (2007) J Biol Chem 282:11446-11455). A similar active site architecture was observed in the enzyme arylalkylamine N-acetyltransferase (AANAT), where two loops corresponding to those covering the GLYAT active site cover serotonin. However, these recognition loops in AANAT adopted substantially altered conformations in the apoenzyme, suggesting a catalytic mechanism involving conformational transition (Vetting et al. (2003) Protein Sci. 12:1954-1959; Hickman et al. (1999) Mol. Cell 3(1):23-32; Hickman et al. (1999) Cell 97(3):361-369).

To gain insights into the conformational transition of GLYAT's active site, molecular dynamics simulations were performed for the apoenzyme. The 3PG structure (PDB:2J DD) was used as the starting coordinates with all the crystal waters kept, but ligands deleted. The empty space left by the removal of the ligands was filled with waters and brought to equilibrium by >200 ps MD simulations with protein heavy atoms under harmonic constraints. A ˜3 ns MD simulation of the R7 GLYAT variant was first run using CHARMm in CHARMm 27 force field and TIP3P waters. The simulation produced a stable trajectory and most significantly, the two binding loops started opening up at ˜200 ps. To confirm the findings, simulations with GROMACS were carried out in OPLS-AA force field and SPC waters up to ˜11 ns including ˜1 ns equilibration phase. The results from the two methods were very similar, consistent with a recent literature report that most of the detected major conformational dynamics behaviors with MD are force field independent (Rueda et al. (2007) Proc. Natl. Acad. Sci. USA 104(3):796-801). In comparing the trajectories between 1.8 and 3.0 ns, we noticed CHARMm produced relatively larger fluctuations and underwent a faster conformational evolution. For CHARMm and Gromacs, respectively, the RMSF of all the protein heavy atoms were 1.01±0.52 and 0.89±0.45, while the average RMSD of heavy atoms compared to their initial structures were 2.68±0.18 and 2.06±0.13. Due to the longer simulation periods enabled by its higher computing speed, only the Gromacs results are reported herein. R11 and YVII GLYATs in the absence of ligand were also simulated (Table 17).

### Overall Structure Evolution

All three trajectory RMSDs of heavy atoms to the initial structures were stabilized after ˜400 ps and the overall values in the 10 ns production phase were less than 3.3, 2.5, and 2.2 Å for R11, R7, and YVII, respectively (FIG. 3A). If the flexible loops were taken away, the backbone RMSDs of the core secondary structure elements for the three variants were all less than 1.0 Å. R11's profile experienced the largest fluctuations, which peaked at ˜5 ns with 3.3 Å and dropped down to ˜0.9 Å at 3.0 and 8.6 ns. A similar fluctuation was also observed for core backbone atoms, suggesting that R11 possessed a relatively higher flexibility. Interestingly, YVII's RMDS was substantially lower and more stable than that of R7 and R11 GLYAT. Further analysis revealed that YVII's active site was stuck in the closed conformation for most of the simulations. The B factors of Cα atoms derived from root means square fluctuations (RMSF) of the trajectory between 3 and 5 ns were calculated (FIG. 3B). The B factor profiles were well correlated with the secondary structure elements and evolutionary sequence conservation within the GNAT superfamily. The loops possessed the higher B factors and the well-known conserved D and A sequence motifs displayed the highest stability. Without the bound ligand, β hairpin and loop20 had the highest value. The helix α2, broken in the middle by Phe31 in the crystal structure, was also highly mobile. The fluctuations observed at helix α4 and the P-loop connecting β4 and α3 were apparently caused by the absence of AcCoA. Overall, the B factors of R11 and R7 were slightly higher than those of YVII.

### Open Active Site Conformations

An overlay of the α-carbon traces of snapshots of the open and closed conformations of R7 GLYAT shows that the β hairpin and loop20 underwent the biggest conformational changes (FIG. 4A and FIG. 4B). Helix α1, moving as a rigid body, also drifted away from the glyphosate site along its own axis and adopted a slightly tighter helix while helices 2a and 2b gradually uncoiled. In concert, the n-hairpin connecting the β6 and β7 untwisted and swayed away from the binding site, allowing the active site to become wide open. Another area experiencing a large displacement was helix α4 and its connecting loops, which comprise the binding elements of the pantetheine moiety of AcCoA. To monitor the conformational transition of the active site, a distance between the alpha carbons of Gln24 and Pro134 was calculated (FIG. 4A). In the liganded crystal structures, the loops closely interact with each other through their tips and the distance is ˜9.0 Å (FIG. 4A). FIG. 4B shows the distance variation over a 10 nanosecond simulation time. The state was defined as open when the distance was >14 Å, the point at which the direct interloop contact disappears. R7's active site gradually opened up in the first 2 ns and remained open until ˜7.3 ns, with a peak inter-loop distance of ˜21 Å at around 5 ns. The closed conformation was revisited for a short period between 7800 and 8300 ps. R11 exhibited a similar conformational transition but with a slightly larger amplitude of ˜24 Å to ˜6.5 Å. Complementary to X-ray data, these MD results provide insights into the catalytic cycle, from substrate intake to product release. The inter-conversion of enzyme active sites between closed and open conformations has been observed in many dynamic simulations (Scott et al. (2000) Structure 8(12):1259-1265; Gunasekaran et al. (2003) J Mol Biol 332(1):143-159; Gunasekaran et al (2007) J Mol Biol 365(0:257-273). For example, Hornak et al (2006) Proc. Natl. Acad. Sci. U.S.A. 103, 915-920 showed that unliganded HIV-1 protease flaps could spontaneously open and reclose within a 30 ns MD simulation.

### Structural Inter-Subdomain Motions:

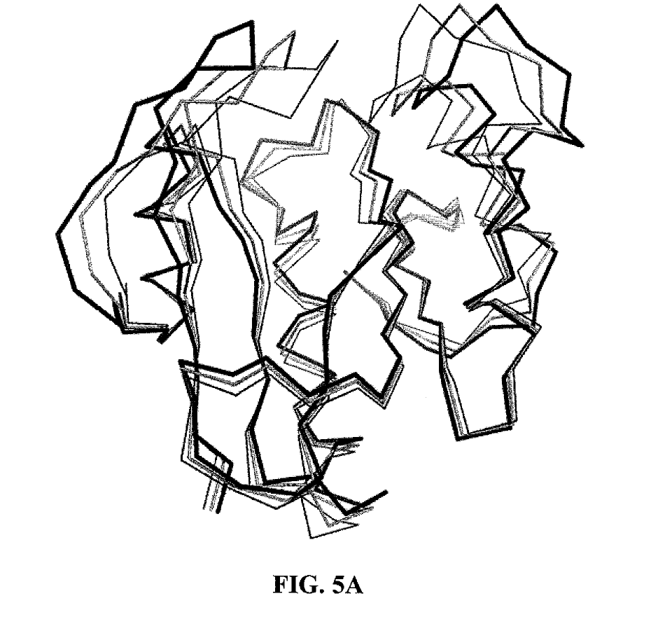

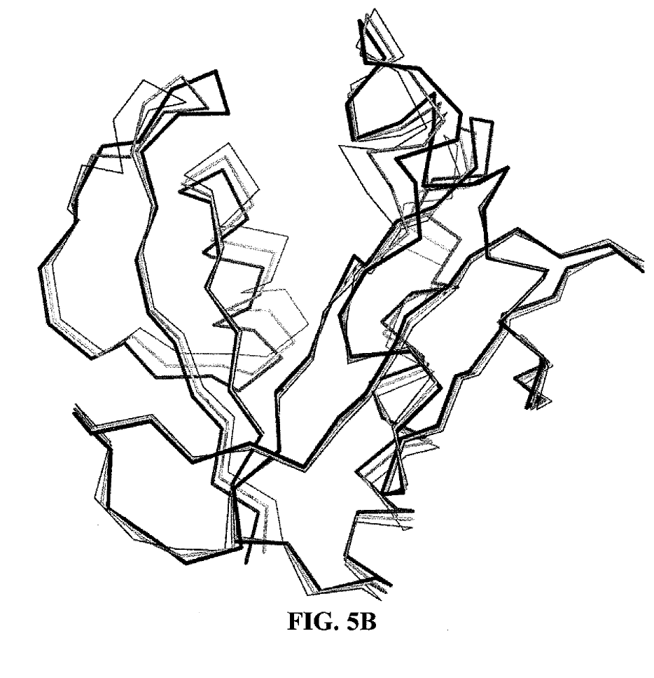

Principal Component Analysis (PCA) of MD trajectory is an efficient way to filter high frequency motion and capture low frequency but highly correlated motions that often have biological significance (Kitao & Go (1999) Curr. Opin. Struc. Biol. 9:164-169; Ota & Agard (2001) Protein Sci 10(7):1403-1414). Covariance matrices were built from backbone atoms of 7,000 frames (<7 ns). The resultant eigenvalues showed that the first two eigenvectors predominated. Their projected motions are delineated in FIG. 5. The motion along the first eigenvector was most pronounced at the glyphosate binding elements, with the β-hairpin and the opposite loop20 moving outward in a concerted way, allowing the active site to open up like a clamshell (FIG. 5A). It also divided the overall structure into two subdomains along the V-shaped wedge. Subdomain I, with residues 1-102, is composed of β1α1α2aαbβ2β3β4α3 and subdomain I1, with residues 103-146, consists of β5α4β7β6. The two subdomains butt together at the N-termini of the parallel β4 and β5 strands, forming an integrated β-sheet. The joint is further secured by a long loop between β4 and β5 which packs against the integrated β sheet. The wedge joint exhibits the least motion, while the AcCoA binding end has a relatively large displacement. As described above, most surface mutations introduced by DNA shuffling were concentrated at the wedge joining end (FIG. 1A), possibly modulating the structure's overall motion. The second eigenvector projection showed a wedge twisting with the β-hairpin and the opposite helix loop20 sliding against one another (FIG. 5B). This motion also used the wedge joint as the hinge, but its direction was perpendicular to the first mode and its amplitude was much smaller. The R11 trajectory PCA analysis revealed identical motion modes, whereas YVII only showed the wedge twisting motion. YVII's active site remained closed, with an inter-loop distance of −12 Å for much of its simulation course. A few more MD simulations were performed on YVII with different parameters such as random seed number, solvent box shape, and size to check the active site conformational transition. Those experiments generally confirmed that the active site of YVII remained in the closed form for relatively longer periods of time.

To probe the stability of the wedge over the MD simulation, we defined a dihedral angle with four Ca atoms, Ala76, Leu72, Cys108 and Argil 1, (FIG. 6A). The wedge opening angle was defined as α+β−180, with 0° being where the two strands (β4 and β5) are ideally parallel, while the wedge twisting angle is dihedral θ, again with θ=0° being the untwisted flat sheet. The crystal structure of the 3PG complex showed α=98.42° and β=114.62°, resulting in a wedge opening angle of 33.04° while the angle for the SO₄²⁻ structure was 31.58°. As the glyphosate binding site was located right on the top of the open wedge, the smaller wedge opening angle of the SO₄²⁻ complex reflected the smaller size of SO₄²⁻, compared to 3PG. In simulations, average wedge angles were observed over the entire 10 ns trajectory of 40.8±3.1, 47.3±4.8, and 45.9±5.1° for YVII, R7, and R11, respectively (FIG. 6B-FIG. 6G), demonstrating that the wedge opened significantly wider in the absence of bound ligands. For the wedge dihedral angle θ, the two crystal structures give roughly the same value with −16.34° for the 3PG complex and −16.61° for the SO₄²⁻ complex. The average wedge twisting angles from MD trajectories were −21.8±5.2°, −9.2±6.2°, and −10.2±6.1° for YVII, R7, and R11, respectively.

### Structural Basis for Inter-Subdomain Motion and Active Site Flexibility:

As hinge-like, broad-range motions are usually determined by a protein's overall structure (Sinha and Nussinov (2001) Proc. Natl. Acad. Sci. USA 98:3139-3144), GLYAT's inter-subdomain motions involving wedge opening and twisting were apparently a feature of its unique topology. In the GLYAT structure, the most stable elements were the helix α3 and the surrounding seven stranded β sheet, which is split by the wedge at one end. The first four strands (β1-β4 in the subdomain I) wrap against helix α3 while the strands β5-β7 in subdomain II interact with α3 only at the wedge joining end. On the other end, helix α4 acts like a spring inserted between the subdomains, enabling the inter-subdomain movements. Conceivably, this inter-subdomain motion involving the well conserved structural elements plays a role in controlling the access of AcCoA, determining bound AcCoA's conformation, and facilitating the egress of CoA.

The motion associated with the active site conformational change is enacted by the 0 hairpin and loop20, the least conserved motifs in the GNAT family. The β-hairpin, comprised of residues 130 to 138 (FDTPPVGPH in R7), connect β6 and β7, with the four middle residues (TPPV) forming a typical Vla β-turn (Richardson (1981) Adv Protein Chem. 1981; 34:167-339). The two consecutive prolines Pro133 and Pro134 reduce its flexibility, with Pro133 adopting a trans- and Pro134 a cis-conformation. Such structural motifs often are associated with molecular recognition and function, including type VI β-turns in HIV-11_{IIB} (Tugarinov et al. (1999) Nat. Struct. Biol. 6(4): 331-335), Bowman-Birk proteinase inhibitor (Brauer et al. (2002) Biochemistry 41(34):10608-10615), and disulfide oxidoreductase (DsbA) (Charbonnier et al. (1999) Protein Sci 8:96-105). Here, the β-hairpin covers glyphosate's phosphono group and also harbors the putative catalytic base His138 (FIG. 8). Amino acid substitutions I132T and I135V, introduced by gene shuffling, had a significant impact on the stability of the β-hairpin by reducing hydrophobic packing strength among the paired side chains (FIG. 7). In the YVII or native enzyme, the side chains of I132, P133, cis-Pro134, and I135 (and possibly H138 as well) form a hydrophobic cluster, stabilizing the type Vla β-turn and hairpin (FIG. 7). In optimized GLYATs, however, two strong hydrophobic isoleucines are replaced by a weaker valine at 135 and even a hydrophilic threonine at 132. As a consequence, the O-hairpin in the optimized GLYAT exhibits greater flexibility (FIG. 3A, FIG. 3B, and FIG. 4B) during the MD simulation. As judged by their bond lengths, the average inter-strand hydrogen bonds in R7 GLYAT were weaker than those in YVII. In YVII GLYAT, the hydrogen bond distances of Ile132N-Gly136O, Ile132O-Ile135N, and Ile132O-Gly136N were 3.1±0.3 Å, 3.1±0.2 Å and 2.9±0.1 Å, respectively, while for R7 GLYAT the corresponding distances (Thr132N-Gly136O, Thr132O-Val135N and Thr132O-Gly136N) were 3.3±0.4 Å, 3.4±0.2 Å and 3.0±0.2 Å; respectively. Similarly, compared to the YVII, the β-hairpin in R7 had slightly less well-defined secondary structure elements on average as measured by DSSP (Holm & Sander (1993) J Mol Biol. 233(1):123-138).

The MD simulations also suggested that the reduced stability of the β-hairpin in optimized GLYAT variants might also be responsible for accelerating the active site opening. In the crystal structure of the R7-3PG complex, both the n-hairpin and the loop20 cover 3PG and make direct van der Waals contacts through their tip regions, including the side chains of Val135 with Arg21 and Pro134 with Gln24. The aliphatic side chain of Arg111 and the β-hairpin also align with each other. The interloop van der Waals contacts of YVII GLYAT were well maintained whereas these same contacts were lost quickly as a consequence of a large conformational adjustment of the β-hairpin in the R7 and R11 simulations. Indeed, revertant mutations at the β-hairpin of R7 significantly elevated the K_{M} for glyphosate by 3.2- and 6.4-fold for T132I and V135I, respectively, reflecting the fact that the enhanced β-hairpin flexibility partially enables optimized GLYAT variants to better associate with glyphosate. In summary, the more optimized GLYAT apparently showed a larger amplitude of fluctuation and inter-subdomain motion in the simulation, associated with and probably a consequence of the selection of an ensemble or downsizing substitutions.

### Liganded System Simulation and Ligand-Protein Interaction:

The partially or fully liganded simulations were carried out in CHARMm 27 force field. The ligand topology and parameters of AcCoA, glyphosate and D-AP3 were generated by InsightI1 (Accelrys, San Diego). The partial charge values were calculated with vcharge (FIG. 2A and FIG. 2B). The simulations were first carried out under harmonic constraints allowing side chain atoms and waters to equilibrate (˜0.3 ns), followed by ˜2.5 ns production phase. The average heavy atom RMSDs over the entire trajectory were 2.01±0.3, 1.65±0.10, and 1.40±0.13 Å for AcCoA+R7, glyphosate+AcCoA+R7, and D-AP3+AcCoA+YVII, respectively.

(1). Binary complex of R7+AcCoA: The recognition mode of the cofactor in all the known structures is extremely similar despite high divergence in their primary sequence and, in Fact, the GNAT fold seems to have been optimized around the binding of the phosphopantetheine motif (Dyda et al. (2000) Annu. Rev. Biophys Biomol. Struct. 29:81-103). The pantetheine arm and β4 form a pseudo β-sheet and the interacting inter-strand hydrogen bonds were well preserved in the simulation. In R7 GLYAT, the average bond length spanning N4P of AcCoA and C═O of Gly75 was 2.91±0.18 Å and that spanning C═O of AcCoA and the amide N of Thr77 was 2.91±0.14 Å. The pyrophosphate moiety of AcCoA also maintained stable interactions with the protein but its 3′ phosphate and ribosyl groups were solvent accessible and fluctuated widely. The kinetic mechanism of well-studied GNAT family members was shown to be ordered with a preference for AcCoA first binding to the free enzyme, followed by the binding of acceptor substrates (Vetting et al. (2005) Protein Sci 12:1954-1959; De Angelis et al. (998) J. Biol. Chem. 273 3045-3050), suggesting a structural role of the cofactor in organizing the active site (Dyda et al. (2000) Annu. Rev. Biophys. Biomol. Struct. 29:81-103). With AcCoA bound in the wedge, the overall fluctuations across the entire protein core decreased but the glyphosate binding loops remained mobile. The flexibility of the β-bulge was reduced apparently due to interaction with the acetyl carbonyl group of AcCoA. Regarding the subdomain motion, the angles of the V-shaped wedge opening and twisting were 36.3±2.8°, and −14.6±6.8°, significantly different from the unliganded values (45.9±5.1° and −9.2±6.2°) but similar to the fully liganded crystal structure (33.04° and −16.34°). Conceivably, AcCoA binding severely restricts inter-subdomain fluctuation around the wedge.

(2) Ternary complexes of R7+AcCoA+glyphosate and YVII+AcCoA+D-AP3: The initial conformations of the substrates were modeled as follows. The atoms of glyphosate were mapped onto the corresponding positions of 3PG, since the two molecules have a similar main chain structure. D-AP3, a primary amine, has a shorter main chain and branched structure. Its phosphono and carboxyl groups were placed in the equivalent positions of 3PG and its amine was directed toward the acetyl of AcCoA. When the docked complex structures were carefully relaxed with energy minimizations in the presence of crystal waters, the initial substrate conformations were well retained. During the subsequent simulations, glyphosate remained in its initial conformation as did the phosphono and carboxyl groups of D-AP3. However, the D-AP3 amine started to sway away from AcCoA after ˜1.5 ns, resulting in an unproductive conformation. Compared with the binding site of glyphosate in R7, the D-AP3 binding site of YVII exhibited much less fluctuation and was more compact. As a consequence, the average trajectory RMSDs against the X-ray structure of backbone atoms were significantly different. The RMSD of D-AP3+AcCoA+YVII was 0.8±0.13 Å, much smaller than the 1.15±0.15 Å observed for glyphosate+AcCoA+R7. The higher stability of D-AP3+AcCoA+YVII apparently resulted from (a) the smaller and more rigid D-AP3 structure, (b) the hydrogen bond of the Y31 phenol to D-AP3's carboxyl, and (c) the increased hydrophobic packing of I132 and I135 in YVII compared to T132 and V135 in R7. These findings again demonstrate the effect of downsizing substitutions in increasing the protein flexibility.

Although glyphosate shares many similar features with 3PG, a difference in their binding mode was observed. During the simulation, the glyphosate structure adjusted at ˜100 ps, responding to the absence of an equivalent of the intramolecular hydrogen bond seen with 3PG between the 2-hydroxyl and a phosphate oxygen (FIG. 8). Consequently, glyphosate adopted a more extended conformation with its phosphono group displaced out and down by about 1.4 Å toward the acetyl group of AcCoA, and the dihedral angle around the O₃P—CH₂ bond rotated ˜15° to allow the phosphono oxygen atoms to avoid close contact with C_{A}. The molecular dimension measured by the distance between the two farthest atoms was ˜8 Å for bound glyphosate and ˜6 Å for bound 3PG. During the adjustment, the carboxyl group, its binding residues (Gly74 and Arg73) and F31 remained in the same place but the phosphono group and β-hairpin moved outward. The average interloop distances between Gln24Cα and Pro 134Cα were 0.57±0.88 Å and 10.29±0.75 Å for R7+glyphosate and YVII+D-AP3 MD simulations, respectively, compared to 9.0 Å in the R7+3PG crystal structure. The side chain of Arg111 and its main chain in the β5 also showed appreciable movement. In the stabilized conformation, the Gln110 amide and/or Gln109 carbonyl groups formed water-mediated hydrogen bonds to the phosphono group of glyphosate. Another stable water molecule at the splaying point between β4 and β5 (also observed in two independent crystal structures) mediated interaction between the 108 amide and 72 carbonyl atoms. The amine group of glyphosate remained accessible to bulk solvent from the direction opposite to the bound AcCoA for the entire simulation. It is possible that a water wire, as previously suggested, serves as the catalytic base ferrying the protons away. The amine group of glyphosate maintained close contact with the acetyl carbon of AcCoA (within 3.8 Å) in position for the nucleophilic attack. The largest structural adjustment was observed at the side chain of Arg21. Its guanidinium, interacting with the hydroxyl and carboxylate groups in the 3PG structure, moved toward the β-hairpin in the glyphosate MD simulation, and formed a salt-bridge with the phosphonyl group of glyphosate.

### Materials and Methods

The starting coordinates of the complex of R7 GLYAT from the 7^{th} round gene shuffling with bound 3-phosphoglycerate (3PG) and AcCoA were taken from the x-ray structure, PDB:2JDD at 1.60-Å resolution. The initial structural coordinates of other GLYAT variants were constructed using InsightII's MODELER module but without invoking its auto energy minimization procedure (Accelrys, San Diego) and/or CHARMm IC facility (Brooks et al. (1983) J. Comput. Chem. 4:187-217). The in silico mutations based on R7-GLYAT included (1) F31Y, A114V, V132I and T135I for YVII GLYAT; (2) E14D, I19V, L36T, G38S, Y45F, I53V, Q67K, M75V, 191V, L105M, L1061 and K119R for the R11-GLYAT; and (3) L15I, V19I, V132I, I26L, F31Y, S33T, R37G, G47R, Q58E, Q65E, Q67E, Q68E, S89T, K82R, I97L, R101K, A114V, K119E, F130Y, T132I, R144K and I145L for the native GLYAT, respectively (FIG. 1A and FIG. 1B). To avoid instability caused by atomic conflict, all the residue side-chains neighboring the mutation points were carefully inspected and their rotamers were manually adjusted to a local minimal with BIOPOLYMER (Accelrys, San Diego) prior to energy minimization. The energy minimizations were carried out on CHARMm under various constraints to relax the structure gradually, first in vacuum with the crystal waters and then in solvent TIP3 water boxes. The topology in a CHARMm force field of cofactor AcCoA, substrate glyphosate and its analogs 3PG and D-2-amino-3-phosphonopropionate (D-AP3) was constructed with InsightII of Accelyres. The charge was calculated with Vcharge (Gilson et al. (2003) J. Chem. Inf. Comput. Sci. 43(6):1982-1997) (FIG. 2A and FIG. 2B). The initial conformations of substrate and analogs were manually docked into the GLYAT active site using PDB:2JDD as reference. The histidine protonation state, either on NE2 or ND 1, was determined based on the hydrogen bonding pattern of the crystal structure. His138 ND1 hydrogen bonded the 137 carbonyl oxygen in the absence of substrate, but in the presence of glyphosate its NE2 was also protonated to provide a key hydrogen bond to the substrate's phosphono group (Sichl et al. (2007) J Biol Chem 282:11446-11455). Periodic boundary conditions were used to perform all the MD simulations, and were defined by using truncated octahedron boxes of dimensions ˜63 Å. All the boxes were first filled with modeled waters (T1P3P (Mahoney et al. (2000) J Chem Phys 112:8910-8922) for CHARMm and SPC (Berendsen et al. (1981) in Intermolecular Forces. Pullman, B. (ed). Rieidel, Dordrecht, The Netherlands, p. 331) for GROMACS (Berendsen et al. (1995) Comp. Phys. Commun. 91:43-56)), followed with energy minimization and equilibration, at >200 ps. The overall charges of all the systems were neutralized with either Na⁺ or Cl⁻ ions by randomly replacing bulk water molecules.

Molecular Dynamics (MD) simulations of all the liganded and unliganded systems (see Table 17 for a list of all the MD simulations that were performed) were carried out for >2,000 picoseconds (ps) by CHAR Mm 31b1 while, as a comparison, GROMACS 3.3.1 was also employed for the unliganded systems, R7-GLYAT, YVII-GLYAT, and R11-GLYAT for longer simulation times (−11,000 ps). For CHARMm simulations, the residue topology and parameter files as generated by CHARM M 27 (MacKerell et al. (2004) Journal of Computational Chemistry 25:1400-1415) were used for protein atoms and ligands. The Verlet-Leapfrog algorithm was used to integrate the equations of motion by using a time step of 2.0 fs. The SHAKE algorithm was used to constrain the bonds containing hydrogen to their equilibrium length. Electrostatic interactions were treated with a cutoff switch of 14 Å. A harmonic constraint of force of 10 kcal·mol⁻¹·Å⁻² was applied to heavy atoms in the heating phase, from 240 to 300 K for ˜200 ps. Then the constraints were only applied to heavy non-water atoms in equilibrium phase lasting >600 ps. Finally, all the constraints were released for the production phase at 300 K. For the GROMACS simulations, an OPLS-AA/L all-atom force field (Jorgensen et al. (1996) J. Am. Chem. Soc. 118:11225-11236; Kaminski et al. (2001) J. Phys. Chem. 105:6474-6487) was used and the NPT ensemble was computed at 300 K using the Berendsen thermostat. Electrostatics was treated as the particle mesh Ewald method with a short range cut-off of 10 Å. The time step for integration was 2 fs, calculated with the leap-frog algorithm. The LINCS algorithm was performed to restrain bond lengths. Each system was subjected to a 600-ps dynamics run with the protein restrained at 4.8 kcal·mol⁻¹·Å⁻² on all heavy atoms, followed by a 10 its free simulation. All of the simulations were performed on a Linux cluster.

TABLE 17
Summary of simulations.

| GLYAT | | | | Duration |
|---|---|---|---|---|
| variant | Ligands | Method | Atoms | (ns) |
| R7 | Empty | Gromacs | 19,203 | 11 |
| | | (OPLS-AA/L) | | |
| R7 | Empty | CHARMm | 19,105 | 2.3 |
| R7 | AcCoA | CHARMm | 18,860 | 2.5 |
| R7 | AcCoA, | CHARMm | 19,238 | 2.6 |
| | glyphosate | | | |
| YVII | Empty | Gromacs | 20,171 | 11 |
| | | (OPLS-AA/L) | | |
| YVII | AcCoA, | CHARMm | 19,334 | 2.5 |
| | D-AP3 | | | |
| R11 | Empty | Gromacs | 22,268 | 11 |
| | | (OPLS-AA/L) | | |

Covariance analysis and principal component analysis (PCA, Tai et al., (2001) Biophys. J. 81:715-724) were performed on trajectories computed by either CHARMm or GROMACS to reduce the data complexity. The backbone atomic average displacements over trajectories were used as covariance variables. The covariance matrix and eigenvector analysis were obtained by applying the g_covar program of the GROMACS package. To capture the large amplitude, slow frequency, and dominant motions, the trajectories were projected into the top two eigenvectors. All the graphs were prepared with Pymol (http://pymol.sourceforge.net/), InsightII, and Gnuplot (http://www.gnuplot.info/).

TABLE 18
The atomic coordinates in Angstroms of the GLYAT R7 variant bound
to glyphosate and acetyl coA, along with surrounding water molecules.

| ResI^{a} | ResN^{b} | AtomI^{c} | AtomN^{d} | X^{c} | Y | Z | ElemN^{f} | SegN^{g} |
|---|---|---|---|---|---|---|---|---|
| 2 | ILE | 1 | N | 19.658 | −1.102 | −7.005 | N | PRO |
| | | 2 | HT1 | 19.144 | −0.191 | −7.071 | H | PRO |
| | | 3 | HT2 | 18.95 | −1.85 | −6.813 | H | PRO |
| | | 4 | HT3 | 20.124 | −1.291 | −7.914 | H | PRO |
| | | 5 | CA | 20.67 | −1.067 | −5.913 | C | PRO |
| | | 6 | HA | 21.302 | −0.211 | −6.099 | H | PRO |
| | | 7 | CB | 20.012 | −0.957 | −4.529 | C | PRO |
| | | 8 | HB | 19.454 | −1.901 | −4.319 | H | PRO |
| | | 9 | CG2 | 21.1 | −0.805 | −3.436 | C | PRO |
| | | 10 | HG21 | 20.631 | −0.641 | −2.446 | H | PRO |
| | | 11 | HG22 | 21.723 | −1.719 | −3.357 | H | PRO |
| | | 12 | HG23 | 21.763 | 0.058 | −3.655 | H | PRO |
| | | 13 | CG1 | 18.969 | 0.191 | −4.445 | C | PRO |
| | | 14 | HG11 | 18.143 | 0.001 | −5.164 | H | PRO |
| | | 15 | HG12 | 18.507 | 0.164 | −3.434 | H | PRO |
| | | 16 | CD1 | 19.53 | 1.598 | −4.681 | C | PRO |
| | | 17 | HD1 | 18.72 | 2.352 | −4.587 | H | PRO |
| | | 18 | HD2 | 20.316 | 1.843 | −3.937 | H | PRO |
| | | 19 | HD3 | 19.962 | 1.689 | −5.699 | H | PRO |
| | | 20 | C | 21.511 | −2.316 | −5.995 | C | PRO |
| | | 21 | O | 20.994 | −3.403 | −6.245 | O | PRO |
| 3 | GLU | 22 | N | 22.837 | −2.181 | −5.779 | N | PRO |
| | | 23 | HN | 23.249 | −1.281 | −5.63 | H | PRO |
| | | 24 | CA | 23.744 | −3.298 | −5.672 | C | PRO |
| | | 25 | HA | 23.255 | −4.228 | −5.928 | H | PRO |
| | | 26 | CB | 25.029 | −3.117 | −6.508 | C | PRO |
| | | 27 | HB1 | 25.506 | −2.155 | −6.219 | H | PRO |
| | | 28 | HB2 | 25.736 | −3.941 | −6.26 | H | PRO |
| | | 29 | CG | 24.828 | −3.116 | −8.032 | C | PRO |
| | | 30 | HG1 | 24.408 | −4.091 | −8.359 | H | PRO |
| | | 31 | HG2 | 24.141 | −2.304 | −8.34 | H | PRO |
| | | 32 | CD | 26.186 | −2.902 | −8.695 | C | PRO |
| | | 33 | OE1 | 26.733 | −1.769 | −8.589 | O | PRO |
| | | 34 | OE2 | 26.727 | −3.882 | −9.269 | O | PRO |
| | | 35 | C | 24.185 | −3.342 | −4.235 | C | PRO |
| | | 36 | O | 24.465 | −2.301 | −3.647 | O | PRO |
| 4 | VAL | 37 | N | 24.263 | −4.553 | −3.64 | N | PRO |
| | | 38 | HN | 24.032 | −5.391 | −4.138 | H | PRO |
| | | 39 | CA | 24.694 | −4.737 | −2.269 | C | PRO |
| | | 40 | HA | 24.81 | −3.778 | −1.783 | H | PRO |
| | | 41 | CB | 23.749 | −5.595 | −1.437 | C | PRO |
| | | 42 | HB | 23.723 | −6.639 | −1.827 | H | PRO |
| | | 43 | CG1 | 24.218 | −5.622 | 0.035 | C | PRO |
| | | 44 | HG11 | 23.468 | −6.147 | 0.663 | H | PRO |
| | | 45 | HG12 | 25.187 | −6.154 | 0.138 | H | PRO |
| | | 46 | HG13 | 24.339 | −4.588 | 0.423 | H | PRO |
| | | 47 | CG2 | 22.327 | −5.013 | −1.55 | C | PRO |
| | | 48 | HG21 | 21.646 | −5.552 | −0.858 | H | PRO |
| | | 49 | HG22 | 22.326 | −3.936 | −1.283 | H | PRO |
| | | 50 | HG23 | 21.93 | −5.12 | −2.581 | H | PRO |
| | | 51 | C | 26.036 | −5.409 | −2.351 | C | PRO |
| | | 52 | O | 26.199 | −6.397 | −3.069 | O | PRO |
| 5 | LYS | 53 | N | 27.048 | −4.857 | −1.643 | N | PRO |
| | | 54 | HN | 26.898 | −4.055 | −1.061 | H | PRO |
| | | 55 | CA | 28.413 | −5.32 | −1.741 | C | PRO |
| | | 56 | HA | 28.41 | −6.337 | −2.104 | H | PRO |
| | | 57 | CB | 29.28 | −4.417 | −2.665 | C | PRO |
| | | 58 | HB1 | 29.428 | −3.425 | −2.186 | H | PRO |
| | | 59 | HB2 | 30.279 | −4.882 | −2.808 | H | PRO |
| | | 60 | CG | 28.627 | −4.191 | −4.036 | C | PRO |
| | | 61 | HG1 | 28.344 | −5.177 | −4.469 | H | PRO |
| | | 62 | HG2 | 27.687 | −3.612 | −3.88 | H | PRO |
| | | 63 | CD | 29.471 | −3.433 | −5.071 | C | PRO |
| | | 64 | HD1 | 29.927 | −2.533 | −4.603 | H | PRO |
| | | 65 | HD2 | 30.288 | −4.098 | −5.429 | H | PRO |
| | | 66 | CE | 28.569 | −3.005 | −6.234 | C | PRO |
| | | 67 | HE1 | 27.98 | −3.881 | −6.583 | H | PRO |
| | | 68 | HE2 | 27.865 | −2.217 | −5.893 | H | PRO |
| | | 69 | NZ | 29.299 | −2.484 | −7.408 | N | PRO |
| | | 70 | HZ1 | 28.576 | −2.164 | −8.1 | H | PRO |
| | | 71 | HZ2 | 29.915 | −1.695 | −7.134 | H | PRO |
| | | 72 | HZ3 | 29.858 | −3.266 | −7.83 | H | PRO |
| | | 73 | C | 28.996 | −5.277 | −0.348 | C | PRO |
| | | 74 | O | 28.545 | −4.464 | 0.461 | O | PRO |
| 6 | PRO | 75 | N | 29.981 | −6.102 | 0 | N | PRO |
| | | 76 | CD | 30.452 | −7.237 | −0.799 | C | PRO |
| | | 77 | HD1 | 29.665 | −8.021 | −0.798 | H | PRO |
| | | 78 | HD2 | 30.692 | −6.929 | −1.84 | H | PRO |
| | | 79 | CA | 30.698 | −5.981 | 1.258 | C | PRO |
| | | 80 | HA | 30.004 | −5.833 | 2.073 | H | PRO |
| | | 81 | CB | 31.48 | −7.299 | 1.367 | C | PRO |
| | | 82 | HB1 | 30.838 | −8.055 | 1.87 | H | PRO |
| | | 83 | HB2 | 32.426 | −7.203 | 1.937 | H | PRO |
| | | 84 | CG | 31.706 | −7.739 | −0.083 | C | PRO |
| | | 85 | HG1 | 31.844 | −8.834 | −0.177 | H | PRO |
| | | 86 | HG2 | 32.6 | −7.218 | −0.493 | H | PRO |
| | | 87 | C | 31.633 | −4.798 | 1.185 | C | PRO |
| | | 88 | O | 32.233 | −4.561 | 0.137 | O | PRO |
| 7 | ILE | 89 | N | 31.755 | −4.033 | 2.288 | N | PRO |
| | | 90 | HN | 31.264 | −4.256 | 3.132 | H | PRO |
| | | 91 | CA | 32.584 | −2.849 | 2.337 | C | PRO |
| | | 92 | HA | 33.306 | −2.879 | 1.535 | H | PRO |
| | | 93 | CB | 31.825 | −1.528 | 2.228 | C | PRO |
| | | 94 | HB | 32.558 | −0.693 | 2.279 | H | PRO |
| | | 95 | CG2 | 31.177 | −1.46 | 0.827 | C | PRO |
| | | 96 | HG21 | 30.75 | −0.454 | 0.637 | H | PRO |
| | | 97 | HG22 | 31.937 | −1.661 | 0.041 | H | PRO |
| | | 98 | HG23 | 30.365 | −2.212 | 0.732 | H | PRO |
| | | 99 | CG1 | 30.801 | −1.323 | 3.368 | C | PRO |
| | | 100 | HG11 | 30.02 | −2.108 | 3.29 | H | PRO |
| | | 101 | HG12 | 31.307 | −1.446 | 4.35 | H | PRO |
| | | 102 | CD1 | 30.141 | 0.059 | 3.355 | C | PRO |
| | | 103 | HD1 | 29.361 | 0.111 | 4.145 | H | PRO |
| | | 104 | HD2 | 30.896 | 0.851 | 3.544 | H | PRO |
| | | 105 | HD3 | 29.653 | 0.266 | 2.381 | H | PRO |
| | | 106 | C | 33.367 | −2.924 | 3.618 | C | PRO |
| | | 107 | O | 33.124 | −3.787 | 4.46 | O | PRO |
| 8 | ASN | 108 | N | 34.377 | −2.046 | 3.772 | N | PRO |
| | | 109 | HN | 34.578 | −1.363 | 3.061 | H | PRO |
| | | 110 | CA | 35.227 | −1.999 | 4.943 | C | PRO |
| | | 111 | HA | 35.289 | −2.989 | 5.376 | H | PRO |
| | | 112 | CB | 36.655 | −1.505 | 4.608 | C | PRO |
| | | 113 | HB1 | 36.64 | −0.439 | 4.299 | H | PRO |
| | | 114 | HB2 | 37.314 | −1.603 | 5.496 | H | PRO |
| | | 115 | CG | 37.239 | −2.367 | 3.486 | C | PRO |
| | | 116 | OD1 | 37.488 | −3.564 | 3.674 | O | PRO |
| | | 117 | ND2 | 37.435 | −1.757 | 2.285 | N | PRO |
| | | 118 | HD21 | 37.806 | −2.284 | 1.523 | H | PRO |
| | | 119 | HD22 | 37.109 | −0.817 | 2.148 | H | PRO |
| | | 120 | C | 34.612 | −1.073 | 5.969 | C | PRO |
| | | 121 | O | 33.657 | −0.352 | 5.683 | O | PRO |
| 9 | ALA | 122 | N | 35.156 | −1.064 | 7.211 | N | PRO |
| | | 123 | HN | 35.912 | −1.677 | 7.454 | H | PRO |
| | | 124 | CA | 34.749 | −0.151 | 8.261 | C | PRO |
| | | 125 | HA | 33.684 | −0.263 | 8.408 | H | PRO |
| | | 126 | CB | 35.489 | −0.441 | 9.578 | C | PRO |
| | | 127 | HB1 | 35.282 | −1.48 | 9.906 | H | PRO |
| | | 128 | HB2 | 36.589 | −0.336 | 9.445 | H | PRO |
| | | 129 | HB3 | 35.162 | 0.25 | 10.385 | H | PRO |
| | | 130 | C | 35.03 | 1.281 | 7.873 | C | PRO |
| | | 131 | O | 34.186 | 2.159 | 8.037 | O | PRO |
| 10 | GLU | 132 | N | 36.216 | 1.516 | 7.269 | N | PRO |
| | | 133 | HN | 36.885 | 0.77 | 7.167 | H | PRO |
| | | 134 | CA | 36.713 | 2.78 | 6.776 | C | PRO |
| | | 135 | HA | 36.767 | 3.461 | 7.609 | H | PRO |
| | | 136 | CB | 38.118 | 2.628 | 6.139 | C | PRO |
| | | 137 | HB1 | 38.085 | 1.889 | 5.309 | H | PRO |
| | | 138 | HB2 | 38.411 | 3.61 | 5.699 | H | PRO |
| | | 139 | CG | 39.246 | 2.248 | 7.13 | C | PRO |
| | | 140 | HG1 | 40.228 | 2.453 | 6.65 | H | PRO |
| | | 141 | HG2 | 39.166 | 2.888 | 8.034 | H | PRO |
| | | 142 | CD | 39.259 | 0.78 | 7.569 | C | PRO |
| | | 143 | OE1 | 38.48 | −0.044 | 7.024 | O | PRO |
| | | 144 | OE2 | 40.065 | 0.476 | 8.489 | O | PRO |
| | | 145 | C | 35.797 | 3.385 | 5.744 | C | PRO |
| | | 146 | O | 35.645 | 4.604 | 5.666 | O | PRO |
| 11 | ASP | 147 | N | 35.145 | 2.521 | 4.938 | N | PRO |
| | | 148 | HN | 35.275 | 1.538 | 5.044 | H | PRO |
| | | 149 | CA | 34.295 | 2.908 | 3.839 | C | PRO |
| | | 150 | HA | 34.806 | 3.667 | 3.263 | H | PRO |
| | | 151 | CB | 33.952 | 1.704 | 2.92 | C | PRO |
| | | 152 | HB1 | 33.386 | 0.948 | 3.507 | H | PRO |
| | | 153 | HB2 | 33.314 | 2.035 | 2.072 | H | PRO |
| | | 154 | CG | 35.178 | 1.004 | 2.331 | C | PRO |
| | | 155 | OD1 | 36.318 | 1.526 | 2.44 | O | PRO |
| | | 156 | OD2 | 34.979 | −0.105 | 1.765 | O | PRO |
| | | 157 | C | 32.979 | 3.48 | 4.325 | C | PRO |
| | | 158 | O | 32.299 | 4.167 | 3.568 | O | PRO |
| 12 | THR | 159 | N | 32.593 | 3.227 | 5.601 | N | PRO |
| | | 160 | HN | 33.173 | 2.677 | 6.202 | H | PRO |
| | | 161 | CA | 31.331 | 3.695 | 6.153 | C | PRO |
| | | 162 | HA | 30.608 | 3.735 | 5.352 | H | PRO |
| | | 163 | CB | 30.754 | 2.778 | 7.236 | C | PRO |
| | | 164 | HB | 29.763 | 3.175 | 7.56 | H | PRO |
| | | 165 | OG1 | 31.567 | 2.691 | 8.403 | O | PRO |
| | | 166 | HG1 | 32.443 | 2.384 | 8.132 | H | PRO |
| | | 167 | CG2 | 30.527 | 1.363 | 6.684 | C | PRO |
| | | 168 | HG21 | 30.073 | 0.715 | 7.465 | H | PRO |
| | | 169 | HG22 | 29.831 | 1.404 | 5.822 | H | PRO |
| | | 170 | HG23 | 31.479 | 0.898 | 6.355 | H | PRO |
| | | 171 | C | 31.428 | 5.088 | 6.739 | C | PRO |
| | | 172 | O | 30.407 | 5.74 | 6.944 | O | PRO |
| 13 | TYR | 173 | N | 32.657 | 5.579 | 7.028 | N | PRO |
| | | 174 | HN | 33.478 | 5.059 | 6.801 | H | PRO |
| | | 175 | CA | 32.88 | 6.77 | 7.832 | C | PRO |
| | | 176 | HA | 32.332 | 6.631 | 8.754 | H | PRO |
| | | 177 | CB | 34.376 | 6.999 | 8.186 | C | PRO |
| | | 178 | HB1 | 34.966 | 7.093 | 7.248 | H | PRO |
| | | 179 | HB2 | 34.49 | 7.938 | 8.77 | H | PRO |
| | | 180 | CG | 35.007 | 5.9 | 9.02 | C | PRO |
| | | 181 | CD1 | 34.273 | 4.961 | 9.774 | C | PRO |
| | | 182 | HD1 | 33.196 | 4.972 | 9.795 | H | PRO |
| | | 183 | CE1 | 34.925 | 3.963 | 10.508 | C | PRO |
| | | 184 | HE1 | 34.345 | 3.234 | 11.054 | H | PRO |
| | | 185 | CZ | 36.32 | 3.888 | 10.504 | C | PRO |
| | | 186 | OH | 36.947 | 2.844 | 11.21 | O | PRO |
| | | 187 | HH | 37.881 | 2.806 | 10.959 | H | PRO |
| | | 188 | CD2 | 36.413 | 5.836 | 9.067 | C | PRO |
| | | 189 | HD2 | 36.997 | 6.558 | 8.515 | H | PRO |
| | | 190 | CE2 | 37.069 | 4.837 | 9.799 | C | PRO |
| | | 191 | HE2 | 38.148 | 4.793 | 9.8 | H | PRO |
| | | 192 | C | 32.322 | 8.039 | 7.227 | C | PRO |
| | | 193 | O | 31.792 | 8.876 | 7.958 | O | PRO |
| 14 | GLU | 194 | N | 32.407 | 8.211 | 5.884 | N | PRO |
| | | 195 | HN | 32.852 | 7.526 | 5.303 | H | PRO |
| | | 196 | CA | 31.902 | 9.389 | 5.205 | C | PRO |
| | | 197 | HA | 32.393 | 10.251 | 5.638 | H | PRO |
| | | 198 | CB | 32.2 | 9.353 | 3.685 | C | PRO |
| | | 199 | HB1 | 33.297 | 9.239 | 3.54 | H | PRO |
| | | 200 | HB2 | 31.717 | 8.451 | 3.251 | H | PRO |
| | | 201 | CG | 31.727 | 10.607 | 2.906 | C | PRO |
| | | 202 | HG1 | 30.652 | 10.804 | 3.107 | H | PRO |
| | | 203 | HG2 | 32.313 | 11.493 | 3.225 | H | PRO |
| | | 204 | CD | 31.867 | 10.445 | 1.394 | C | PRO |
| | | 205 | OE1 | 32.45 | 9.429 | 0.933 | O | PRO |
| | | 206 | OE2 | 31.355 | 11.337 | 0.665 | O | PRO |
| | | 207 | C | 30.408 | 9.569 | 5.373 | C | PRO |
| | | 208 | O | 29.964 | 10.658 | 5.728 | O | PRO |
| 15 | LEU | 209 | N | 29.595 | 8.507 | 5.157 | N | PRO |
| | | 210 | HN | 29.958 | 7.622 | 4.854 | H | PRO |
| | | 211 | CA | 28.152 | 8.612 | 5.245 | C | PRO |
| | | 212 | HA | 27.855 | 9.576 | 4.863 | H | PRO |
| | | 213 | CB | 27.429 | 7.519 | 4.425 | C | PRO |
| | | 214 | HB1 | 27.847 | 6.529 | 4.707 | H | PRO |
| | | 215 | HB2 | 26.344 | 7.514 | 4.672 | H | PRO |
| | | 216 | CG | 27.554 | 7.683 | 2.892 | C | PRO |
| | | 217 | HG | 28.637 | 7.73 | 2.633 | H | PRO |
| | | 218 | CD1 | 26.963 | 6.456 | 2.179 | C | PRO |
| | | 219 | HD11 | 27.055 | 6.569 | 1.081 | H | PRO |
| | | 220 | HD12 | 27.507 | 5.538 | 2.483 | H | PRO |
| | | 221 | HD13 | 25.889 | 6.337 | 2.435 | H | PRO |
| | | 222 | CD2 | 26.899 | 8.975 | 2.372 | C | PRO |
| | | 223 | HD21 | 26.96 | 9.016 | 1.264 | H | PRO |
| | | 224 | HD22 | 25.829 | 9.004 | 2.663 | H | PRO |
| | | 225 | HD23 | 27.406 | 9.874 | 2.779 | H | PRO |
| | | 226 | C | 27.668 | 8.559 | 6.672 | C | PRO |
| | | 227 | O | 26.607 | 9.101 | 6.98 | O | PRO |
| 16 | ARG | 228 | N | 28.456 | 7.975 | 7.608 | N | PRO |
| | | 229 | HN | 29.286 | 7.478 | 7.351 | H | PRO |
| | | 230 | CA | 28.187 | 8.097 | 9.029 | C | PRO |
| | | 231 | HA | 27.172 | 7.78 | 9.214 | H | PRO |
| | | 232 | CB | 29.16 | 7.269 | 9.903 | C | PRO |
| | | 233 | HB1 | 30.2 | 7.461 | 9.558 | H | PRO |
| | | 234 | HB2 | 29.087 | 7.605 | 10.964 | H | PRO |
| | | 235 | CG | 28.913 | 5.75 | 9.917 | C | PRO |
| | | 236 | HG1 | 27.917 | 5.553 | 10.37 | H | PRO |
| | | 237 | HG2 | 28.903 | 5.361 | 8.878 | H | PRO |
| | | 238 | CD | 30.004 | 5.024 | 10.715 | C | PRO |
| | | 239 | HD1 | 30.985 | 5.129 | 10.202 | H | PRO |
| | | 240 | HD2 | 30.071 | 5.443 | 11.742 | H | PRO |
| | | 241 | NE | 29.665 | 3.571 | 10.815 | N | PRO |
| | | 242 | HE | 28.901 | 3.2 | 10.276 | H | PRO |
| | | 243 | CZ | 30.467 | 2.673 | 11.443 | C | PRO |
| | | 244 | NH1 | 31.635 | 3.022 | 12.021 | N | PRO |
| | | 245 | HH11 | 32.184 | 2.305 | 12.486 | H | PRO |
| | | 246 | HH12 | 31.92 | 3.983 | 12.062 | H | PRO |
| | | 247 | NH2 | 30.068 | 1.383 | 11.521 | N | PRO |
| | | 248 | HH21 | 30.666 | 0.72 | 12.003 | H | PRO |
| | | 249 | HH22 | 29.14 | 1.164 | 11.235 | H | PRO |
| | | 250 | C | 28.31 | 9.542 | 9.465 | C | PRO |
| | | 251 | O | 27.462 | 10.053 | 10.19 | O | PRO |
| 17 | HIS | 252 | N | 29.368 | 10.245 | 9.006 | N | PRO |
| | | 253 | HN | 30.062 | 9.813 | 8.427 | H | PRO |
| | | 254 | CA | 29.583 | 11.638 | 9.333 | C | PRO |
| | | 255 | HA | 29.46 | 11.754 | 10.402 | H | PRO |
| | | 256 | CB | 31.011 | 12.077 | 8.943 | C | PRO |
| | | 257 | HB1 | 31.729 | 11.355 | 9.386 | H | PRO |
| | | 258 | HB2 | 31.14 | 12.03 | 7.841 | H | PRO |
| | | 259 | ND1 | 31.472 | 13.792 | 10.762 | N | PRO |
| | | 260 | HD1 | 31.299 | 13.188 | 11.548 | H | PRO |
| | | 261 | CG | 31.384 | 13.449 | 9.432 | C | PRO |
| | | 262 | CE1 | 31.805 | 15.106 | 10.809 | C | PRO |
| | | 263 | HE1 | 31.937 | 15.648 | 11.745 | H | PRO |
| | | 264 | NE2 | 31.938 | 15.628 | 9.604 | N | PRO |
| | | 265 | CD2 | 31.671 | 14.583 | 8.736 | C | PRO |
| | | 266 | HD2 | 31.706 | 14.744 | 7.667 | H | PRO |
| | | 267 | C | 28.59 | 12.547 | 8.659 | C | PRO |
| | | 268 | O | 28.031 | 13.438 | 9.288 | O | PRO |
| 18 | ARG | 269 | N | 28.334 | 12.326 | 7.353 | N | PRO |
| | | 270 | HN | 28.799 | 11.586 | 6.865 | H | PRO |
| | | 271 | CA | 27.496 | 13.175 | 6.539 | C | PRO |
| | | 272 | HA | 27.864 | 14.187 | 6.648 | H | PRO |
| | | 273 | CB | 27.602 | 12.765 | 5.049 | C | PRO |
| | | 274 | HB1 | 28.683 | 12.697 | 4.796 | H | PRO |
| | | 275 | HB2 | 27.16 | 11.757 | 4.897 | H | PRO |
| | | 276 | CG | 26.968 | 13.768 | 4.074 | C | PRO |
| | | 277 | HG1 | 25.865 | 13.773 | 4.223 | H | PRO |
| | | 278 | HG2 | 27.353 | 14.783 | 4.326 | H | PRO |
| | | 279 | CD | 27.282 | 13.473 | 2.603 | C | PRO |
| | | 280 | HD1 | 28.378 | 13.503 | 2.413 | H | PRO |
| | | 281 | HD2 | 26.872 | 12.482 | 2.308 | H | PRO |
| | | 282 | NE | 26.621 | 14.552 | 1.804 | N | PRO |
| | | 283 | HE | 26.223 | 15.321 | 2.3 | H | PRO |
| | | 284 | CZ | 26.408 | 14.483 | 0.466 | C | PRO |
| | | 285 | NH1 | 26.898 | 13.481 | −0.297 | N | PRO |
| | | 286 | HH11 | 26.621 | 13.45 | −1.259 | H | PRO |
| | | 287 | HH12 | 27.496 | 12.773 | 0.088 | H | PRO |
| | | 288 | NH2 | 25.655 | 15.435 | −0.135 | N | PRO |
| | | 289 | HH21 | 25.433 | 15.319 | −1.105 | H | PRO |
| | | 290 | HH22 | 25.254 | 16.172 | 0.401 | H | PRO |
| | | 291 | C | 26.047 | 13.162 | 6.972 | C | PRO |
| | | 292 | O | 25.398 | 14.207 | 7.02 | O | PRO |
| 19 | ILE | 293 | N | 25.513 | 11.964 | 7.297 | N | PRO |
| | | 294 | HN | 26.065 | 11.131 | 7.275 | H | PRO |
| | | 295 | CA | 24.104 | 11.785 | 7.564 | C | PRO |
| | | 296 | HA | 23.547 | 12.617 | 7.154 | H | PRO |
| | | 297 | CB | 23.568 | 10.498 | 6.933 | C | PRO |
| | | 298 | HB | 24.079 | 9.619 | 7.389 | H | PRO |
| | | 299 | CG2 | 22.049 | 10.379 | 7.207 | C | PRO |
| | | 300 | HG21 | 21.622 | 9.493 | 6.694 | H | PRO |
| | | 301 | HG22 | 21.84 | 10.269 | 8.291 | H | PRO |
| | | 302 | HG23 | 21.525 | 11.283 | 6.835 | H | PRO |
| | | 303 | CG1 | 23.885 | 10.465 | 5.414 | C | PRO |
| | | 304 | HG11 | 23.28 | 11.243 | 4.902 | H | PRO |
| | | 305 | HG12 | 24.955 | 10.706 | 5.242 | H | PRO |
| | | 306 | CD1 | 23.631 | 9.102 | 4.769 | C | PRO |
| | | 307 | HD1 | 23.918 | 9.123 | 3.698 | H | PRO |
| | | 308 | HD2 | 24.234 | 8.318 | 5.274 | H | PRO |
| | | 309 | HD3 | 22.56 | 8.824 | 4.836 | H | PRO |
| | | 310 | C | 23.872 | 11.762 | 9.056 | C | PRO |
| | | 311 | O | 23.114 | 12.573 | 9.587 | O | PRO |
| 20 | LEU | 312 | N | 24.502 | 10.801 | 9.77 | N | PRO |
| | | 313 | HN | 25.185 | 10.209 | 9.348 | H | PRO |
| | | 314 | CA | 24.15 | 10.474 | 11.135 | C | PRO |
| | | 315 | HA | 23.075 | 10.549 | 11.227 | H | PRO |
| | | 316 | CB | 24.558 | 9.025 | 11.492 | C | PRO |
| | | 317 | HB1 | 25.65 | 8.903 | 11.333 | H | PRO |
| | | 318 | HB2 | 24.35 | 8.822 | 12.566 | H | PRO |
| | | 319 | CG | 23.813 | 7.97 | 10.646 | C | PRO |
| | | 320 | HG | 23.808 | 8.312 | 9.584 | H | PRO |
| | | 321 | CD1 | 24.536 | 6.614 | 10.651 | C | PRO |
| | | 322 | HD11 | 23.986 | 5.881 | 10.023 | H | PRO |
| | | 323 | HD12 | 25.563 | 6.719 | 10.246 | H | PRO |
| | | 324 | HD13 | 24.604 | 6.213 | 11.678 | H | PRO |
| | | 325 | CD2 | 22.347 | 7.823 | 11.092 | C | PRO |
| | | 326 | HD21 | 21.84 | 7.05 | 10.479 | H | PRO |
| | | 327 | HD22 | 22.284 | 7.527 | 12.158 | H | PRO |
| | | 328 | HD23 | 21.8 | 8.78 | 10.965 | H | PRO |
| | | 329 | C | 24.733 | 11.419 | 12.154 | C | PRO |
| | | 330 | O | 24.031 | 11.823 | 13.079 | O | PRO |
| 21 | ARG | 331 | N | 26.021 | 11.804 | 12.012 | N | PRO |
| | | 332 | HN | 26.576 | 11.476 | 11.247 | H | PRO |
| | | 333 | CA | 26.711 | 12.554 | 13.043 | C | PRO |
| | | 334 | HA | 26.007 | 12.89 | 13.789 | H | PRO |
| | | 335 | CB | 27.767 | 11.672 | 13.761 | C | PRO |
| | | 336 | HB1 | 28.562 | 11.372 | 13.044 | H | PRO |
| | | 337 | HB2 | 28.237 | 12.274 | 14.57 | H | PRO |
| | | 338 | CG | 27.198 | 10.398 | 14.409 | C | PRO |
| | | 339 | HG1 | 26.24 | 10.667 | 14.906 | H | PRO |
| | | 340 | HG2 | 26.97 | 9.649 | 13.619 | H | PRO |
| | | 341 | CD | 28.161 | 9.788 | 15.444 | C | PRO |
| | | 342 | HD1 | 29.097 | 9.457 | 14.94 | H | PRO |
| | | 343 | HD2 | 28.426 | 10.53 | 16.227 | H | PRO |
| | | 344 | NE | 27.535 | 8.589 | 16.102 | N | PRO |
| | | 345 | HE | 27.874 | 7.681 | 15.863 | H | PRO |
| | | 346 | CZ | 26.489 | 8.646 | 16.97 | C | PRO |
| | | 347 | NH1 | 25.954 | 9.814 | 17.387 | N | PRO |
| | | 348 | HH11 | 25.13 | 9.789 | 17.946 | H | PRO |
| | | 349 | HH12 | 26.32 | 10.684 | 17.05 | H | PRO |
| | | 350 | NH2 | 25.945 | 7.487 | 17.411 | N | PRO |
| | | 351 | HH21 | 24.971 | 7.491 | 17.687 | H | PRO |
| | | 352 | HH22 | 26.283 | 6.612 | 17.031 | H | PRO |
| | | 353 | C | 27.41 | 13.798 | 12.506 | C | PRO |
| | | 354 | O | 28.622 | 13.923 | 12.696 | O | PRO |
| 22 | PRO | 355 | N | 26.755 | 14.766 | 11.857 | N | PRO |
| | | 356 | CD | 25.3 | 14.834 | 11.678 | C | PRO |
| | | 357 | HD1 | 24.989 | 14.028 | 10.978 | H | PRO |
| | | 358 | HD2 | 24.772 | 14.731 | 12.652 | H | PRO |
| | | 359 | CA | 27.431 | 15.873 | 11.192 | C | PRO |
| | | 360 | HA | 28.293 | 15.502 | 10.657 | H | PRO |
| | | 361 | CB | 26.344 | 16.45 | 10.271 | C | PRO |
| | | 362 | HB1 | 26.334 | 15.863 | 9.325 | H | PRO |
| | | 363 | HB2 | 26.492 | 17.52 | 10.026 | H | PRO |
| | | 364 | CG | 25.037 | 16.191 | 11.026 | C | PRO |
| | | 365 | HG1 | 24.154 | 16.179 | 10.357 | H | PRO |
| | | 366 | HG2 | 24.901 | 16.963 | 11.813 | H | PRO |
| | | 367 | C | 27.911 | 16.925 | 12.164 | C | PRO |
| | | 368 | O | 28.608 | 17.84 | 11.731 | O | PRO |
| 23 | ASN | 369 | N | 27.54 | 16.845 | 13.461 | N | PRO |
| | | 370 | HN | 26.966 | 16.093 | 13.786 | H | PRO |
| | | 371 | CA | 27.847 | 17.874 | 14.432 | C | PRO |
| | | 372 | HA | 28.286 | 18.733 | 13.943 | H | PRO |
| | | 373 | CB | 26.584 | 18.312 | 15.217 | C | PRO |
| | | 374 | HB1 | 26.193 | 17.466 | 15.82 | H | PRO |
| | | 375 | HB2 | 26.821 | 19.152 | 15.903 | H | PRO |
| | | 376 | CG | 25.478 | 18.752 | 14.256 | C | PRO |
| | | 377 | OD1 | 24.424 | 18.114 | 14.176 | O | PRO |
| | | 378 | ND2 | 25.723 | 19.868 | 13.513 | N | PRO |
| | | 379 | HD21 | 25.024 | 20.182 | 12.874 | H | PRO |
| | | 380 | HD22 | 26.595 | 20.346 | 13.598 | H | PRO |
| | | 381 | C | 28.859 | 17.355 | 15.425 | C | PRO |
| | | 382 | O | 29.045 | 17.945 | 16.488 | O | PRO |
| 24 | GLN | 383 | N | 29.54 | 16.233 | 15.102 | N | PRO |
| | | 384 | HN | 29.408 | 15.796 | 14.214 | H | PRO |
| | | 385 | CA | 30.461 | 15.575 | 16.002 | C | PRO |
| | | 386 | HA | 30.633 | 16.21 | 16.857 | H | PRO |
| | | 387 | CB | 29.902 | 14.207 | 16.475 | C | PRO |
| | | 388 | HB1 | 29.679 | 13.587 | 15.58 | H | PRO |
| | | 389 | HB2 | 30.671 | 13.675 | 17.077 | H | PRO |
| | | 390 | CG | 28.618 | 14.349 | 17.322 | C | PRO |
| | | 391 | HG1 | 28.845 | 14.879 | 18.27 | H | PRO |
| | | 392 | HG2 | 27.869 | 14.951 | 16.767 | H | PRO |
| | | 393 | CD | 27.988 | 12.988 | 17.634 | C | PRO |
| | | 394 | OE1 | 26.93 | 12.635 | 17.098 | O | PRO |
| | | 395 | NE2 | 28.653 | 12.209 | 18.532 | N | PRO |
| | | 396 | HE21 | 28.271 | 11.323 | 18.789 | H | PRO |
| | | 397 | HE22 | 29.501 | 12.539 | 18.941 | H | PRO |
| | | 398 | C | 31.764 | 15.349 | 15.252 | C | PRO |
| | | 399 | O | 31.732 | 15.275 | 14.023 | O | PRO |
| 25 | PRO | 400 | N | 32.923 | 15.238 | 15.918 | N | PRO |
| | | 401 | CD | 33.074 | 15.594 | 17.333 | C | PRO |
| | | 402 | HD1 | 32.627 | 16.589 | 17.551 | H | PRO |
| | | 403 | HD2 | 32.598 | 14.806 | 17.957 | H | PRO |
| | | 404 | CA | 34.197 | 14.789 | 15.349 | C | PRO |
| | | 405 | HA | 34.541 | 15.577 | 14.693 | H | PRO |
| | | 406 | CB | 35.093 | 14.586 | 16.579 | C | PRO |
| | | 407 | HB1 | 36.167 | 14.733 | 16.348 | H | PRO |
| | | 408 | HB2 | 34.946 | 13.569 | 17.007 | H | PRO |
| | | 409 | CG | 34.581 | 15.623 | 17.576 | C | PRO |
| | | 410 | HG1 | 34.985 | 16.624 | 17.307 | H | PRO |
| | | 411 | HG2 | 34.854 | 15.385 | 18.622 | H | PRO |
| | | 412 | C | 34.126 | 13.52 | 14.522 | C | PRO |
| | | 413 | O | 33.234 | 12.71 | 14.762 | O | PRO |
| 26 | ILE | 414 | N | 35.063 | 13.304 | 13.566 | N | PRO |
| | | 415 | HN | 35.806 | 13.959 | 13.425 | H | PRO |
| | | 416 | CA | 35.06 | 12.146 | 12.679 | C | PRO |
| | | 417 | HA | 34.049 | 12.053 | 12.308 | H | PRO |
| | | 418 | CB | 36.003 | 12.33 | 11.483 | C | PRO |
| | | 419 | HB | 35.742 | 13.309 | 11.012 | H | PRO |
| | | 420 | CG2 | 37.478 | 12.417 | 11.939 | C | PRO |
| | | 421 | HG21 | 38.13 | 12.668 | 11.076 | H | PRO |
| | | 422 | HG22 | 37.616 | 13.198 | 12.71 | H | PRO |
| | | 423 | HG23 | 37.823 | 11.449 | 12.356 | H | PRO |
| | | 424 | CG1 | 35.847 | 11.247 | 10.382 | C | PRO |
| | | 425 | HG11 | 36.57 | 11.482 | 9.569 | H | PRO |
| | | 426 | HG12 | 36.133 | 10.256 | 10.793 | H | PRO |
| | | 427 | CD1 | 34.447 | 11.161 | 9.77 | C | PRO |
| | | 428 | HD1 | 34.44 | 10.446 | 8.92 | H | PRO |
| | | 429 | HD2 | 33.709 | 10.807 | 10.521 | H | PRO |
| | | 430 | HD3 | 34.126 | 12.157 | 9.397 | H | PRO |
| | | 431 | C | 35.377 | 10.866 | 13.433 | C | PRO |
| | | 432 | O | 34.981 | 9.771 | 13.036 | O | PRO |
| 27 | GLU | 433 | N | 36.038 | 10.987 | 14.605 | N | PRO |
| | | 434 | HN | 36.404 | 11.877 | 14.895 | H | PRO |
| | | 435 | CA | 36.382 | 9.9 | 15.491 | C | PRO |
| | | 436 | HA | 36.826 | 9.112 | 14.901 | H | PRO |
| | | 437 | CB | 37.393 | 10.356 | 16.574 | C | PRO |
| | | 438 | HB1 | 36.957 | 11.171 | 17.19 | H | PRO |
| | | 439 | HB2 | 37.588 | 9.491 | 17.251 | H | PRO |
| | | 440 | CG | 38.77 | 10.808 | 16.021 | C | PRO |
| | | 441 | HG1 | 39.509 | 10.799 | 16.851 | H | PRO |
| | | 442 | HG2 | 39.108 | 10.082 | 15.252 | H | PRO |
| | | 443 | CD | 38.797 | 12.214 | 15.411 | C | PRO |
| | | 444 | OE1 | 37.789 | 12.961 | 15.519 | O | PRO |
| | | 445 | OE2 | 39.85 | 12.561 | 14.813 | O | PRO |
| | | 446 | C | 35.152 | 9.336 | 16.177 | C | PRO |
| | | 447 | O | 35.169 | 8.208 | 16.665 | O | PRO |
| 28 | ALA | 448 | N | 34.022 | 10.085 | 16.171 | N | PRO |
| | | 449 | HN | 34.015 | 10.996 | 15.76 | H | PRO |
| | | 450 | CA | 32.752 | 9.623 | 16.684 | C | PRO |
| | | 451 | HA | 32.929 | 9.119 | 17.624 | H | PRO |
| | | 452 | CB | 31.775 | 10.792 | 16.915 | C | PRO |
| | | 453 | HB1 | 32.241 | 11.545 | 17.587 | H | PRO |
| | | 454 | HB2 | 31.517 | 11.292 | 15.958 | H | PRO |
| | | 455 | HB3 | 30.837 | 10.433 | 17.39 | H | PRO |
| | | 456 | C | 32.095 | 8.645 | 15.732 | C | PRO |
| | | 457 | O | 31.199 | 7.903 | 16.13 | O | PRO |
| 29 | CYS | 458 | N | 32.562 | 8.588 | 14.462 | N | PRO |
| | | 459 | HN | 33.295 | 9.199 | 14.159 | H | PRO |
| | | 460 | CA | 32.058 | 7.683 | 13.45 | C | PRO |
| | | 461 | HA | 31.041 | 7.406 | 13.682 | H | PRO |
| | | 462 | CB | 32.105 | 8.307 | 12.04 | C | PRO |
| | | 463 | HB1 | 33.144 | 8.616 | 11.797 | H | PRO |
| | | 464 | HB2 | 31.795 | 7.559 | 11.279 | H | PRO |
| | | 465 | SG | 30.998 | 9.728 | 11.948 | S | PRO |
| | | 466 | HG1 | 31.186 | 9.948 | 10.654 | H | PRO |
| | | 467 | C | 32.888 | 6.432 | 13.391 | C | PRO |
| | | 468 | O | 32.624 | 5.55 | 12.578 | O | PRO |
| 30 | MET | 469 | N | 33.891 | 6.31 | 14.28 | N | PRO |
| | | 470 | HN | 34.093 | 7.038 | 14.933 | H | PRO |
| | | 471 | CA | 34.723 | 5.143 | 14.388 | C | PRO |
| | | 472 | HA | 34.586 | 4.488 | 13.537 | H | PRO |
| | | 473 | CB | 36.216 | 5.523 | 14.52 | C | PRO |
| | | 474 | HB1 | 36.38 | 6.082 | 15.468 | H | PRO |
| | | 475 | HB2 | 36.833 | 4.599 | 14.554 | H | PRO |
| | | 476 | CG | 36.699 | 6.412 | 13.36 | C | PRO |
| | | 477 | HG1 | 36.515 | 5.876 | 12.405 | H | PRO |
| | | 478 | HG2 | 36.078 | 7.333 | 13.338 | H | PRO |
| | | 479 | SD | 38.446 | 6.892 | 13.465 | S | PRO |
| | | 480 | CE | 38.358 | 8.066 | 12.082 | C | PRO |
| | | 481 | HE1 | 39.346 | 8.539 | 11.898 | H | PRO |
| | | 482 | HE2 | 38.043 | 7.556 | 11.146 | H | PRO |
| | | 483 | HE3 | 37.627 | 8.876 | 12.295 | H | PRO |
| | | 484 | C | 34.239 | 4.457 | 15.635 | C | PRO |
| | | 485 | O | 34.336 | 5.008 | 16.732 | O | PRO |
| 31 | PHE | 486 | N | 33.641 | 3.255 | 15.493 | N | PRO |
| | | 487 | HN | 33.596 | 2.77 | 14.62 | H | PRO |
| | | 488 | CA | 32.928 | 2.631 | 16.584 | C | PRO |
| | | 489 | HA | 32.75 | 3.342 | 17.379 | H | PRO |
| | | 490 | CB | 31.566 | 2.001 | 16.174 | C | PRO |
| | | 491 | HB1 | 31.747 | 1.154 | 15.477 | H | PRO |
| | | 492 | HB2 | 31.055 | 1.609 | 17.079 | H | PRO |
| | | 493 | CG | 30.578 | 2.923 | 15.496 | C | PRO |
| | | 494 | CD1 | 30.627 | 4.329 | 15.559 | C | PRO |
| | | 495 | HD1 | 31.398 | 4.831 | 16.116 | H | PRO |
| | | 496 | CE1 | 29.676 | 5.11 | 14.889 | C | PRO |
| | | 497 | HE1 | 29.722 | 6.185 | 14.945 | H | PRO |
| | | 498 | CZ | 28.662 | 4.498 | 14.149 | C | PRO |
| | | 499 | HZ | 27.934 | 5.1 | 13.627 | H | PRO |
| | | 500 | CD2 | 29.524 | 2.326 | 14.78 | C | PRO |
| | | 501 | HD2 | 29.456 | 1.25 | 14.733 | H | PRO |
| | | 502 | CE2 | 28.573 | 3.105 | 14.111 | C | PRO |
| | | 503 | HE2 | 27.788 | 2.63 | 13.544 | H | PRO |
| | | 504 | C | 33.771 | 1.51 | 17.127 | C | PRO |
| | | 505 | O | 34.5 | 0.836 | 16.399 | O | PRO |
| 32 | GLU | 506 | N | 33.652 | 1.247 | 18.445 | N | PRO |
| | | 507 | HN | 33.11 | 1.839 | 19.046 | H | PRO |
| | | 508 | CA | 34.278 | 0.112 | 19.093 | C | PRO |
| | | 509 | HA | 35.318 | 0.065 | 18.796 | H | PRO |
| | | 510 | CB | 34.18 | 0.222 | 20.63 | C | PRO |
| | | 511 | HB1 | 33.107 | 0.316 | 20.914 | H | PRO |
| | | 512 | HB2 | 34.578 | −0.706 | 21.1 | H | PRO |
| | | 513 | CG | 34.957 | 1.423 | 21.21 | C | PRO |
| | | 514 | HG1 | 36.047 | 1.286 | 21.044 | H | PRO |
| | | 515 | HG2 | 34.646 | 2.361 | 20.705 | H | PRO |
| | | 516 | CD | 34.709 | 1.59 | 22.71 | C | PRO |
| | | 517 | OE1 | 33.99 | 0.744 | 23.307 | O | PRO |
| | | 518 | OE2 | 35.238 | 2.584 | 23.273 | O | PRO |
| | | 519 | C | 33.598 | −1.174 | 18.675 | C | PRO |
| | | 520 | O | 34.19 | −2.249 | 18.737 | O | PRO |
| 33 | SER | 521 | N | 32.341 | −1.073 | 18.184 | N | PRO |
| | | 522 | HN | 31.882 | −0.187 | 18.161 | H | PRO |
| | | 523 | CA | 31.548 | −2.176 | 17.697 | C | PRO |
| | | 524 | HA | 31.654 | −2.991 | 18.397 | H | PRO |
| | | 525 | CB | 30.05 | −1.798 | 17.616 | C | PRO |
| | | 526 | HB1 | 29.463 | −2.657 | 17.236 | H | PRO |
| | | 527 | HB2 | 29.69 | −1.556 | 18.64 | H | PRO |
| | | 528 | OG | 29.818 | −0.672 | 16.774 | O | PRO |
| | | 529 | HG1 | 28.99 | −0.278 | 17.067 | H | PRO |
| | | 530 | C | 32.008 | −2.68 | 16.348 | C | PRO |
| | | 531 | O | 31.748 | −3.83 | 16 | O | PRO |
| 34 | ASP | 532 | N | 32.763 | −1.857 | 15.576 | N | PRO |
| | | 533 | HN | 32.971 | −0.922 | 15.858 | H | PRO |
| | | 534 | CA | 33.366 | −2.294 | 14.33 | C | PRO |
| | | 535 | HA | 32.639 | −2.869 | 13.77 | H | PRO |
| | | 536 | CB | 33.9 | −1.122 | 13.46 | C | PRO |
| | | 537 | HB1 | 34.727 | −0.605 | 13.995 | H | PRO |
| | | 538 | HB2 | 34.299 | −1.522 | 12.502 | H | PRO |
| | | 539 | CG | 32.829 | −0.092 | 13.118 | C | PRO |
| | | 540 | OD1 | 31.709 | −0.486 | 12.693 | O | PRO |
| | | 541 | OD2 | 33.129 | 1.127 | 13.249 | O | PRO |
| | | 542 | C | 34.565 | −3.175 | 14.623 | C | PRO |
| | | 543 | O | 34.999 | −3.958 | 13.779 | O | PRO |
| 35 | LEU | 544 | N | 35.112 | −3.067 | 15.854 | N | PRO |
| | | 545 | HN | 34.717 | −2.442 | 16.525 | H | PRO |
| | | 546 | CA | 36.306 | −3.75 | 16.29 | C | PRO |
| | | 547 | HA | 36.843 | −4.136 | 15.434 | H | PRO |
| | | 548 | CB | 37.226 | −2.793 | 17.088 | C | PRO |
| | | 549 | HB1 | 36.696 | −2.466 | 18.009 | H | PRO |
| | | 550 | HB2 | 38.147 | −3.334 | 17.401 | H | PRO |
| | | 551 | CG | 37.653 | −1.516 | 16.327 | C | PRO |
| | | 552 | HG | 36.733 | −0.984 | 15.99 | H | PRO |
| | | 553 | CD1 | 38.402 | −0.558 | 17.271 | C | PRO |
| | | 554 | HD11 | 38.684 | 0.372 | 16.734 | H | PRO |
| | | 555 | HD12 | 37.758 | −0.285 | 18.134 | H | PRO |
| | | 556 | HD13 | 39.326 | −1.039 | 17.655 | H | PRO |
| | | 557 | CD2 | 38.498 | −1.824 | 15.078 | C | PRO |
| | | 558 | HD21 | 38.807 | −0.877 | 14.585 | H | PRO |
| | | 559 | HD22 | 39.414 | −2.385 | 15.36 | H | PRO |
| | | 560 | HD23 | 37.922 | −2.424 | 14.343 | H | PRO |
| | | 561 | C | 35.949 | −4.921 | 17.181 | C | PRO |
| | | 562 | O | 36.834 | −5.581 | 17.722 | O | PRO |
| 36 | LEU | 563 | N | 34.639 | −5.228 | 17.349 | N | PRO |
| | | 564 | HN | 33.925 | −4.675 | 16.925 | H | PRO |
| | | 565 | CA | 34.193 | −6.411 | 18.059 | C | PRO |
| | | 566 | HA | 34.919 | −6.647 | 18.822 | H | PRO |
| | | 567 | CB | 32.808 | −6.247 | 18.735 | C | PRO |
| | | 568 | HB1 | 32.093 | −5.836 | 17.988 | H | PRO |
| | | 569 | HB2 | 32.423 | −7.236 | 19.065 | H | PRO |
| | | 570 | CG | 32.829 | −5.321 | 19.977 | C | PRO |
| | | 571 | HG | 33.283 | −4.352 | 19.671 | H | PRO |
| | | 572 | CD1 | 31.409 | −5.017 | 20.482 | C | PRO |
| | | 573 | HD11 | 31.45 | −4.322 | 21.348 | H | PRO |
| | | 574 | HD12 | 30.806 | −4.541 | 19.683 | H | PRO |
| | | 575 | HD13 | 30.903 | −5.952 | 20.803 | H | PRO |
| | | 576 | CD2 | 33.677 | −5.887 | 21.133 | C | PRO |
| | | 577 | HD21 | 33.638 | −5.196 | 22.002 | H | PRO |
| | | 578 | HD22 | 33.283 | −6.875 | 21.452 | H | PRO |
| | | 579 | HD23 | 34.739 | −6.002 | 20.837 | H | PRO |
| | | 580 | C | 34.176 | −7.581 | 17.105 | C | PRO |
| | | 581 | O | 34.098 | −7.415 | 15.888 | O | PRO |
| 37 | ARG | 582 | N | 34.315 | −8.807 | 17.66 | N | PRO |
| | | 583 | HN | 34.323 | −8.913 | 18.651 | H | PRO |
| | | 584 | CA | 34.56 | −10.024 | 16.914 | C | PRO |
| | | 585 | HA | 35.486 | −9.877 | 16.376 | H | PRO |
| | | 586 | CB | 34.735 | −11.227 | 17.876 | C | PRO |
| | | 587 | HB1 | 35.495 | −10.941 | 18.635 | H | PRO |
| | | 588 | HB2 | 33.779 | −11.402 | 18.417 | H | PRO |
| | | 589 | CG | 35.183 | −12.539 | 17.201 | C | PRO |
| | | 590 | HG1 | 34.423 | −12.831 | 16.442 | H | PRO |
| | | 591 | HG2 | 36.142 | −12.37 | 16.664 | H | PRO |
| | | 592 | CD | 35.335 | −13.722 | 18.169 | C | PRO |
| | | 593 | HD1 | 34.393 | −13.873 | 18.742 | H | PRO |
| | | 594 | HD2 | 35.587 | −14.648 | 17.606 | H | PRO |
| | | 595 | NE | 36.452 | −13.42 | 19.123 | N | PRO |
| | | 596 | HE | 36.965 | −12.574 | 19.003 | H | PRO |
| | | 597 | CZ | 36.799 | −14.242 | 20.148 | C | PRO |
| | | 598 | NH1 | 36.155 | −15.411 | 20.36 | N | PRO |
| | | 599 | HH11 | 36.428 | −16.001 | 21.115 | H | PRO |
| | | 600 | HH12 | 35.414 | −15.678 | 19.75 | H | PRO |
| | | 601 | NH2 | 37.812 | −13.885 | 20.972 | N | PRO |
| | | 602 | HH21 | 38.072 | −14.483 | 21.725 | H | PRO |
| | | 603 | HH22 | 38.293 | −13.025 | 20.824 | H | PRO |
| | | 604 | C | 33.475 | −10.349 | 15.91 | C | PRO |
| | | 605 | O | 32.287 | −10.374 | 16.231 | O | PRO |
| 38 | GLY | 606 | N | 33.885 | −10.626 | 14.648 | N | PRO |
| | | 607 | HN | 34.851 | −10.587 | 14.388 | H | PRO |
| | | 608 | CA | 32.989 | −11.086 | 13.612 | C | PRO |
| | | 609 | HA1 | 32.325 | −11.827 | 14.034 | H | PRO |
| | | 610 | HA2 | 33.611 | −11.495 | 12.831 | H | PRO |
| | | 611 | C | 32.164 | −9.991 | 12.996 | C | PRO |
| | | 612 | O | 31.191 | −10.28 | 12.302 | O | PRO |
| 39 | ALA | 613 | N | 32.491 | −8.705 | 13.258 | N | PRO |
| | | 614 | HN | 33.24 | −8.485 | 13.881 | H | PRO |
| | | 615 | CA | 31.814 | −7.569 | 12.668 | C | PRO |
| | | 616 | HA | 30.767 | −7.684 | 12.906 | H | PRO |
| | | 617 | CB | 32.294 | −6.223 | 13.243 | C | PRO |
| | | 618 | HB1 | 32.208 | −6.229 | 14.351 | H | PRO |
| | | 619 | HB2 | 33.355 | −6.036 | 12.977 | H | PRO |
| | | 620 | HB3 | 31.677 | −5.385 | 12.853 | H | PRO |
| | | 621 | C | 31.942 | −7.538 | 11.157 | C | PRO |
| | | 622 | O | 32.925 | −8.027 | 10.598 | O | PRO |
| 40 | PHE | 623 | N | 30.925 | −6.982 | 10.465 | N | PRO |
| | | 624 | HN | 30.123 | −6.599 | 10.931 | H | PRO |
| | | 625 | CA | 30.942 | −6.865 | 9.026 | C | PRO |
| | | 626 | HA | 31.96 | −6.648 | 8.727 | H | PRO |
| | | 627 | CB | 30.473 | −8.135 | 8.252 | C | PRO |
| | | 628 | HB1 | 30.603 | −7.978 | 7.161 | H | PRO |
| | | 629 | HB2 | 31.119 | −8.988 | 8.543 | H | PRO |
| | | 630 | CG | 29.04 | −8.542 | 8.504 | C | PRO |
| | | 631 | CD1 | 28.7 | −9.324 | 9.621 | C | PRO |
| | | 632 | HD1 | 29.466 | −9.601 | 10.328 | H | PRO |
| | | 633 | CE1 | 27.382 | −9.758 | 9.815 | C | PRO |
| | | 634 | HE1 | 27.133 | −10.362 | 10.675 | H | PRO |
| | | 635 | CZ | 26.387 | −9.402 | 8.895 | C | PRO |
| | | 636 | HZ | 25.369 | −9.73 | 9.042 | H | PRO |
| | | 637 | CD2 | 28.032 | −8.194 | 7.586 | C | PRO |
| | | 638 | HD2 | 28.28 | −7.602 | 6.717 | H | PRO |
| | | 639 | CE2 | 26.712 | −8.618 | 7.781 | C | PRO |
| | | 640 | HE2 | 25.944 | −8.341 | 7.074 | H | PRO |
| | | 641 | C | 30.126 | −5.662 | 8.64 | C | PRO |
| | | 642 | O | 29.352 | −5.135 | 9.438 | O | PRO |
| 41 | HIS | 643 | N | 30.326 | −5.187 | 7.393 | N | PRO |
| | | 644 | HN | 30.946 | −5.646 | 6.759 | H | PRO |
| | | 645 | CA | 29.796 | −3.929 | 6.933 | C | PRO |
| | | 646 | HA | 28.953 | −3.639 | 7.544 | H | PRO |
| | | 647 | CB | 30.876 | −2.82 | 6.942 | C | PRO |
| | | 648 | HB1 | 31.593 | −2.983 | 6.114 | H | PRO |
| | | 649 | HB2 | 30.403 | −1.824 | 6.795 | H | PRO |
| | | 650 | ND1 | 31.24 | −2.354 | 9.409 | N | PRO |
| | | 651 | HD1 | 30.34 | −1.934 | 9.56 | H | PRO |
| | | 652 | CG | 31.695 | −2.824 | 8.202 | C | PRO |
| | | 653 | CE1 | 32.2 | −2.612 | 10.328 | C | PRO |
| | | 654 | HE1 | 32.104 | −2.336 | 11.379 | H | PRO |
| | | 655 | NE2 | 33.239 | −3.225 | 9.797 | N | PRO |
| | | 656 | CD2 | 32.922 | −3.355 | 8.455 | C | PRO |
| | | 657 | HD2 | 33.612 | −3.843 | 7.78 | H | PRO |
| | | 658 | C | 29.324 | −4.139 | 5.52 | C | PRO |
| | | 659 | O | 30.021 | −4.752 | 4.711 | O | PRO |
| 42 | LEU | 660 | N | 28.112 | −3.637 | 5.196 | N | PRO |
| | | 661 | HN | 27.582 | −3.103 | 5.857 | H | PRO |
| | | 662 | CA | 27.504 | −3.798 | 3.893 | C | PRO |
| | | 663 | HA | 28.185 | −4.304 | 3.223 | H | PRO |
| | | 664 | CB | 26.151 | −4.548 | 3.925 | C | PRO |
| | | 665 | HB1 | 25.441 | −3.987 | 4.573 | H | PRO |
| | | 666 | HB2 | 25.724 | −4.574 | 2.897 | H | PRO |
| | | 667 | CG | 26.222 | −6.005 | 4.437 | C | PRO |
| | | 668 | HG | 26.608 | −5.984 | 5.483 | H | PRO |
| | | 669 | CD1 | 24.813 | −6.622 | 4.475 | C | PRO |
| | | 670 | HD11 | 24.851 | −7.655 | 4.88 | H | PRO |
| | | 671 | HD12 | 24.144 | −6.014 | 5.119 | H | PRO |
| | | 672 | HD13 | 24.378 | −6.654 | 3.454 | H | PRO |
| | | 673 | CD2 | 27.173 | −6.887 | 3.606 | C | PRO |
| | | 674 | HD21 | 27.156 | −7.93 | 3.988 | H | PRO |
| | | 675 | HD22 | 26.863 | −6.894 | 2.54 | H | PRO |
| | | 676 | HD23 | 28.217 | −6.517 | 3.67 | H | PRO |
| | | 677 | C | 27.257 | −2.426 | 3.335 | C | PRO |
| | | 678 | O | 26.919 | −1.495 | 4.066 | O | PRO |
| 43 | GLY | 679 | N | 27.454 | −2.277 | 2.007 | N | PRO |
| | | 680 | HN | 27.743 | −3.05 | 1.437 | H | PRO |
| | | 681 | CA | 27.289 | −1.02 | 1.318 | C | PRO |
| | | 682 | HA1 | 28.244 | −0.775 | 0.877 | H | PRO |
| | | 683 | HA2 | 26.921 | −0.262 | 1.996 | H | PRO |
| | | 684 | C | 26.308 | −1.181 | 0.207 | C | PRO |
| | | 685 | O | 26.282 | −2.207 | −0.471 | O | PRO |
| 44 | GLY | 686 | N | 25.481 | −0.135 | −0.01 | N | PRO |
| | | 687 | HN | 25.54 | 0.679 | 0.572 | H | PRO |
| | | 688 | CA | 24.504 | −0.083 | −1.074 | C | PRO |
| | | 689 | HA1 | 23.579 | 0.278 | −0.652 | H | PRO |
| | | 690 | HA2 | 24.406 | −1.053 | −1.54 | H | PRO |
| | | 691 | C | 24.974 | 0.907 | −2.081 | C | PRO |
| | | 692 | O | 25.326 | 2.031 | −1.733 | O | PRO |
| 45 | TYR | 693 | N | 24.988 | 0.504 | −3.365 | N | PRO |
| | | 694 | HN | 24.692 | −0.421 | −3.61 | H | PRO |
| | | 695 | CA | 25.54 | 1.296 | −4.437 | C | PRO |
| | | 696 | HA | 25.886 | 2.243 | −4.055 | H | PRO |
| | | 697 | CB | 26.692 | 0.588 | −5.196 | C | PRO |
| | | 698 | HB1 | 26.399 | −0.447 | −5.464 | H | PRO |
| | | 699 | HB2 | 26.941 | 1.136 | −6.132 | H | PRO |
| | | 700 | CG | 27.937 | 0.521 | −4.354 | C | PRO |
| | | 701 | CD1 | 28.065 | −0.415 | −3.311 | C | PRO |
| | | 702 | HD1 | 27.259 | −1.104 | −3.1 | H | PRO |
| | | 703 | CE1 | 29.229 | −0.463 | −2.534 | C | PRO |
| | | 704 | HE1 | 29.309 | −1.174 | −1.726 | H | PRO |
| | | 705 | CZ | 30.286 | 0.415 | −2.804 | C | PRO |
| | | 706 | OH | 31.454 | 0.368 | −2.014 | O | PRO |
| | | 707 | HH | 32.074 | 1.016 | −2.354 | H | PRO |
| | | 708 | CD2 | 29.007 | 1.393 | −4.616 | C | PRO |
| | | 709 | HD2 | 28.927 | 2.114 | −5.416 | H | PRO |
| | | 710 | CE2 | 30.177 | 1.342 | −3.847 | C | PRO |
| | | 711 | HE2 | 30.984 | 2.028 | −4.06 | H | PRO |
| | | 712 | C | 24.45 | 1.572 | −5.432 | C | PRO |
| | | 713 | O | 23.644 | 0.7 | −5.755 | O | PRO |
| 46 | TYR | 714 | N | 24.418 | 2.821 | −5.943 | N | PRO |
| | | 715 | HN | 25.058 | 3.526 | −5.631 | H | PRO |
| | | 716 | CA | 23.544 | 3.205 | −7.021 | C | PRO |
| | | 717 | HA | 23.345 | 2.338 | −7.637 | H | PRO |
| | | 718 | CB | 22.224 | 3.846 | −6.519 | C | PRO |
| | | 719 | HB1 | 21.752 | 3.171 | −5.773 | H | PRO |
| | | 720 | HB2 | 22.421 | 4.821 | −6.025 | H | PRO |
| | | 721 | CG | 21.234 | 4.044 | −7.638 | C | PRO |
| | | 722 | CD1 | 20.956 | 5.331 | −8.129 | C | PRO |
| | | 723 | HD1 | 21.461 | 6.187 | −7.709 | H | PRO |
| | | 724 | CE1 | 20.029 | 5.515 | −9.163 | C | PRO |
| | | 725 | HE1 | 19.816 | 6.511 | −9.525 | H | PRO |
| | | 726 | CZ | 19.371 | 4.411 | −9.717 | C | PRO |
| | | 727 | OH | 18.415 | 4.599 | −10.737 | O | PRO |
| | | 728 | HH | 17.973 | 3.76 | −10.883 | H | PRO |
| | | 729 | CD2 | 20.581 | 2.941 | −8.213 | C | PRO |
| | | 730 | HD2 | 20.792 | 1.946 | −7.854 | H | PRO |
| | | 731 | CE2 | 19.654 | 3.122 | −9.248 | C | PRO |
| | | 732 | HE2 | 19.156 | 2.265 | −9.674 | H | PRO |
| | | 733 | C | 24.326 | 4.201 | −7.834 | C | PRO |
| | | 734 | O | 24.921 | 5.135 | −7.294 | O | PRO |
| 47 | GLY | 735 | N | 24.364 | 4 | −9.173 | N | PRO |
| | | 736 | HN | 23.852 | 3.243 | −9.591 | H | PRO |
| | | 737 | CA | 25.085 | 4.848 | −10.102 | C | PRO |
| | | 738 | HA1 | 24.72 | 5.858 | −9.983 | H | PRO |
| | | 739 | HA2 | 24.906 | 4.454 | −11.092 | H | PRO |
| | | 740 | C | 26.573 | 4.851 | −9.876 | C | PRO |
| | | 741 | O | 27.241 | 5.843 | −10.163 | O | PRO |
| 48 | GLY | 742 | N | 27.12 | 3.742 | −9.323 | N | PRO |
| | | 743 | HN | 26.536 | 2.968 | −9.087 | H | PRO |
| | | 744 | CA | 28.537 | 3.585 | −9.062 | C | PRO |
| | | 745 | HA1 | 29.088 | 4.005 | −9.892 | H | PRO |
| | | 746 | HA2 | 28.713 | 2.526 | −8.943 | H | PRO |
| | | 747 | C | 29.014 | 4.262 | −7.802 | C | PRO |
| | | 748 | O | 30.214 | 4.295 | −7.545 | O | PRO |
| 49 | LYS | 749 | N | 28.092 | 4.819 | −6.985 | N | PRO |
| | | 750 | HN | 27.118 | 4.769 | −7.201 | H | PRO |
| | | 751 | CA | 28.431 | 5.573 | −5.799 | C | PRO |
| | | 752 | HA | 29.504 | 5.631 | −5.68 | H | PRO |
| | | 753 | CB | 27.84 | 7.006 | −5.849 | C | PRO |
| | | 754 | HB1 | 28.199 | 7.498 | −6.781 | H | PRO |
| | | 755 | HB2 | 26.731 | 6.939 | −5.919 | H | PRO |
| | | 756 | CG | 28.202 | 7.912 | −4.658 | C | PRO |
| | | 757 | HG1 | 27.872 | 7.418 | −3.718 | H | PRO |
| | | 758 | HG2 | 29.306 | 8.036 | −4.612 | H | PRO |
| | | 759 | CD | 27.517 | 9.287 | −4.728 | C | PRO |
| | | 760 | HD1 | 27.941 | 9.878 | −5.569 | H | PRO |
| | | 761 | HD2 | 26.439 | 9.116 | −4.955 | H | PRO |
| | | 762 | CE | 27.588 | 10.082 | −3.417 | C | PRO |
| | | 763 | HE1 | 26.979 | 11.009 | −3.495 | H | PRO |
| | | 764 | HE2 | 27.201 | 9.464 | −2.578 | H | PRO |
| | | 765 | NZ | 28.975 | 10.482 | −3.081 | N | PRO |
| | | 766 | HZ1 | 28.98 | 10.947 | −2.144 | H | PRO |
| | | 767 | HZ2 | 29.589 | 9.634 | −3.035 | H | PRO |
| | | 768 | HZ3 | 29.34 | 11.133 | −3.803 | H | PRO |
| | | 769 | C | 27.847 | 4.853 | −4.613 | C | PRO |
| | | 770 | O | 26.706 | 4.397 | −4.66 | O | PRO |
| 50 | LEU | 771 | N | 28.632 | 4.737 | −3.512 | N | PRO |
| | | 772 | HN | 29.573 | 5.089 | −3.524 | H | PRO |
| | | 773 | CA | 28.185 | 4.231 | −2.229 | C | PRO |
| | | 774 | HA | 27.686 | 3.287 | −2.4 | H | PRO |
| | | 775 | CB | 29.374 | 4.035 | −1.251 | C | PRO |
| | | 776 | HB1 | 30.105 | 3.343 | −1.724 | H | PRO |
| | | 777 | HB2 | 29.891 | 5.012 | −1.118 | H | PRO |
| | | 778 | CG | 29.018 | 3.477 | 0.149 | C | PRO |
| | | 779 | HG | 28.256 | 4.146 | 0.611 | H | PRO |
| | | 780 | CD1 | 28.429 | 2.062 | 0.091 | C | PRO |
| | | 781 | HD11 | 28.172 | 1.726 | 1.118 | H | PRO |
| | | 782 | HD12 | 27.51 | 2.032 | −0.527 | H | PRO |
| | | 783 | HD13 | 29.169 | 1.353 | −0.334 | H | PRO |
| | | 784 | CD2 | 30.244 | 3.484 | 1.076 | C | PRO |
| | | 785 | HD21 | 29.969 | 3.116 | 2.087 | H | PRO |
| | | 786 | HD22 | 31.044 | 2.831 | 0.668 | H | PRO |
| | | 787 | HD23 | 30.643 | 4.514 | 1.175 | H | PRO |
| | | 788 | C | 27.208 | 5.212 | −1.622 | C | PRO |
| | | 789 | O | 27.545 | 6.376 | −1.404 | O | PRO |
| 51 | ILE | 790 | N | 25.959 | 4.765 | −1.366 | N | PRO |
| | | 791 | HN | 25.705 | 3.813 | −1.558 | H | PRO |
| | | 792 | CA | 24.88 | 5.654 | −0.991 | C | PRO |
| | | 793 | HA | 25.29 | 6.576 | −0.602 | H | PRO |
| | | 794 | CB | 23.968 | 6.003 | −2.17 | C | PRO |
| | | 795 | HB | 23.072 | 6.554 | −1.801 | H | PRO |
| | | 796 | CG2 | 24.745 | 6.986 | −3.072 | C | PRO |
| | | 797 | HG21 | 24.097 | 7.356 | −3.891 | H | PRO |
| | | 798 | HG22 | 25.104 | 7.855 | −2.484 | H | PRO |
| | | 799 | HG23 | 25.62 | 6.485 | −3.534 | H | PRO |
| | | 800 | CG1 | 23.477 | 4.797 | −3.017 | C | PRO |
| | | 801 | HG11 | 23.009 | 5.223 | −3.933 | H | PRO |
| | | 802 | HG12 | 24.347 | 4.195 | −3.354 | H | PRO |
| | | 803 | CD1 | 22.436 | 3.877 | −2.367 | C | PRO |
| | | 804 | HD1 | 22.033 | 3.167 | −3.121 | H | PRO |
| | | 805 | HD2 | 22.889 | 3.28 | −1.55 | H | PRO |
| | | 806 | HD3 | 21.594 | 4.47 | −1.951 | H | PRO |
| | | 807 | C | 24.064 | 5.078 | 0.137 | C | PRO |
| | | 808 | O | 23.087 | 5.692 | 0.558 | O | PRO |
| 52 | SER | 809 | N | 24.45 | 3.911 | 0.693 | N | PRO |
| | | 810 | HN | 25.245 | 3.403 | 0.365 | H | PRO |
| | | 811 | CA | 23.771 | 3.357 | 1.841 | C | PRO |
| | | 812 | HA | 23.474 | 4.165 | 2.492 | H | PRO |
| | | 813 | CB | 22.541 | 2.482 | 1.473 | C | PRO |
| | | 814 | HB1 | 21.88 | 3.06 | 0.792 | H | PRO |
| | | 815 | HB2 | 22.867 | 1.566 | 0.939 | H | PRO |
| | | 816 | OG | 21.776 | 2.104 | 2.615 | O | PRO |
| | | 817 | HG1 | 21.153 | 2.827 | 2.792 | H | PRO |
| | | 818 | C | 24.797 | 2.538 | 2.567 | C | PRO |
| | | 819 | O | 25.711 | 2.001 | 1.945 | O | PRO |
| 53 | ILE | 820 | N | 24.675 | 2.451 | 3.907 | N | PRO |
| | | 821 | HN | 23.895 | 2.872 | 4.376 | H | PRO |
| | | 822 | CA | 25.652 | 1.814 | 4.76 | C | PRO |
| | | 823 | HA | 26.171 | 1.045 | 4.204 | H | PRO |
| | | 824 | CB | 26.664 | 2.78 | 5.383 | C | PRO |
| | | 825 | HB | 27.198 | 2.267 | 6.217 | H | PRO |
| | | 826 | CG2 | 27.728 | 3.129 | 4.321 | C | PRO |
| | | 827 | HG21 | 28.497 | 3.807 | 4.745 | H | PRO |
| | | 828 | HG22 | 28.229 | 2.209 | 3.957 | H | PRO |
| | | 829 | HG23 | 27.259 | 3.637 | 3.455 | H | PRO |
| | | 830 | CG1 | 25.982 | 4.048 | 5.96 | C | PRO |
| | | 831 | HG11 | 25.659 | 4.702 | 5.121 | H | PRO |
| | | 832 | HG12 | 25.071 | 3.759 | 6.528 | H | PRO |
| | | 833 | CD1 | 26.88 | 4.841 | 6.912 | C | PRO |
| | | 834 | HD1 | 26.35 | 5.746 | 7.276 | H | PRO |
| | | 835 | HD2 | 27.149 | 4.219 | 7.792 | H | PRO |
| | | 836 | HD3 | 27.818 | 5.156 | 6.41 | H | PRO |
| | | 837 | C | 24.903 | 1.134 | 5.876 | C | PRO |
| | | 838 | O | 23.851 | 1.595 | 6.317 | O | PRO |
| 54 | ALA | 839 | N | 25.448 | −0.007 | 6.346 | N | PRO |
| | | 840 | HN | 26.255 | −0.417 | 5.92 | H | PRO |
| | | 841 | CA | 24.943 | −0.709 | 7.494 | C | PRO |
| | | 842 | HA | 24.604 | 0.006 | 8.233 | H | PRO |
| | | 843 | CB | 23.825 | −1.704 | 7.129 | C | PRO |
| | | 844 | HB1 | 22.995 | −1.168 | 6.624 | H | PRO |
| | | 845 | HB2 | 24.199 | −2.48 | 6.428 | H | PRO |
| | | 846 | HB3 | 23.421 | −2.2 | 8.036 | H | PRO |
| | | 847 | C | 26.109 | −1.476 | 8.052 | C | PRO |
| | | 848 | O | 27.004 | −1.87 | 7.305 | O | PRO |
| 55 | SER | 849 | N | 26.129 | −1.707 | 9.384 | N | PRO |
| | | 850 | HN | 25.41 | −1.373 | 9.992 | H | PRO |
| | | 851 | CA | 27.231 | −2.385 | 10.031 | C | PRO |
| | | 852 | HA | 27.72 | −3.041 | 9.326 | H | PRO |
| | | 853 | CB | 28.267 | −1.428 | 10.665 | C | PRO |
| | | 854 | HB1 | 27.769 | −0.745 | 11.388 | H | PRO |
| | | 855 | HB2 | 29.045 | −2.011 | 11.207 | H | PRO |
| | | 856 | OG | 28.9 | −0.654 | 9.65 | O | PRO |
| | | 857 | HG1 | 29.638 | −0.184 | 10.057 | H | PRO |
| | | 858 | C | 26.666 | −3.235 | 11.127 | C | PRO |
| | | 859 | O | 25.742 | −2.833 | 11.832 | O | PRO |
| 56 | PHE | 860 | N | 27.22 | −4.455 | 11.283 | N | PRO |
| | | 861 | HN | 28.015 | −4.736 | 10.741 | H | PRO |
| | | 862 | CA | 26.624 | −5.503 | 12.08 | C | PRO |
| | | 863 | HA | 25.875 | −5.094 | 12.743 | H | PRO |
| | | 864 | CB | 26.027 | −6.638 | 11.208 | C | PRO |
| | | 865 | HB1 | 26.829 | −7.123 | 10.609 | H | PRO |
| | | 866 | HB2 | 25.534 | −7.408 | 11.839 | H | PRO |
| | | 867 | CG | 25.013 | −6.089 | 10.244 | C | PRO |
| | | 868 | CD1 | 25.407 | −5.658 | 8.964 | C | PRO |
| | | 869 | HD1 | 26.443 | −5.734 | 8.669 | H | PRO |
| | | 870 | CE1 | 24.476 | −5.102 | 8.081 | C | PRO |
| | | 871 | HE1 | 24.793 | −4.762 | 7.107 | H | PRO |
| | | 872 | CZ | 23.134 | −4.989 | 8.465 | C | PRO |
| | | 873 | HZ | 22.414 | −4.57 | 7.778 | H | PRO |
| | | 874 | CD2 | 23.664 | −5.977 | 10.616 | C | PRO |
| | | 875 | HD2 | 23.353 | −6.302 | 11.597 | H | PRO |
| | | 876 | CE2 | 22.725 | −5.439 | 9.727 | C | PRO |
| | | 877 | HE2 | 21.689 | −5.357 | 10.018 | H | PRO |
| | | 878 | C | 27.716 | −6.127 | 12.902 | C | PRO |
| | | 879 | O | 28.834 | −6.299 | 12.42 | O | PRO |
| 57 | HIS | 880 | N | 27.413 | −6.49 | 14.165 | N | PRO |
| | | 881 | HN | 26.519 | −6.302 | 14.571 | H | PRO |
| | | 882 | CA | 28.348 | −7.192 | 15.014 | C | PRO |
| | | 883 | HA | 28.908 | −7.895 | 14.411 | H | PRO |
| | | 884 | CB | 29.314 | −6.26 | 15.792 | C | PRO |
| | | 885 | HB1 | 30.112 | −6.875 | 16.261 | H | PRO |
| | | 886 | HB2 | 29.805 | −5.568 | 15.073 | H | PRO |
| | | 887 | ND1 | 27.72 | −4.465 | 16.677 | N | PRO |
| | | 888 | HD1 | 27.415 | −4.113 | 15.781 | H | PRO |
| | | 889 | CG | 28.666 | −5.443 | 16.882 | C | PRO |
| | | 890 | CE1 | 27.369 | −3.992 | 17.896 | C | PRO |
| | | 891 | HE1 | 26.633 | −3.2 | 18.032 | H | PRO |
| | | 892 | NE2 | 28.021 | −4.597 | 18.869 | N | PRO |
| | | 893 | CD2 | 28.829 | −5.519 | 18.229 | C | PRO |
| | | 894 | HD2 | 29.459 | −6.183 | 18.804 | H | PRO |
| | | 895 | C | 27.53 | −7.969 | 16.005 | C | PRO |
| | | 896 | O | 26.395 | −7.594 | 16.3 | O | PRO |
| 58 | GLN | 897 | N | 28.074 | −9.092 | 16.535 | N | PRO |
| | | 898 | HN | 28.976 | −9.42 | 16.267 | H | PRO |
| | | 899 | CA | 27.412 | −9.861 | 17.566 | C | PRO |
| | | 900 | HA | 26.39 | −10.002 | 17.245 | H | PRO |
| | | 901 | CB | 28.027 | −11.27 | 17.769 | C | PRO |
| | | 902 | HB1 | 28.23 | −11.702 | 16.766 | H | PRO |
| | | 903 | HB2 | 28.999 | −11.189 | 18.302 | H | PRO |
| | | 904 | CG | 27.077 | −12.231 | 18.51 | C | PRO |
| | | 905 | HG1 | 26.83 | −11.806 | 19.505 | H | PRO |
| | | 906 | HG2 | 26.134 | −12.337 | 17.934 | H | PRO |
| | | 907 | CD | 27.679 | −13.625 | 18.688 | C | PRO |
| | | 908 | OE1 | 28.264 | −14.197 | 17.761 | O | PRO |
| | | 909 | NE2 | 27.481 | −14.205 | 19.904 | N | PRO |
| | | 910 | HE21 | 27.86 | −15.11 | 20.087 | H | PRO |
| | | 911 | HE22 | 26.883 | −13.747 | 20.57 | H | PRO |
| | | 912 | C | 27.398 | −9.102 | 18.877 | C | PRO |
| | | 913 | O | 28.382 | −8.455 | 19.234 | O | PRO |
| 59 | ALA | 914 | N | 26.278 | −9.184 | 19.62 | N | PRO |
| | | 915 | HN | 25.5 | −9.742 | 19.317 | H | PRO |
| | | 916 | CA | 26.093 | −8.463 | 20.851 | C | PRO |
| | | 917 | HA | 26.875 | −8.748 | 21.541 | H | PRO |
| | | 918 | CB | 26.042 | −6.93 | 20.68 | C | PRO |
| | | 919 | HB1 | 27.026 | −6.56 | 20.324 | H | PRO |
| | | 920 | HB2 | 25.273 | −6.639 | 19.933 | H | PRO |
| | | 921 | HB3 | 25.817 | −6.43 | 21.645 | H | PRO |
| | | 922 | C | 24.768 | −8.898 | 21.4 | C | PRO |
| | | 923 | O | 23.725 | −8.683 | 20.787 | O | PRO |
| 60 | GLU | 924 | N | 24.79 | −9.549 | 22.58 | N | PRO |
| | | 925 | HN | 25.649 | −9.707 | 23.068 | H | PRO |
| | | 926 | CA | 23.619 | −10.099 | 23.217 | C | PRO |
| | | 927 | HA | 22.874 | −10.339 | 22.476 | H | PRO |
| | | 928 | CB | 23.933 | −11.38 | 24.033 | C | PRO |
| | | 929 | HB1 | 24.832 | −11.202 | 24.662 | H | PRO |
| | | 930 | HB2 | 23.081 | −11.591 | 24.718 | H | PRO |
| | | 931 | CG | 24.135 | −12.665 | 23.19 | C | PRO |
| | | 932 | HG1 | 24.327 | −13.519 | 23.876 | H | PRO |
| | | 933 | HG2 | 23.203 | −12.885 | 22.627 | H | PRO |
| | | 934 | CD | 25.299 | −12.589 | 22.204 | C | PRO |
| | | 935 | OE1 | 26.458 | −12.351 | 22.64 | O | PRO |
| | | 936 | OE2 | 25.048 | −12.779 | 20.985 | O | PRO |
| | | 937 | C | 23.056 | −9.043 | 24.131 | C | PRO |
| | | 938 | O | 23.737 | −8.548 | 25.027 | O | PRO |
| 61 | HIS | 939 | N | 21.778 | −8.661 | 23.907 | N | PRO |
| | | 940 | HN | 21.227 | −9.077 | 23.18 | H | PRO |
| | | 941 | CA | 21.113 | −7.658 | 24.703 | C | PRO |
| | | 942 | HA | 21.853 | −6.958 | 25.066 | H | PRO |
| | | 943 | CB | 20.055 | −6.878 | 23.89 | C | PRO |
| | | 944 | HB1 | 20.488 | −6.659 | 22.889 | H | PRO |
| | | 945 | HB2 | 19.157 | −7.506 | 23.717 | H | PRO |
| | | 946 | ND1 | 18.409 | −5.214 | 24.928 | N | PRO |
| | | 947 | HD1 | 17.599 | −5.801 | 24.911 | H | PRO |
| | | 948 | CG | 19.67 | −5.549 | 24.486 | C | PRO |
| | | 949 | CE1 | 18.46 | −3.913 | 25.312 | C | PRO |
| | | 950 | HE1 | 17.598 | −3.376 | 25.704 | H | PRO |
| | | 951 | NE2 | 19.66 | −3.389 | 25.143 | N | PRO |
| | | 952 | CD2 | 20.422 | −4.424 | 24.629 | C | PRO |
| | | 953 | HD2 | 21.47 | −4.266 | 24.407 | H | PRO |
| | | 954 | C | 20.46 | −8.337 | 25.88 | C | PRO |
| | | 955 | O | 19.998 | −9.472 | 25.78 | O | PRO |
| 62 | SER | 956 | N | 20.428 | −7.651 | 27.043 | N | PRO |
| | | 957 | HN | 20.798 | −6.727 | 27.109 | H | PRO |
| | | 958 | CA | 19.879 | −8.157 | 28.286 | C | PRO |
| | | 959 | HA | 20.337 | −9.117 | 28.481 | H | PRO |
| | | 960 | CB | 20.201 | −7.211 | 29.474 | C | PRO |
| | | 961 | HB1 | 19.668 | −7.539 | 30.392 | H | PRO |
| | | 962 | HB2 | 21.294 | −7.254 | 29.675 | H | PRO |
| | | 963 | OG | 19.863 | −5.858 | 29.172 | O | PRO |
| | | 964 | HG1 | 20.043 | −5.341 | 29.963 | H | PRO |
| | | 965 | C | 18.383 | −8.385 | 28.219 | C | PRO |
| | | 966 | O | 17.87 | −9.366 | 28.753 | O | PRO |
| 63 | GLU | 967 | N | 17.656 | −7.471 | 27.54 | N | PRO |
| | | 968 | HN | 18.11 | −6.689 | 27.123 | H | PRO |
| | | 969 | CA | 16.212 | −7.43 | 27.559 | C | PRO |
| | | 970 | HA | 15.85 | −7.851 | 28.487 | H | PRO |
| | | 971 | CB | 15.71 | −5.975 | 27.422 | C | PRO |
| | | 972 | HB1 | 15.934 | −5.607 | 26.394 | H | PRO |
| | | 973 | HB2 | 14.604 | −5.957 | 27.555 | H | PRO |
| | | 974 | CG | 16.335 | −4.988 | 28.43 | C | PRO |
| | | 975 | HG1 | 16.096 | −5.288 | 29.471 | H | PRO |
| | | 976 | HG2 | 17.437 | −4.953 | 28.309 | H | PRO |
| | | 977 | CD | 15.786 | −3.592 | 28.162 | C | PRO |
| | | 978 | OE1 | 15.897 | −3.136 | 26.992 | O | PRO |
| | | 979 | OE2 | 15.221 | −2.966 | 29.096 | O | PRO |
| | | 980 | C | 15.619 | −8.2 | 26.401 | C | PRO |
| | | 981 | O | 14.399 | −8.293 | 26.276 | O | PRO |
| 64 | LEU | 982 | N | 16.471 | −8.789 | 25.535 | N | PRO |
| | | 983 | HN | 17.457 | −8.756 | 25.688 | H | PRO |
| | | 984 | CA | 16.047 | −9.544 | 24.378 | C | PRO |
| | | 985 | HA | 14.97 | −9.591 | 24.328 | H | PRO |
| | | 986 | CB | 16.604 | −8.998 | 23.046 | C | PRO |
| | | 987 | HB1 | 17.715 | −9.01 | 23.086 | H | PRO |
| | | 988 | HB2 | 16.291 | −9.659 | 22.207 | H | PRO |
| | | 989 | CG | 16.139 | −7.561 | 22.728 | C | PRO |
| | | 990 | HG | 16.398 | −6.913 | 23.596 | H | PRO |
| | | 991 | CD1 | 16.89 | −7.011 | 21.509 | C | PRO |
| | | 992 | HD11 | 16.568 | −5.97 | 21.294 | H | PRO |
| | | 993 | HD12 | 17.986 | −7.013 | 21.688 | H | PRO |
| | | 994 | HD13 | 16.679 | −7.638 | 20.618 | H | PRO |
| | | 995 | CD2 | 14.618 | −7.469 | 22.513 | C | PRO |
| | | 996 | HD21 | 14.325 | −6.425 | 22.274 | H | PRO |
| | | 997 | HD22 | 14.309 | −8.124 | 21.672 | H | PRO |
| | | 998 | HD23 | 14.066 | −7.782 | 23.424 | H | PRO |
| | | 999 | C | 16.555 | −10.934 | 24.58 | C | PRO |
| | | 1000 | O | 17.606 | −11.134 | 25.184 | O | PRO |
| 65 | GLN | 1001 | N | 15.791 | −11.943 | 24.115 | N | PRO |
| | | 1002 | HN | 14.96 | −11.769 | 23.584 | H | PRO |
| | | 1003 | CA | 16.065 | −13.319 | 24.452 | C | PRO |
| | | 1004 | HA | 16.902 | −13.371 | 25.13 | H | PRO |
| | | 1005 | CB | 14.864 | −14.018 | 25.134 | C | PRO |
| | | 1006 | HB1 | 14.007 | −14.064 | 24.425 | H | PRO |
| | | 1007 | HB2 | 15.148 | −15.064 | 25.39 | H | PRO |
| | | 1008 | CG | 14.383 | −13.308 | 26.42 | C | PRO |
| | | 1009 | HG1 | 13.95 | −12.32 | 26.158 | H | PRO |
| | | 1010 | HG2 | 13.59 | −13.917 | 26.902 | H | PRO |
| | | 1011 | CD | 15.536 | −13.134 | 27.418 | C | PRO |
| | | 1012 | OE1 | 16.239 | −14.096 | 27.747 | O | PRO |
| | | 1013 | NE2 | 15.744 | −11.874 | 27.895 | N | PRO |
| | | 1014 | HE21 | 16.512 | −11.706 | 28.513 | H | PRO |
| | | 1015 | HE22 | 15.156 | −11.123 | 27.604 | H | PRO |
| | | 1016 | C | 16.461 | −14.066 | 23.213 | C | PRO |
| | | 1017 | O | 15.833 | −13.949 | 22.162 | O | PRO |
| 66 | GLY | 1018 | N | 17.557 | −14.841 | 23.322 | N | PRO |
| | | 1019 | HN | 18.04 | −14.93 | 24.196 | H | PRO |
| | | 1020 | CA | 18.153 | −15.527 | 22.203 | C | PRO |
| | | 1021 | HA1 | 18.069 | −14.919 | 21.313 | H | PRO |
| | | 1022 | HA2 | 17.699 | −16.504 | 22.127 | H | PRO |
| | | 1023 | C | 19.605 | −15.678 | 22.523 | C | PRO |
| | | 1024 | O | 20.224 | −14.768 | 23.07 | O | PRO |
| 67 | GLN | 1025 | N | 20.181 | −16.853 | 22.2 | N | PRO |
| | | 1026 | HN | 19.655 | −17.564 | 21.729 | H | PRO |
| | | 1027 | CA | 21.557 | −17.193 | 22.488 | C | PRO |
| | | 1028 | HA | 21.715 | −17.024 | 23.544 | H | PRO |
| | | 1029 | CB | 21.823 | −18.687 | 22.178 | C | PRO |
| | | 1030 | HB1 | 21.04 | −19.282 | 22.702 | H | PRO |
| | | 1031 | HB2 | 21.701 | −18.868 | 21.087 | H | PRO |
| | | 1032 | CG | 23.207 | −19.184 | 22.643 | C | PRO |
| | | 1033 | HG1 | 24.008 | −18.645 | 22.092 | H | PRO |
| | | 1034 | HG2 | 23.325 | −18.986 | 23.729 | H | PRO |
| | | 1035 | CD | 23.362 | −20.687 | 22.39 | C | PRO |
| | | 1036 | OE1 | 22.457 | −21.367 | 21.898 | O | PRO |
| | | 1037 | NE2 | 24.566 | −21.221 | 22.745 | N | PRO |
| | | 1038 | HE21 | 24.718 | −22.197 | 22.602 | H | PRO |
| | | 1039 | HE22 | 25.273 | −20.639 | 23.142 | H | PRO |
| | | 1040 | C | 22.556 | −16.343 | 21.73 | C | PRO |
| | | 1041 | O | 23.563 | −15.918 | 22.293 | O | PRO |
| 68 | LYS | 1042 | N | 22.294 | −16.087 | 20.43 | N | PRO |
| | | 1043 | HN | 21.448 | −16.427 | 19.997 | H | PRO |
| | | 1044 | CA | 23.226 | −15.419 | 19.554 | C | PRO |
| | | 1045 | HA | 24.062 | −15.024 | 20.116 | H | PRO |
| | | 1046 | CB | 23.722 | −16.424 | 18.489 | C | PRO |
| | | 1047 | HB1 | 23.979 | −17.368 | 19.024 | H | PRO |
| | | 1048 | HB2 | 22.896 | −16.676 | 17.789 | H | PRO |
| | | 1049 | CG | 24.974 | −16.006 | 17.704 | C | PRO |
| | | 1050 | HG1 | 24.81 | −15.033 | 17.194 | H | PRO |
| | | 1051 | HG2 | 25.803 | −15.882 | 18.437 | H | PRO |
| | | 1052 | CD | 25.369 | −17.084 | 16.678 | C | PRO |
| | | 1053 | HD1 | 25.109 | −18.078 | 17.111 | H | PRO |
| | | 1054 | HD2 | 24.753 | −16.962 | 15.76 | H | PRO |
| | | 1055 | CE | 26.861 | −17.141 | 16.32 | C | PRO |
| | | 1056 | HE1 | 27.474 | −17.33 | 17.227 | H | PRO |
| | | 1057 | HE2 | 27.038 | −17.96 | 15.589 | H | PRO |
| | | 1058 | NZ | 27.332 | −15.881 | 15.707 | N | PRO |
| | | 1059 | HZ1 | 28.243 | −16.045 | 15.237 | H | PRO |
| | | 1060 | HZ2 | 26.634 | −15.547 | 15.015 | H | PRO |
| | | 1061 | HZ3 | 27.469 | −15.162 | 16.458 | H | PRO |
| | | 1062 | C | 22.488 | −14.279 | 18.901 | C | PRO |
| | | 1063 | O | 21.556 | −14.496 | 18.132 | O | PRO |
| 69 | GLN | 1064 | N | 22.867 | −13.019 | 19.205 | N | PRO |
| | | 1065 | HN | 23.653 | −12.839 | 19.812 | H | PRO |
| | | 1066 | CA | 22.11 | −11.859 | 18.778 | C | PRO |
| | | 1067 | HA | 21.356 | −12.142 | 18.057 | H | PRO |
| | | 1068 | CB | 21.439 | −11.121 | 19.961 | C | PRO |
| | | 1069 | HB1 | 22.236 | −10.657 | 20.579 | H | PRO |
| | | 1070 | HB2 | 20.797 | −10.305 | 19.562 | H | PRO |
| | | 1071 | CG | 20.597 | −12.027 | 20.882 | C | PRO |
| | | 1072 | HG1 | 19.76 | −12.482 | 20.315 | H | PRO |
| | | 1073 | HG2 | 21.231 | −12.844 | 21.29 | H | PRO |
| | | 1074 | CD | 20.032 | −11.208 | 22.047 | C | PRO |
| | | 1075 | OE1 | 19.94 | −9.977 | 21.992 | O | PRO |
| | | 1076 | NE2 | 19.654 | −11.919 | 23.144 | N | PRO |
| | | 1077 | HE21 | 19.252 | −11.44 | 23.925 | H | PRO |
| | | 1078 | HE22 | 19.798 | −12.913 | 23.169 | H | PRO |
| | | 1079 | C | 23.055 | −10.896 | 18.116 | C | PRO |
| | | 1080 | O | 24.225 | −10.821 | 18.476 | O | PRO |
| 70 | TYR | 1081 | N | 22.57 | −10.133 | 17.112 | N | PRO |
| | | 1082 | HN | 21.617 | −10.206 | 16.817 | H | PRO |
| | | 1083 | CA | 23.39 | −9.192 | 16.378 | C | PRO |
| | | 1084 | HA | 24.373 | −9.119 | 16.822 | H | PRO |
| | | 1085 | CB | 23.504 | −9.536 | 14.872 | C | PRO |
| | | 1086 | HB1 | 22.564 | −10.004 | 14.509 | H | PRO |
| | | 1087 | HB2 | 23.71 | −8.631 | 14.26 | H | PRO |
| | | 1088 | CG | 24.64 | −10.496 | 14.661 | C | PRO |
| | | 1089 | CD1 | 24.557 | −11.834 | 15.08 | C | PRO |
| | | 1090 | HD1 | 23.655 | −12.195 | 15.551 | H | PRO |
| | | 1091 | CE1 | 25.642 | −12.703 | 14.91 | C | PRO |
| | | 1092 | HE1 | 25.567 | −13.717 | 15.269 | H | PRO |
| | | 1093 | CZ | 26.826 | −12.237 | 14.325 | C | PRO |
| | | 1094 | OH | 27.963 | −13.069 | 14.252 | O | PRO |
| | | 1095 | HH | 28.729 | −12.508 | 14.458 | H | PRO |
| | | 1096 | CD2 | 25.821 | −10.051 | 14.043 | C | PRO |
| | | 1097 | HD2 | 25.896 | −9.027 | 13.708 | H | PRO |
| | | 1098 | CE2 | 26.908 | −10.914 | 13.875 | C | PRO |
| | | 1099 | HE2 | 27.814 | −10.54 | 13.422 | H | PRO |
| | | 1100 | C | 22.779 | −7.83 | 16.498 | C | PRO |
| | | 1101 | O | 21.561 | −7.683 | 16.522 | O | PRO |
| 71 | GLN | 1102 | N | 23.637 | −6.791 | 16.567 | N | PRO |
| | | 1103 | HN | 24.627 | −6.939 | 16.525 | H | PRO |
| | | 1104 | CA | 23.217 | −5.421 | 16.722 | C | PRO |
| | | 1105 | HA | 22.147 | −5.377 | 16.854 | H | PRO |
| | | 1106 | CB | 23.884 | −4.744 | 17.943 | C | PRO |
| | | 1107 | HB1 | 23.659 | −5.369 | 18.837 | H | PRO |
| | | 1108 | HB2 | 24.985 | −4.752 | 17.805 | H | PRO |
| | | 1109 | CG | 23.385 | −3.309 | 18.21 | C | PRO |
| | | 1110 | HG1 | 23.596 | −2.66 | 17.334 | H | PRO |
| | | 1111 | HG2 | 22.287 | −3.329 | 18.382 | H | PRO |
| | | 1112 | CD | 24.075 | −2.694 | 19.428 | C | PRO |
| | | 1113 | OE1 | 25.037 | −3.235 | 19.984 | O | PRO |
| | | 1114 | NE2 | 23.574 | −1.5 | 19.851 | N | PRO |
| | | 1115 | HE21 | 23.956 | −1.075 | 20.67 | H | PRO |
| | | 1116 | HE22 | 22.804 | −1.086 | 19.361 | H | PRO |
| | | 1117 | C | 23.586 | −4.662 | 15.473 | C | PRO |
| | | 1118 | O | 24.686 | −4.802 | 14.94 | O | PRO |
| 72 | LEU | 1119 | N | 22.636 | −3.833 | 14.985 | N | PRO |
| | | 1120 | HN | 21.735 | −3.802 | 15.423 | H | PRO |
| | | 1121 | CA | 22.799 | −2.939 | 13.866 | C | PRO |
| | | 1122 | HA | 23.531 | −3.35 | 13.183 | H | PRO |
| | | 1123 | CB | 21.444 | −2.743 | 13.142 | C | PRO |
| | | 1124 | HB1 | 21.125 | −3.727 | 12.737 | H | PRO |
| | | 1125 | HB2 | 20.687 | −2.445 | 13.902 | H | PRO |
| | | 1126 | CG | 21.392 | −1.708 | 11.992 | C | PRO |
| | | 1127 | HG | 21.646 | −0.705 | 12.408 | H | PRO |
| | | 1128 | CD1 | 22.372 | −2.004 | 10.844 | C | PRO |
| | | 1129 | HD11 | 22.305 | −1.212 | 10.069 | H | PRO |
| | | 1130 | HD12 | 23.418 | −2.046 | 11.205 | H | PRO |
| | | 1131 | HD13 | 22.118 | −2.971 | 10.369 | H | PRO |
| | | 1132 | CD2 | 19.957 | −1.628 | 11.452 | C | PRO |
| | | 1133 | HD21 | 19.888 | −0.88 | 10.635 | H | PRO |
| | | 1134 | HD22 | 19.64 | −2.616 | 11.057 | H | PRO |
| | | 1135 | HD23 | 19.256 | −1.333 | 12.261 | H | PRO |
| | | 1136 | C | 23.287 | −1.603 | 14.374 | C | PRO |
| | | 1137 | O | 22.799 | −1.082 | 15.378 | O | PRO |
| 73 | ARG | 1138 | N | 24.275 | −1.015 | 13.666 | N | PRO |
| | | 1139 | HN | 24.672 | −1.468 | 12.866 | H | PRO |
| | | 1140 | CA | 24.838 | 0.273 | 13.985 | C | PRO |
| | | 1141 | HA | 24.124 | 0.874 | 14.529 | H | PRO |
| | | 1142 | CB | 26.192 | 0.176 | 14.732 | C | PRO |
| | | 1143 | HB1 | 26.876 | −0.477 | 14.146 | H | PRO |
| | | 1144 | HB2 | 26.666 | 1.179 | 14.785 | H | PRO |
| | | 1145 | CG | 26.105 | −0.382 | 16.165 | C | PRO |
| | | 1146 | HG1 | 25.588 | −1.366 | 16.142 | H | PRO |
| | | 1147 | HG2 | 27.138 | −0.569 | 16.528 | H | PRO |
| | | 1148 | CD | 25.389 | 0.539 | 17.166 | C | PRO |
| | | 1149 | HD1 | 24.338 | 0.704 | 16.845 | H | PRO |
| | | 1150 | HD2 | 25.381 | 0.1 | 18.186 | H | PRO |
| | | 1151 | NE | 26.068 | 1.879 | 17.199 | N | PRO |
| | | 1152 | HE | 25.684 | 2.624 | 16.63 | H | PRO |
| | | 1153 | CZ | 27.236 | 2.135 | 17.848 | C | PRO |
| | | 1154 | NH1 | 27.833 | 1.213 | 18.634 | N | PRO |
| | | 1155 | HH11 | 28.729 | 1.406 | 19.057 | H | PRO |
| | | 1156 | HH12 | 27.38 | 0.337 | 18.84 | H | PRO |
| | | 1157 | NH2 | 27.81 | 3.351 | 17.699 | N | PRO |
| | | 1158 | HH21 | 28.653 | 3.592 | 18.189 | H | PRO |
| | | 1159 | HH22 | 27.358 | 4.032 | 17.096 | H | PRO |
| | | 1160 | C | 25.125 | 0.949 | 12.676 | C | PRO |
| | | 1161 | O | 25.445 | 0.298 | 11.683 | O | PRO |
| 74 | GLY | 1162 | N | 25.021 | 2.297 | 12.655 | N | PRO |
| | | 1163 | HN | 24.717 | 2.791 | 13.47 | H | PRO |
| | | 1164 | CA | 25.447 | 3.1 | 11.526 | C | PRO |
| | | 1165 | HA1 | 26.461 | 2.818 | 11.287 | H | PRO |
| | | 1166 | HA2 | 25.369 | 4.13 | 11.833 | H | PRO |
| | | 1167 | C | 24.608 | 2.933 | 10.287 | C | PRO |
| | | 1168 | O | 25.142 | 2.947 | 9.18 | O | PRO |
| 75 | MET | 1169 | N | 23.278 | 2.754 | 10.44 | N | PRO |
| | | 1170 | HN | 22.863 | 2.75 | 11.349 | H | PRO |
| | | 1171 | CA | 22.378 | 2.562 | 9.322 | C | PRO |
| | | 1172 | HA | 22.885 | 1.986 | 8.561 | H | PRO |
| | | 1173 | CB | 21.098 | 1.805 | 9.746 | C | PRO |
| | | 1174 | HB1 | 21.403 | 0.849 | 10.222 | H | PRO |
| | | 1175 | HB2 | 20.566 | 2.393 | 10.524 | H | PRO |
| | | 1176 | CG | 20.102 | 1.49 | 8.611 | C | PRO |
| | | 1177 | HG1 | 19.178 | 1.101 | 9.089 | H | PRO |
| | | 1178 | HG2 | 19.816 | 2.43 | 8.093 | H | PRO |
| | | 1179 | SD | 20.693 | 0.255 | 7.416 | S | PRO |
| | | 1180 | CE | 20.527 | 1.269 | 5.919 | C | PRO |
| | | 1181 | HE1 | 20.844 | 0.695 | 5.023 | H | PRO |
| | | 1182 | HE2 | 19.478 | 1.6 | 5.766 | H | PRO |
| | | 1183 | HE3 | 21.167 | 2.174 | 5.971 | H | PRO |
| | | 1184 | C | 21.982 | 3.901 | 8.746 | C | PRO |
| | | 1185 | O | 21.429 | 4.751 | 9.443 | O | PRO |
| 76 | ALA | 1186 | N | 22.252 | 4.128 | 7.445 | N | PRO |
| | | 1187 | HN | 22.723 | 3.458 | 6.87 | H | PRO |
| | | 1188 | CA | 21.833 | 5.351 | 6.811 | C | PRO |
| | | 1189 | HA | 20.826 | 5.587 | 7.126 | H | PRO |
| | | 1190 | CB | 22.774 | 6.527 | 7.131 | C | PRO |
| | | 1191 | HB1 | 22.799 | 6.699 | 8.226 | H | PRO |
| | | 1192 | HB2 | 23.81 | 6.316 | 6.79 | H | PRO |
| | | 1193 | HB3 | 22.416 | 7.458 | 6.65 | H | PRO |
| | | 1194 | C | 21.813 | 5.155 | 5.324 | C | PRO |
| | | 1195 | O | 22.506 | 4.289 | 4.795 | O | PRO |
| 77 | THR | 1196 | N | 21.007 | 5.984 | 4.626 | N | PRO |
| | | 1197 | HN | 20.421 | 6.647 | 5.096 | H | PRO |
| | | 1198 | CA | 20.936 | 6.024 | 3.182 | C | PRO |
| | | 1199 | HA | 21.809 | 5.548 | 2.764 | H | PRO |
| | | 1200 | CB | 19.677 | 5.398 | 2.597 | C | PRO |
| | | 1201 | HB | 18.776 | 5.871 | 3.046 | H | PRO |
| | | 1202 | OG1 | 19.637 | 4.006 | 2.889 | O | PRO |
| | | 1203 | HG1 | 18.742 | 3.718 | 2.64 | H | PRO |
| | | 1204 | CG2 | 19.629 | 5.545 | 1.065 | C | PRO |
| | | 1205 | HG21 | 18.74 | 5.019 | 0.659 | H | PRO |
| | | 1206 | HG22 | 19.556 | 6.608 | 0.761 | H | PRO |
| | | 1207 | HG23 | 20.536 | 5.103 | 0.602 | H | PRO |
| | | 1208 | C | 20.99 | 7.488 | 2.836 | C | PRO |
| | | 1209 | O | 20.308 | 8.311 | 3.442 | O | PRO |
| 78 | LEU | 1210 | N | 21.856 | 7.858 | 1.869 | N | PRO |
| | | 1211 | HN | 22.393 | 7.159 | 1.39 | H | PRO |
| | | 1212 | CA | 22.063 | 9.21 | 1.403 | C | PRO |
| | | 1213 | HA | 22.295 | 9.806 | 2.272 | H | PRO |
| | | 1214 | CB | 23.261 | 9.233 | 0.42 | C | PRO |
| | | 1215 | HB1 | 24.11 | 8.704 | 0.907 | H | PRO |
| | | 1216 | HB2 | 22.989 | 8.644 | −0.484 | H | PRO |
| | | 1217 | CG | 23.762 | 10.624 | −0.022 | C | PRO |
| | | 1218 | HG | 22.914 | 11.156 | −0.511 | H | PRO |
| | | 1219 | CD1 | 24.247 | 11.481 | 1.158 | C | PRO |
| | | 1220 | HD11 | 24.488 | 12.506 | 0.81 | H | PRO |
| | | 1221 | HD12 | 23.47 | 11.56 | 1.941 | H | PRO |
| | | 1222 | HD13 | 25.156 | 11.037 | 1.612 | H | PRO |
| | | 1223 | CD2 | 24.882 | 10.487 | −1.065 | C | PRO |
| | | 1224 | HD21 | 25.196 | 11.488 | −1.424 | H | PRO |
| | | 1225 | HD22 | 25.762 | 9.977 | −0.621 | H | PRO |
| | | 1226 | HD23 | 24.53 | 9.896 | −1.934 | H | PRO |
| | | 1227 | C | 20.821 | 9.793 | 0.756 | C | PRO |
| | | 1228 | O | 20.092 | 9.099 | 0.05 | O | PRO |
| 79 | GLU | 1229 | N | 20.543 | 11.101 | 0.991 | N | PRO |
| | | 1230 | HN | 21.119 | 11.651 | 1.594 | H | PRO |
| | | 1231 | CA | 19.423 | 11.805 | 0.396 | C | PRO |
| | | 1232 | HA | 18.524 | 11.273 | 0.675 | H | PRO |
| | | 1233 | CB | 19.308 | 13.256 | 0.905 | C | PRO |
| | | 1234 | HB1 | 20.297 | 13.744 | 0.77 | H | PRO |
| | | 1235 | HB2 | 18.562 | 13.829 | 0.31 | H | PRO |
| | | 1236 | CG | 18.879 | 13.324 | 2.384 | C | PRO |
| | | 1237 | HG1 | 17.802 | 13.071 | 2.473 | H | PRO |
| | | 1238 | HG2 | 19.46 | 12.58 | 2.967 | H | PRO |
| | | 1239 | CD | 19.127 | 14.692 | 3.015 | C | PRO |
| | | 1240 | OE1 | 19.696 | 15.588 | 2.338 | O | PRO |
| | | 1241 | OE2 | 18.796 | 14.829 | 4.225 | O | PRO |
| | | 1242 | C | 19.5 | 11.834 | −1.114 | C | PRO |
| | | 1243 | O | 20.57 | 12.011 | −1.696 | O | PRO |
| 80 | GLY | 1244 | N | 18.333 | 11.64 | −1.769 | N | PRO |
| | | 1245 | HN | 17.481 | 11.558 | −1.258 | H | PRO |
| | | 1246 | CA | 18.2 | 11.502 | −3.206 | C | PRO |
| | | 1247 | HA1 | 18.936 | 12.129 | −3.69 | H | PRO |
| | | 1248 | HA2 | 17.186 | 11.773 | −3.459 | H | PRO |
| | | 1249 | C | 18.418 | 10.087 | −3.669 | C | PRO |
| | | 1250 | O | 18.277 | 9.797 | −4.854 | O | PRO |
| 81 | TYR | 1251 | N | 18.736 | 9.165 | −2.734 | N | PRO |
| | | 1252 | HN | 18.927 | 9.441 | −1.793 | H | PRO |
| | | 1253 | CA | 18.907 | 7.754 | −3.012 | C | PRO |
| | | 1254 | HA | 18.568 | 7.525 | −4.014 | H | PRO |
| | | 1255 | CB | 20.375 | 7.291 | −2.829 | C | PRO |
| | | 1256 | HB1 | 20.704 | 7.42 | −1.776 | H | PRO |
| | | 1257 | HB2 | 20.487 | 6.221 | −3.107 | H | PRO |
| | | 1258 | CG | 21.288 | 8.105 | −3.707 | C | PRO |
| | | 1259 | CD1 | 21.564 | 7.709 | −5.027 | C | PRO |
| | | 1260 | HD1 | 21.12 | 6.804 | −5.413 | H | PRO |
| | | 1261 | CE1 | 22.411 | 8.478 | −5.839 | C | PRO |
| | | 1262 | HE1 | 22.625 | 8.162 | −6.849 | H | PRO |
| | | 1263 | CZ | 22.975 | 9.661 | −5.343 | C | PRO |
| | | 1264 | OH | 23.818 | 10.443 | −6.161 | O | PRO |
| | | 1265 | HH | 23.954 | 11.294 | −5.723 | H | PRO |
| | | 1266 | CD2 | 21.87 | 9.288 | −3.22 | C | PRO |
| | | 1267 | HD2 | 21.657 | 9.609 | −2.212 | H | PRO |
| | | 1268 | CE2 | 22.697 | 10.069 | −4.034 | C | PRO |
| | | 1269 | HE2 | 23.124 | 10.978 | −3.639 | H | PRO |
| | | 1270 | C | 18.037 | 6.986 | −2.045 | C | PRO |
| | | 1271 | O | 18.131 | 5.764 | −1.934 | O | PRO |
| 82 | ARG | 1272 | N | 17.149 | 7.708 | −1.326 | N | PRO |
| | | 1273 | HN | 17.082 | 8.69 | −1.468 | H | PRO |
| | | 1274 | CA | 16.211 | 7.161 | −0.379 | C | PRO |
| | | 1275 | HA | 16.646 | 6.289 | 0.089 | H | PRO |
| | | 1276 | CB | 15.841 | 8.18 | 0.718 | C | PRO |
| | | 1277 | HB1 | 15.496 | 9.128 | 0.253 | H | PRO |
| | | 1278 | HB2 | 15.003 | 7.78 | 1.327 | H | PRO |
| | | 1279 | CG | 17.012 | 8.465 | 1.669 | C | PRO |
| | | 1280 | HG1 | 17.372 | 7.495 | 2.079 | H | PRO |
| | | 1281 | HG2 | 17.843 | 8.923 | 1.092 | H | PRO |
| | | 1282 | CD | 16.629 | 9.382 | 2.834 | C | PRO |
| | | 1283 | HD1 | 16.258 | 10.359 | 2.453 | H | PRO |
| | | 1284 | HD2 | 15.854 | 8.9 | 3.466 | H | PRO |
| | | 1285 | NE | 17.859 | 9.601 | 3.657 | N | PRO |
| | | 1286 | HE | 18.681 | 9.054 | 3.465 | H | PRO |
| | | 1287 | CZ | 17.995 | 10.615 | 4.549 | C | PRO |
| | | 1288 | NH1 | 16.938 | 11.361 | 4.933 | N | PRO |
| | | 1289 | HH11 | 17.076 | 12.161 | 5.531 | H | PRO |
| | | 1290 | HH12 | 16.008 | 11.05 | 4.713 | H | PRO |
| | | 1291 | NH2 | 19.228 | 10.885 | 5.037 | N | PRO |
| | | 1292 | HH21 | 19.385 | 11.716 | 5.575 | H | PRO |
| | | 1293 | HH22 | 19.994 | 10.319 | 4.738 | H | PRO |
| | | 1294 | C | 14.954 | 6.739 | −1.092 | C | PRO |
| | | 1295 | O | 14.655 | 7.227 | −2.183 | O | PRO |
| 83 | GLU | 1296 | N | 14.21 | 5.789 | −0.472 | N | PRO |
| | | 1297 | HN | 14.509 | 5.427 | 0.414 | H | PRO |
| | | 1298 | CA | 12.946 | 5.26 | −0.949 | C | PRO |
| | | 1299 | HA | 12.603 | 4.59 | −0.175 | H | PRO |
| | | 1300 | CB | 11.844 | 6.338 | −1.156 | C | PRO |
| | | 1301 | HB1 | 12.156 | 7.024 | −1.973 | H | PRO |
| | | 1302 | HB2 | 10.897 | 5.849 | −1.48 | H | PRO |
| | | 1303 | CG | 11.549 | 7.19 | 0.098 | C | PRO |
| | | 1304 | HG1 | 12.478 | 7.675 | 0.463 | H | PRO |
| | | 1305 | HG2 | 10.816 | 7.985 | −0.158 | H | PRO |
| | | 1306 | CD | 10.954 | 6.33 | 1.205 | C | PRO |
| | | 1307 | OE1 | 9.847 | 5.768 | 0.982 | O | PRO |
| | | 1308 | OE2 | 11.59 | 6.213 | 2.286 | O | PRO |
| | | 1309 | C | 13.112 | 4.419 | −2.2 | C | PRO |
| | | 1310 | O | 12.202 | 4.318 | −3.02 | O | PRO |
| 84 | GLN | 1311 | N | 14.29 | 3.775 | −2.362 | N | PRO |
| | | 1312 | HN | 15.022 | 3.867 | −1.68 | H | PRO |
| | | 1313 | CA | 14.619 | 2.977 | −3.523 | C | PRO |
| | | 1314 | HA | 13.794 | 2.97 | −4.222 | H | PRO |
| | | 1315 | CB | 15.906 | 3.481 | −4.233 | C | PRO |
| | | 1316 | HB1 | 16.783 | 3.308 | −3.567 | H | PRO |
| | | 1317 | HB2 | 16.068 | 2.891 | −5.163 | H | PRO |
| | | 1318 | CG | 15.913 | 4.979 | −4.6 | C | PRO |
| | | 1319 | HG1 | 15.914 | 5.585 | −3.671 | H | PRO |
| | | 1320 | HG2 | 16.842 | 5.215 | −5.162 | H | PRO |
| | | 1321 | CD | 14.711 | 5.344 | −5.475 | C | PRO |
| | | 1322 | OE1 | 14.493 | 4.748 | −6.536 | O | PRO |
| | | 1323 | NE2 | 13.915 | 6.35 | −5.016 | N | PRO |
| | | 1324 | HE21 | 13.109 | 6.611 | −5.543 | H | PRO |
| | | 1325 | HE22 | 14.108 | 6.772 | −4.127 | H | PRO |
| | | 1326 | C | 14.891 | 1.552 | −3.109 | C | PRO |
| | | 1327 | O | 15.29 | 0.745 | −3.947 | O | PRO |
| 85 | LYS | 1328 | N | 14.704 | 1.227 | −1.806 | N | PRO |
| | | 1329 | HN | 14.376 | 1.921 | −1.16 | H | PRO |
| | | 1330 | CA | 14.937 | −0.074 | −1.208 | C | PRO |
| | | 1331 | HA | 14.473 | −0.023 | −0.237 | H | PRO |
| | | 1332 | CB | 14.319 | −1.304 | −1.94 | C | PRO |
| | | 1333 | HB1 | 14.73 | −1.366 | −2.969 | H | PRO |
| | | 1334 | HB2 | 14.61 | −2.241 | −1.416 | H | PRO |
| | | 1335 | CG | 12.784 | −1.29 | −2.013 | C | PRO |
| | | 1336 | HG1 | 12.438 | −0.299 | −2.38 | H | PRO |
| | | 1337 | HG2 | 12.464 | −2.058 | −2.754 | H | PRO |
| | | 1338 | CD | 12.13 | −1.627 | −0.663 | C | PRO |
| | | 1339 | HD1 | 12.528 | −2.61 | −0.321 | H | PRO |
| | | 1340 | HD2 | 12.428 | −0.869 | 0.095 | H | PRO |
| | | 1341 | CE | 10.603 | −1.73 | −0.712 | C | PRO |
| | | 1342 | HE1 | 10.27 | −2.443 | −1.494 | H | PRO |
| | | 1343 | HE2 | 10.223 | −2.071 | 0.275 | H | PRO |
| | | 1344 | NZ | 9.998 | −0.412 | −0.993 | N | PRO |
| | | 1345 | HZ1 | 9.216 | −0.25 | −0.31 | H | PRO |
| | | 1346 | HZ2 | 10.712 | 0.347 | −0.855 | H | PRO |
| | | 1347 | HZ3 | 9.627 | −0.362 | −1.963 | H | PRO |
| | | 1348 | C | 16.405 | −0.293 | −0.942 | C | PRO |
| | | 1349 | O | 16.844 | −1.433 | −0.814 | O | PRO |
| 86 | ALA | 1350 | N | 17.218 | 0.788 | −0.833 | N | PRO |
| | | 1351 | HN | 16.865 | 1.722 | −0.92 | H | PRO |
| | | 1352 | CA | 18.64 | 0.666 | −0.585 | C | PRO |
| | | 1353 | HA | 19.028 | −0.049 | −1.292 | H | PRO |
| | | 1354 | CB | 19.39 | 1.999 | −0.771 | C | PRO |
| | | 1355 | HB1 | 19.17 | 2.422 | −1.774 | H | PRO |
| | | 1356 | HB2 | 19.081 | 2.741 | −0.005 | H | PRO |
| | | 1357 | HB3 | 20.488 | 1.845 | −0.693 | H | PRO |
| | | 1358 | C | 18.923 | 0.13 | 0.791 | C | PRO |
| | | 1359 | O | 19.7 | −0.81 | 0.948 | O | PRO |
| 87 | GLY | 1360 | N | 18.227 | 0.671 | 1.816 | N | PRO |
| | | 1361 | HN | 17.613 | 1.454 | 1.676 | H | PRO |
| | | 1362 | CA | 18.391 | 0.221 | 3.178 | C | PRO |
| | | 1363 | HA1 | 17.857 | 0.915 | 3.811 | H | PRO |
| | | 1364 | HA2 | 19.448 | 0.168 | 3.394 | H | PRO |
| | | 1365 | C | 17.801 | −1.142 | 3.379 | C | PRO |
| | | 1366 | O | 18.408 | −1.993 | 4.025 | O | PRO |
| 88 | SER | 1367 | N | 16.619 | −1.404 | 2.773 | N | PRO |
| | | 1368 | HN | 16.122 | −0.671 | 2.302 | H | PRO |
| | | 1369 | CA | 15.942 | −2.687 | 2.835 | C | PRO |
| | | 1370 | HA | 15.742 | −2.908 | 3.874 | H | PRO |
| | | 1371 | CB | 14.624 | −2.708 | 2.027 | C | PRO |
| | | 1372 | HB1 | 14.805 | −2.387 | 0.979 | H | PRO |
| | | 1373 | HB2 | 14.192 | −3.733 | 2.014 | H | PRO |
| | | 1374 | OG | 13.658 | −1.847 | 2.616 | O | PRO |
| | | 1375 | HG1 | 13.99 | −0.935 | 2.493 | H | PRO |
| | | 1376 | C | 16.785 | −3.801 | 2.278 | C | PRO |
| | | 1377 | O | 16.852 | −4.878 | 2.86 | O | PRO |
| 89 | SER | 1378 | N | 17.475 | −3.55 | 1.143 | N | PRO |
| | | 1379 | HN | 17.401 | −2.66 | 0.691 | H | PRO |
| | | 1380 | CA | 18.335 | −4.518 | 0.493 | C | PRO |
| | | 1381 | HA | 17.741 | −5.406 | 0.334 | H | PRO |
| | | 1382 | CB | 18.874 | −4.03 | −0.871 | C | PRO |
| | | 1383 | HB1 | 19.464 | −3.096 | −0.751 | H | PRO |
| | | 1384 | HB2 | 19.525 | −4.809 | −1.322 | H | PRO |
| | | 1385 | OG | 17.801 | −3.798 | −1.778 | O | PRO |
| | | 1386 | HG1 | 17.434 | −2.93 | −1.543 | H | PRO |
| | | 1387 | C | 19.506 | −4.922 | 1.354 | C | PRO |
| | | 1388 | O | 19.862 | −6.098 | 1.4 | O | PRO |
| 90 | LEU | 1389 | N | 20.116 | −3.95 | 2.074 | N | PRO |
| | | 1390 | HN | 19.816 | −2.999 | 2.005 | H | PRO |
| | | 1391 | CA | 21.204 | −4.191 | 3.002 | C | PRO |
| | | 1392 | HA | 21.961 | −4.767 | 2.487 | H | PRO |
| | | 1393 | CB | 21.84 | −2.869 | 3.502 | C | PRO |
| | | 1394 | HB1 | 21.066 | −2.072 | 3.525 | H | PRO |
| | | 1395 | HB2 | 22.219 | −2.989 | 4.542 | H | PRO |
| | | 1396 | CG | 23.043 | −2.402 | 2.65 | C | PRO |
| | | 1397 | HG | 23.814 | −3.206 | 2.711 | H | PRO |
| | | 1398 | CD1 | 22.722 | −2.205 | 1.16 | C | PRO |
| | | 1399 | HD11 | 22.083 | −1.311 | 1.015 | H | PRO |
| | | 1400 | HD12 | 23.664 | −2.064 | 0.596 | H | PRO |
| | | 1401 | HD13 | 22.213 | −3.089 | 0.73 | H | PRO |
| | | 1402 | CD2 | 23.662 | −1.125 | 3.239 | C | PRO |
| | | 1403 | HD21 | 24.546 | −0.81 | 2.648 | H | PRO |
| | | 1404 | HD22 | 22.921 | −0.297 | 3.234 | H | PRO |
| | | 1405 | HD23 | 23.986 | −1.303 | 4.284 | H | PRO |
| | | 1406 | C | 20.77 | −5.016 | 4.188 | C | PRO |
| | | 1407 | O | 21.466 | −5.955 | 4.567 | O | PRO |
| 91 | ILE | 1408 | N | 19.595 | −4.707 | 4.79 | N | PRO |
| | | 1409 | HN | 19.051 | −3.926 | 4.48 | H | PRO |
| | | 1410 | CA | 19.062 | −5.458 | 5.914 | C | PRO |
| | | 1411 | HA | 19.852 | −5.549 | 6.645 | H | PRO |
| | | 1412 | CB | 17.87 | −4.763 | 6.573 | C | PRO |
| | | 1413 | HB | 17.093 | −4.581 | 5.793 | H | PRO |
| | | 1414 | CG2 | 17.234 | −5.651 | 7.671 | C | PRO |
| | | 1415 | HG21 | 16.461 | −5.076 | 8.221 | H | PRO |
| | | 1416 | HG22 | 16.743 | −6.545 | 7.235 | H | PRO |
| | | 1417 | HG23 | 18.004 | −5.988 | 8.396 | H | PRO |
| | | 1418 | CG1 | 18.277 | −3.378 | 7.143 | C | PRO |
| | | 1419 | HG11 | 18.671 | −2.742 | 6.324 | H | PRO |
| | | 1420 | HG12 | 17.358 | −2.879 | 7.524 | H | PRO |
| | | 1421 | CD1 | 19.313 | −3.407 | 8.273 | C | PRO |
| | | 1422 | HD1 | 19.512 | −2.374 | 8.63 | H | PRO |
| | | 1423 | HD2 | 18.951 | −4.006 | 9.134 | H | PRO |
| | | 1424 | HD3 | 20.274 | −3.834 | 7.921 | H | PRO |
| | | 1425 | C | 18.705 | −6.87 | 5.508 | C | PRO |
| | | 1426 | O | 19.065 | −7.816 | 6.2 | O | PRO |
| 92 | LYS | 1427 | N | 18.035 | −7.059 | 4.349 | N | PRO |
| | | 1428 | HN | 17.739 | −6.279 | 3.793 | H | PRO |
| | | 1429 | CA | 17.654 | −8.367 | 3.853 | C | PRO |
| | | 1430 | HA | 17.127 | −8.875 | 4.65 | H | PRO |
| | | 1431 | CB | 16.702 | −8.267 | 2.645 | C | PRO |
| | | 1432 | HB1 | 17.148 | −7.591 | 1.882 | H | PRO |
| | | 1433 | HB2 | 16.564 | −9.269 | 2.181 | H | PRO |
| | | 1434 | CG | 15.315 | −7.742 | 3.045 | C | PRO |
| | | 1435 | HG1 | 14.837 | −8.472 | 3.736 | H | PRO |
| | | 1436 | HG2 | 15.426 | −6.784 | 3.6 | H | PRO |
| | | 1437 | CD | 14.4 | −7.504 | 1.837 | C | PRO |
| | | 1438 | HD1 | 14.919 | −6.807 | 1.138 | H | PRO |
| | | 1439 | HD2 | 14.252 | −8.473 | 1.31 | H | PRO |
| | | 1440 | CE | 13.045 | −6.91 | 2.235 | C | PRO |
| | | 1441 | HE1 | 12.51 | −7.59 | 2.932 | H | PRO |
| | | 1442 | HE2 | 13.183 | −5.921 | 2.724 | H | PRO |
| | | 1443 | NZ | 12.195 | −6.715 | 1.041 | N | PRO |
| | | 1444 | HZ1 | 11.274 | −6.306 | 1.338 | H | PRO |
| | | 1445 | HZ2 | 12.677 | −6.063 | 0.374 | H | PRO |
| | | 1446 | HZ3 | 12.033 | −7.629 | 0.577 | H | PRO |
| | | 1447 | C | 18.842 | −9.233 | 3.5 | C | PRO |
| | | 1448 | O | 18.85 | −10.422 | 3.806 | O | PRO |
| 93 | HIS | 1449 | N | 19.9 | −8.656 | 2.882 | N | PRO |
| | | 1450 | HN | 19.874 | −7.694 | 2.604 | H | PRO |
| | | 1451 | CA | 21.141 | −9.362 | 2.62 | C | PRO |
| | | 1452 | HA | 20.887 | −10.281 | 2.106 | H | PRO |
| | | 1453 | CB | 22.099 | −8.558 | 1.714 | C | PRO |
| | | 1454 | HB1 | 21.557 | −8.282 | 0.783 | H | PRO |
| | | 1455 | HB2 | 22.402 | −7.614 | 2.218 | H | PRO |
| | | 1456 | ND1 | 24.584 | −9.146 | 1.812 | N | PRO |
| | | 1457 | HD1 | 24.857 | −8.439 | 2.466 | H | PRO |
| | | 1458 | CG | 23.316 | −9.349 | 1.311 | C | PRO |
| | | 1459 | CE1 | 25.376 | −10.105 | 1.272 | C | PRO |
| | | 1460 | HE1 | 26.435 | −10.198 | 1.501 | H | PRO |
| | | 1461 | NE2 | 24.71 | −10.91 | 0.469 | N | PRO |
| | | 1462 | CD2 | 23.411 | −10.433 | 0.492 | C | PRO |
| | | 1463 | HD2 | 22.64 | −10.935 | −0.08 | H | PRO |
| | | 1464 | C | 21.854 | −9.74 | 3.901 | C | PRO |
| | | 1465 | O | 22.405 | −10.832 | 4.018 | O | PRO |
| 94 | ALA | 1466 | N | 21.831 | −8.846 | 4.917 | N | PRO |
| | | 1467 | HN | 21.414 | −7.944 | 4.8 | H | PRO |
| | | 1468 | CA | 22.365 | −9.11 | 6.233 | C | PRO |
| | | 1469 | HA | 23.401 | −9.397 | 6.11 | H | PRO |
| | | 1470 | CB | 22.305 | −7.877 | 7.144 | C | PRO |
| | | 1471 | HB1 | 22.825 | −7.028 | 6.652 | H | PRO |
| | | 1472 | HB2 | 21.258 | −7.567 | 7.343 | H | PRO |
| | | 1473 | HB3 | 22.807 | −8.075 | 8.115 | H | PRO |
| | | 1474 | C | 21.655 | −10.248 | 6.919 | C | PRO |
| | | 1475 | O | 22.303 | −11.079 | 7.543 | O | PRO |
| 95 | GLU | 1476 | N | 20.312 | −10.353 | 6.78 | N | PRO |
| | | 1477 | HN | 19.796 | −9.645 | 6.295 | H | PRO |
| | | 1478 | CA | 19.522 | −11.439 | 7.328 | C | PRO |
| | | 1479 | HA | 19.645 | −11.431 | 8.402 | H | PRO |
| | | 1480 | CB | 18.017 | −11.34 | 6.986 | C | PRO |
| | | 1481 | HB1 | 17.902 | −11.233 | 5.886 | H | PRO |
| | | 1482 | HB2 | 17.527 | −12.291 | 7.288 | H | PRO |
| | | 1483 | CG | 17.24 | −10.218 | 7.697 | C | PRO |
| | | 1484 | HG1 | 17.175 | −10.431 | 8.784 | H | PRO |
| | | 1485 | HG2 | 17.736 | −9.239 | 7.56 | H | PRO |
| | | 1486 | CD | 15.827 | −10.148 | 7.12 | C | PRO |
| | | 1487 | OE1 | 15.138 | −11.205 | 7.097 | O | PRO |
| | | 1488 | OE2 | 15.406 | −9.039 | 6.697 | O | PRO |
| | | 1489 | C | 19.981 | −12.789 | 6.82 | C | PRO |
| | | 1490 | O | 20.022 | −13.745 | 7.587 | O | PRO |
| 96 | GLU | 1491 | N | 20.365 | −12.897 | 5.523 | N | PRO |
| | | 1492 | HN | 20.311 | −12.113 | 4.907 | H | PRO |
| | | 1493 | CA | 20.911 | −14.114 | 4.944 | C | PRO |
| | | 1494 | HA | 20.183 | −14.904 | 5.065 | H | PRO |
| | | 1495 | CB | 21.26 | −13.942 | 3.447 | C | PRO |
| | | 1496 | HB1 | 22.067 | −13.18 | 3.355 | H | PRO |
| | | 1497 | HB2 | 21.659 | −14.902 | 3.047 | H | PRO |
| | | 1498 | CG | 20.089 | −13.493 | 2.551 | C | PRO |
| | | 1499 | HG1 | 19.314 | −14.285 | 2.495 | H | PRO |
| | | 1500 | HG2 | 19.626 | −12.574 | 2.962 | H | PRO |
| | | 1501 | CD | 20.58 | −13.174 | 1.138 | C | PRO |
| | | 1502 | OE1 | 21.764 | −13.466 | 0.817 | O | PRO |
| | | 1503 | OE2 | 19.771 | −12.609 | 0.353 | O | PRO |
| | | 1504 | C | 22.2 | −14.529 | 5.622 | C | PRO |
| | | 1505 | O | 22.384 | −15.695 | 5.969 | O | PRO |
| 97 | ILE | 1506 | N | 23.108 | −13.551 | 5.858 | N | PRO |
| | | 1507 | HN | 22.914 | −12.615 | 5.568 | H | PRO |
| | | 1508 | CA | 24.386 | −13.743 | 6.517 | C | PRO |
| | | 1509 | HA | 24.907 | −14.535 | 5.999 | H | PRO |
| | | 1510 | CB | 25.234 | −12.465 | 6.47 | C | PRO |
| | | 1511 | HB | 24.642 | −11.626 | 6.9 | H | PRO |
| | | 1512 | CG2 | 26.524 | −12.613 | 7.314 | C | PRO |
| | | 1513 | HG21 | 27.15 | −11.701 | 7.232 | H | PRO |
| | | 1514 | HG22 | 26.297 | −12.754 | 8.39 | H | PRO |
| | | 1515 | HG23 | 27.122 | −13.475 | 6.957 | H | PRO |
| | | 1516 | CG1 | 25.561 | −12.098 | 4.999 | C | PRO |
| | | 1517 | HG11 | 26.258 | −12.861 | 4.587 | H | PRO |
| | | 1518 | HG12 | 24.634 | −12.132 | 4.387 | H | PRO |
| | | 1519 | CD1 | 26.178 | −10.706 | 4.831 | C | PRO |
| | | 1520 | HD1 | 26.368 | −10.499 | 3.758 | H | PRO |
| | | 1521 | HD2 | 25.488 | −9.929 | 5.223 | H | PRO |
| | | 1522 | HD3 | 27.144 | −10.627 | 5.371 | H | PRO |
| | | 1523 | C | 24.18 | −14.184 | 7.948 | C | PRO |
| | | 1524 | O | 24.786 | −15.151 | 8.399 | O | PRO |
| 98 | LEU | 1525 | N | 23.277 | −13.5 | 8.684 | N | PRO |
| | | 1526 | HN | 22.795 | −12.717 | 8.283 | H | PRO |
| | | 1527 | CA | 22.98 | −13.769 | 10.072 | C | PRO |
| | | 1528 | HA | 23.924 | −13.834 | 10.594 | H | PRO |
| | | 1529 | CB | 22.139 | −12.639 | 10.704 | C | PRO |
| | | 1530 | HB1 | 21.184 | −12.533 | 10.143 | H | PRO |
| | | 1531 | HB2 | 21.888 | −12.912 | 11.752 | H | PRO |
| | | 1532 | CG | 22.841 | −11.262 | 10.748 | C | PRO |
| | | 1533 | HG | 22.991 | −10.899 | 9.71 | H | PRO |
| | | 1534 | CD1 | 21.947 | −10.227 | 11.451 | C | PRO |
| | | 1535 | HD11 | 22.441 | −9.233 | 11.457 | H | PRO |
| | | 1536 | HD12 | 20.975 | −10.135 | 10.922 | H | PRO |
| | | 1537 | HD13 | 21.75 | −10.537 | 12.499 | H | PRO |
| | | 1538 | CD2 | 24.241 | −11.328 | 11.377 | C | PRO |
| | | 1539 | HD21 | 24.645 | −10.302 | 11.514 | H | PRO |
| | | 1540 | HD22 | 24.188 | −11.834 | 12.362 | H | PRO |
| | | 1541 | HD23 | 24.944 | −11.888 | 10.727 | H | PRO |
| | | 1542 | C | 22.305 | −15.101 | 10.292 | C | PRO |
| | | 1543 | O | 22.651 | −15.814 | 11.232 | O | PRO |
| 99 | ARG | 1544 | N | 21.356 | −15.495 | 9.407 | N | PRO |
| | | 1545 | HN | 21.054 | −14.881 | 8.673 | H | PRO |
| | | 1546 | CA | 20.72 | −16.801 | 9.422 | C | PRO |
| | | 1547 | HA | 20.305 | −16.951 | 10.407 | H | PRO |
| | | 1548 | CB | 19.581 | −16.931 | 8.378 | C | PRO |
| | | 1549 | HB1 | 19.931 | −16.527 | 7.403 | H | PRO |
| | | 1550 | HB2 | 19.33 | −18.007 | 8.231 | H | PRO |
| | | 1551 | CG | 18.274 | −16.229 | 8.797 | C | PRO |
| | | 1552 | HG1 | 17.931 | −16.686 | 9.754 | H | PRO |
| | | 1553 | HG2 | 18.472 | −15.153 | 8.99 | H | PRO |
| | | 1554 | CD | 17.164 | −16.356 | 7.743 | C | PRO |
| | | 1555 | HD1 | 17.443 | −15.818 | 6.813 | H | PRO |
| | | 1556 | HD2 | 16.997 | −17.428 | 7.499 | H | PRO |
| | | 1557 | NE | 15.883 | −15.822 | 8.325 | N | PRO |
| | | 1558 | HE | 15.55 | −16.235 | 9.182 | H | PRO |
| | | 1559 | CZ | 15.217 | −14.719 | 7.895 | C | PRO |
| | | 1560 | NH1 | 15.682 | −13.952 | 6.888 | N | PRO |
| | | 1561 | HH11 | 15.312 | −13.018 | 6.781 | H | PRO |
| | | 1562 | HH12 | 16.547 | −14.189 | 6.459 | H | PRO |
| | | 1563 | NH2 | 14.061 | −14.362 | 8.503 | N | PRO |
| | | 1564 | HH21 | 13.531 | −13.592 | 8.126 | H | PRO |
| | | 1565 | HH22 | 13.629 | −14.978 | 9.177 | H | PRO |
| | | 1566 | C | 21.721 | −17.911 | 9.185 | C | PRO |
| | | 1567 | O | 21.656 | −18.961 | 9.821 | O | PRO |
| 100 | LYS | 1568 | N | 22.707 | −17.683 | 8.286 | N | PRO |
| | | 1569 | HN | 22.738 | −16.83 | 7.764 | H | PRO |
| | | 1570 | CA | 23.745 | −18.646 | 7.975 | C | PRO |
| | | 1571 | HA | 23.269 | −19.606 | 7.825 | H | PRO |
| | | 1572 | CB | 24.477 | −18.243 | 6.672 | C | PRO |
| | | 1573 | HB1 | 23.706 | −17.931 | 5.931 | H | PRO |
| | | 1574 | HB2 | 25.128 | −17.362 | 6.859 | H | PRO |
| | | 1575 | CG | 25.291 | −19.376 | 6.026 | C | PRO |
| | | 1576 | HG1 | 26.085 | −19.716 | 6.727 | H | PRO |
| | | 1577 | HG2 | 24.601 | −20.229 | 5.849 | H | PRO |
| | | 1578 | CD | 25.943 | −18.959 | 4.697 | C | PRO |
| | | 1579 | HD1 | 25.165 | −18.542 | 4.02 | H | PRO |
| | | 1580 | HD2 | 26.665 | −18.14 | 4.912 | H | PRO |
| | | 1581 | CE | 26.701 | −20.087 | 3.984 | C | PRO |
| | | 1582 | HE1 | 27.235 | −19.681 | 3.097 | H | PRO |
| | | 1583 | HE2 | 27.438 | −20.557 | 4.668 | H | PRO |
| | | 1584 | NZ | 25.777 | −21.142 | 3.504 | N | PRO |
| | | 1585 | HZ1 | 26.31 | −21.847 | 2.957 | H | PRO |
| | | 1586 | HZ2 | 25.319 | −21.62 | 4.32 | H | PRO |
| | | 1587 | HZ3 | 25.046 | −20.712 | 2.905 | H | PRO |
| | | 1588 | C | 24.749 | −18.784 | 9.109 | C | PRO |
| | | 1589 | O | 25.434 | −19.799 | 9.225 | O | PRO |
| 101 | ARG | 1590 | N | 24.813 | −17.777 | 10.012 | N | PRO |
| | | 1591 | HN | 24.264 | −16.953 | 9.884 | H | PRO |
| | | 1592 | CA | 25.648 | −17.801 | 11.194 | C | PRO |
| | | 1593 | HA | 26.481 | −18.472 | 11.046 | H | PRO |
| | | 1594 | CB | 26.177 | −16.389 | 11.542 | C | PRO |
| | | 1595 | HB1 | 25.317 | −15.684 | 11.594 | H | PRO |
| | | 1596 | HB2 | 26.666 | −16.403 | 12.543 | H | PRO |
| | | 1597 | CG | 27.213 | −15.839 | 10.548 | C | PRO |
| | | 1598 | HG1 | 28.113 | −16.495 | 10.572 | H | PRO |
| | | 1599 | HG2 | 26.802 | −15.869 | 9.517 | H | PRO |
| | | 1600 | CD | 27.61 | −14.403 | 10.904 | C | PRO |
| | | 1601 | HD1 | 26.733 | −13.723 | 10.833 | H | PRO |
| | | 1602 | HD2 | 28.016 | −14.39 | 11.939 | H | PRO |
| | | 1603 | NE | 28.66 | −13.933 | 9.945 | N | PRO |
| | | 1604 | HE | 28.761 | −14.388 | 9.05 | H | PRO |
| | | 1605 | CZ | 29.526 | −12.925 | 10.225 | C | PRO |
| | | 1606 | NH1 | 29.489 | −12.282 | 11.411 | N | PRO |
| | | 1607 | HH11 | 30.152 | −11.55 | 11.606 | H | PRO |
| | | 1608 | HH12 | 28.814 | −12.555 | 12.091 | H | PRO |
| | | 1609 | NH2 | 30.445 | −12.575 | 9.297 | N | PRO |
| | | 1610 | HH21 | 31.14 | −11.878 | 9.507 | H | PRO |
| | | 1611 | HH22 | 30.448 | −13.064 | 8.424 | H | PRO |
| | | 1612 | C | 24.858 | −18.283 | 12.393 | C | PRO |
| | | 1613 | O | 25.4 | −18.364 | 13.494 | O | PRO |
| 102 | GLY | 1614 | N | 23.562 | −18.639 | 12.213 | N | PRO |
| | | 1615 | HN | 23.128 | −18.563 | 11.315 | H | PRO |
| | | 1616 | CA | 22.752 | −19.234 | 13.257 | C | PRO |
| | | 1617 | HA1 | 23.357 | −19.947 | 13.799 | H | PRO |
| | | 1618 | HA2 | 21.909 | −19.697 | 12.764 | H | PRO |
| | | 1619 | C | 22.196 | −18.249 | 14.248 | C | PRO |
| | | 1620 | O | 21.72 | −18.654 | 15.306 | O | PRO |
| 103 | ALA | 1621 | N | 22.264 | −16.929 | 13.957 | N | PRO |
| | | 1622 | HN | 22.624 | −16.618 | 13.077 | H | PRO |
| | | 1623 | CA | 21.767 | −15.892 | 14.837 | C | PRO |
| | | 1624 | HA | 22.237 | −16.044 | 15.798 | H | PRO |
| | | 1625 | CB | 22.148 | −14.486 | 14.343 | C | PRO |
| | | 1626 | HB1 | 23.25 | −14.411 | 14.227 | H | PRO |
| | | 1627 | HB2 | 21.683 | −14.288 | 13.357 | H | PRO |
| | | 1628 | HB3 | 21.816 | −13.705 | 15.061 | H | PRO |
| | | 1629 | C | 20.268 | −15.954 | 15.045 | C | PRO |
| | | 1630 | O | 19.502 | −16.216 | 14.118 | O | PRO |
| 104 | ASP | 1631 | N | 19.831 | −15.716 | 16.3 | N | PRO |
| | | 1632 | HN | 20.482 | −15.522 | 17.038 | H | PRO |
| | | 1633 | CA | 18.447 | −15.683 | 16.709 | C | PRO |
| | | 1634 | HA | 17.949 | −16.553 | 16.305 | H | PRO |
| | | 1635 | CB | 18.329 | −15.666 | 18.252 | C | PRO |
| | | 1636 | HB1 | 18.964 | −14.863 | 18.68 | H | PRO |
| | | 1637 | HB2 | 17.279 | −15.491 | 18.567 | H | PRO |
| | | 1638 | CG | 18.773 | −17.007 | 18.819 | C | PRO |
| | | 1639 | OD1 | 17.913 | −17.927 | 18.895 | O | PRO |
| | | 1640 | OD2 | 19.962 | −17.125 | 19.214 | O | PRO |
| | | 1641 | C | 17.733 | −14.459 | 16.192 | C | PRO |
| | | 1642 | O | 16.609 | −14.548 | 15.697 | O | PRO |
| 105 | LEU | 1643 | N | 18.376 | −13.275 | 16.3 | N | PRO |
| | | 1644 | HN | 19.313 | −13.209 | 16.636 | H | PRO |
| | | 1645 | CA | 17.69 | −12.033 | 16.052 | C | PRO |
| | | 1646 | HA | 17.074 | −12.16 | 15.171 | H | PRO |
| | | 1647 | CB | 16.775 | −11.602 | 17.238 | C | PRO |
| | | 1648 | HB1 | 16.189 | −10.7 | 16.964 | H | PRO |
| | | 1649 | HB2 | 16.039 | −12.426 | 17.38 | H | PRO |
| | | 1650 | CG | 17.463 | −11.356 | 18.601 | C | PRO |
| | | 1651 | HG | 18.393 | −11.968 | 18.637 | H | PRO |
| | | 1652 | CD1 | 17.845 | −9.881 | 18.808 | C | PRO |
| | | 1653 | HD11 | 18.335 | −9.743 | 19.795 | H | PRO |
| | | 1654 | HD12 | 18.543 | −9.538 | 18.018 | H | PRO |
| | | 1655 | HD13 | 16.937 | −9.243 | 18.776 | H | PRO |
| | | 1656 | CD2 | 16.567 | −11.827 | 19.759 | C | PRO |
| | | 1657 | HD21 | 17.079 | −11.675 | 20.733 | H | PRO |
| | | 1658 | HD22 | 15.613 | −11.258 | 19.768 | H | PRO |
| | | 1659 | HD23 | 16.335 | −12.908 | 19.65 | H | PRO |
| | | 1660 | C | 18.679 | −10.952 | 15.721 | C | PRO |
| | | 1661 | O | 19.874 | −11.052 | 16.006 | O | PRO |
| 106 | LEU | 1662 | N | 18.155 | −9.88 | 15.093 | N | PRO |
| | | 1663 | HN | 17.178 | −9.862 | 14.869 | H | PRO |
| | | 1664 | CA | 18.87 | −8.68 | 14.746 | C | PRO |
| | | 1665 | HA | 19.891 | −8.738 | 15.093 | H | PRO |
| | | 1666 | CB | 18.817 | −8.416 | 13.22 | C | PRO |
| | | 1667 | HB1 | 19.359 | −9.245 | 12.715 | H | PRO |
| | | 1668 | HB2 | 17.756 | −8.47 | 12.887 | H | PRO |
| | | 1669 | CG | 19.409 | −7.073 | 12.723 | C | PRO |
| | | 1670 | HG | 18.801 | −6.246 | 13.16 | H | PRO |
| | | 1671 | CD1 | 20.87 | −6.853 | 13.154 | C | PRO |
| | | 1672 | HD11 | 21.264 | −5.919 | 12.703 | H | PRO |
| | | 1673 | HD12 | 20.95 | −6.759 | 14.255 | H | PRO |
| | | 1674 | HD13 | 21.506 | −7.697 | 12.821 | H | PRO |
| | | 1675 | CD2 | 19.275 | −6.969 | 11.192 | C | PRO |
| | | 1676 | HD21 | 19.605 | −5.969 | 10.842 | H | PRO |
| | | 1677 | HD22 | 19.9 | −7.742 | 10.696 | H | PRO |
| | | 1678 | HD23 | 18.219 | −7.119 | 10.884 | H | PRO |
| | | 1679 | C | 18.148 | −7.588 | 15.486 | C | PRO |
| | | 1680 | O | 16.921 | −7.525 | 15.448 | O | PRO |
| 107 | TRP | 1681 | N | 18.887 | −6.714 | 16.203 | N | PRO |
| | | 1682 | HN | 19.885 | −6.792 | 16.252 | H | PRO |
| | | 1683 | CA | 18.29 | −5.68 | 17.012 | C | PRO |
| | | 1684 | HA | 17.264 | −5.54 | 16.707 | H | PRO |
| | | 1685 | CB | 18.29 | −6.007 | 18.529 | C | PRO |
| | | 1686 | HB1 | 17.736 | −5.213 | 19.075 | H | PRO |
| | | 1687 | HB2 | 17.713 | −6.948 | 18.657 | H | PRO |
| | | 1688 | CG | 19.637 | −6.215 | 19.211 | C | PRO |
| | | 1689 | CD1 | 20.407 | −7.342 | 19.273 | C | PRO |
| | | 1690 | HD1 | 20.201 | −8.259 | 18.746 | H | PRO |
| | | 1691 | NE1 | 21.511 | −7.125 | 20.063 | N | PRO |
| | | 1692 | HE1 | 22.229 | −7.77 | 20.259 | H | PRO |
| | | 1693 | CE2 | 21.461 | −5.842 | 20.546 | C | PRO |
| | | 1694 | CD2 | 20.299 | −5.232 | 20.025 | C | PRO |
| | | 1695 | CE3 | 19.977 | −3.918 | 20.342 | C | PRO |
| | | 1696 | HE3 | 19.104 | −3.428 | 19.94 | H | PRO |
| | | 1697 | CZ3 | 20.822 | −3.228 | 21.22 | C | PRO |
| | | 1698 | HZ3 | 20.577 | −2.216 | 21.501 | H | PRO |
| | | 1699 | CZ2 | 22.312 | −5.151 | 21.4 | C | PRO |
| | | 1700 | HZ2 | 23.202 | −5.609 | 21.803 | H | PRO |
| | | 1701 | CH2 | 21.974 | −3.834 | 21.742 | C | PRO |
| | | 1702 | HH2 | 22.613 | −3.273 | 22.409 | H | PRO |
| | | 1703 | C | 18.967 | −4.367 | 16.755 | C | PRO |
| | | 1704 | O | 20.059 | −4.297 | 16.197 | O | PRO |
| 108 | CYS | 1705 | N | 18.273 | −3.275 | 17.124 | N | PRO |
| | | 1706 | HN | 17.379 | −3.356 | 17.571 | H | PRO |
| | | 1707 | CA | 18.704 | −1.942 | 16.816 | C | PRO |
| | | 1708 | HA | 19.786 | −1.893 | 16.823 | H | PRO |
| | | 1709 | CB | 18.122 | −1.526 | 15.435 | C | PRO |
| | | 1710 | HB1 | 18.405 | −2.319 | 14.707 | H | PRO |
| | | 1711 | HB2 | 17.011 | −1.531 | 15.492 | H | PRO |
| | | 1712 | SG | 18.71 | 0.072 | 14.799 | S | PRO |
| | | 1713 | HG1 | 18.091 | −0.02 | 13.629 | H | PRO |
| | | 1714 | C | 18.168 | −1.039 | 17.897 | C | PRO |
| | | 1715 | O | 17.033 | −1.201 | 18.338 | O | PRO |
| 109 | ASN | 1716 | N | 18.963 | −0.031 | 18.322 | N | PRO |
| | | 1717 | HN | 19.924 | 0.017 | 18.038 | H | PRO |
| | | 1718 | CA | 18.479 | 1.098 | 19.09 | C | PRO |
| | | 1719 | HA | 17.571 | 0.831 | 19.618 | H | PRO |
| | | 1720 | CB | 19.521 | 1.674 | 20.079 | C | PRO |
| | | 1721 | HB1 | 20.459 | 1.919 | 19.538 | H | PRO |
| | | 1722 | HB2 | 19.129 | 2.602 | 20.547 | H | PRO |
| | | 1723 | CG | 19.801 | 0.67 | 21.199 | C | PRO |
| | | 1724 | OD1 | 18.88 | 0.12 | 21.812 | O | PRO |
| | | 1725 | ND2 | 21.112 | 0.452 | 21.497 | N | PRO |
| | | 1726 | HD21 | 21.332 | −0.152 | 22.267 | H | PRO |
| | | 1727 | HD22 | 21.825 | 0.92 | 20.975 | H | PRO |
| | | 1728 | C | 18.143 | 2.162 | 18.075 | C | PRO |
| | | 1729 | O | 18.883 | 3.125 | 17.874 | O | PRO |
| 110 | ALA | 1730 | N | 17.004 | 1.962 | 17.38 | N | PRO |
| | | 1731 | HN | 16.415 | 1.189 | 17.609 | H | PRO |
| | | 1732 | CA | 16.52 | 2.785 | 16.303 | C | PRO |
| | | 1733 | HA | 17.284 | 2.815 | 15.542 | H | PRO |
| | | 1734 | CB | 15.227 | 2.197 | 15.714 | C | PRO |
| | | 1735 | HB1 | 15.413 | 1.166 | 15.345 | H | PRO |
| | | 1736 | HB2 | 14.435 | 2.147 | 16.493 | H | PRO |
| | | 1737 | HB3 | 14.852 | 2.813 | 14.868 | H | PRO |
| | | 1738 | C | 16.216 | 4.19 | 16.741 | C | PRO |
| | | 1739 | O | 15.734 | 4.41 | 17.847 | O | PRO |
| 111 | ARG | 1740 | N | 16.454 | 5.187 | 15.86 | N | PRO |
| | | 1741 | HN | 16.911 | 5.016 | 14.994 | H | PRO |
| | | 1742 | CA | 15.878 | 6.507 | 16.015 | C | PRO |
| | | 1743 | HA | 16.123 | 6.863 | 17.007 | H | PRO |
| | | 1744 | CB | 16.397 | 7.528 | 14.977 | C | PRO |
| | | 1745 | HB1 | 16.184 | 7.162 | 13.949 | H | PRO |
| | | 1746 | HB2 | 15.855 | 8.493 | 15.113 | H | PRO |
| | | 1747 | CG | 17.902 | 7.819 | 15.109 | C | PRO |
| | | 1748 | HG1 | 18.107 | 8.112 | 16.163 | H | PRO |
| | | 1749 | HG2 | 18.484 | 6.899 | 14.887 | H | PRO |
| | | 1750 | CD | 18.364 | 8.959 | 14.194 | C | PRO |
| | | 1751 | HD1 | 18.195 | 8.696 | 13.128 | H | PRO |
| | | 1752 | HD2 | 17.814 | 9.893 | 14.445 | H | PRO |
| | | 1753 | NE | 19.827 | 9.185 | 14.409 | N | PRO |
| | | 1754 | HE | 20.315 | 8.658 | 15.135 | H | PRO |
| | | 1755 | CZ | 20.527 | 10.16 | 13.774 | C | PRO |
| | | 1756 | NH1 | 19.943 | 10.991 | 12.881 | N | PRO |
| | | 1757 | HH11 | 20.489 | 11.729 | 12.461 | H | PRO |
| | | 1758 | HH12 | 18.954 | 10.937 | 12.696 | H | PRO |
| | | 1759 | NH2 | 21.845 | 10.311 | 14.038 | N | PRO |
| | | 1760 | HH21 | 22.383 | 11.033 | 13.599 | H | PRO |
| | | 1761 | HH22 | 22.332 | 9.634 | 14.635 | H | PRO |
| | | 1762 | C | 14.371 | 6.421 | 15.897 | C | PRO |
| | | 1763 | O | 13.845 | 5.598 | 15.15 | O | PRO |
| 112 | THR | 1764 | N | 13.628 | 7.278 | 16.633 | N | PRO |
| | | 1765 | HN | 14.046 | 7.946 | 17.248 | H | PRO |
| | | 1766 | CA | 12.173 | 7.241 | 16.659 | C | PRO |
| | | 1767 | HA | 11.867 | 6.208 | 16.747 | H | PRO |
| | | 1768 | CB | 11.578 | 8.006 | 17.831 | C | PRO |
| | | 1769 | HB | 10.468 | 8.059 | 17.742 | H | PRO |
| | | 1770 | OG1 | 12.101 | 9.329 | 17.905 | O | PRO |
| | | 1771 | HG1 | 11.594 | 9.768 | 18.593 | H | PRO |
| | | 1772 | CG2 | 11.922 | 7.261 | 19.133 | C | PRO |
| | | 1773 | HG21 | 11.492 | 7.788 | 20.01 | H | PRO |
| | | 1774 | HG22 | 11.507 | 6.232 | 19.11 | H | PRO |
| | | 1775 | HG23 | 13.02 | 7.191 | 19.273 | H | PRO |
| | | 1776 | C | 11.571 | 7.771 | 15.375 | C | PRO |
| | | 1777 | O | 10.4 | 7.536 | 15.09 | O | PRO |
| 113 | SER | 1778 | N | 12.379 | 8.471 | 14.55 | N | PRO |
| | | 1779 | HN | 13.314 | 8.69 | 14.821 | H | PRO |
| | | 1780 | CA | 11.982 | 8.987 | 13.26 | C | PRO |
| | | 1781 | HA | 10.91 | 9.124 | 13.234 | H | PRO |
| | | 1782 | CB | 12.679 | 10.344 | 12.987 | C | PRO |
| | | 1783 | HB1 | 12.379 | 10.747 | 11.995 | H | PRO |
| | | 1784 | HB2 | 12.355 | 11.068 | 13.767 | H | PRO |
| | | 1785 | OG | 14.101 | 10.219 | 13.043 | O | PRO |
| | | 1786 | HG1 | 14.444 | 11.073 | 13.351 | H | PRO |
| | | 1787 | C | 12.363 | 8.021 | 12.16 | C | PRO |
| | | 1788 | O | 12.123 | 8.296 | 10.986 | O | PRO |
| 114 | ALA | 1789 | N | 12.96 | 6.864 | 12.524 | N | PRO |
| | | 1790 | HN | 13.173 | 6.681 | 13.483 | H | PRO |
| | | 1791 | CA | 13.311 | 5.813 | 11.601 | C | PRO |
| | | 1792 | HA | 12.935 | 6.035 | 10.613 | H | PRO |
| | | 1793 | CB | 14.837 | 5.615 | 11.544 | C | PRO |
| | | 1794 | HB1 | 15.322 | 6.557 | 11.214 | H | PRO |
| | | 1795 | HB2 | 15.237 | 5.349 | 12.546 | H | PRO |
| | | 1796 | HB3 | 15.111 | 4.816 | 10.823 | H | PRO |
| | | 1797 | C | 12.679 | 4.519 | 12.05 | C | PRO |
| | | 1798 | O | 12.985 | 3.458 | 11.513 | O | PRO |
| 115 | SER | 1799 | N | 11.757 | 4.558 | 13.042 | N | PRO |
| | | 1800 | HN | 11.466 | 5.424 | 13.445 | H | PRO |
| | | 1801 | CA | 11.099 | 3.375 | 13.563 | C | PRO |
| | | 1802 | HA | 11.877 | 2.66 | 13.783 | H | PRO |
| | | 1803 | CB | 10.331 | 3.616 | 14.889 | C | PRO |
| | | 1804 | HB1 | 9.91 | 2.657 | 15.266 | H | PRO |
| | | 1805 | HB2 | 11.049 | 3.993 | 15.649 | H | PRO |
| | | 1806 | OG | 9.276 | 4.564 | 14.76 | O | PRO |
| | | 1807 | HG1 | 8.911 | 4.673 | 15.656 | H | PRO |
| | | 1808 | C | 10.202 | 2.731 | 12.533 | C | PRO |
| | | 1809 | O | 10.111 | 1.508 | 12.459 | O | PRO |
| 116 | GLY | 1810 | N | 9.543 | 3.556 | 11.687 | N | PRO |
| | | 1811 | HN | 9.631 | 4.551 | 11.794 | H | PRO |
| | | 1812 | CA | 8.649 | 3.106 | 10.642 | C | PRO |
| | | 1813 | HA1 | 8.203 | 3.989 | 10.21 | H | PRO |
| | | 1814 | HA2 | 7.918 | 2.451 | 11.092 | H | PRO |
| | | 1815 | C | 9.333 | 2.355 | 9.533 | C | PRO |
| | | 1816 | O | 8.712 | 1.52 | 8.881 | O | PRO |
| 117 | TYR | 1817 | N | 10.639 | 2.622 | 9.301 | N | PRO |
| | | 1818 | HN | 11.107 | 3.321 | 9.838 | H | PRO |
| | | 1819 | CA | 11.472 | 1.899 | 8.36 | C | PRO |
| | | 1820 | HA | 10.966 | 1.859 | 7.407 | H | PRO |
| | | 1821 | CB | 12.818 | 2.669 | 8.204 | C | PRO |
| | | 1822 | HB1 | 12.67 | 3.534 | 7.53 | H | PRO |
| | | 1823 | HB2 | 13.114 | 3.079 | 9.189 | H | PRO |
| | | 1824 | CG | 13.986 | 1.884 | 7.671 | C | PRO |
| | | 1825 | CD1 | 14.03 | 1.412 | 6.349 | C | PRO |
| | | 1826 | HD1 | 13.204 | 1.603 | 5.68 | H | PRO |
| | | 1827 | CE1 | 15.155 | 0.713 | 5.887 | C | PRO |
| | | 1828 | HE1 | 15.19 | 0.361 | 4.867 | H | PRO |
| | | 1829 | CZ | 16.236 | 0.485 | 6.75 | C | PRO |
| | | 1830 | OH | 17.376 | −0.208 | 6.313 | O | PRO |
| | | 1831 | HH | 18.018 | −0.213 | 7.026 | H | PRO |
| | | 1832 | CD2 | 15.071 | 1.632 | 8.527 | C | PRO |
| | | 1833 | HD2 | 15.043 | 1.987 | 9.546 | H | PRO |
| | | 1834 | CE2 | 16.193 | 0.942 | 8.068 | C | PRO |
| | | 1835 | HE2 | 17.02 | 0.766 | 8.737 | H | PRO |
| | | 1836 | C | 11.676 | 0.473 | 8.826 | C | PRO |
| | | 1837 | O | 11.494 | −0.479 | 8.069 | O | PRO |
| 118 | TYR | 1838 | N | 12.016 | 0.305 | 10.121 | N | PRO |
| | | 1839 | HN | 12.156 | 1.097 | 10.711 | H | PRO |
| | | 1840 | CA | 12.211 | −0.981 | 10.751 | C | PRO |
| | | 1841 | HA | 12.855 | −1.568 | 10.11 | H | PRO |
| | | 1842 | CB | 12.86 | −0.829 | 12.143 | C | PRO |
| | | 1843 | HB1 | 12.263 | −0.133 | 12.769 | H | PRO |
| | | 1844 | HB2 | 12.946 | −1.802 | 12.661 | H | PRO |
| | | 1845 | CG | 14.256 | −0.295 | 12.007 | C | PRO |
| | | 1846 | CD1 | 15.246 | −1.057 | 11.365 | C | PRO |
| | | 1847 | HD1 | 15.007 | −2.037 | 10.976 | H | PRO |
| | | 1848 | CE1 | 16.542 | −0.553 | 11.215 | C | PRO |
| | | 1849 | HE1 | 17.287 | −1.143 | 10.704 | H | PRO |
| | | 1850 | CZ | 16.861 | 0.718 | 11.71 | C | PRO |
| | | 1851 | OH | 18.162 | 1.233 | 11.554 | O | PRO |
| | | 1852 | HH | 18.194 | 2.087 | 11.989 | H | PRO |
| | | 1853 | CD2 | 14.591 | 0.969 | 12.514 | C | PRO |
| | | 1854 | HD2 | 13.84 | 1.557 | 13.019 | H | PRO |
| | | 1855 | CE2 | 15.886 | 1.48 | 12.361 | C | PRO |
| | | 1856 | HE2 | 16.12 | 2.462 | 12.746 | H | PRO |
| | | 1857 | C | 10.91 | −1.741 | 10.864 | C | PRO |
| | | 1858 | O | 10.876 | −2.957 | 10.706 | O | PRO |
| 119 | LYS | 1859 | N | 9.788 | −1.025 | 11.096 | N | PRO |
| | | 1860 | HN | 9.855 | −0.042 | 11.278 | H | PRO |
| | | 1861 | CA | 8.446 | −1.567 | 11.143 | C | PRO |
| | | 1862 | HA | 8.425 | −2.308 | 11.93 | H | PRO |
| | | 1863 | CB | 7.441 | −0.443 | 11.467 | C | PRO |
| | | 1864 | HB1 | 7.829 | 0.119 | 12.348 | H | PRO |
| | | 1865 | HB2 | 7.393 | 0.272 | 10.617 | H | PRO |
| | | 1866 | CG | 6.015 | −0.902 | 11.802 | C | PRO |
| | | 1867 | HG1 | 5.579 | −1.462 | 10.946 | H | PRO |
| | | 1868 | HG2 | 6.057 | −1.585 | 12.679 | H | PRO |
| | | 1869 | CD | 5.118 | 0.3 | 12.136 | C | PRO |
| | | 1870 | HD1 | 5.648 | 0.918 | 12.896 | H | PRO |
| | | 1871 | HD2 | 4.999 | 0.921 | 11.22 | H | PRO |
| | | 1872 | CE | 3.747 | −0.095 | 12.69 | C | PRO |
| | | 1873 | HE1 | 3.155 | −0.641 | 11.925 | H | PRO |
| | | 1874 | HE2 | 3.863 | −0.735 | 13.59 | H | PRO |
| | | 1875 | NZ | 2.992 | 1.115 | 13.087 | N | PRO |
| | | 1876 | HZ1 | 2.068 | 0.839 | 13.475 | H | PRO |
| | | 1877 | HZ2 | 3.538 | 1.631 | 13.807 | H | PRO |
| | | 1878 | HZ3 | 2.858 | 1.723 | 12.254 | H | PRO |
| | | 1879 | C | 8.045 | −2.233 | 9.84 | C | PRO |
| | | 1880 | O | 7.451 | −3.311 | 9.849 | O | PRO |
| 120 | LYS | 1881 | N | 8.419 | −1.626 | 8.686 | N | PRO |
| | | 1882 | HN | 8.898 | −0.749 | 8.708 | H | PRO |
| | | 1883 | CA | 8.163 | −2.158 | 7.36 | C | PRO |
| | | 1884 | HA | 7.144 | −2.516 | 7.327 | H | PRO |
| | | 1885 | CB | 8.381 | −1.086 | 6.264 | C | PRO |
| | | 1886 | HB1 | 9.382 | −0.623 | 6.412 | H | PRO |
| | | 1887 | HB2 | 8.374 | −1.557 | 5.255 | H | PRO |
| | | 1888 | CG | 7.322 | 0.029 | 6.247 | C | PRO |
| | | 1889 | HG1 | 7.218 | 0.46 | 7.263 | H | PRO |
| | | 1890 | HG2 | 7.695 | 0.831 | 5.572 | H | PRO |
| | | 1891 | CD | 5.943 | −0.445 | 5.757 | C | PRO |
| | | 1892 | HD1 | 6.07 | −0.93 | 4.765 | H | PRO |
| | | 1893 | HD2 | 5.555 | −1.218 | 6.456 | H | PRO |
| | | 1894 | CE | 4.888 | 0.664 | 5.637 | C | PRO |
| | | 1895 | HE1 | 3.932 | 0.24 | 5.261 | H | PRO |
| | | 1896 | HE2 | 4.712 | 1.149 | 6.619 | H | PRO |
| | | 1897 | NZ | 5.328 | 1.704 | 4.683 | N | PRO |
| | | 1898 | HZ1 | 4.555 | 2.387 | 4.498 | H | PRO |
| | | 1899 | HZ2 | 6.136 | 2.226 | 5.091 | H | PRO |
| | | 1900 | HZ3 | 5.618 | 1.252 | 3.781 | H | PRO |
| | | 1901 | C | 9.061 | −3.333 | 7.032 | C | PRO |
| | | 1902 | O | 8.768 | −4.102 | 6.117 | O | PRO |
| 121 | LEU | 1903 | N | 10.153 | −3.52 | 7.806 | N | PRO |
| | | 1904 | HN | 10.38 | −2.862 | 8.523 | H | PRO |
| | | 1905 | CA | 11.049 | −4.649 | 7.698 | C | PRO |
| | | 1906 | HA | 10.953 | −5.101 | 6.721 | H | PRO |
| | | 1907 | CB | 12.518 | −4.221 | 7.936 | C | PRO |
| | | 1908 | HB1 | 12.57 | −3.646 | 8.886 | H | PRO |
| | | 1909 | HB2 | 13.173 | −5.113 | 8.044 | H | PRO |
| | | 1910 | CG | 13.102 | −3.349 | 6.804 | C | PRO |
| | | 1911 | HG | 12.358 | −2.558 | 6.55 | H | PRO |
| | | 1912 | CD1 | 14.381 | −2.633 | 7.27 | C | PRO |
| | | 1913 | HD11 | 14.811 | −2.038 | 6.437 | H | PRO |
| | | 1914 | HD12 | 14.156 | −1.946 | 8.114 | H | PRO |
| | | 1915 | HD13 | 15.135 | −3.373 | 7.604 | H | PRO |
| | | 1916 | CD2 | 13.374 | −4.173 | 5.533 | C | PRO |
| | | 1917 | HD21 | 13.759 | −3.51 | 4.733 | H | PRO |
| | | 1918 | HD22 | 14.134 | −4.958 | 5.735 | H | PRO |
| | | 1919 | HD23 | 12.446 | −4.656 | 5.163 | H | PRO |
| | | 1920 | C | 10.677 | −5.7 | 8.723 | C | PRO |
| | | 1921 | O | 11.382 | −6.693 | 8.882 | O | PRO |
| 122 | GLY | 1922 | N | 9.53 | −5.533 | 9.424 | N | PRO |
| | | 1923 | HN | 8.975 | −4.711 | 9.294 | H | PRO |
| | | 1924 | CA | 8.979 | −6.54 | 10.307 | C | PRO |
| | | 1925 | HA1 | 9.159 | −7.515 | 9.876 | H | PRO |
| | | 1926 | HA2 | 7.925 | −6.322 | 10.394 | H | PRO |
| | | 1927 | C | 9.546 | −6.54 | 11.699 | C | PRO |
| | | 1928 | O | 9.289 | −7.471 | 12.46 | O | PRO |
| 123 | PHE | 1929 | N | 10.335 | −5.509 | 12.079 | N | PRO |
| | | 1930 | HN | 10.547 | −4.765 | 11.444 | H | PRO |
| | | 1931 | CA | 10.834 | −5.334 | 13.431 | C | PRO |
| | | 1932 | HA | 11.232 | −6.282 | 13.765 | H | PRO |
| | | 1933 | CB | 11.93 | −4.242 | 13.588 | C | PRO |
| | | 1934 | HB1 | 11.57 | −3.301 | 13.127 | H | PRO |
| | | 1935 | HB2 | 12.105 | −4.046 | 14.669 | H | PRO |
| | | 1936 | CG | 13.277 | −4.57 | 12.978 | C | PRO |
| | | 1937 | CD1 | 13.448 | −4.826 | 11.603 | C | PRO |
| | | 1938 | HD1 | 12.6 | −4.83 | 10.941 | H | PRO |
| | | 1939 | CE1 | 14.719 | −5.068 | 11.067 | C | PRO |
| | | 1940 | HE1 | 14.831 | −5.265 | 10.011 | H | PRO |
| | | 1941 | CZ | 15.844 | −5.052 | 11.9 | C | PRO |
| | | 1942 | HZ | 16.825 | −5.234 | 11.486 | H | PRO |
| | | 1943 | CD2 | 14.425 | −4.542 | 13.794 | C | PRO |
| | | 1944 | HD2 | 14.327 | −4.319 | 14.845 | H | PRO |
| | | 1945 | CE2 | 15.697 | −4.787 | 13.266 | C | PRO |
| | | 1946 | HE2 | 16.564 | −4.769 | 13.911 | H | PRO |
| | | 1947 | C | 9.705 | −4.945 | 14.371 | C | PRO |
| | | 1948 | O | 8.735 | −4.295 | 13.977 | O | PRO |
| 124 | SER | 1949 | N | 9.84 | −5.333 | 15.656 | N | PRO |
| | | 1950 | HN | 10.643 | −5.866 | 15.936 | H | PRO |
| | | 1951 | CA | 8.905 | −5.028 | 16.718 | C | PRO |
| | | 1952 | HA | 8.065 | −4.466 | 16.333 | H | PRO |
| | | 1953 | CB | 8.401 | −6.283 | 17.471 | C | PRO |
| | | 1954 | HB1 | 9.269 | −6.869 | 17.845 | H | PRO |
| | | 1955 | HB2 | 7.77 | −5.994 | 18.341 | H | PRO |
| | | 1956 | OG | 7.638 | −7.12 | 16.608 | O | PRO |
| | | 1957 | HG1 | 6.765 | −6.719 | 16.542 | H | PRO |
| | | 1958 | C | 9.637 | −4.181 | 17.722 | C | PRO |
| | | 1959 | O | 10.815 | −4.409 | 17.988 | O | PRO |
| 125 | GLU | 1960 | N | 8.95 | −3.166 | 18.294 | N | PRO |
| | | 1961 | HN | 7.984 | −3.007 | 18.072 | H | PRO |
| | | 1962 | CA | 9.488 | −2.309 | 19.329 | C | PRO |
| | | 1963 | HA | 10.517 | −2.089 | 19.089 | H | PRO |
| | | 1964 | CB | 8.717 | −0.973 | 19.481 | C | PRO |
| | | 1965 | HB1 | 7.663 | −1.189 | 19.768 | H | PRO |
| | | 1966 | HB2 | 9.18 | −0.377 | 20.3 | H | PRO |
| | | 1967 | CG | 8.716 | −0.115 | 18.202 | C | PRO |
| | | 1968 | HG1 | 9.761 | 0.095 | 17.893 | H | PRO |
| | | 1969 | HG2 | 8.203 | −0.663 | 17.385 | H | PRO |
| | | 1970 | CD | 8.011 | 1.226 | 18.392 | C | PRO |
| | | 1971 | OE1 | 7.597 | 1.557 | 19.532 | O | PRO |
| | | 1972 | OE2 | 7.89 | 1.949 | 17.364 | O | PRO |
| | | 1973 | C | 9.445 | −3.008 | 20.665 | C | PRO |
| | | 1974 | O | 8.646 | −3.918 | 20.879 | O | PRO |
| 126 | GLN | 1975 | N | 10.314 | −2.58 | 21.603 | N | PRO |
| | | 1976 | HN | 11.009 | −1.886 | 21.405 | H | PRO |
| | | 1977 | CA | 10.261 | −3.059 | 22.958 | C | PRO |
| | | 1978 | HA | 9.232 | −3.278 | 23.21 | H | PRO |
| | | 1979 | CB | 11.135 | −4.318 | 23.181 | C | PRO |
| | | 1980 | HB1 | 10.998 | −4.988 | 22.302 | H | PRO |
| | | 1981 | HB2 | 12.208 | −4.029 | 23.209 | H | PRO |
| | | 1982 | CG | 10.743 | −5.111 | 24.441 | C | PRO |
| | | 1983 | HG1 | 10.764 | −4.458 | 25.339 | H | PRO |
| | | 1984 | HG2 | 9.713 | −5.51 | 24.322 | H | PRO |
| | | 1985 | CD | 11.7 | −6.282 | 24.654 | C | PRO |
| | | 1986 | OE1 | 11.421 | −7.421 | 24.267 | O | PRO |
| | | 1987 | NE2 | 12.858 | −5.987 | 25.307 | N | PRO |
| | | 1988 | HE21 | 13.486 | −6.73 | 25.541 | H | PRO |
| | | 1989 | HE22 | 13.033 | −5.042 | 25.605 | H | PRO |
| | | 1990 | C | 10.737 | −1.944 | 23.855 | C | PRO |
| | | 1991 | O | 11.608 | −1.156 | 23.485 | O | PRO |
| 127 | GLY | 1992 | N | 10.158 | −1.849 | 25.074 | N | PRO |
| | | 1993 | HN | 9.398 | −2.45 | 25.319 | H | PRO |
| | | 1994 | CA | 10.553 | −0.885 | 26.079 | C | PRO |
| | | 1995 | HA1 | 11.632 | −0.899 | 26.155 | H | PRO |
| | | 1996 | HA2 | 10.066 | −1.177 | 26.997 | H | PRO |
| | | 1997 | C | 10.126 | 0.519 | 25.763 | C | PRO |
| | | 1998 | O | 9.445 | 0.785 | 24.774 | O | PRO |
| 128 | GLU | 1999 | N | 10.517 | 1.459 | 26.646 | N | PRO |
| | | 2000 | HN | 11.06 | 1.219 | 27.446 | H | PRO |
| | | 2001 | CA | 10.186 | 2.858 | 26.521 | C | PRO |
| | | 2002 | HA | 9.21 | 2.945 | 26.062 | H | PRO |
| | | 2003 | CB | 10.169 | 3.578 | 27.892 | C | PRO |
| | | 2004 | HB1 | 11.176 | 3.5 | 28.357 | H | PRO |
| | | 2005 | HB2 | 9.951 | 4.661 | 27.747 | H | PRO |
| | | 2006 | CG | 9.132 | 3.012 | 28.886 | C | PRO |
| | | 2007 | HG1 | 9.346 | 1.946 | 29.106 | H | PRO |
| | | 2008 | HG2 | 9.179 | 3.584 | 29.837 | H | PRO |
| | | 2009 | CD | 7.718 | 3.133 | 28.324 | C | PRO |
| | | 2010 | OE1 | 7.304 | 4.282 | 28.01 | O | PRO |
| | | 2011 | OE2 | 7.039 | 2.081 | 28.191 | O | PRO |
| | | 2012 | C | 11.184 | 3.561 | 25.635 | C | PRO |
| | | 2013 | O | 12.298 | 3.085 | 25.415 | O | PRO |
| 129 | VAL | 2014 | N | 10.781 | 4.735 | 25.098 | N | PRO |
| | | 2015 | HN | 9.847 | 5.064 | 25.257 | H | PRO |
| | | 2016 | CA | 11.645 | 5.672 | 24.406 | C | PRO |
| | | 2017 | HA | 12.17 | 5.12 | 23.64 | H | PRO |
| | | 2018 | CB | 10.831 | 6.785 | 23.744 | C | PRO |
| | | 2019 | HB | 10.224 | 7.305 | 24.521 | H | PRO |
| | | 2020 | CG1 | 11.722 | 7.832 | 23.043 | C | PRO |
| | | 2021 | HG11 | 11.086 | 8.568 | 22.506 | H | PRO |
| | | 2022 | HG12 | 12.345 | 8.391 | 23.771 | H | PRO |
| | | 2023 | HG13 | 12.387 | 7.342 | 22.302 | H | PRO |
| | | 2024 | CG2 | 9.866 | 6.144 | 22.723 | C | PRO |
| | | 2025 | HG21 | 9.278 | 6.934 | 22.21 | H | PRO |
| | | 2026 | HG22 | 10.439 | 5.579 | 21.958 | H | PRO |
| | | 2027 | HG23 | 9.154 | 5.451 | 23.216 | H | PRO |
| | | 2028 | C | 12.673 | 6.233 | 25.376 | C | PRO |
| | | 2029 | O | 12.37 | 6.469 | 26.545 | O | PRO |
| 130 | PHE | 2030 | N | 13.923 | 6.45 | 24.909 | N | PRO |
| | | 2031 | HN | 14.165 | 6.239 | 23.96 | H | PRO |
| | | 2032 | CA | 14.997 | 6.95 | 25.737 | C | PRO |
| | | 2033 | HA | 14.577 | 7.546 | 26.536 | H | PRO |
| | | 2034 | CB | 15.882 | 5.826 | 26.36 | C | PRO |
| | | 2035 | HB1 | 16.689 | 6.274 | 26.979 | H | PRO |
| | | 2036 | HB2 | 15.249 | 5.199 | 27.024 | H | PRO |
| | | 2037 | CG | 16.514 | 4.912 | 25.331 | C | PRO |
| | | 2038 | CD1 | 15.818 | 3.795 | 24.834 | C | PRO |
| | | 2039 | HD1 | 14.821 | 3.585 | 25.191 | H | PRO |
| | | 2040 | CE1 | 16.402 | 2.954 | 23.88 | C | PRO |
| | | 2041 | HE1 | 15.859 | 2.097 | 23.509 | H | PRO |
| | | 2042 | CZ | 17.696 | 3.217 | 23.417 | C | PRO |
| | | 2043 | HZ | 18.147 | 2.563 | 22.689 | H | PRO |
| | | 2044 | CD2 | 17.815 | 5.163 | 24.857 | C | PRO |
| | | 2045 | HD2 | 18.363 | 6.019 | 25.225 | H | PRO |
| | | 2046 | CE2 | 18.404 | 4.321 | 23.905 | C | PRO |
| | | 2047 | HE2 | 19.404 | 4.526 | 23.55 | H | PRO |
| | | 2048 | C | 15.832 | 7.875 | 24.889 | C | PRO |
| | | 2049 | O | 15.999 | 7.648 | 23.695 | O | PRO |
| 131 | ASP | 2050 | N | 16.369 | 8.958 | 25.488 | N | PRO |
| | | 2051 | HN | 16.236 | 9.131 | 26.467 | H | PRO |
| | | 2052 | CA | 17.144 | 9.947 | 24.773 | C | PRO |
| | | 2053 | HA | 16.867 | 9.94 | 23.729 | H | PRO |
| | | 2054 | CB | 16.946 | 11.376 | 25.33 | C | PRO |
| | | 2055 | HB1 | 17.115 | 11.387 | 26.427 | H | PRO |
| | | 2056 | HB2 | 17.651 | 12.09 | 24.854 | H | PRO |
| | | 2057 | CG | 15.529 | 11.843 | 25.026 | C | PRO |
| | | 2058 | OD1 | 14.744 | 12.033 | 25.992 | O | PRO |
| | | 2059 | OD2 | 15.219 | 12.023 | 23.819 | O | PRO |
| | | 2060 | C | 18.609 | 9.61 | 24.855 | C | PRO |
| | | 2061 | O | 19.124 | 9.255 | 25.914 | O | PRO |
| 132 | THR | 2062 | N | 19.315 | 9.739 | 23.71 | N | PRO |
| | | 2063 | HN | 18.865 | 9.996 | 22.853 | H | PRO |
| | | 2064 | CA | 20.757 | 9.62 | 23.654 | C | PRO |
| | | 2065 | HA | 21.161 | 9.44 | 24.638 | H | PRO |
| | | 2066 | CB | 21.263 | 8.53 | 22.714 | C | PRO |
| | | 2067 | HB | 20.968 | 8.741 | 21.662 | H | PRO |
| | | 2068 | OG1 | 20.707 | 7.268 | 23.058 | O | PRO |
| | | 2069 | HG1 | 20.972 | 6.675 | 22.35 | H | PRO |
| | | 2070 | CG2 | 22.798 | 8.423 | 22.815 | C | PRO |
| | | 2071 | HG21 | 23.172 | 7.604 | 22.164 | H | PRO |
| | | 2072 | HG22 | 23.287 | 9.365 | 22.491 | H | PRO |
| | | 2073 | HG23 | 23.106 | 8.205 | 23.859 | H | PRO |
| | | 2074 | C | 21.217 | 10.948 | 23.105 | C | PRO |
| | | 2075 | O | 20.971 | 11.193 | 21.926 | O | PRO |
| 133 | PRO | 2076 | N | 21.865 | 11.852 | 23.831 | N | PRO |
| | | 2077 | CD | 22.031 | 11.796 | 25.284 | C | PRO |
| | | 2078 | HD1 | 21.034 | 11.923 | 25.759 | H | PRO |
| | | 2079 | HD2 | 22.492 | 10.836 | 25.6 | H | PRO |
| | | 2080 | CA | 22.306 | 13.123 | 23.275 | C | PRO |
| | | 2081 | HA | 21.512 | 13.544 | 22.674 | H | PRO |
| | | 2082 | CB | 22.619 | 13.986 | 24.51 | C | PRO |
| | | 2083 | HB1 | 21.699 | 14.543 | 24.797 | H | PRO |
| | | 2084 | HB2 | 23.439 | 14.712 | 24.34 | H | PRO |
| | | 2085 | CG | 22.944 | 12.978 | 25.617 | C | PRO |
| | | 2086 | HG1 | 22.765 | 13.385 | 26.631 | H | PRO |
| | | 2087 | HG2 | 24.005 | 12.659 | 25.527 | H | PRO |
| | | 2088 | C | 23.542 | 12.9 | 22.418 | C | PRO |
| | | 2089 | O | 24.323 | 12.02 | 22.781 | O | PRO |
| 134 | PRO | 2090 | N | 23.779 | 13.584 | 21.303 | N | PRO |
| | | 2091 | CD | 25.085 | 13.457 | 20.649 | C | PRO |
| | | 2092 | HD1 | 25.888 | 13.634 | 21.399 | H | PRO |
| | | 2093 | HD2 | 25.199 | 12.446 | 20.202 | H | PRO |
| | | 2094 | CA | 23.033 | 14.738 | 20.815 | C | PRO |
| | | 2095 | HA | 22.523 | 15.256 | 21.615 | H | PRO |
| | | 2096 | CB | 24.126 | 15.585 | 20.144 | C | PRO |
| | | 2097 | HB1 | 24.642 | 16.186 | 20.926 | H | PRO |
| | | 2098 | HB2 | 23.74 | 16.273 | 19.366 | H | PRO |
| | | 2099 | CG | 25.112 | 14.554 | 19.585 | C | PRO |
| | | 2100 | HG1 | 26.126 | 14.975 | 19.433 | H | PRO |
| | | 2101 | HG2 | 24.73 | 14.152 | 18.622 | H | PRO |
| | | 2102 | C | 22.024 | 14.283 | 19.783 | C | PRO |
| | | 2103 | O | 21.441 | 15.13 | 19.108 | O | PRO |
| 135 | VAL | 2104 | N | 21.797 | 12.959 | 19.653 | N | PRO |
| | | 2105 | HN | 22.219 | 12.314 | 20.287 | H | PRO |
| | | 2106 | CA | 21.067 | 12.34 | 18.567 | C | PRO |
| | | 2107 | HA | 20.948 | 13.063 | 17.771 | H | PRO |
| | | 2108 | CB | 21.825 | 11.148 | 17.99 | C | PRO |
| | | 2109 | HB | 21.177 | 10.584 | 17.279 | H | PRO |
| | | 2110 | CG1 | 23.022 | 11.685 | 17.176 | C | PRO |
| | | 2111 | HG11 | 23.53 | 10.845 | 16.656 | H | PRO |
| | | 2112 | HG12 | 22.678 | 12.406 | 16.405 | H | PRO |
| | | 2113 | HG13 | 23.756 | 12.191 | 17.836 | H | PRO |
| | | 2114 | CG2 | 22.288 | 10.194 | 19.112 | C | PRO |
| | | 2115 | HG21 | 22.781 | 9.304 | 18.672 | H | PRO |
| | | 2116 | HG22 | 23.019 | 10.683 | 19.788 | H | PRO |
| | | 2117 | HG23 | 21.426 | 9.846 | 19.715 | H | PRO |
| | | 2118 | C | 19.661 | 11.946 | 18.986 | C | PRO |
| | | 2119 | O | 19.032 | 11.096 | 18.356 | O | PRO |
| 136 | GLY | 2120 | N | 19.115 | 12.611 | 20.034 | N | PRO |
| | | 2121 | HN | 19.675 | 13.253 | 20.553 | H | PRO |
| | | 2122 | CA | 17.713 | 12.581 | 20.415 | C | PRO |
| | | 2123 | HA1 | 17.195 | 12.987 | 19.561 | H | PRO |
| | | 2124 | HA2 | 17.625 | 13.203 | 21.293 | H | PRO |
| | | 2125 | C | 17.116 | 11.231 | 20.766 | C | PRO |
| | | 2126 | O | 17.833 | 10.33 | 21.202 | O | PRO |
| 137 | PRO | 2127 | N | 15.796 | 11.068 | 20.642 | N | PRO |
| | | 2128 | CD | 14.877 | 12.168 | 20.341 | C | PRO |
| | | 2129 | HD1 | 14.89 | 12.889 | 21.189 | H | PRO |
| | | 2130 | HD2 | 15.138 | 12.674 | 19.386 | H | PRO |
| | | 2131 | CA | 15.085 | 9.916 | 21.182 | C | PRO |
| | | 2132 | HA | 15.452 | 9.706 | 22.175 | H | PRO |
| | | 2133 | CB | 13.61 | 10.351 | 21.225 | C | PRO |
| | | 2134 | HB1 | 13.391 | 10.74 | 22.246 | H | PRO |
| | | 2135 | HB2 | 12.899 | 9.533 | 20.997 | H | PRO |
| | | 2136 | CG | 13.507 | 11.507 | 20.23 | C | PRO |
| | | 2137 | HG1 | 12.677 | 12.202 | 20.466 | H | PRO |
| | | 2138 | HG2 | 13.385 | 11.11 | 19.201 | H | PRO |
| | | 2139 | C | 15.267 | 8.673 | 20.341 | C | PRO |
| | | 2140 | O | 15.28 | 8.747 | 19.112 | O | PRO |
| 138 | HIS | 2141 | N | 15.427 | 7.514 | 21.011 | N | PRO |
| | | 2142 | HN | 15.432 | 7.508 | 22.016 | H | PRO |
| | | 2143 | CA | 15.634 | 6.227 | 20.399 | C | PRO |
| | | 2144 | HA | 15.379 | 6.284 | 19.351 | H | PRO |
| | | 2145 | CB | 17.08 | 5.696 | 20.575 | C | PRO |
| | | 2146 | HB1 | 17.35 | 5.697 | 21.654 | H | PRO |
| | | 2147 | HB2 | 17.15 | 4.652 | 20.199 | H | PRO |
| | | 2148 | CD2 | 18.74 | 6.225 | 18.658 | C | PRO |
| | | 2149 | HD2 | 18.711 | 5.335 | 18.04 | H | PRO |
| | | 2150 | CG | 18.099 | 6.499 | 19.818 | C | PRO |
| | | 2151 | NE2 | 19.486 | 7.328 | 18.349 | N | PRO |
| | | 2152 | HE2 | 20.115 | 7.455 | 17.546 | H | PRO |
| | | 2153 | ND1 | 18.473 | 7.76 | 20.191 | N | PRO |
| | | 2154 | HD1 | 18.12 | 8.29 | 20.968 | H | PRO |
| | | 2155 | CE1 | 19.306 | 8.241 | 19.285 | C | PRO |
| | | 2156 | HE1 | 19.735 | 9.226 | 19.29 | H | PRO |
| | | 2157 | C | 14.696 | 5.247 | 21.049 | C | PRO |
| | | 2158 | O | 14.151 | 5.502 | 22.12 | O | PRO |
| 139 | ILE | 2159 | N | 14.489 | 4.091 | 20.389 | N | PRO |
| | | 2160 | HN | 14.935 | 3.916 | 19.507 | H | PRO |
| | | 2161 | CA | 13.593 | 3.053 | 20.835 | C | PRO |
| | | 2162 | HA | 13.523 | 3.088 | 21.914 | H | PRO |
| | | 2163 | CB | 12.195 | 3.194 | 20.212 | C | PRO |
| | | 2164 | HB | 11.777 | 4.162 | 20.58 | H | PRO |
| | | 2165 | CG2 | 12.261 | 3.304 | 18.668 | C | PRO |
| | | 2166 | HG21 | 11.241 | 3.486 | 18.265 | H | PRO |
| | | 2167 | HG22 | 12.91 | 4.143 | 18.345 | H | PRO |
| | | 2168 | HG23 | 12.64 | 2.363 | 18.22 | H | PRO |
| | | 2169 | CG1 | 11.201 | 2.088 | 20.641 | C | PRO |
| | | 2170 | HG11 | 10.258 | 2.229 | 20.064 | H | PRO |
| | | 2171 | HG12 | 11.602 | 1.091 | 20.362 | H | PRO |
| | | 2172 | CD1 | 10.847 | 2.104 | 22.129 | C | PRO |
| | | 2173 | HD1 | 10.13 | 1.286 | 22.354 | H | PRO |
| | | 2174 | HD2 | 11.746 | 1.959 | 22.758 | H | PRO |
| | | 2175 | HD3 | 10.374 | 3.069 | 22.408 | H | PRO |
| | | 2176 | C | 14.261 | 1.749 | 20.462 | C | PRO |
| | | 2177 | O | 14.773 | 1.595 | 19.354 | O | PRO |
| 140 | LEU | 2178 | N | 14.31 | 0.771 | 21.401 | N | PRO |
| | | 2179 | HN | 13.948 | 0.907 | 22.325 | H | PRO |
| | | 2180 | CA | 14.796 | −0.566 | 21.123 | C | PRO |
| | | 2181 | HA | 15.755 | −0.47 | 20.631 | H | PRO |
| | | 2182 | CB | 14.968 | −1.408 | 22.412 | C | PRO |
| | | 2183 | HB1 | 15.636 | −0.851 | 23.104 | H | PRO |
| | | 2184 | HB2 | 13.981 | −1.493 | 22.915 | H | PRO |
| | | 2185 | CG | 15.539 | −2.834 | 22.231 | C | PRO |
| | | 2186 | HG | 14.905 | −3.382 | 21.497 | H | PRO |
| | | 2187 | CD1 | 16.98 | −2.829 | 21.697 | C | PRO |
| | | 2188 | HD11 | 17.354 | −3.869 | 21.598 | H | PRO |
| | | 2189 | HD12 | 17.035 | −2.341 | 20.703 | H | PRO |
| | | 2190 | HD13 | 17.649 | −2.285 | 22.396 | H | PRO |
| | | 2191 | CD2 | 15.458 | −3.613 | 23.553 | C | PRO |
| | | 2192 | HD21 | 15.877 | −4.632 | 23.43 | H | PRO |
| | | 2193 | HD22 | 16.029 | −3.084 | 24.345 | H | PRO |
| | | 2194 | HD23 | 14.402 | −3.703 | 23.881 | H | PRO |
| | | 2195 | C | 13.842 | −1.278 | 20.196 | C | PRO |
| | | 2196 | O | 12.628 | −1.249 | 20.394 | O | PRO |
| 141 | MET | 2197 | N | 14.382 | −1.938 | 19.153 | N | PRO |
| | | 2198 | HN | 15.369 | −1.923 | 18.981 | H | PRO |
| | | 2199 | CA | 13.596 | −2.664 | 18.193 | C | PRO |
| | | 2200 | HA | 12.653 | −2.945 | 18.636 | H | PRO |
| | | 2201 | CB | 13.365 | −1.881 | 16.876 | C | PRO |
| | | 2202 | HB1 | 14.341 | −1.663 | 16.39 | H | PRO |
| | | 2203 | HB2 | 12.767 | −2.511 | 16.184 | H | PRO |
| | | 2204 | CG | 12.629 | −0.547 | 17.107 | C | PRO |
| | | 2205 | HG1 | 11.751 | −0.749 | 17.756 | H | PRO |
| | | 2206 | HG2 | 13.305 | 0.121 | 17.685 | H | PRO |
| | | 2207 | SD | 12.08 | 0.324 | 15.612 | S | PRO |
| | | 2208 | CE | 10.773 | −0.834 | 15.119 | C | PRO |
| | | 2209 | HE1 | 10.142 | −0.402 | 14.312 | H | PRO |
| | | 2210 | HE2 | 11.203 | −1.782 | 14.741 | H | PRO |
| | | 2211 | HE3 | 10.114 | −1.08 | 15.975 | H | PRO |
| | | 2212 | C | 14.359 | −3.917 | 17.885 | C | PRO |
| | | 2213 | O | 15.584 | −3.948 | 17.986 | O | PRO |
| 142 | TYR | 2214 | N | 13.64 | −4.996 | 17.515 | N | PRO |
| | | 2215 | HN | 12.639 | −4.971 | 17.471 | H | PRO |
| | | 2216 | CA | 14.258 | −6.271 | 17.247 | C | PRO |
| | | 2217 | HA | 15.229 | −6.089 | 16.81 | H | PRO |
| | | 2218 | CB | 14.441 | −7.156 | 18.52 | C | PRO |
| | | 2219 | HB1 | 15.081 | −8.031 | 18.279 | H | PRO |
| | | 2220 | HB2 | 14.952 | −6.561 | 19.307 | H | PRO |
| | | 2221 | CG | 13.133 | −7.659 | 19.087 | C | PRO |
| | | 2222 | CD1 | 12.31 | −6.837 | 19.877 | C | PRO |
| | | 2223 | HD1 | 12.633 | −5.84 | 20.136 | H | PRO |
| | | 2224 | CE1 | 11.048 | −7.286 | 20.292 | C | PRO |
| | | 2225 | HE1 | 10.405 | −6.636 | 20.867 | H | PRO |
| | | 2226 | CZ | 10.599 | −8.557 | 19.913 | C | PRO |
| | | 2227 | OH | 9.296 | −8.976 | 20.255 | O | PRO |
| | | 2228 | HH | 9.027 | −9.648 | 19.612 | H | PRO |
| | | 2229 | CD2 | 12.687 | −8.949 | 18.75 | C | PRO |
| | | 2230 | HD2 | 13.303 | −9.584 | 18.129 | H | PRO |
| | | 2231 | CE2 | 11.423 | −9.393 | 19.151 | C | PRO |
| | | 2232 | HE2 | 11.081 | −10.372 | 18.849 | H | PRO |
| | | 2233 | C | 13.446 | −6.991 | 16.203 | C | PRO |
| | | 2234 | O | 12.239 | −6.788 | 16.085 | O | PRO |
| 143 | LYS | 2235 | N | 14.114 | −7.861 | 15.424 | N | PRO |
| | | 2236 | HN | 15.112 | −7.943 | 15.489 | H | PRO |
| | | 2237 | CA | 13.484 | −8.736 | 14.472 | C | PRO |
| | | 2238 | HA | 12.415 | −8.773 | 14.633 | H | PRO |
| | | 2239 | CB | 13.811 | −8.312 | 13.025 | C | PRO |
| | | 2240 | HB1 | 13.446 | −7.273 | 12.879 | H | PRO |
| | | 2241 | HB2 | 14.915 | −8.292 | 12.884 | H | PRO |
| | | 2242 | CG | 13.179 | −9.19 | 11.937 | C | PRO |
| | | 2243 | HG1 | 13.451 | −10.257 | 12.101 | H | PRO |
| | | 2244 | HG2 | 12.074 | −9.099 | 12.01 | H | PRO |
| | | 2245 | CD | 13.649 | −8.788 | 10.533 | C | PRO |
| | | 2246 | HD1 | 13.398 | −7.72 | 10.349 | H | PRO |
| | | 2247 | HD2 | 14.759 | −8.876 | 10.503 | H | PRO |
| | | 2248 | CE | 13.075 | −9.659 | 9.412 | C | PRO |
| | | 2249 | HE1 | 13.558 | −9.386 | 8.449 | H | PRO |
| | | 2250 | HE2 | 13.247 | −10.737 | 9.614 | H | PRO |
| | | 2251 | NZ | 11.622 | −9.439 | 9.243 | N | PRO |
| | | 2252 | HZ1 | 11.302 | −9.912 | 8.367 | H | PRO |
| | | 2253 | HZ2 | 11.085 | −9.8 | 10.068 | H | PRO |
| | | 2254 | HZ3 | 11.46 | −8.409 | 9.145 | H | PRO |
| | | 2255 | C | 14.071 | −10.097 | 14.705 | C | PRO |
| | | 2256 | O | 15.282 | −10.278 | 14.592 | O | PRO |
| 144 | ARG | 2257 | N | 13.222 | −11.099 | 15.026 | N | PRO |
| | | 2258 | HN | 12.243 | −10.941 | 15.157 | H | PRO |
| | | 2259 | CA | 13.655 | −12.47 | 15.172 | C | PRO |
| | | 2260 | HA | 14.658 | −12.478 | 15.571 | H | PRO |
| | | 2261 | CB | 12.749 | −13.262 | 16.143 | C | PRO |
| | | 2262 | HB1 | 12.63 | −12.636 | 17.056 | H | PRO |
| | | 2263 | HB2 | 11.739 | −13.396 | 15.701 | H | PRO |
| | | 2264 | CG | 13.324 | −14.628 | 16.56 | C | PRO |
| | | 2265 | HG1 | 13.365 | −15.288 | 15.666 | H | PRO |
| | | 2266 | HG2 | 14.367 | −14.485 | 16.92 | H | PRO |
| | | 2267 | CD | 12.524 | −15.354 | 17.656 | C | PRO |
| | | 2268 | HD1 | 11.479 | −15.523 | 17.315 | H | PRO |
| | | 2269 | HD2 | 12.978 | −16.338 | 17.903 | H | PRO |
| | | 2270 | NE | 12.481 | −14.489 | 18.885 | N | PRO |
| | | 2271 | HE | 11.703 | −13.875 | 18.997 | H | PRO |
| | | 2272 | CZ | 13.482 | −14.397 | 19.797 | C | PRO |
| | | 2273 | NH1 | 14.578 | −15.186 | 19.769 | N | PRO |
| | | 2274 | HH11 | 15.262 | −15.074 | 20.491 | H | PRO |
| | | 2275 | HH12 | 14.677 | −15.93 | 19.093 | H | PRO |
| | | 2276 | NH2 | 13.393 | −13.477 | 20.788 | N | PRO |
| | | 2277 | HH21 | 14.153 | −13.41 | 21.441 | H | PRO |
| | | 2278 | HH22 | 12.615 | −12.86 | 20.858 | H | PRO |
| | | 2279 | C | 13.678 | −13.106 | 13.805 | C | PRO |
| | | 2280 | O | 12.722 | −12.982 | 13.04 | O | PRO |
| 145 | ILE | 2281 | N | 14.809 | −13.759 | 13.455 | N | PRO |
| | | 2282 | HN | 15.555 | −13.888 | 14.11 | H | PRO |
| | | 2283 | CA | 15.068 | −14.223 | 12.106 | C | PRO |
| | | 2284 | HA | 14.261 | −13.916 | 11.456 | H | PRO |
| | | 2285 | CB | 16.361 | −13.65 | 11.532 | C | PRO |
| | | 2286 | HB | 16.528 | −14.075 | 10.515 | H | PRO |
| | | 2287 | CG2 | 16.167 | −12.126 | 11.357 | C | PRO |
| | | 2288 | HG21 | 17.038 | −11.679 | 10.833 | H | PRO |
| | | 2289 | HG22 | 15.258 | −11.921 | 10.752 | H | PRO |
| | | 2290 | HG23 | 16.057 | −11.626 | 12.342 | H | PRO |
| | | 2291 | CG1 | 17.594 | −14 | 12.399 | C | PRO |
| | | 2292 | HG11 | 17.474 | −13.556 | 13.409 | H | PRO |
| | | 2293 | HG12 | 17.655 | −15.104 | 12.521 | H | PRO |
| | | 2294 | CD1 | 18.919 | −13.513 | 11.809 | C | PRO |
| | | 2295 | HD1 | 19.763 | −13.884 | 12.427 | H | PRO |
| | | 2296 | HD2 | 19.05 | −13.894 | 10.775 | H | PRO |
| | | 2297 | HD3 | 18.964 | −12.403 | 11.79 | H | PRO |
| | | 2298 | C | 15.093 | −15.732 | 12.061 | C | PRO |
| | | 2299 | O | 15.38 | −16.324 | 11.022 | O | PRO |
| 146 | THR | 2300 | N | 14.741 | −16.398 | 13.179 | N | PRO |
| | | 2301 | HN | 14.533 | −15.921 | 14.026 | H | PRO |
| | | 2302 | CA | 14.501 | −17.828 | 13.204 | C | PRO |
| | | 2303 | HA | 15.079 | −18.303 | 12.424 | H | PRO |
| | | 2304 | CB | 14.896 | −18.479 | 14.525 | C | PRO |
| | | 2305 | HB | 14.625 | −19.56 | 14.523 | H | PRO |
| | | 2306 | OG1 | 14.266 | −17.852 | 15.639 | O | PRO |
| | | 2307 | HG1 | 13.34 | −18.143 | 15.617 | H | PRO |
| | | 2308 | CG2 | 16.422 | −18.367 | 14.687 | C | PRO |
| | | 2309 | HG21 | 16.747 | −18.827 | 15.643 | H | PRO |
| | | 2310 | HG22 | 16.93 | −18.894 | 13.853 | H | PRO |
| | | 2311 | HG23 | 16.743 | −17.305 | 14.677 | H | PRO |
| | | 2312 | C | 13.01 | −18.11 | 12.908 | C | PRO |
| | | 2313 | OT1 | 12.251 | −17.162 | 12.577 | O | PRO |
| | | 2314 | OT2 | 12.607 | −19.298 | 13.03 | O | PRO |
| 150 | ACO | 2315 | N1A | 10.178 | 6.578 | 8.578 | N | LIG |
| | | 2316 | C2A | 9.23 | 5.72 | 8.179 | C | LIG |
| | | 2317 | H2 | 8.322 | 5.723 | 8.783 | H | LIG |
| | | 2318 | N3A | 9.225 | 4.857 | 7.155 | N | LIG |
| | | 2319 | C4A | 10.381 | 4.933 | 6.479 | C | LIG |
| | | 2320 | C5A | 11.445 | 5.775 | 6.768 | C | LIG |
| | | 2321 | C6A | 11.349 | 6.641 | 7.871 | C | LIG |
| | | 2322 | N6A | 12.387 | 7.458 | 8.201 | N | LIG |
| | | 2323 | H61 | 12.394 | 7.921 | 9.091 | H | LIG |
| | | 2324 | H62 | 13.197 | 7.534 | 7.62 | H | LIG |
| | | 2325 | N7A | 12.459 | 5.58 | 5.867 | N | LIG |
| | | 2326 | C8A | 12.022 | 4.649 | 5.06 | C | LIG |
| | | 2327 | H8 | 12.579 | 4.246 | 4.212 | H | LIG |
| | | 2328 | N9A | 10.76 | 4.208 | 5.378 | N | LIG |
| | | 2329 | C1B | 9.974 | 3.167 | 4.717 | C | LIG |
| | | 2330 | H1′ | 8.962 | 3.079 | 5.169 | H | LIG |
| | | 2331 | C4B | 10.461 | 1.143 | 3.639 | C | LIG |
| | | 2332 | H4′ | 9.95 | 0.181 | 3.859 | H | LIG |
| | | 2333 | O4B | 10.565 | 1.891 | 4.881 | O | LIG |
| | | 2334 | C2B | 9.852 | 3.411 | 3.245 | C | LIG |
| | | 2335 | H2′ | 10.813 | 3.76 | 2.805 | H | LIG |
| | | 2336 | O2B | 8.829 | 4.338 | 2.953 | O | LIG |
| | | 2337 | HO2′ | 9.158 | 4.922 | 2.24 | H | LIG |
| | | 2338 | C3B | 9.547 | 2.005 | 2.752 | C | LIG |
| | | 2339 | H3′ | 9.895 | 1.912 | 1.696 | H | LIG |
| | | 2340 | O3B | 8.166 | 1.638 | 2.925 | O | LIG |
| | | 2341 | P3B | 7.401 | 0.898 | 1.77 | P | LIG |
| | | 2342 | O7A | 8.295 | −0.076 | 1.107 | O | LIG |
| | | 2343 | O8A | 6.105 | 0.41 | 2.295 | O | LIG |
| | | 2344 | O9A | 7.081 | 2.021 | 0.705 | O | LIG |
| | | 2345 | C5B | 11.847 | 0.843 | 3.036 | C | LIG |
| | | 2346 | H5′1 | 12.365 | 0.141 | 3.729 | H | LIG |
| | | 2347 | H5′2 | 11.696 | 0.325 | 2.06 | H | LIG |
| | | 2348 | O5B | 12.642 | 2.027 | 2.874 | O | LIG |
| | | 2349 | P1A | 14.022 | 1.952 | 2.113 | P | LIG |
| | | 2350 | O1A | 13.841 | 2.518 | 0.756 | O | LIG |
| | | 2351 | O2A | 14.579 | 0.584 | 2.202 | O | LIG |
| | | 2352 | O3A | 14.907 | 2.911 | 3.003 | O | LIG |
| | | 2353 | P2A | 16.058 | 3.949 | 2.708 | P | LIG |
| | | 2354 | O4A | 15.45 | 5.211 | 2.235 | O | LIG |
| | | 2355 | O5A | 17.078 | 3.324 | 1.834 | O | LIG |
| | | 2356 | O6A | 16.716 | 4.174 | 4.12 | O | LIG |
| | | 2357 | CBP | 16.666 | 5.235 | 6.316 | C | LIG |
| | | 2358 | CCP | 15.925 | 4.372 | 5.298 | C | LIG |
| | | 2359 | H121 | 14.928 | 4.819 | 5.082 | H | LIG |
| | | 2360 | H122 | 15.756 | 3.382 | 5.766 | H | LIG |
| | | 2361 | CDP | 18.025 | 4.54 | 6.574 | C | LIG |
| | | 2362 | H131 | 17.86 | 3.475 | 6.842 | H | LIG |
| | | 2363 | H132 | 18.668 | 4.577 | 5.67 | H | LIG |
| | | 2364 | H133 | 18.562 | 5.027 | 7.414 | H | LIG |
| | | 2365 | CEP | 15.786 | 5.24 | 7.588 | C | LIG |
| | | 2366 | H141 | 15.66 | 4.204 | 7.96 | H | LIG |
| | | 2367 | H142 | 16.26 | 5.832 | 8.397 | H | LIG |
| | | 2368 | H143 | 14.78 | 5.657 | 7.379 | H | LIG |
| | | 2369 | CAP | 16.829 | 6.638 | 5.703 | C | LIG |
| | | 2370 | H10 | 17.244 | 6.52 | 4.676 | H | LIG |
| | | 2371 | OAP | 15.573 | 7.32 | 5.626 | O | LIG |
| | | 2372 | HO10 | 15.073 | 6.936 | 4.888 | H | LIG |
| | | 2373 | C9P | 17.83 | 7.476 | 6.468 | C | LIG |
| | | 2374 | O9P | 18.983 | 7.613 | 6.057 | O | LIG |
| | | 2375 | N8P | 17.408 | 8.075 | 7.6 | N | LIG |
| | | 2376 | HN8 | 16.461 | 7.972 | 7.896 | H | LIG |
| | | 2377 | C7P | 18.239 | 8.966 | 8.357 | C | LIG |
| | | 2378 | H71 | 18.811 | 9.624 | 7.662 | H | LIG |
| | | 2379 | H72 | 17.585 | 9.638 | 8.957 | H | LIG |
| | | 2380 | C6P | 19.236 | 8.255 | 9.286 | C | LIG |
| | | 2381 | HC1 | 19.856 | 7.61 | 8.682 | H | LIG |
| | | 2382 | HC2 | 19.795 | 9.013 | 9.811 | H | LIG |
| | | 2383 | C5P | 18.534 | 7.422 | 10.307 | C | LIG |
| | | 2384 | O5P | 17.518 | 7.823 | 10.871 | O | LIG |
| | | 2385 | N4P | 19.106 | 6.241 | 10.598 | N | LIG |
| | | 2386 | H4 | 19.912 | 5.917 | 10.1 | H | LIG |
| | | 2387 | C3P | 18.687 | 5.399 | 11.681 | C | LIG |
| | | 2388 | H31 | 18.025 | 4.599 | 11.281 | H | LIG |
| | | 2389 | H32 | 18.114 | 5.979 | 12.441 | H | LIG |
| | | 2390 | C2P | 19.926 | 4.782 | 12.338 | C | LIG |
| | | 2391 | H151 | 20.535 | 5.606 | 12.677 | H | LIG |
| | | 2392 | H152 | 20.433 | 4.221 | 11.573 | H | LIG |
| | | 2393 | S1P | 19.609 | 3.671 | 13.738 | S | LIG |
| | | 2394 | C | 21.28 | 3.079 | 14.029 | C | LIG |
| | | 2395 | O | 22.204 | 3.338 | 13.259 | O | LIG |
| | | 2396 | CH3 | 21.525 | 2.265 | 15.257 | C | LIG |
| | | 2397 | HB21 | 20.948 | 2.648 | 16.126 | H | LIG |
| | | 2398 | HB22 | 21.254 | 1.203 | 15.08 | H | LIG |
| | | 2399 | HB23 | 22.605 | 2.303 | 15.518 | H | LIG |
| 151 | GLF | 2400 | C | 25.417 | 4.856 | 15.508 | C | LIG |
| | | 2401 | OC2 | 26.476 | 5.094 | 16.147 | O | LIG |
| | | 2402 | OC1 | 24.753 | 3.796 | 15.664 | O | LIG |
| | | 2403 | C1 | 24.961 | 5.944 | 14.532 | C | LIG |
| | | 2404 | H11 | 25.601 | 5.877 | 13.629 | H | LIG |
| | | 2405 | H12 | 25.127 | 6.939 | 14.986 | H | LIG |
| | | 2406 | N | 23.529 | 5.878 | 14.08 | N | LIG |
| | | 2407 | HN1 | 23.361 | 4.963 | 13.6 | H | LIG |
| | | 2408 | HN2 | 23.381 | 6.66 | 13.407 | H | LIG |
| | | 2409 | C2 | 22.472 | 6.018 | 15.147 | C | LIG |
| | | 2410 | H21 | 21.496 | 6 | 14.624 | H | LIG |
| | | 2411 | H22 | 22.522 | 5.125 | 15.795 | H | LIG |
| | | 2412 | P | 22.542 | 7.577 | 16.221 | P | LIG |
| | | 2413 | OP2 | 23.276 | 8.52 | 15.356 | O | LIG |
| | | 2414 | OP1 | 21.096 | 7.835 | 16.36 | O | LIG |
| | | 2415 | OP3 | 23.247 | 7.075 | 17.426 | O | LIG |
| 161 | HOH | 2416 | OH2 | 16.688 | 3.703 | −0.773 | O | WAT |
| | | 2417 | H1 | 16.855 | 3.626 | 0.186 | H | WAT |
| | | 2418 | H2 | 17.295 | 4.397 | −1.067 | H | WAT |
| 162 | HOH | 2419 | OH2 | 32.978 | −6 | 6.008 | O | WAT |
| | | 2420 | H1 | 32.414 | −6.632 | 5.53 | H | WAT |
| | | 2421 | H2 | 32.941 | −5.209 | 5.447 | H | WAT |
| 163 | HOH | 2422 | OH2 | 27.781 | 2.001 | 9.24 | O | WAT |
| | | 2423 | H1 | 27.852 | 1.062 | 9.032 | H | WAT |
| | | 2424 | H2 | 26.858 | 2.214 | 9.043 | H | WAT |
| 164 | HOH | 2425 | OH2 | 35.002 | −4.151 | 0.093 | O | WAT |
| | | 2426 | H1 | 35.229 | −4.611 | 0.916 | H | WAT |
| | | 2427 | H2 | 34.063 | −4.354 | −0.022 | H | WAT |
| 165 | HOH | 2428 | OH2 | 35.028 | −1.375 | −0.596 | O | WAT |
| | | 2429 | H1 | 35.015 | −0.9 | 0.256 | H | WAT |
| | | 2430 | H2 | 35.087 | −2.306 | −0.344 | H | WAT |
| 166 | HOH | 2431 | OH2 | 27.311 | −3.014 | 14.273 | O | WAT |
| | | 2432 | H1 | 26.749 | −2.922 | 13.496 | H | WAT |
| | | 2433 | H2 | 28.089 | −2.469 | 14.073 | H | WAT |
| 167 | HOH | 2434 | OH2 | 30.494 | 6.157 | 3.588 | O | WAT |
| | | 2435 | H1 | 30.583 | 6.493 | 2.685 | H | WAT |
| | | 2436 | H2 | 31.116 | 5.416 | 3.611 | H | WAT |
| 168 | HOH | 2437 | OH2 | 34.413 | 6.632 | 4.262 | O | WAT |
| | | 2438 | H1 | 34.761 | 5.886 | 4.771 | H | WAT |
| | | 2439 | H2 | 34.779 | 6.471 | 3.378 | H | WAT |
| 169 | HOH | 2440 | OH2 | 37.779 | 6.46 | 5.881 | O | WAT |
| | | 2441 | H1 | 37.308 | 7.29 | 5.723 | H | WAT |
| | | 2442 | H2 | 37.095 | 5.788 | 5.792 | H | WAT |
| 170 | HOH | 2443 | OH2 | 35.344 | −4.638 | 11.125 | O | WAT |
| | | 2444 | H1 | 34.622 | −4.181 | 10.66 | H | WAT |
| | | 2445 | H2 | 35.182 | −4.414 | 12.052 | H | WAT |
| 171 | HOH | 2446 | OH2 | 11.585 | 1.867 | −0.537 | O | WAT |
| | | 2447 | H1 | 12.37 | 2.188 | −0.053 | H | WAT |
| | | 2448 | H2 | 10.972 | 2.617 | −0.508 | H | WAT |
| 172 | HOH | 2449 | OH2 | 21.801 | −0.15 | 17.783 | O | WAT |
| | | 2450 | H1 | 22.157 | −0.415 | 16.922 | H | WAT |
| | | 2451 | H2 | 22.287 | 0.654 | 18.017 | H | WAT |
| 173 | HOH | 2452 | OH2 | 13.884 | 6.976 | 3.402 | O | WAT |
| | | 2453 | H1 | 14.439 | 6.325 | 2.93 | H | WAT |
| | | 2454 | H2 | 12.989 | 6.75 | 3.08 | H | WAT |
| 174 | HOH | 2455 | OH2 | 35.759 | −6.639 | 13.873 | O | WAT |
| | | 2456 | H1 | 35.64 | −5.68 | 13.859 | H | WAT |
| | | 2457 | H2 | 35.244 | −6.912 | 14.647 | H | WAT |
| 175 | HOH | 2458 | OH2 | 35.513 | −5.257 | 2.634 | O | WAT |
| | | 2459 | H1 | 36.269 | −4.756 | 2.973 | H | WAT |
| | | 2460 | H2 | 34.944 | −5.35 | 3.4 | H | WAT |
| 176 | HOH | 2461 | OH2 | 29.563 | −1.39 | 13.98 | O | WAT |
| | | 2462 | H1 | 30.373 | −1.125 | 13.505 | H | WAT |
| | | 2463 | H2 | 29.779 | −1.191 | 14.901 | H | WAT |
| 177 | HOH | 2464 | OH2 | 13.445 | 0.83 | 24.322 | O | WAT |
| | | 2465 | H1 | 12.992 | 1.579 | 24.741 | H | WAT |
| | | 2466 | H2 | 12.734 | 0.2 | 24.134 | H | WAT |
| 178 | HOH | 2467 | OH2 | 31.282 | 6.009 | −3.628 | O | WAT |
| | | 2468 | H1 | 32.211 | 5.925 | −3.848 | H | WAT |
| | | 2469 | H2 | 31.216 | 6.899 | −3.247 | H | WAT |
| 179 | HOH | 2470 | OH2 | 37.572 | 3.836 | 2.66 | O | WAT |
| | | 2471 | H1 | 38.462 | 3.571 | 2.895 | H | WAT |
| | | 2472 | H2 | 37.107 | 2.978 | 2.565 | H | WAT |
| 180 | HOH | 2473 | OH2 | 22.042 | 12.937 | 3.359 | O | WAT |
| | | 2474 | H1 | 21.609 | 13.169 | 4.192 | H | WAT |
| | | 2475 | H2 | 22.142 | 13.782 | 2.903 | H | WAT |
| 181 | HOH | 2476 | OH2 | 42.316 | 1.171 | 9.782 | O | WAT |
| | | 2477 | H1 | 41.798 | 1.576 | 10.481 | H | WAT |
| | | 2478 | H2 | 41.63 | 0.816 | 9.19 | H | WAT |
| 182 | HOH | 2479 | OH2 | 31.118 | 7.143 | 0.968 | O | WAT |
| | | 2480 | H1 | 31.818 | 6.477 | 0.861 | H | WAT |
| | | 2481 | H2 | 31.631 | 7.977 | 0.982 | H | WAT |
| 183 | HOH | 2482 | OH2 | 31.371 | −7.826 | 4.7 | O | WAT |
| | | 2483 | H1 | 32.091 | −8.459 | 4.561 | H | WAT |
| | | 2484 | H2 | 30.591 | −8.318 | 4.402 | H | WAT |
| 184 | HOH | 2485 | OH2 | 10.19 | −11.045 | 15.686 | O | WAT |
| | | 2486 | H1 | 9.975 | −10.129 | 15.432 | H | WAT |
| | | 2487 | H2 | 10.033 | −11.536 | 14.866 | H | WAT |
| 185 | HOH | 2488 | OH2 | 9.384 | 6.489 | 11.965 | O | WAT |
| | | 2489 | H1 | 8.925 | 6.749 | 12.776 | H | WAT |
| | | 2490 | H2 | 8.692 | 6.59 | 11.297 | H | WAT |
| 186 | HOH | 2491 | OH2 | 29.964 | −11.206 | 14.913 | O | WAT |
| | | 2492 | H1 | 30.233 | −10.664 | 14.165 | H | WAT |
| | | 2493 | H2 | 30.68 | −11.064 | 15.55 | H | WAT |
| 187 | HOH | 2494 | OH2 | 29.037 | 8.42 | −0.375 | O | WAT |
| | | 2495 | H1 | 28.457 | 7.709 | −0.693 | H | WAT |
| | | 2496 | H2 | 29.643 | 7.951 | 0.223 | H | WAT |
| 188 | HOH | 2497 | OH2 | 25.717 | 14.863 | 15.366 | O | WAT |
| | | 2498 | H1 | 24.813 | 15.111 | 15.57 | H | WAT |
| | | 2499 | H2 | 25.899 | 14.12 | 15.951 | H | WAT |
| 189 | HOH | 2500 | OH2 | 21.453 | 13.063 | 11.708 | O | WAT |
| | | 2501 | H1 | 22.034 | 12.928 | 10.943 | H | WAT |
| | | 2502 | H2 | 21.595 | 13.984 | 11.934 | H | WAT |
| 190 | HOH | 2503 | OH2 | 23.479 | −6.943 | −5.335 | O | WAT |
| | | 2504 | H1 | 23.341 | −7.758 | −4.844 | H | WAT |
| | | 2505 | H2 | 22.723 | −6.903 | −5.925 | H | WAT |
| 191 | HOH | 2506 | OH2 | 9.321 | 3.572 | −0.408 | O | WAT |
| | | 2507 | H1 | 8.562 | 3.256 | 0.099 | H | WAT |
| | | 2508 | H2 | 9.521 | 4.431 | 0.013 | H | WAT |
| 192 | HOH | 2509 | OH2 | 35.776 | 1.748 | 13.506 | O | WAT |
| | | 2510 | H1 | 36.074 | 2.082 | 12.648 | H | WAT |
| | | 2511 | H2 | 34.853 | 1.483 | 13.351 | H | WAT |
| 193 | HOH | 2512 | OH2 | 32.702 | −10.744 | 9.66 | O | WAT |
| | | 2513 | H1 | 32.735 | −9.837 | 9.998 | H | WAT |
| | | 2514 | H2 | 33.516 | −11.138 | 10.025 | H | WAT |
| 194 | HOH | 2515 | OH2 | 9.446 | −8.532 | 14.963 | O | WAT |
| | | 2516 | H1 | 9.361 | −8.103 | 14.099 | H | WAT |
| | | 2517 | H2 | 8.875 | −7.994 | 15.534 | H | WAT |
| 195 | HOH | 2518 | OH2 | 37.042 | −3.285 | 7.927 | O | WAT |
| | | 2519 | H1 | 37.869 | −3.382 | 7.443 | H | WAT |
| | | 2520 | H2 | 36.524 | −4.042 | 7.621 | H | WAT |
| 196 | HOH | 2521 | OH2 | 13.082 | −7.981 | 6.013 | O | WAT |
| | | 2522 | H1 | 13.944 | −8.405 | 6.212 | H | WAT |
| | | 2523 | H2 | 13.237 | −7.066 | 6.25 | H | WAT |
| 197 | HOH | 2524 | OH2 | 6.784 | 3.448 | 13.598 | O | WAT |
| | | 2525 | H1 | 6.92 | 2.59 | 14.029 | H | WAT |
| | | 2526 | H2 | 7.593 | 3.926 | 13.814 | H | WAT |
| 198 | HOH | 2527 | OH2 | 7.557 | −1.843 | 15.045 | O | WAT |
| | | 2528 | H1 | 7.689 | −2.605 | 14.469 | H | WAT |
| | | 2529 | H2 | 8.228 | −1.965 | 15.719 | H | WAT |
| 199 | HOH | 2530 | OH2 | 35.83 | 5.872 | 1.955 | O | WAT |
| | | 2531 | H1 | 36.378 | 6.435 | 1.403 | H | WAT |
| | | 2532 | H2 | 36.444 | 5.159 | 2.213 | H | WAT |
| 200 | HOH | 2533 | OH2 | 25.278 | 0.465 | −8.774 | O | WAT |
| | | 2534 | H1 | 25.333 | 0.608 | −9.72 | H | WAT |
| | | 2535 | H2 | 25.84 | −0.323 | −8.638 | H | WAT |
| 201 | HOH | 2536 | OH2 | 33.194 | 5.301 | 1.14 | O | WAT |
| | | 2537 | H1 | 32.933 | 4.771 | 1.904 | H | WAT |
| | | 2538 | H2 | 34.133 | 5.48 | 1.302 | H | WAT |
| 202 | HOH | 2539 | OH2 | 12.371 | −16.012 | 10.149 | O | WAT |
| | | 2540 | H1 | 12.411 | −16.37 | 11.057 | H | WAT |
| | | 2541 | H2 | 11.628 | −16.487 | 9.775 | H | WAT |
| 203 | HOH | 2542 | OH2 | 7.929 | 5.484 | 25.736 | O | WAT |
| | | 2543 | H1 | 7.742 | 5.073 | 26.605 | H | WAT |
| | | 2544 | H2 | 7.055 | 5.578 | 25.357 | H | WAT |
| 204 | HOH | 2545 | OH2 | 25.376 | 16.465 | 5.367 | O | WAT |
| | | 2546 | H1 | 25.653 | 17.174 | 5.948 | H | WAT |
| | | 2547 | H2 | 25.304 | 15.7 | 5.955 | H | WAT |
| 205 | HOH | 2548 | OH2 | 27.433 | −10.197 | 23.868 | O | WAT |
| | | 2549 | H1 | 28.309 | −10.205 | 23.479 | H | WAT |
| | | 2550 | H2 | 27.05 | −11.039 | 23.562 | H | WAT |
| 206 | HOH | 2551 | OH2 | 16.635 | 10.274 | 17.324 | O | WAT |
| | | 2552 | H1 | 16.319 | 9.725 | 18.057 | H | WAT |
| | | 2553 | H2 | 17.506 | 10.576 | 17.625 | H | WAT |
| 207 | HOH | 2554 | OH2 | 24.264 | −13.52 | 1.79 | O | WAT |
| | | 2555 | H1 | 23.341 | −13.592 | 1.476 | H | WAT |
| | | 2556 | H2 | 24.541 | −12.682 | 1.393 | H | WAT |
| 208 | HOH | 2557 | OH2 | 15.363 | −17.412 | 18.219 | O | WAT |
| | | 2558 | H1 | 15.205 | −17.701 | 17.309 | H | WAT |
| | | 2559 | H2 | 16.279 | −17.695 | 18.404 | H | WAT |
| 209 | HOH | 2560 | OH2 | 28.683 | −3.045 | 21.234 | O | WAT |
| | | 2561 | H1 | 27.968 | −3.307 | 21.837 | H | WAT |
| | | 2562 | H2 | 28.598 | −3.692 | 20.516 | H | WAT |
| 212 | HOH | 2563 | OH2 | 25.665 | 7.713 | −8.322 | O | WAT |
| | | 2564 | H1 | 25.29 | 6.967 | −7.838 | H | WAT |
| | | 2565 | H2 | 26.347 | 7.31 | −8.865 | H | WAT |
| 213 | HOH | 2566 | OH2 | 35.951 | −6.691 | 9.319 | O | WAT |
| | | 2567 | H1 | 36.016 | −7.445 | 9.916 | H | WAT |
| | | 2568 | H2 | 35.798 | −5.953 | 9.929 | H | WAT |
| 214 | HOH | 2569 | OH2 | 24.966 | 13.876 | −2.593 | O | WAT |
| | | 2570 | H1 | 24.122 | 13.837 | −2.114 | H | WAT |
| | | 2571 | H2 | 24.721 | 13.624 | −3.496 | H | WAT |
| 215 | HOH | 2572 | OH2 | 18.582 | −14.829 | 26.317 | O | WAT |
| | | 2573 | H1 | 17.832 | −14.68 | 26.907 | H | WAT |
| | | 2574 | H2 | 19.133 | −14.046 | 26.467 | H | WAT |
| 216 | HOH | 2575 | OH2 | 5.994 | 2.382 | 8.65 | O | WAT |
| | | 2576 | H1 | 6.094 | 2.941 | 7.868 | H | WAT |
| | | 2577 | H2 | 6.872 | 1.999 | 8.759 | H | WAT |
| 217 | HOH | 2578 | OH2 | 19.593 | −12.204 | 26.715 | O | WAT |
| | | 2579 | H1 | 18.819 | −11.816 | 26.281 | H | WAT |
| | | 2580 | H2 | 20.219 | −11.475 | 26.695 | H | WAT |
| 218 | HOH | 2581 | OH2 | 18.099 | −19.071 | 21.325 | O | WAT |
| | | 2582 | H1 | 18.247 | −19.998 | 21.125 | H | WAT |
| | | 2583 | H2 | 18.042 | −18.659 | 20.443 | H | WAT |
| 219 | HOH | 2584 | OH2 | 7.822 | 7.293 | 14.187 | O | WAT |
| | | 2585 | H1 | 7.311 | 6.795 | 14.826 | H | WAT |
| | | 2586 | H2 | 8.673 | 7.429 | 14.633 | H | WAT |
| 220 | HOH | 2587 | OH2 | 14.122 | 10.792 | 16.291 | O | WAT |
| | | 2588 | H1 | 14.967 | 10.584 | 16.722 | H | WAT |
| | | 2589 | H2 | 13.462 | 10.367 | 16.854 | H | WAT |
| 221 | HOH | 2590 | OH2 | 33.532 | −9.621 | 4.655 | O | WAT |
| | | 2591 | H1 | 34.4 | −9.305 | 4.918 | H | WAT |
| | | 2592 | H2 | 33.25 | −10.164 | 5.409 | H | WAT |
| 222 | HOH | 2593 | OH2 | 15.826 | 9.21 | 28.401 | O | WAT |
| | | 2594 | H1 | 15.377 | 10.068 | 28.478 | H | WAT |
| | | 2595 | H2 | 15.826 | 8.878 | 29.299 | H | WAT |
| 223 | HOH | 2596 | OH2 | 30.427 | 1.161 | 19.704 | O | WAT |
| | | 2597 | H1 | 31.065 | 1.821 | 20.018 | H | WAT |
| | | 2598 | H2 | 30.571 | 0.402 | 20.298 | H | WAT |
| 224 | HOH | 2599 | OH2 | 11.568 | −18.798 | 15.527 | O | WAT |
| | | 2600 | H1 | 11.811 | −18.956 | 14.596 | H | WAT |
| | | 2601 | H2 | 10.652 | −19.073 | 15.563 | H | WAT |
| 225 | HOH | 2602 | OH2 | 31.025 | 8.545 | −2.451 | O | WAT |
| | | 2603 | H1 | 30.595 | 8.432 | −1.592 | H | WAT |
| | | 2604 | H2 | 31.868 | 8.975 | −2.218 | H | WAT |
| 226 | HOH | 2605 | OH2 | 13.286 | −10.982 | 22.6 | O | WAT |
| | | 2606 | H1 | 13.027 | −10.263 | 22.02 | H | WAT |
| | | 2607 | H2 | 12.925 | −10.709 | 23.46 | H | WAT |
| 227 | HOH | 2608 | OH2 | 30.931 | 12.546 | 13.362 | O | WAT |
| | | 2609 | H1 | 31.678 | 12.832 | 13.906 | H | WAT |
| | | 2610 | H2 | 30.315 | 13.288 | 13.37 | H | WAT |
| 228 | HOH | 2611 | OH2 | 12.693 | −12.17 | 7.239 | O | WAT |
| | | 2612 | H1 | 12.076 | −11.558 | 6.816 | H | WAT |
| | | 2613 | H2 | 13.555 | −11.72 | 7.13 | H | WAT |
| 229 | HOH | 2614 | OH2 | 22.665 | 13.98 | −0.903 | O | WAT |
| | | 2615 | H1 | 21.987 | 13.296 | −0.897 | H | WAT |
| | | 2616 | H2 | 22.541 | 14.434 | −0.055 | H | WAT |
| 230 | HOH | 2617 | OH2 | 22.85 | 1.886 | −10.526 | O | WAT |
| | | 2618 | H1 | 22.545 | 1.163 | −9.962 | H | WAT |
| | | 2619 | H2 | 22.181 | 1.943 | −11.211 | H | WAT |
| 231 | HOH | 2620 | OH2 | 6.134 | −2.864 | 17.498 | O | WAT |
| | | 2621 | H1 | 6.027 | −2.346 | 16.694 | H | WAT |
| | | 2622 | H2 | 5.402 | −2.592 | 18.056 | H | WAT |
| 233 | HOH | 2623 | OH2 | 11.364 | −3.517 | 2.756 | O | WAT |
| | | 2624 | H1 | 12.124 | −2.917 | 2.805 | H | WAT |
| | | 2625 | H2 | 10.599 | −2.925 | 2.845 | H | WAT |
| 234 | HOH | 2626 | OH2 | 18.203 | −10.485 | 0.168 | O | WAT |
| | | 2627 | H1 | 17.378 | −10.958 | 0.287 | H | WAT |
| | | 2628 | H2 | 18.863 | −11.205 | 0.227 | H | WAT |
| 235 | HOH | 2629 | OH2 | 17.958 | −0.548 | 24.412 | O | WAT |
| | | 2630 | H1 | 18.282 | −0.295 | 23.536 | H | WAT |
| | | 2631 | H2 | 18.457 | −1.342 | 24.619 | H | WAT |
| 236 | HOH | 2632 | OH2 | 30.555 | −9.784 | 20.327 | O | WAT |
| | | 2633 | H1 | 29.89 | −9.199 | 19.939 | H | WAT |
| | | 2634 | H2 | 30.017 | −10.43 | 20.807 | H | WAT |
| 237 | HOH | 2635 | OH2 | 13.841 | −0.932 | 27.954 | O | WAT |
| | | 2636 | H1 | 14.338 | −1.623 | 28.433 | H | WAT |
| | | 2637 | H2 | 14.516 | −0.558 | 27.368 | H | WAT |
| 238 | HOH | 2638 | OH2 | 21.565 | −12.958 | −1.84 | O | WAT |
| | | 2639 | H1 | 20.67 | −12.696 | −1.603 | H | WAT |
| | | 2640 | H2 | 21.897 | −13.235 | −0.97 | H | WAT |
| 239 | HOH | 2641 | OH2 | 8.931 | −2.198 | 2.659 | O | WAT |
| | | 2642 | H1 | 8.841 | −1.41 | 2.1 | H | WAT |
| | | 2643 | H2 | 8.009 | −2.466 | 2.772 | H | WAT |
| 240 | HOH | 2644 | OH2 | 14.301 | 10.09 | 5.163 | O | WAT |
| | | 2645 | H1 | 14.4 | 9.963 | 6.119 | H | WAT |
| | | 2646 | H2 | 14.04 | 9.217 | 4.854 | H | WAT |
| 241 | HOH | 2647 | OH2 | 13.332 | −3.184 | 26.211 | O | WAT |
| | | 2648 | H1 | 13.081 | −2.427 | 26.753 | H | WAT |
| | | 2649 | H2 | 14.292 | −3.223 | 26.381 | H | WAT |
| 242 | HOH | 2650 | OH2 | 37.52 | −2.858 | 10.71 | O | WAT |
| | | 2651 | H1 | 36.888 | −3.554 | 10.934 | H | WAT |
| | | 2652 | H2 | 37.497 | −2.862 | 9.742 | H | WAT |
| 243 | HOH | 2653 | OH2 | 36.128 | −3.562 | 20.347 | O | WAT |
| | | 2654 | H1 | 36.451 | −4.204 | 19.711 | H | WAT |
| | | 2655 | H2 | 35.409 | −3.114 | 19.884 | H | WAT |
| 244 | HOH | 2656 | OH2 | 11.146 | −9.952 | 6.389 | O | WAT |
| | | 2657 | H1 | 11.788 | −9.256 | 6.157 | H | WAT |
| | | 2658 | H2 | 10.475 | −9.872 | 5.708 | H | WAT |
| 245 | HOH | 2659 | OH2 | 36.842 | 4.433 | 17.867 | O | WAT |
| | | 2660 | H1 | 36.997 | 3.698 | 17.256 | H | WAT |
| | | 2661 | H2 | 35.979 | 4.768 | 17.598 | H | WAT |
| 246 | HOH | 2662 | OH2 | 23.911 | 13.122 | −5.157 | O | WAT |
| | | 2663 | H1 | 23.944 | 13.713 | −5.913 | H | WAT |
| | | 2664 | H2 | 23.019 | 13.272 | −4.793 | H | WAT |
| 247 | HOH | 2665 | OH2 | 29.462 | −9.725 | 3.912 | O | WAT |
| | | 2666 | H1 | 29.706 | −10.371 | 3.233 | H | WAT |
| | | 2667 | H2 | 29.496 | −10.243 | 4.733 | H | WAT |
| 248 | HOH | 2668 | OH2 | 22.451 | −0.276 | −8.706 | O | WAT |
| | | 2669 | H1 | 23.315 | 0.017 | −8.38 | H | WAT |
| | | 2670 | H2 | 22.656 | −1.102 | −9.15 | H | WAT |
| 249 | HOH | 2671 | OH2 | 27.073 | −1.35 | 19.609 | O | WAT |
| | | 2672 | H1 | 27.764 | −1.722 | 20.175 | H | WAT |
| | | 2673 | H2 | 26.344 | −1.972 | 19.745 | H | WAT |
| 250 | HOH | 2674 | OH2 | 32.451 | −1.391 | 23.391 | O | WAT |
| | | 2675 | H1 | 32.999 | −0.579 | 23.34 | H | WAT |
| | | 2676 | H2 | 32.787 | −1.82 | 24.178 | H | WAT |
| 251 | HOH | 2677 | OH2 | 28.836 | 11.223 | −0.168 | O | WAT |
| | | 2678 | H1 | 29.749 | 11.399 | 0.144 | H | WAT |
| | | 2679 | H2 | 28.784 | 10.258 | −0.077 | H | WAT |
| 252 | HOH | 2680 | OH2 | 6.548 | −4.529 | 12.243 | O | WAT |
| | | 2681 | H1 | 7.344 | −4.524 | 12.789 | H | WAT |
| | | 2682 | H2 | 6.832 | −4.157 | 11.399 | H | WAT |
| 253 | HOH | 2683 | OH2 | 9.812 | −10.094 | 11.365 | O | WAT |
| | | 2684 | H1 | 9.795 | −10.83 | 11.996 | H | WAT |
| | | 2685 | H2 | 9.441 | −9.357 | 11.865 | H | WAT |
| 254 | HOH | 2686 | OH2 | 31.281 | 13.143 | 5.46 | O | WAT |
| | | 2687 | H1 | 30.831 | 12.291 | 5.515 | H | WAT |
| | | 2688 | H2 | 31.569 | 13.201 | 4.549 | H | WAT |
| 255 | HOH | 2689 | OH2 | 28.349 | −9.09 | 1.325 | O | WAT |
| | | 2690 | H1 | 28.931 | −9.84 | 1.149 | H | WAT |
| | | 2691 | H2 | 28.52 | −8.907 | 2.255 | H | WAT |
| 256 | HOH | 2692 | OH2 | 15.617 | 0.091 | 25.98 | O | WAT |
| | | 2693 | H1 | 16.409 | −0.135 | 25.47 | H | WAT |
| | | 2694 | H2 | 14.967 | 0.342 | 25.307 | H | WAT |
| 257 | HOH | 2695 | OH2 | 7.953 | 1.553 | −1.997 | O | WAT |
| | | 2696 | H1 | 8.498 | 2.346 | −1.933 | H | WAT |
| | | 2697 | H2 | 7.405 | 1.614 | −1.204 | H | WAT |
| 258 | HOH | 2698 | OH2 | 34.707 | −9.477 | 0.268 | O | WAT |
| | | 2699 | H1 | 34.401 | −9.52 | −0.654 | H | WAT |
| | | 2700 | H2 | 35.415 | −8.831 | 0.24 | H | WAT |
| 259 | HOH | 2701 | OH2 | 33.287 | 9.864 | −1.548 | O | WAT |
| | | 2702 | H1 | 33.015 | 9.728 | −0.618 | H | WAT |
| | | 2703 | H2 | 33.809 | 10.665 | −1.503 | H | WAT |
| 260 | HOH | 2704 | OH2 | 35.644 | −5.562 | 6.812 | O | WAT |
| | | 2705 | H1 | 34.734 | −5.771 | 6.548 | H | WAT |
| | | 2706 | H2 | 35.748 | −6.066 | 7.64 | H | WAT |
| 261 | HOH | 2707 | OH2 | 15.372 | 9.534 | 10.595 | O | WAT |
| | | 2708 | H1 | 14.829 | 9.569 | 11.396 | H | WAT |
| | | 2709 | H2 | 16.042 | 8.863 | 10.798 | H | WAT |
| 262 | HOH | 2710 | OH2 | 22.236 | 15.324 | 1.629 | O | WAT |
| | | 2711 | H1 | 21.279 | 15.436 | 1.813 | H | WAT |
| | | 2712 | H2 | 22.607 | 16.128 | 1.998 | H | WAT |
| 263 | HOH | 2713 | OH2 | 28.82 | −11.654 | 21.572 | O | WAT |
| | | 2714 | H1 | 27.911 | −11.866 | 21.861 | H | WAT |
| | | 2715 | H2 | 29.249 | −12.511 | 21.589 | H | WAT |
| 264 | HOH | 2716 | OH2 | 38.266 | −5.063 | 5.843 | O | WAT |
| | | 2717 | H1 | 37.385 | −5.392 | 6.068 | H | WAT |
| | | 2718 | H2 | 38.121 | −4.57 | 5.023 | H | WAT |
| 265 | HOH | 2719 | OH2 | 35.499 | −8.296 | 11.666 | O | WAT |
| | | 2720 | H1 | 35.621 | −7.639 | 12.374 | H | WAT |
| | | 2721 | H2 | 34.595 | −8.138 | 11.36 | H | WAT |
| 266 | HOH | 2722 | OH2 | 21.563 | 13.587 | −3.757 | O | WAT |
| | | 2723 | H1 | 21.164 | 12.913 | −3.189 | H | WAT |
| | | 2724 | H2 | 21.571 | 14.365 | −3.193 | H | WAT |
| 267 | HOH | 2725 | OH2 | 19.033 | −18.174 | 12.239 | O | WAT |
| | | 2726 | H1 | 19.187 | −17.452 | 12.865 | H | WAT |
| | | 2727 | H2 | 19.01 | −18.955 | 12.791 | H | WAT |
| 268 | HOH | 2728 | OH2 | 20.56 | 13.513 | 5.733 | O | WAT |
| | | 2729 | H1 | 20.766 | 14.072 | 6.485 | H | WAT |
| | | 2730 | H2 | 19.931 | 4.055 | 5.218 | H | WAT |
| 270 | HOH | 2731 | OH2 | 36.858 | 2.284 | 16.02 | O | WAT |
| | | 2732 | H1 | 36.698 | 2.196 | 15.068 | H | WAT |
| | | 2733 | H2 | 36.182 | 1.697 | 16.38 | H | WAT |
| 271 | HOH | 2734 | OH2 | 14.459 | 9.903 | 7.985 | O | WAT |
| | | 2735 | H1 | 14.832 | 9.776 | 8.872 | H | WAT |
| | | 2736 | H2 | 13.669 | 10.42 | 8.156 | H | WAT |
| 272 | HOH | 2737 | OH2 | 17.177 | 11.277 | 12.177 | O | WAT |
| | | 2738 | H1 | 16.553 | 10.979 | 11.506 | H | WAT |
| | | 2739 | H2 | 16.614 | 11.696 | 12.845 | H | WAT |
| 273 | HOH | 2740 | OH2 | 36.421 | −10.62 | 12.859 | O | WAT |
| | | 2741 | H1 | 37.356 | −10.514 | 13.049 | H | WAT |
| | | 2742 | H2 | 36.179 | −9.776 | 12.441 | H | WAT |
| 274 | HOH | 2743 | OH2 | 15.389 | −0.063 | −6.538 | O | WAT |
| | | 2744 | H1 | 15.352 | 0.13 | −5.587 | H | WAT |
| | | 2745 | H2 | 14.753 | 0.574 | −6.901 | H | WAT |
| 275 | HOH | 2746 | OH2 | 32.693 | −11.162 | 6.884 | O | WAT |
| | | 2747 | H1 | 33.22 | −11.963 | 6.83 | H | WAT |
| | | 2748 | H2 | 32.761 | −10.917 | 7.822 | H | WAT |
| 276 | HOH | 2749 | OH2 | 6.66 | 3.811 | 6.272 | O | WAT |
| | | 2750 | H1 | 6.297 | 4.598 | 5.858 | H | WAT |
| | | 2751 | H2 | 7.555 | 4.092 | 6.523 | H | WAT |
| 277 | HOH | 2752 | OH2 | 9.753 | −5.674 | 2.078 | O | WAT |
| | | 2753 | H1 | 10.212 | −4.881 | 2.404 | H | WAT |
| | | 2754 | H2 | 8.886 | −5.612 | 2.483 | H | WAT |
| 278 | HOH | 2755 | OH2 | 35.062 | −11.832 | 10.66 | O | WAT |
| | | 2756 | H1 | 35.538 | −11.484 | 11.428 | H | WAT |
| | | 2757 | H2 | 35.682 | −12.456 | 10.279 | H | WAT |
| 279 | HOH | 2758 | OH2 | 26.25 | −3.776 | 22.399 | O | WAT |
| | | 2759 | H1 | 25.652 | −4.223 | 22.998 | H | WAT |
| | | 2760 | H2 | 25.746 | −3.701 | 21.574 | H | WAT |
| 280 | HOH | 2761 | OH2 | 16.165 | −6.177 | −1.585 | O | WAT |
| | | 2762 | H1 | 16.809 | −6.851 | −1.318 | H | WAT |
| | | 2763 | H2 | 16.728 | −5.42 | −1.8 | H | WAT |
| 281 | HOH | 2764 | OH2 | 7.151 | −2.396 | 24.719 | O | WAT |
| | | 2765 | H1 | 6.4 | −2.041 | 25.195 | H | WAT |
| | | 2766 | H2 | 7.301 | −1.746 | 24.014 | H | WAT |
| 282 | HOH | 2767 | OH2 | 21.329 | −1.188 | 24.048 | O | WAT |
| | | 2768 | H1 | 21.433 | −0.649 | 24.834 | H | WAT |
| | | 2769 | H2 | 20.803 | −1.938 | 24.359 | H | WAT |
| 283 | HOH | 2770 | OH2 | 33.77 | −9.563 | −2.367 | O | WAT |
| | | 2771 | H1 | 33.913 | −8.965 | −3.102 | H | WAT |
| | | 2772 | H2 | 33.243 | −10.271 | −2.744 | H | WAT |
| 284 | HOH | 2773 | OH2 | 6.055 | 4.112 | 2.421 | O | WAT |
| | | 2774 | H1 | 6.961 | 4.288 | 2.717 | H | WAT |
| | | 2775 | H2 | 6.202 | 3.493 | 1.691 | H | WAT |
| 285 | HOH | 2776 | OH2 | 10.076 | −12.294 | 13.147 | O | WAT |
| | | 2777 | H1 | 11.004 | −12.564 | 13.046 | H | WAT |
| | | 2778 | H2 | 9.592 | −13.099 | 12.947 | H | WAT |
| 286 | HOH | 2779 | OH2 | 12.463 | −9.839 | 25.005 | O | WAT |
| | | 2780 | H1 | 11.913 | −9.085 | 24.741 | H | WAT |
| | | 2781 | H2 | 13.096 | −9.43 | 25.61 | H | WAT |
| 287 | HOH | 2782 | OH2 | 14.529 | 11.669 | 28.581 | O | WAT |
| | | 2783 | H1 | 14.6 | 11.801 | 27.611 | H | WAT |
| | | 2784 | H2 | 14 | 12.417 | 28.857 | H | WAT |
| 288 | HOH | 2785 | OH2 | 17.766 | 0.927 | −7.652 | O | WAT |
| | | 2786 | H1 | 17.445 | 1.772 | −8.007 | H | WAT |
| | | 2787 | H2 | 16.952 | 0.527 | −7.31 | H | WAT |
| 289 | HOH | 2788 | OH2 | 38.047 | −0.382 | 11.944 | O | WAT |
| | | 2789 | H1 | 37.937 | −1.248 | 11.516 | H | WAT |
| | | 2790 | H2 | 37.317 | −0.339 | 12.563 | H | WAT |
| 290 | HOH | 2791 | OH2 | 23.562 | 4.513 | 18.038 | O | WAT |
| | | 2792 | H1 | 23.447 | 5.465 | 17.84 | H | WAT |
| | | 2793 | H2 | 24.017 | 4.198 | 17.239 | H | WAT |
| 291 | HOH | 2794 | OH2 | 7.705 | −0.3 | 22.901 | O | WAT |
| | | 2795 | H1 | 8.319 | 0.167 | 23.489 | H | WAT |
| | | 2796 | H2 | 7.63 | 0.281 | 22.139 | H | WAT |
| 292 | HOH | 2797 | OH2 | 32.203 | 6.182 | 18.147 | O | WAT |
| | | 2798 | H1 | 33.054 | 5.866 | 17.812 | H | WAT |
| | | 2799 | H2 | 31.919 | 6.815 | 17.475 | H | WAT |
| 293 | HOH | 2800 | OH2 | 16.263 | 3.137 | −8.473 | O | WAT |
| | | 2801 | H1 | 16.163 | 4.056 | −8.21 | H | WAT |
| | | 2802 | H2 | 15.4 | 2.756 | −8.266 | H | WAT |
| 294 | HOH | 2803 | OH2 | 29.439 | −14.942 | 7.353 | O | WAT |
| | | 2804 | H1 | 30.134 | −15.535 | 7.063 | H | WAT |
| | | 2805 | H2 | 28.729 | −15.113 | 6.711 | H | WAT |
| 297 | HOH | 2806 | OH2 | 26.244 | −6.523 | −5.871 | O | WAT |
| | | 2807 | H1 | 26.439 | −6.549 | −4.924 | H | WAT |
| | | 2808 | H2 | 25.295 | −6.71 | −5.888 | H | WAT |
| 298 | HOH | 2809 | OH2 | 9.693 | 9.144 | 9.908 | O | WAT |
| | | 2810 | H1 | 9.66 | 8.362 | 9.343 | H | WAT |
| | | 2811 | H2 | 10.487 | 8.98 | 10.43 | H | WAT |
| 299 | HOH | 2812 | OH2 | 39.483 | 1.707 | 10.782 | O | WAT |
| | | 2813 | H1 | 39.665 | 1.28 | 9.924 | H | WAT |
| | | 2814 | H2 | 39.062 | 0.987 | 11.281 | H | WAT |
| 301 | HOH | 2815 | OH2 | 32.86 | −10.558 | 2.066 | O | WAT |
| | | 2816 | H1 | 33.529 | −10.215 | 1.449 | H | WAT |
| | | 2817 | H2 | 33.163 | −10.232 | 2.926 | H | WAT |
| 302 | HOH | 2818 | OH2 | 15.511 | 12.298 | 14.276 | O | WAT |
| | | 2819 | H1 | 15.105 | 11.873 | 15.048 | H | WAT |
| | | 2820 | H2 | 15.711 | 13.183 | 14.59 | H | WAT |
| 305 | HOH | 2821 | OH2 | 32.183 | 3.255 | 20.168 | O | WAT |
| | | 2822 | H1 | 31.512 | 3.889 | 19.879 | H | WAT |
| | | 2823 | H2 | 32.667 | 3.723 | 20.854 | H | WAT |
| 307 | HOH | 2824 | OH2 | 37.511 | 15.247 | 14.152 | O | WAT |
| | | 2825 | H1 | 38.39 | 15.307 | 13.751 | H | WAT |
| | | 2826 | H2 | 37.605 | 14.464 | 14.726 | H | WAT |
| 308 | HOH | 2827 | OH2 | 18.289 | −7.798 | −0.578 | O | WAT |
| | | 2828 | H1 | 18.269 | −8.752 | −0.389 | H | WAT |
| | | 2829 | H2 | 18.976 | −7.471 | 0.01 | H | WAT |
| 310 | HOH | 2830 | OH2 | 17.1 | −2.35 | −6.549 | O | WAT |
| | | 2831 | H1 | 16.425 | −1.655 | −6.588 | H | WAT |
| | | 2832 | H2 | 16.592 | −3.15 | −6.4 | H | WAT |
| 311 | HOH | 2833 | OH2 | 29.902 | −11.011 | 6.346 | O | WAT |
| | | 2834 | H1 | 30.86 | −11.067 | 6.483 | H | WAT |
| | | 2835 | H2 | 29.581 | −10.564 | 7.133 | H | WAT |
| 314 | HOH | 2836 | OH2 | 30.698 | −1.107 | 21.254 | O | WAT |
| | | 2837 | H1 | 31.295 | −1.249 | 22.01 | H | WAT |
| | | 2838 | H2 | 30.049 | −1.821 | 21.346 | H | WAT |
| 316 | HOH | 2839 | OH2 | 27.351 | −15.684 | 5.615 | O | WAT |
| | | 2840 | H1 | 26.592 | −15.936 | 6.143 | H | WAT |
| | | 2841 | H2 | 26.959 | −15.49 | 4.748 | H | WAT |
| 317 | HOH | 2842 | OH2 | 29.998 | 4.776 | 19.097 | O | WAT |
| | | 2843 | H1 | 30.709 | 5.289 | 18.674 | H | WAT |
| | | 2844 | H2 | 29.61 | 5.404 | 19.711 | H | WAT |
| 318 | HOH | 2845 | OH2 | 30.223 | −11.221 | 1.618 | O | WAT |
| | | 2846 | H1 | 31.167 | −10.999 | 1.725 | H | WAT |
| | | 2847 | H2 | 30.248 | −12.087 | 1.206 | H | WAT |
| 319 | HOH | 2848 | OH2 | 8.497 | −10.393 | 17.914 | O | WAT |
| | | 2849 | H1 | 7.946 | −9.763 | 17.443 | H | WAT |
| | | 2850 | H2 | 9.069 | −10.755 | 17.223 | H | WAT |
| 323 | HOH | 2851 | OH2 | 36.061 | 8.648 | 5.326 | O | WAT |
| | | 2852 | H1 | 35.382 | 8.092 | 4.915 | H | WAT |
| | | 2853 | H2 | 35.902 | 9.518 | 4.958 | H | WAT |
| 324 | HOH | 2854 | OH2 | 13.807 | 2.188 | −7.28 | O | WAT |
| | | 2855 | H1 | 12.877 | 2.163 | −7.518 | H | WAT |
| | | 2856 | H2 | 13.919 | 3.081 | −6.914 | H | WAT |
| 326 | HOH | 2857 | OH2 | 25.932 | −15.265 | 3.212 | O | WAT |
| | | 2858 | H1 | 25.286 | −14.623 | 2.868 | H | WAT |
| | | 2859 | H2 | 26.295 | −15.639 | 2.408 | H | WAT |
| 327 | HOH | 2860 | OH2 | 6.898 | 6.459 | 10.638 | O | WAT |
| | | 2861 | H1 | 6.36 | 5.99 | 11.28 | H | WAT |
| | | 2862 | H2 | 6.46 | 7.308 | 10.543 | H | WAT |
| 329 | HOH | 2863 | OH2 | 17.344 | 13.854 | 6.224 | O | WAT |
| | | 2864 | H1 | 17.01 | 14.599 | 6.727 | H | WAT |
| | | 2865 | H2 | 17.815 | 14.282 | 5.48 | H | WAT |
| 335 | HOH | 2866 | OH2 | 15.43 | −16.736 | 27.936 | O | WAT |
| | | 2867 | H1 | 16.224 | −17.157 | 28.264 | H | WAT |
| | | 2868 | H2 | 15.663 | −15.798 | 27.901 | H | WAT |
| 341 | HOH | 2869 | OH2 | 28.641 | 19.245 | 9.343 | O | WAT |
| | | 2870 | H1 | 29.495 | 18.953 | 8.988 | H | WAT |
| | | 2871 | H2 | 28.609 | 18.814 | 10.207 | H | WAT |
| 342 | HOH | 2872 | OH2 | 13.654 | −5.185 | −0.895 | O | WAT |
| | | 2873 | H1 | 13.462 | −4.66 | −1.674 | H | WAT |
| | | 2874 | H2 | 14.525 | −5.567 | −1.102 | H | WAT |
| 343 | HOH | 2875 | OH2 | 24.424 | −22.806 | 5.414 | O | WAT |
| | | 2876 | H1 | 24.677 | −23.037 | 6.312 | H | WAT |
| | | 2877 | H2 | 23.594 | −23.274 | 5.289 | H | WAT |
| 346 | HOH | 2878 | OH2 | 40.157 | 14.707 | 13.3 | O | WAT |
| | | 2879 | H1 | 40.036 | 13.911 | 13.858 | H | WAT |
| | | 2880 | H2 | 41.093 | 14.69 | 13.102 | H | WAT |
| 348 | HOH | 2881 | OH2 | 6.162 | −3.979 | 4.99 | O | WAT |
| | | 2882 | H1 | 6.205 | −3.432 | 4.196 | H | WAT |
| | | 2883 | H2 | 7.08 | −4.064 | 5.278 | H | WAT |
| 349 | HOH | 2884 | OH2 | 3.727 | 3.95 | 3.919 | O | WAT |
| | | 2885 | H1 | 3.067 | 4.605 | 3.685 | H | WAT |
| | | 2886 | H2 | 4.472 | 4.155 | 3.327 | H | WAT |
| 350 | HOH | 2887 | OH2 | 30.424 | −4.927 | −8.429 | O | WAT |
| | | 2888 | H1 | 30.993 | −5.526 | −8.915 | H | WAT |
| | | 2889 | H2 | 29.583 | −5.413 | −8.372 | H | WAT |
| 351 | HOH | 2890 | OH2 | 26.352 | 7.545 | −12.195 | O | WAT |
| | | 2891 | H1 | 26.295 | 8.393 | −11.755 | H | WAT |
| | | 2892 | H2 | 26.777 | 6.971 | −11.545 | H | WAT |
| 370 | HOH | 2893 | OH2 | 31.137 | 18.223 | 8.592 | O | WAT |
| | | 2894 | H1 | 31.987 | 18.533 | 8.28 | H | WAT |
| | | 2895 | H2 | 31.339 | 17.347 | 8.956 | H | WAT |
| 378 | HOH | 2896 | OH2 | 14.005 | −21.458 | 12.25 | O | WAT |
| | | 2897 | H1 | 13.732 | −21.521 | 11.336 | H | WAT |
| | | 2898 | H2 | 13.489 | −20.704 | 12.586 | H | WAT |
| 386 | HOH | 2899 | OH2 | 6.092 | −2.362 | 2.613 | O | WAT |
| | | 2900 | H1 | 5.99 | −1.405 | 2.483 | H | WAT |
| | | 2901 | H2 | 5.408 | −2.734 | 2.051 | H | WAT |
| 400 | HOH | 2902 | OH2 | 27.875 | −6.022 | −8.158 | O | WAT |
| | | 2903 | H1 | 27.283 | −6.258 | −7.427 | H | WAT |
| | | 2904 | H2 | 27.389 | −5.312 | −8.623 | H | WAT |
| 414 | HOH | 2905 | OH2 | 23.119 | 1.999 | 19.149 | O | WAT |
| | | 2906 | H1 | 23.978 | 1.965 | 19.573 | H | WAT |
| | | 2907 | H2 | 23.092 | 2.908 | 18.804 | H | WAT |
| 416 | HOH | 2908 | OH2 | 7.28 | 1.041 | 14.93 | O | WAT |
| | | 2909 | H1 | 7.503 | 1.347 | 15.832 | H | WAT |
| | | 2910 | H2 | 7.371 | 0.08 | 14.985 | H | WAT |
| 417 | HOH | 2911 | OH2 | 22.231 | 6.596 | 19.839 | O | WAT |
| | | 2912 | H1 | 22.499 | 5.676 | 19.798 | H | WAT |
| | | 2913 | H2 | 22.531 | 6.938 | 18.974 | H | WAT |
| 418 | HOH | 2914 | OH2 | 8.236 | 4.576 | 17.373 | O | WAT |
| | | 2915 | H1 | 8.09 | 3.607 | 17.378 | H | WAT |
| | | 2916 | H2 | 7.972 | 4.828 | 18.259 | H | WAT |
| 419 | HOH | 2917 | OH2 | 19.508 | 15.547 | 21.289 | O | WAT |
| | | 2918 | H1 | 19.997 | 15.68 | 20.469 | H | WAT |
| | | 2919 | H2 | 18.984 | 16.344 | 21.382 | H | WAT |

^{a}ResI: The residue ids in the structure
^{b}ResN: The residue names; the common amino acid residue with three letter representation; GLF representing Glyphosate; ACO representing Acetyl Co-enzyme A; and HOH representing water.
^{c}AtomI: The atom ids in structure.
^{d}AtomN: The atom name.
^{e}X, Y, Z: The atom coordinates of X, Y, and Z axes in angstroms.
^{f}ElemN: The corresponding element symbol for each atom.
^{g}SegN: The segment names in the complex, Pro representing peptide, LIG representing the bound ligands, and WAT representing surrounding waters.

TABLE 19
The atomic coordinates in Angstroms of the GLYATR11 variant bound to
glyphosate and acetyl coA, along with surrounding water molecules.

| ResI^{a} | ResN^{b} | AtomI^{c} | AtomN^{d} | X^{e} | Y | Z | ElemN^{f} | SegN^{g} |
|---|---|---|---|---|---|---|---|---|
| 2 | ILE | 1 | N | 19.62 | −2.278 | −6.619 | N | PRO |
| | | 2 | HT1 | 18.996 | −2.869 | −6.033 | H | PRO |
| | | 3 | HT2 | 20.042 | −2.853 | −7.375 | H | PRO |
| | | 4 | HT3 | 19.061 | −1.518 | −7.057 | H | PRO |
| | | 5 | CA | 20.712 | −1.723 | −5.771 | C | PRO |
| | | 6 | HA | 21.31 | −1.082 | −6.402 | H | PRO |
| | | 7 | CB | 20.182 | −0.939 | −4.563 | C | PRO |
| | | 8 | HB | 19.677 | −1.642 | −3.866 | H | PRO |
| | | 9 | CG2 | 21.379 | −0.312 | −3.808 | C | PRO |
| | | 10 | HG21 | 21.996 | 0.307 | −4.494 | H | PRO |
| | | 11 | HG22 | 22.028 | −1.095 | −3.362 | H | PRO |
| | | 12 | HG23 | 21.014 | 0.338 | −2.984 | H | PRO |
| | | 13 | CG1 | 19.118 | 0.129 | −4.943 | C | PRO |
| | | 14 | HG11 | 18.743 | 0.593 | −4.006 | H | PRO |
| | | 15 | HG12 | 18.244 | −0.369 | −5.415 | H | PRO |
| | | 16 | CD1 | 19.614 | 1.251 | −5.862 | C | PRO |
| | | 17 | HD1 | 19.974 | 0.846 | −6.832 | H | PRO |
| | | 18 | HD2 | 20.438 | 1.821 | −5.382 | H | PRO |
| | | 19 | HD3 | 18.786 | 1.961 | −6.074 | H | PRO |
| | | 20 | C | 21.552 | −2.884 | −5.314 | C | PRO |
| | | 21 | O | 21.053 | −3.81 | −4.677 | O | PRO |
| 3 | GLU | 22 | N | 22.856 | −2.877 | −5.668 | N | PRO |
| | | 23 | HN | 23.261 | −2.11 | −6.161 | H | PRO |
| | | 24 | CA | 23.798 | −3.916 | −5.319 | C | PRO |
| | | 25 | HA | 23.357 | −4.873 | −5.556 | H | PRO |
| | | 26 | CB | 25.122 | −3.742 | −6.092 | C | PRO |
| | | 27 | HB1 | 25.844 | −4.534 | −5.801 | H | PRO |
| | | 28 | HB2 | 25.553 | −2.758 | −5.808 | H | PRO |
| | | 29 | CG | 24.934 | −3.768 | −7.622 | C | PRO |
| | | 30 | HG1 | 24.121 | −3.074 | −7.923 | H | PRO |
| | | 31 | HG2 | 24.654 | −4.794 | −7.943 | H | PRO |
| | | 32 | CD | 26.192 | −3.338 | −8.369 | C | PRO |
| | | 33 | OE1 | 27.197 | −2.935 | −7.726 | O | PRO |
| | | 34 | OE2 | 26.163 | −3.388 | −9.632 | O | PRO |
| | | 35 | C | 24.111 | −3.871 | −3.846 | C | PRO |
| | | 36 | O | 24.141 | −2.793 | −3.259 | O | PRO |
| 4 | VAL | 37 | N | 24.365 | −5.041 | −3.221 | N | PRO |
| | | 38 | HN | 24.303 | −5.913 | −3.701 | H | PRO |
| | | 39 | CA | 24.784 | −5.119 | −1.838 | C | PRO |
| | | 40 | HA | 24.972 | −4.127 | −1.453 | H | PRO |
| | | 41 | CB | 23.8 | −5.823 | −0.916 | C | PRO |
| | | 42 | HB | 23.684 | −6.888 | −1.207 | H | PRO |
| | | 43 | CG1 | 24.315 | −5.747 | 0.537 | C | PRO |
| | | 44 | HG11 | 24.528 | −4.693 | 0.817 | H | PRO |
| | | 45 | HG12 | 25.24 | −6.348 | 0.671 | H | PRO |
| | | 46 | HG13 | 23.546 | −6.145 | 1.233 | H | PRO |
| | | 47 | CG2 | 22.427 | −5.141 | −1.038 | C | PRO |
| | | 48 | HG21 | 22.014 | −5.251 | −2.064 | H | PRO |
| | | 49 | HG22 | 22.508 | −4.059 | −0.8 | H | PRO |
| | | 50 | HG23 | 21.714 | −5.611 | −0.328 | H | PRO |
| | | 51 | C | 26.078 | −5.878 | −1.847 | C | PRO |
| | | 52 | O | 26.141 | −7.02 | −2.304 | O | PRO |
| 5 | LYS | 53 | N | 27.153 | −5.234 | −1.349 | N | PRO |
| | | 54 | HN | 27.075 | −4.315 | −0.97 | H | PRO |
| | | 55 | CA | 28.488 | −5.774 | −1.386 | C | PRO |
| | | 56 | HA | 28.444 | −6.846 | −1.516 | H | PRO |
| | | 57 | CB | 29.333 | −5.147 | −2.524 | C | PRO |
| | | 58 | HB1 | 30.41 | −5.378 | −2.376 | H | PRO |
| | | 59 | HB2 | 29.212 | −4.043 | −2.49 | H | PRO |
| | | 60 | CG | 28.929 | −5.655 | −3.921 | C | PRO |
| | | 61 | HG1 | 27.821 | −5.663 | −4.005 | H | PRO |
| | | 62 | HG2 | 29.281 | −6.703 | −4.028 | H | PRO |
| | | 63 | CD | 29.474 | −4.799 | −5.077 | C | PRO |
| | | 64 | HD1 | 30.572 | −4.679 | −4.96 | H | PRO |
| | | 65 | HD2 | 29.008 | −3.793 | −5.003 | H | PRO |
| | | 66 | CE | 29.171 | −5.415 | −6.451 | C | PRO |
| | | 67 | HE1 | 28.101 | −5.704 | −6.518 | H | PRO |
| | | 68 | HE2 | 29.803 | −6.314 | −6.619 | H | PRO |
| | | 69 | NZ | 29.437 | −4.451 | −7.542 | N | PRO |
| | | 70 | HZ1 | 30.369 | −4.004 | −7.425 | H | PRO |
| | | 71 | HZ2 | 28.694 | −3.724 | −7.526 | H | PRO |
| | | 72 | HZ3 | 29.383 | −4.927 | −8.465 | H | PRO |
| | | 73 | C | 29.105 | −5.415 | −0.052 | C | PRO |
| | | 74 | O | 28.886 | −4.291 | 0.402 | O | PRO |
| 6 | PRO | 75 | N | 29.844 | −6.292 | 0.639 | N | PRO |
| | | 76 | CD | 29.943 | −7.717 | 0.318 | C | PRO |
| | | 77 | HD1 | 30.568 | −7.834 | −0.593 | H | PRO |
| | | 78 | HD2 | 28.938 | −8.163 | 0.16 | H | PRO |
| | | 79 | CA | 30.699 | −5.95 | 1.772 | C | PRO |
| | | 80 | HA | 30.038 | −5.713 | 2.592 | H | PRO |
| | | 81 | CB | 31.531 | −7.214 | 2.027 | C | PRO |
| | | 82 | HB1 | 31.8 | −7.326 | 3.099 | H | PRO |
| | | 83 | HB2 | 32.458 | −7.206 | 1.415 | H | PRO |
| | | 84 | CG | 30.63 | −8.345 | 1.529 | C | PRO |
| | | 85 | HG1 | 31.2 | −9.263 | 1.267 | H | PRO |
| | | 86 | HG2 | 29.868 | −8.586 | 2.301 | H | PRO |
| | | 87 | C | 31.583 | −4.745 | 1.557 | C | PRO |
| | | 88 | O | 32.11 | −4.58 | 0.457 | O | PRO |
| 7 | ILE | 89 | N | 31.765 | −3.911 | 2.599 | N | PRO |
| | | 90 | HN | 31.332 | −4.08 | 3.481 | H | PRO |
| | | 91 | CA | 32.655 | −2.775 | 2.547 | C | PRO |
| | | 92 | HA | 33.367 | −2.913 | 1.747 | H | PRO |
| | | 93 | CB | 31.983 | −1.412 | 2.378 | C | PRO |
| | | 94 | HB | 32.769 | −0.627 | 2.374 | H | PRO |
| | | 95 | CG2 | 31.317 | −1.372 | 0.987 | C | PRO |
| | | 96 | HG21 | 30.464 | −2.083 | 0.941 | H | PRO |
| | | 97 | HG22 | 32.052 | −1.651 | 0.202 | H | PRO |
| | | 98 | HG23 | 30.94 | −0.352 | 0.762 | H | PRO |
| | | 99 | CG1 | 30.997 | −1.082 | 3.524 | C | PRO |
| | | 100 | HG11 | 31.539 | −1.123 | 4.493 | H | PRO |
| | | 101 | HG12 | 30.191 | −1.847 | 3.55 | H | PRO |
| | | 102 | CD1 | 30.37 | 0.308 | 3.406 | C | PRO |
| | | 103 | HD1 | 29.696 | 0.37 | 2.526 | H | PRO |
| | | 104 | HD2 | 31.154 | 1.089 | 3.309 | H | PRO |
| | | 105 | HD3 | 29.777 | 0.529 | 4.319 | H | PRO |
| | | 106 | C | 33.432 | −2.807 | 3.829 | C | PRO |
| | | 107 | O | 33.123 | −3.577 | 4.739 | O | PRO |
| 8 | ASN | 108 | N | 34.496 | −1.986 | 3.915 | N | PRO |
| | | 109 | HN | 34.729 | −1.355 | 3.179 | H | PRO |
| | | 110 | CA | 35.337 | −1.902 | 5.082 | C | PRO |
| | | 111 | HA | 35.216 | −2.792 | 5.682 | H | PRO |
| | | 112 | CB | 36.831 | −1.741 | 4.72 | C | PRO |
| | | 113 | HB1 | 37.458 | −1.762 | 5.638 | H | PRO |
| | | 114 | HB2 | 36.995 | −0.776 | 4.195 | H | PRO |
| | | 115 | CG | 37.251 | −2.912 | 3.826 | C | PRO |
| | | 116 | OD1 | 37.015 | −4.081 | 4.155 | O | PRO |
| | | 117 | ND2 | 37.878 | −2.593 | 2.661 | N | PRO |
| | | 118 | HD21 | 38.015 | −1.634 | 2.411 | H | PRO |
| | | 119 | HD22 | 38.169 | −3.328 | 2.049 | H | PRO |
| | | 120 | C | 34.874 | −0.728 | 5.905 | C | PRO |
| | | 121 | O | 34.068 | 0.085 | 5.455 | O | PRO |
| 9 | ALA | 122 | N | 35.367 | −0.623 | 7.161 | N | PRO |
| | | 123 | HN | 36.013 | −1.299 | 7.508 | H | PRO |
| | | 124 | CA | 35.001 | 0.411 | 8.108 | C | PRO |
| | | 125 | HA | 33.93 | 0.362 | 8.235 | H | PRO |
| | | 126 | CB | 35.661 | 0.18 | 9.479 | C | PRO |
| | | 127 | HB1 | 36.767 | 0.245 | 9.403 | H | PRO |
| | | 128 | HB2 | 35.397 | −0.83 | 9.859 | H | PRO |
| | | 129 | HB3 | 35.309 | 0.93 | 10.219 | H | PRO |
| | | 130 | C | 35.346 | 1.801 | 7.617 | C | PRO |
| | | 131 | O | 34.615 | 2.754 | 7.872 | O | PRO |
| 10 | GLU | 132 | N | 36.449 | 1.932 | 6.845 | N | PRO |
| | | 133 | HN | 37.033 | 1.143 | 6.674 | H | PRO |
| | | 134 | CA | 36.931 | 3.151 | 6.228 | C | PRO |
| | | 135 | HA | 37.18 | 3.858 | 7.006 | H | PRO |
| | | 136 | CB | 38.186 | 2.872 | 5.359 | C | PRO |
| | | 137 | HB1 | 38.422 | 3.782 | 4.766 | H | PRO |
| | | 138 | HB2 | 37.963 | 2.056 | 4.638 | H | PRO |
| | | 139 | CG | 39.485 | 2.543 | 6.14 | C | PRO |
| | | 140 | HG1 | 39.735 | 3.402 | 6.798 | H | PRO |
| | | 141 | HG2 | 40.311 | 2.417 | 5.408 | H | PRO |
| | | 142 | CD | 39.427 | 1.276 | 6.99 | C | PRO |
| | | 143 | OE1 | 38.811 | 0.27 | 6.547 | O | PRO |
| | | 144 | OE2 | 40.008 | 1.291 | 8.109 | O | PRO |
| | | 145 | C | 35.891 | 3.789 | 5.332 | C | PRO |
| | | 146 | O | 35.734 | 5.009 | 5.309 | O | PRO |
| 11 | ASP | 147 | N | 35.132 | 2.955 | 4.589 | N | PRO |
| | | 148 | HN | 35.254 | 1.967 | 4.656 | H | PRO |
| | | 149 | CA | 34.138 | 3.385 | 3.633 | C | PRO |
| | | 150 | HA | 34.553 | 4.182 | 3.034 | H | PRO |
| | | 151 | CB | 33.734 | 2.199 | 2.726 | C | PRO |
| | | 152 | HB1 | 32.974 | 2.517 | 1.981 | H | PRO |
| | | 153 | HB2 | 33.31 | 1.381 | 3.347 | H | PRO |
| | | 154 | CG | 34.942 | 1.661 | 1.974 | C | PRO |
| | | 155 | OD1 | 35.483 | 2.403 | 1.111 | O | PRO |
| | | 156 | OD2 | 35.341 | 0.496 | 2.243 | O | PRO |
| | | 157 | C | 32.877 | 3.903 | 4.298 | C | PRO |
| | | 158 | O | 32.101 | 4.638 | 3.689 | O | PRO |
| 12 | THR | 159 | N | 32.645 | 3.543 | 5.583 | N | PRO |
| | | 160 | HN | 33.315 | 2.997 | 6.08 | H | PRO |
| | | 161 | CA | 31.402 | 3.838 | 6.276 | C | PRO |
| | | 162 | HA | 30.594 | 3.659 | 5.582 | H | PRO |
| | | 163 | CB | 31.164 | 2.944 | 7.498 | C | PRO |
| | | 164 | HB | 30.142 | 3.137 | 7.886 | H | PRO |
| | | 165 | OG1 | 32.069 | 3.187 | 8.569 | O | PRO |
| | | 166 | HG1 | 32.953 | 2.983 | 8.253 | H | PRO |
| | | 167 | CG2 | 31.253 | 1.457 | 7.111 | C | PRO |
| | | 168 | HG21 | 32.278 | 1.19 | 6.773 | H | PRO |
| | | 169 | HG22 | 30.543 | 1.223 | 6.29 | H | PRO |
| | | 170 | HG23 | 31.003 | 0.82 | 7.985 | H | PRO |
| | | 171 | C | 31.304 | 5.284 | 6.715 | C | PRO |
| | | 172 | O | 30.213 | 5.79 | 6.972 | O | PRO |
| 13 | TYR | 173 | N | 32.455 | 5.99 | 6.807 | N | PRO |
| | | 174 | HN | 33.323 | 5.579 | 6.537 | H | PRO |
| | | 175 | CA | 32.554 | 7.266 | 7.482 | C | PRO |
| | | 176 | HA | 31.971 | 7.202 | 8.389 | H | PRO |
| | | 177 | CB | 34.021 | 7.608 | 7.871 | C | PRO |
| | | 178 | HB1 | 34.054 | 8.546 | 8.465 | H | PRO |
| | | 179 | HB2 | 34.629 | 7.741 | 6.951 | H | PRO |
| | | 180 | CG | 34.665 | 6.524 | 8.718 | C | PRO |
| | | 181 | CD1 | 33.966 | 5.879 | 9.756 | C | PRO |
| | | 182 | HD1 | 32.946 | 6.149 | 9.985 | H | PRO |
| | | 183 | CE1 | 34.568 | 4.862 | 10.504 | C | PRO |
| | | 184 | HE1 | 34.007 | 4.353 | 11.275 | H | PRO |
| | | 185 | CZ | 35.89 | 4.491 | 10.251 | C | PRO |
| | | 186 | OH | 36.465 | 3.459 | 11.021 | O | PRO |
| | | 187 | HH | 37.303 | 3.203 | 10.627 | H | PRO |
| | | 188 | CD2 | 36.004 | 6.155 | 8.489 | C | PRO |
| | | 189 | HD2 | 36.568 | 6.643 | 7.707 | H | PRO |
| | | 190 | CE2 | 36.618 | 5.152 | 9.255 | C | PRO |
| | | 191 | HE2 | 37.645 | 4.88 | 9.062 | H | PRO |
| | | 192 | C | 31.96 | 8.407 | 6.681 | C | PRO |
| | | 193 | O | 31.649 | 9.453 | 7.249 | O | PRO |
| 14 | ASP | 194 | N | 31.738 | 8.209 | 5.354 | N | PRO |
| | | 195 | HN | 32.003 | 7.349 | 4.926 | H | PRO |
| | | 196 | CA | 31.115 | 9.178 | 4.47 | C | PRO |
| | | 197 | HA | 31.694 | 10.088 | 4.535 | H | PRO |
| | | 198 | CB | 31.138 | 8.659 | 2.996 | C | PRO |
| | | 199 | HB1 | 30.588 | 7.695 | 2.934 | H | PRO |
| | | 200 | HB2 | 32.193 | 8.463 | 2.709 | H | PRO |
| | | 201 | CG | 30.549 | 9.619 | 1.957 | C | PRO |
| | | 202 | OD1 | 29.71 | 9.15 | 1.139 | O | PRO |
| | | 203 | OD2 | 30.941 | 10.818 | 1.934 | O | PRO |
| | | 204 | C | 29.695 | 9.501 | 4.909 | C | PRO |
| | | 205 | O | 29.338 | 10.673 | 5.017 | O | PRO |
| 15 | LEU | 206 | N | 28.872 | 8.474 | 5.229 | N | PRO |
| | | 207 | HN | 29.16 | 7.525 | 5.129 | H | PRO |
| | | 208 | CA | 27.496 | 8.698 | 5.62 | C | PRO |
| | | 209 | HA | 27.156 | 9.652 | 5.245 | H | PRO |
| | | 210 | CB | 26.569 | 7.584 | 5.078 | C | PRO |
| | | 211 | HB1 | 25.57 | 7.647 | 5.56 | H | PRO |
| | | 212 | HB2 | 27.014 | 6.599 | 5.336 | H | PRO |
| | | 213 | CG | 26.349 | 7.643 | 3.55 | C | PRO |
| | | 214 | HG | 27.347 | 7.683 | 3.063 | H | PRO |
| | | 215 | CD1 | 25.639 | 6.379 | 3.036 | C | PRO |
| | | 216 | HD11 | 24.644 | 6.27 | 3.518 | H | PRO |
| | | 217 | HD12 | 26.248 | 5.477 | 3.261 | H | PRO |
| | | 218 | HD13 | 25.498 | 6.437 | 1.935 | H | PRO |
| | | 219 | CD2 | 25.568 | 8.898 | 3.128 | C | PRO |
| | | 220 | HD21 | 26.127 | 9.826 | 3.374 | H | PRO |
| | | 221 | HD22 | 24.578 | 8.927 | 3.631 | H | PRO |
| | | 222 | HD23 | 25.404 | 8.873 | 2.029 | H | PRO |
| | | 223 | C | 27.34 | 8.74 | 7.119 | C | PRO |
| | | 224 | O | 26.386 | 9.339 | 7.614 | O | PRO |
| 16 | ARG | 225 | N | 28.289 | 8.16 | 7.894 | N | PRO |
| | | 226 | HN | 29.045 | 7.649 | 7.493 | H | PRO |
| | | 227 | CA | 28.258 | 8.249 | 9.342 | C | PRO |
| | | 228 | HA | 27.277 | 7.941 | 9.673 | H | PRO |
| | | 229 | CB | 29.3 | 7.357 | 10.046 | C | PRO |
| | | 230 | HB1 | 29.384 | 7.651 | 11.114 | H | PRO |
| | | 231 | HB2 | 30.293 | 7.52 | 9.576 | H | PRO |
| | | 232 | CG | 28.961 | 5.857 | 10.035 | C | PRO |
| | | 233 | HG1 | 28.85 | 5.5 | 8.988 | H | PRO |
| | | 234 | HG2 | 27.992 | 5.699 | 10.554 | H | PRO |
| | | 235 | CD | 30.059 | 5.058 | 10.744 | C | PRO |
| | | 236 | HD1 | 30.163 | 5.422 | 11.788 | H | PRO |
| | | 237 | HD2 | 31.024 | 5.177 | 10.207 | H | PRO |
| | | 238 | NE | 29.714 | 3.604 | 10.77 | N | PRO |
| | | 239 | HE | 28.967 | 3.26 | 10.202 | H | PRO |
| | | 240 | CZ | 30.5 | 2.707 | 11.419 | C | PRO |
| | | 241 | NH1 | 31.559 | 3.099 | 12.162 | N | PRO |
| | | 242 | HH11 | 31.761 | 4.07 | 12.295 | H | PRO |
| | | 243 | HH12 | 32.11 | 2.412 | 12.634 | H | PRO |
| | | 244 | NH2 | 30.228 | 1.387 | 11.356 | N | PRO |
| | | 245 | HH21 | 29.473 | 1.036 | 10.802 | H | PRO |
| | | 246 | HH22 | 30.774 | 0.772 | 11.926 | H | PRO |
| | | 247 | C | 28.452 | 9.663 | 9.828 | C | PRO |
| | | 248 | O | 27.763 | 10.102 | 10.744 | O | PRO |
| 17 | HIS | 249 | N | 29.389 | 10.427 | 9.222 | N | PRO |
| | | 250 | HN | 29.976 | 10.075 | 8.496 | H | PRO |
| | | 251 | CA | 29.56 | 11.811 | 9.599 | C | PRO |
| | | 252 | HA | 29.515 | 11.868 | 10.676 | H | PRO |
| | | 253 | CB | 30.922 | 12.388 | 9.152 | C | PRO |
| | | 254 | HB1 | 30.928 | 12.59 | 8.059 | H | PRO |
| | | 255 | HB2 | 31.713 | 11.633 | 9.347 | H | PRO |
| | | 256 | ND1 | 31.528 | 13.656 | 11.275 | N | PRO |
| | | 257 | HD1 | 31.508 | 12.87 | 11.893 | H | PRO |
| | | 258 | CG | 31.286 | 13.632 | 9.916 | C | PRO |
| | | 259 | CE1 | 31.756 | 14.949 | 11.613 | C | PRO |
| | | 260 | HE1 | 31.969 | 15.266 | 12.634 | H | PRO |
| | | 261 | NE2 | 31.677 | 15.755 | 10.571 | N | PRO |
| | | 262 | CD2 | 31.38 | 14.926 | 9.504 | C | PRO |
| | | 263 | HD2 | 31.243 | 15.344 | 8.514 | H | PRO |
| | | 264 | C | 28.459 | 12.685 | 9.05 | C | PRO |
| | | 265 | O | 27.925 | 13.533 | 9.755 | O | PRO |
| 18 | ARG | 266 | N | 28.096 | 12.5 | 7.762 | N | PRO |
| | | 267 | HN | 28.496 | 11.773 | 7.21 | H | PRO |
| | | 268 | CA | 27.237 | 13.429 | 7.064 | C | PRO |
| | | 269 | HA | 27.615 | 14.423 | 7.253 | H | PRO |
| | | 270 | CB | 27.325 | 13.174 | 5.543 | C | PRO |
| | | 271 | HB1 | 26.944 | 12.156 | 5.316 | H | PRO |
| | | 272 | HB2 | 28.399 | 13.195 | 5.261 | H | PRO |
| | | 273 | CG | 26.59 | 14.195 | 4.657 | C | PRO |
| | | 274 | HG1 | 26.93 | 15.224 | 4.904 | H | PRO |
| | | 275 | HG2 | 25.502 | 14.141 | 4.873 | H | PRO |
| | | 276 | CD | 26.841 | 13.923 | 3.17 | C | PRO |
| | | 277 | HD1 | 26.727 | 12.839 | 2.958 | H | PRO |
| | | 278 | HD2 | 27.862 | 14.257 | 2.883 | H | PRO |
| | | 279 | NE | 25.823 | 14.668 | 2.365 | N | PRO |
| | | 280 | HE | 25.105 | 15.177 | 2.84 | H | PRO |
| | | 281 | CZ | 25.736 | 14.544 | 1.016 | C | PRO |
| | | 282 | NH1 | 26.746 | 14.006 | 0.296 | N | PRO |
| | | 283 | HH11 | 27.587 | 13.708 | 0.747 | H | PRO |
| | | 284 | HH12 | 26.65 | 13.899 | −0.694 | H | PRO |
| | | 285 | NH2 | 24.61 | 14.938 | 0.378 | N | PRO |
| | | 286 | HH21 | 23.819 | 15.233 | 0.914 | H | PRO |
| | | 287 | HH22 | 24.501 | 14.71 | −0.589 | H | PRO |
| | | 288 | C | 25.785 | 13.415 | 7.497 | C | PRO |
| | | 289 | O | 25.214 | 14.478 | 7.731 | O | PRO |
| 19 | VAL | 290 | N | 25.148 | 12.222 | 7.613 | N | PRO |
| | | 291 | HN | 25.622 | 11.356 | 7.477 | H | PRO |
| | | 292 | CA | 23.702 | 12.152 | 7.777 | C | PRO |
| | | 293 | HA | 23.298 | 13.147 | 7.893 | H | PRO |
| | | 294 | CB | 22.987 | 11.531 | 6.573 | C | PRO |
| | | 295 | HB | 21.893 | 11.514 | 6.77 | H | PRO |
| | | 296 | CG1 | 23.217 | 12.414 | 5.33 | C | PRO |
| | | 297 | HG11 | 24.283 | 12.372 | 5.02 | H | PRO |
| | | 298 | HG12 | 22.945 | 13.47 | 5.544 | H | PRO |
| | | 299 | HG13 | 22.6 | 12.055 | 4.48 | H | PRO |
| | | 300 | CG2 | 23.46 | 10.09 | 6.307 | C | PRO |
| | | 301 | HG21 | 23.296 | 9.443 | 7.195 | H | PRO |
| | | 302 | HG22 | 24.539 | 10.074 | 6.043 | H | PRO |
| | | 303 | HG23 | 22.893 | 9.661 | 5.454 | H | PRO |
| | | 304 | C | 23.311 | 11.413 | 9.036 | C | PRO |
| | | 305 | O | 22.128 | 11.152 | 9.251 | O | PRO |
| 20 | LEU | 306 | N | 24.281 | 11.077 | 9.917 | N | PRO |
| | | 307 | HN | 25.236 | 11.295 | 9.733 | H | PRO |
| | | 308 | CA | 23.996 | 10.383 | 11.158 | C | PRO |
| | | 309 | HA | 22.929 | 10.32 | 11.308 | H | PRO |
| | | 310 | CB | 24.585 | 8.952 | 11.213 | C | PRO |
| | | 311 | HB1 | 24.516 | 8.556 | 12.248 | H | PRO |
| | | 312 | HB2 | 25.659 | 8.985 | 10.93 | H | PRO |
| | | 313 | CG | 23.866 | 7.963 | 10.269 | C | PRO |
| | | 314 | HG | 23.846 | 8.416 | 9.254 | H | PRO |
| | | 315 | CD1 | 24.625 | 6.631 | 10.144 | C | PRO |
| | | 316 | HD11 | 24.023 | 5.902 | 9.561 | H | PRO |
| | | 317 | HD12 | 24.837 | 6.202 | 11.146 | H | PRO |
| | | 318 | HD13 | 25.588 | 6.779 | 9.611 | H | PRO |
| | | 319 | CD2 | 22.408 | 7.721 | 10.691 | C | PRO |
| | | 320 | HD21 | 21.815 | 8.659 | 10.633 | H | PRO |
| | | 321 | HD22 | 22.342 | 7.336 | 11.732 | H | PRO |
| | | 322 | HD23 | 21.94 | 6.978 | 10.01 | H | PRO |
| | | 323 | C | 24.497 | 11.212 | 12.304 | C | PRO |
| | | 324 | O | 23.691 | 11.797 | 13.026 | O | PRO |
| 21 | ARG | 325 | N | 25.832 | 11.284 | 12.511 | N | PRO |
| | | 326 | HN | 26.476 | 10.819 | 11.909 | H | PRO |
| | | 327 | CA | 26.425 | 12.011 | 13.614 | C | PRO |
| | | 328 | HA | 25.656 | 12.386 | 14.274 | H | PRO |
| | | 329 | CB | 27.349 | 11.085 | 14.446 | C | PRO |
| | | 330 | HB1 | 27.818 | 11.674 | 15.264 | H | PRO |
| | | 331 | HB2 | 28.157 | 10.699 | 13.789 | H | PRO |
| | | 332 | CG | 26.659 | 9.868 | 15.091 | C | PRO |
| | | 333 | HG1 | 26.231 | 9.222 | 14.295 | H | PRO |
| | | 334 | HG2 | 25.822 | 10.226 | 15.728 | H | PRO |
| | | 335 | CD | 27.643 | 9.043 | 15.941 | C | PRO |
| | | 336 | HD1 | 28.076 | 9.646 | 16.767 | H | PRO |
| | | 337 | HD2 | 28.479 | 8.687 | 15.301 | H | PRO |
| | | 338 | NE | 26.951 | 7.838 | 16.508 | N | PRO |
| | | 339 | HE | 27.102 | 6.943 | 16.088 | H | PRO |
| | | 340 | CZ | 26.11 | 7.854 | 17.576 | C | PRO |
| | | 341 | NH1 | 25.841 | 8.973 | 18.282 | N | PRO |
| | | 342 | HH11 | 26.319 | 9.826 | 18.074 | H | PRO |
| | | 343 | HH12 | 25.204 | 8.923 | 19.051 | H | PRO |
| | | 344 | NH2 | 25.52 | 6.69 | 17.933 | N | PRO |
| | | 345 | HH21 | 25.661 | 5.898 | 17.339 | H | PRO |
| | | 346 | HH22 | 24.78 | 6.683 | 18.606 | H | PRO |
| | | 347 | C | 27.249 | 13.205 | 13.131 | C | PRO |
| | | 348 | O | 28.476 | 13.144 | 13.226 | O | PRO |
| 22 | PRO | 349 | N | 26.688 | 14.319 | 12.636 | N | PRO |
| | | 350 | CD | 25.268 | 14.478 | 12.311 | C | PRO |
| | | 351 | HD1 | 24.63 | 14.284 | 13.2 | H | PRO |
| | | 352 | HD2 | 25.015 | 13.781 | 11.483 | H | PRO |
| | | 353 | CA | 27.469 | 15.471 | 12.193 | C | PRO |
| | | 354 | HA | 28.381 | 15.149 | 11.714 | H | PRO |
| | | 355 | CB | 26.51 | 16.217 | 11.251 | C | PRO |
| | | 356 | HB1 | 26.586 | 15.769 | 10.237 | H | PRO |
| | | 357 | HB2 | 26.719 | 17.305 | 11.175 | H | PRO |
| | | 358 | CG | 25.119 | 15.918 | 11.816 | C | PRO |
| | | 359 | HG1 | 24.907 | 16.59 | 12.675 | H | PRO |
| | | 360 | HG2 | 24.318 | 16.023 | 11.053 | H | PRO |
| | | 361 | C | 27.819 | 16.35 | 13.368 | C | PRO |
| | | 362 | O | 28.587 | 17.294 | 13.198 | O | PRO |
| 23 | ASN | 363 | N | 27.243 | 16.071 | 14.556 | N | PRO |
| | | 364 | HN | 26.623 | 15.294 | 14.63 | H | PRO |
| | | 365 | CA | 27.365 | 16.895 | 15.738 | C | PRO |
| | | 366 | HA | 27.654 | 17.897 | 15.456 | H | PRO |
| | | 367 | CB | 26.036 | 16.923 | 16.535 | C | PRO |
| | | 368 | HB1 | 26.122 | 17.581 | 17.426 | H | PRO |
| | | 369 | HB2 | 25.777 | 15.898 | 16.874 | H | PRO |
| | | 370 | CG | 24.893 | 17.422 | 15.649 | C | PRO |
| | | 371 | OD1 | 23.995 | 16.653 | 15.293 | O | PRO |
| | | 372 | ND2 | 24.93 | 18.733 | 15.285 | N | PRO |
| | | 373 | HD21 | 24.201 | 19.092 | 14.702 | H | PRO |
| | | 374 | HD22 | 25.68 | 19.319 | 15.594 | H | PRO |
| | | 375 | C | 28.435 | 16.337 | 16.644 | C | PRO |
| | | 376 | O | 28.567 | 16.758 | 17.792 | O | PRO |
| 24 | GLN | 377 | N | 29.23 | 15.37 | 16.137 | N | PRO |
| | | 378 | HN | 29.134 | 15.077 | 15.189 | H | PRO |
| | | 379 | CA | 30.294 | 14.733 | 16.867 | C | PRO |
| | | 380 | HA | 30.561 | 15.354 | 17.71 | H | PRO |
| | | 381 | CB | 29.898 | 13.308 | 17.334 | C | PRO |
| | | 382 | HB1 | 30.782 | 12.805 | 17.781 | H | PRO |
| | | 383 | HB2 | 29.582 | 12.723 | 16.444 | H | PRO |
| | | 384 | CG | 28.747 | 13.286 | 18.358 | C | PRO |
| | | 385 | HG1 | 27.866 | 13.817 | 17.939 | H | PRO |
| | | 386 | HG2 | 29.051 | 13.795 | 19.297 | H | PRO |
| | | 387 | CD | 28.321 | 11.848 | 18.663 | C | PRO |
| | | 388 | OE1 | 27.217 | 11.429 | 18.3 | O | PRO |
| | | 389 | NE2 | 29.216 | 11.078 | 19.343 | N | PRO |
| | | 390 | HE21 | 28.978 | 10.132 | 19.563 | H | PRO |
| | | 391 | HE22 | 30.098 | 11.46 | 19.619 | H | PRO |
| | | 392 | C | 31.454 | 14.643 | 15.892 | C | PRO |
| | | 393 | O | 31.206 | 14.688 | 14.687 | O | PRO |
| 25 | PRO | 394 | N | 32.713 | 14.534 | 16.333 | N | PRO |
| | | 395 | CD | 33.094 | 14.714 | 17.739 | C | PRO |
| | | 396 | HD1 | 32.877 | 13.774 | 18.29 | H | PRO |
| | | 397 | HD2 | 32.564 | 15.571 | 18.207 | H | PRO |
| | | 398 | CA | 33.896 | 14.456 | 15.474 | C | PRO |
| | | 399 | HA | 33.964 | 15.401 | 14.955 | H | PRO |
| | | 400 | CB | 35.048 | 14.238 | 16.464 | C | PRO |
| | | 401 | HB1 | 36.009 | 14.635 | 16.075 | H | PRO |
| | | 402 | HB2 | 35.163 | 13.16 | 16.711 | H | PRO |
| | | 403 | CG | 34.597 | 14.984 | 17.717 | C | PRO |
| | | 404 | HG1 | 35.114 | 14.631 | 18.635 | H | PRO |
| | | 405 | HG2 | 34.777 | 16.072 | 17.582 | H | PRO |
| | | 406 | C | 33.901 | 13.365 | 14.422 | C | PRO |
| | | 407 | O | 33.107 | 12.43 | 14.498 | O | PRO |
| 26 | ILE | 408 | N | 34.824 | 13.449 | 13.436 | N | PRO |
| | | 409 | HN | 35.45 | 14.224 | 13.393 | H | PRO |
| | | 410 | CA | 35.048 | 12.421 | 12.432 | C | PRO |
| | | 411 | HA | 34.075 | 12.178 | 12.03 | H | PRO |
| | | 412 | CB | 35.917 | 12.923 | 11.275 | C | PRO |
| | | 413 | HB | 35.418 | 13.828 | 10.867 | H | PRO |
| | | 414 | CG2 | 37.311 | 13.365 | 11.774 | C | PRO |
| | | 415 | HG21 | 37.889 | 12.495 | 12.153 | H | PRO |
| | | 416 | HG22 | 37.236 | 14.121 | 12.584 | H | PRO |
| | | 417 | HG23 | 37.887 | 13.817 | 10.938 | H | PRO |
| | | 418 | CG1 | 36.038 | 11.914 | 10.103 | C | PRO |
| | | 419 | HG11 | 36.527 | 10.983 | 10.461 | H | PRO |
| | | 420 | HG12 | 36.708 | 12.36 | 9.337 | H | PRO |
| | | 421 | CD1 | 34.707 | 11.564 | 9.428 | C | PRO |
| | | 422 | HD1 | 34.176 | 12.488 | 9.116 | H | PRO |
| | | 423 | HD2 | 34.053 | 10.991 | 10.12 | H | PRO |
| | | 424 | HD3 | 34.887 | 10.94 | 8.527 | H | PRO |
| | | 425 | C | 35.589 | 11.146 | 13.065 | C | PRO |
| | | 426 | O | 35.279 | 10.037 | 12.635 | O | PRO |
| 27 | GLU | 427 | N | 36.365 | 11.279 | 14.164 | N | PRO |
| | | 428 | HN | 36.665 | 12.177 | 14.476 | H | PRO |
| | | 429 | CA | 36.906 | 10.187 | 14.942 | C | PRO |
| | | 430 | HA | 37.31 | 9.454 | 14.26 | H | PRO |
| | | 431 | CB | 38.029 | 10.657 | 15.905 | C | PRO |
| | | 432 | HB1 | 38.419 | 9.763 | 16.436 | H | PRO |
| | | 433 | HB2 | 37.615 | 11.345 | 16.674 | H | PRO |
| | | 434 | CG | 39.241 | 11.332 | 15.215 | C | PRO |
| | | 435 | HG1 | 39.506 | 10.772 | 14.292 | H | PRO |
| | | 436 | HG2 | 40.11 | 11.292 | 15.906 | H | PRO |
| | | 437 | CD | 39.022 | 12.804 | 14.857 | C | PRO |
| | | 438 | OE1 | 37.936 | 13.361 | 15.169 | O | PRO |
| | | 439 | OE2 | 39.95 | 13.402 | 14.252 | O | PRO |
| | | 440 | C | 35.834 | 9.519 | 15.778 | C | PRO |
| | | 441 | O | 36.017 | 8.4 | 16.252 | O | PRO |
| 28 | ALA | 442 | N | 34.659 | 10.173 | 15.943 | N | PRO |
| | | 443 | HN | 34.515 | 11.072 | 15.537 | H | PRO |
| | | 444 | CA | 33.537 | 9.637 | 16.682 | C | PRO |
| | | 445 | HA | 33.891 | 8.928 | 17.415 | H | PRO |
| | | 446 | CB | 32.765 | 10.748 | 17.416 | C | PRO |
| | | 447 | HB1 | 32.338 | 11.478 | 16.695 | H | PRO |
| | | 448 | HB2 | 33.448 | 11.291 | 18.103 | H | PRO |
| | | 449 | HB3 | 31.936 | 10.318 | 18.018 | H | PRO |
| | | 450 | C | 32.593 | 8.916 | 15.744 | C | PRO |
| | | 451 | O | 31.54 | 8.433 | 16.159 | O | PRO |
| 29 | CYS | 452 | N | 32.993 | 8.766 | 14.457 | N | PRO |
| | | 453 | HN | 33.813 | 9.224 | 14.122 | H | PRO |
| | | 454 | CA | 32.312 | 7.923 | 13.498 | C | PRO |
| | | 455 | HA | 31.265 | 7.844 | 13.752 | H | PRO |
| | | 456 | CB | 32.465 | 8.434 | 12.046 | C | PRO |
| | | 457 | HB1 | 31.94 | 7.741 | 11.354 | H | PRO |
| | | 458 | HB2 | 33.539 | 8.431 | 11.762 | H | PRO |
| | | 459 | SG | 31.795 | 10.102 | 11.842 | S | PRO |
| | | 460 | HG1 | 32.089 | 10.195 | 10.554 | H | PRO |
| | | 461 | C | 32.933 | 6.551 | 13.521 | C | PRO |
| | | 462 | O | 32.426 | 5.631 | 12.884 | O | PRO |
| 30 | MET | 463 | N | 34.037 | 6.381 | 14.281 | N | PRO |
| | | 464 | HN | 34.418 | 7.144 | 14.797 | H | PRO |
| | | 465 | CA | 34.724 | 5.127 | 14.452 | C | PRO |
| | | 466 | HA | 34.52 | 4.473 | 13.618 | H | PRO |
| | | 467 | CB | 36.251 | 5.332 | 14.609 | C | PRO |
| | | 468 | HB1 | 36.752 | 4.341 | 14.634 | H | PRO |
| | | 469 | HB2 | 36.466 | 5.847 | 15.57 | H | PRO |
| | | 470 | CG | 36.86 | 6.182 | 13.479 | C | PRO |
| | | 471 | HG1 | 36.365 | 7.177 | 13.464 | H | PRO |
| | | 472 | HG2 | 36.631 | 5.689 | 12.511 | H | PRO |
| | | 473 | SD | 38.653 | 6.428 | 13.623 | S | PRO |
| | | 474 | CE | 38.777 | 7.408 | 12.099 | C | PRO |
| | | 475 | HE1 | 38.161 | 8.33 | 12.166 | H | PRO |
| | | 476 | HE2 | 38.426 | 6.823 | 11.223 | H | PRO |
| | | 477 | HE3 | 39.828 | 7.711 | 11.908 | H | PRO |
| | | 478 | C | 34.167 | 4.536 | 15.715 | C | PRO |
| | | 479 | O | 34.295 | 5.126 | 16.787 | O | PRO |
| 31 | PHE | 480 | N | 33.479 | 3.379 | 15.62 | N | PRO |
| | | 481 | HN | 33.413 | 2.848 | 14.779 | H | PRO |
| | | 482 | CA | 32.687 | 2.894 | 16.725 | C | PRO |
| | | 483 | HA | 32.548 | 3.669 | 17.464 | H | PRO |
| | | 484 | CB | 31.288 | 2.372 | 16.298 | C | PRO |
| | | 485 | HB1 | 30.715 | 2.043 | 17.191 | H | PRO |
| | | 486 | HB2 | 31.41 | 1.506 | 15.613 | H | PRO |
| | | 487 | CG | 30.422 | 3.387 | 15.591 | C | PRO |
| | | 488 | CD1 | 30.535 | 4.778 | 15.783 | C | PRO |
| | | 489 | HD1 | 31.27 | 5.189 | 16.46 | H | PRO |
| | | 490 | CE1 | 29.697 | 5.664 | 15.095 | C | PRO |
| | | 491 | HE1 | 29.802 | 6.728 | 15.246 | H | PRO |
| | | 492 | CZ | 28.728 | 5.173 | 14.215 | C | PRO |
| | | 493 | HZ | 28.088 | 5.859 | 13.68 | H | PRO |
| | | 494 | CD2 | 29.418 | 2.909 | 14.729 | C | PRO |
| | | 495 | HD2 | 29.305 | 1.845 | 14.583 | H | PRO |
| | | 496 | CE2 | 28.577 | 3.793 | 14.044 | C | PRO |
| | | 497 | HE2 | 27.819 | 3.408 | 13.378 | H | PRO |
| | | 498 | C | 33.399 | 1.753 | 17.388 | C | PRO |
| | | 499 | O | 34.202 | 1.041 | 16.786 | O | PRO |
| 32 | GLU | 500 | N | 33.067 | 1.54 | 18.678 | N | PRO |
| | | 501 | HN | 32.467 | 2.173 | 19.161 | H | PRO |
| | | 502 | CA | 33.469 | 0.419 | 19.493 | C | PRO |
| | | 503 | HA | 34.546 | 0.35 | 19.456 | H | PRO |
| | | 504 | CB | 33.003 | 0.588 | 20.963 | C | PRO |
| | | 505 | HB1 | 33.461 | −0.231 | 21.558 | H | PRO |
| | | 506 | HB2 | 31.901 | 0.469 | 21.039 | H | PRO |
| | | 507 | CG | 33.414 | 1.928 | 21.626 | C | PRO |
| | | 508 | HG1 | 34.45 | 2.193 | 21.325 | H | PRO |
| | | 509 | HG2 | 33.4 | 1.793 | 22.728 | H | PRO |
| | | 510 | CD | 32.491 | 3.113 | 21.311 | C | PRO |
| | | 511 | OE1 | 31.527 | 2.955 | 20.513 | O | PRO |
| | | 512 | OE2 | 32.749 | 4.205 | 21.877 | O | PRO |
| | | 513 | C | 32.876 | −0.858 | 18.94 | C | PRO |
| | | 514 | O | 33.488 | −1.923 | 18.983 | O | PRO |
| 33 | SER | 515 | N | 31.655 | −0.742 | 18.367 | N | PRO |
| | | 516 | HN | 31.206 | 0.148 | 18.342 | H | PRO |
| | | 517 | CA | 30.881 | −1.813 | 17.785 | C | PRO |
| | | 518 | HA | 30.871 | −2.629 | 18.493 | H | PRO |
| | | 519 | CB | 29.412 | −1.381 | 17.549 | C | PRO |
| | | 520 | HB1 | 28.938 | −1.191 | 18.536 | H | PRO |
| | | 521 | HB2 | 28.848 | −2.195 | 17.046 | H | PRO |
| | | 522 | OG | 29.312 | −0.196 | 16.764 | O | PRO |
| | | 523 | HG1 | 28.479 | 0.217 | 17.001 | H | PRO |
| | | 524 | C | 31.464 | −2.339 | 16.491 | C | PRO |
| | | 525 | O | 31.156 | −3.456 | 16.083 | O | PRO |
| 34 | ASP | 526 | N | 32.364 | −1.574 | 15.829 | N | PRO |
| | | 527 | HN | 32.607 | −0.661 | 16.149 | H | PRO |
| | | 528 | CA | 33.051 | −2.033 | 14.637 | C | PRO |
| | | 529 | HA | 32.341 | −2.52 | 13.986 | H | PRO |
| | | 530 | CB | 33.76 | −0.879 | 13.881 | C | PRO |
| | | 531 | HB1 | 34.232 | −1.268 | 12.953 | H | PRO |
| | | 532 | HB2 | 34.552 | −0.434 | 14.52 | H | PRO |
| | | 533 | CG | 32.789 | 0.221 | 13.48 | C | PRO |
| | | 534 | OD1 | 31.618 | −0.089 | 13.137 | O | PRO |
| | | 535 | OD2 | 33.214 | 1.409 | 13.486 | O | PRO |
| | | 536 | C | 34.139 | −3.025 | 14.997 | C | PRO |
| | | 537 | O | 34.596 | −3.794 | 14.152 | O | PRO |
| 35 | LEU | 538 | N | 34.575 | −3.024 | 16.276 | N | PRO |
| | | 539 | HN | 34.166 | −2.413 | 16.95 | H | PRO |
| | | 540 | CA | 35.704 | −3.792 | 16.747 | C | PRO |
| | | 541 | HA | 36.332 | −4.073 | 15.914 | H | PRO |
| | | 542 | CB | 36.531 | −2.965 | 17.765 | C | PRO |
| | | 543 | HB1 | 37.428 | −3.544 | 18.073 | H | PRO |
| | | 544 | HB2 | 35.917 | −2.79 | 18.673 | H | PRO |
| | | 545 | CG | 36.995 | −1.584 | 17.241 | C | PRO |
| | | 546 | HG | 36.097 | −1.016 | 16.917 | H | PRO |
| | | 547 | CD1 | 37.648 | −0.765 | 18.369 | C | PRO |
| | | 548 | HD11 | 38.566 | −1.274 | 18.735 | H | PRO |
| | | 549 | HD12 | 36.942 | −0.647 | 19.219 | H | PRO |
| | | 550 | HD13 | 37.926 | 0.245 | 17.998 | H | PRO |
| | | 551 | CD2 | 37.937 | −1.692 | 16.027 | C | PRO |
| | | 552 | HD21 | 37.429 | −2.188 | 15.172 | H | PRO |
| | | 553 | HD22 | 38.843 | −2.278 | 16.292 | H | PRO |
| | | 554 | HD23 | 38.254 | −0.679 | 15.701 | H | PRO |
| | | 555 | C | 35.238 | −5.053 | 17.438 | C | PRO |
| | | 556 | O | 36.052 | −5.883 | 17.838 | O | PRO |
| 36 | THR | 557 | N | 33.903 | −5.244 | 17.572 | N | PRO |
| | | 558 | HN | 33.256 | −4.559 | 17.245 | H | PRO |
| | | 559 | CA | 33.302 | −6.414 | 18.19 | C | PRO |
| | | 560 | HA | 33.851 | −6.593 | 19.103 | H | PRO |
| | | 561 | CB | 31.834 | −6.228 | 18.562 | C | PRO |
| | | 562 | HB | 31.189 | −6.35 | 17.665 | H | PRO |
| | | 563 | OG1 | 31.609 | −4.915 | 19.049 | O | PRO |
| | | 564 | HG1 | 32.233 | −4.781 | 19.767 | H | PRO |
| | | 565 | CG2 | 31.387 | −7.208 | 19.663 | C | PRO |
| | | 566 | HG21 | 32.079 | −7.166 | 20.531 | H | PRO |
| | | 567 | HG22 | 31.344 | −8.251 | 19.282 | H | PRO |
| | | 568 | HG23 | 30.37 | −6.94 | 20.021 | H | PRO |
| | | 569 | C | 33.466 | −7.627 | 17.298 | C | PRO |
| | | 570 | O | 33.6 | −7.507 | 16.079 | O | PRO |
| 37 | ARG | 571 | N | 33.483 | −8.839 | 17.902 | N | PRO |
| | | 572 | HN | 33.382 | −8.906 | 18.891 | H | PRO |
| | | 573 | CA | 33.629 | −10.105 | 17.217 | C | PRO |
| | | 574 | HA | 34.596 | −10.097 | 16.737 | H | PRO |
| | | 575 | CB | 33.567 | −11.281 | 18.224 | C | PRO |
| | | 576 | HB1 | 32.554 | −11.317 | 18.679 | H | PRO |
| | | 577 | HB2 | 34.287 | −11.066 | 19.043 | H | PRO |
| | | 578 | CG | 33.901 | −12.663 | 17.627 | C | PRO |
| | | 579 | HG1 | 34.95 | −12.662 | 17.26 | H | PRO |
| | | 580 | HG2 | 33.243 | −12.851 | 16.752 | H | PRO |
| | | 581 | CD | 33.688 | −13.835 | 18.597 | C | PRO |
| | | 582 | HD1 | 33.918 | −14.796 | 18.088 | H | PRO |
| | | 583 | HD2 | 32.638 | −13.845 | 18.96 | H | PRO |
| | | 584 | NE | 34.61 | −13.669 | 19.767 | N | PRO |
| | | 585 | HE | 35.193 | −12.858 | 19.81 | H | PRO |
| | | 586 | CZ | 34.675 | −14.562 | 20.786 | C | PRO |
| | | 587 | NH1 | 33.929 | −15.689 | 20.794 | N | PRO |
| | | 588 | HH11 | 33.323 | −15.894 | 20.025 | H | PRO |
| | | 589 | HH12 | 34.029 | −16.311 | 21.57 | H | PRO |
| | | 590 | NH2 | 35.504 | −14.337 | 21.832 | N | PRO |
| | | 591 | HH21 | 36.07 | −13.513 | 21.864 | H | PRO |
| | | 592 | HH22 | 35.531 | −15.018 | 22.563 | H | PRO |
| | | 593 | C | 32.56 | −10.326 | 16.166 | C | PRO |
| | | 594 | O | 31.366 | −10.209 | 16.438 | O | PRO |
| 38 | SER | 595 | N | 33.004 | −10.659 | 14.931 | N | PRO |
| | | 596 | HN | 33.986 | −10.683 | 14.763 | H | PRO |
| | | 597 | CA | 32.196 | −11.058 | 13.795 | C | PRO |
| | | 598 | HA | 32.924 | −11.332 | 13.046 | H | PRO |
| | | 599 | CB | 31.343 | −12.342 | 14.02 | C | PRO |
| | | 600 | HB1 | 31.985 | −13.122 | 14.482 | H | PRO |
| | | 601 | HB2 | 30.988 | −12.731 | 13.042 | H | PRO |
| | | 602 | OG | 30.213 | −12.123 | 14.859 | O | PRO |
| | | 603 | HG1 | 30.538 | −11.605 | 15.599 | H | PRO |
| | | 604 | C | 31.415 | −9.913 | 13.183 | C | PRO |
| | | 605 | O | 30.436 | −10.128 | 12.468 | O | PRO |
| 39 | ALA | 606 | N | 31.867 | −8.661 | 13.43 | N | PRO |
| | | 607 | HN | 32.641 | −8.517 | 14.041 | H | PRO |
| | | 608 | CA | 31.325 | −7.454 | 12.847 | C | PRO |
| | | 609 | HA | 30.265 | −7.453 | 13.054 | H | PRO |
| | | 610 | CB | 31.95 | −6.187 | 13.462 | C | PRO |
| | | 611 | HB1 | 33.043 | −6.153 | 13.264 | H | PRO |
| | | 612 | HB2 | 31.794 | −6.18 | 14.562 | H | PRO |
| | | 613 | HB3 | 31.489 | −5.267 | 13.043 | H | PRO |
| | | 614 | C | 31.495 | −7.407 | 11.345 | C | PRO |
| | | 615 | O | 32.464 | −7.94 | 10.802 | O | PRO |
| 40 | PHE | 616 | N | 30.538 | −6.767 | 10.64 | N | PRO |
| | | 617 | HN | 29.758 | −6.333 | 11.085 | H | PRO |
| | | 618 | CA | 30.614 | −6.612 | 9.208 | C | PRO |
| | | 619 | HA | 31.647 | −6.429 | 8.954 | H | PRO |
| | | 620 | CB | 30.137 | −7.841 | 8.375 | C | PRO |
| | | 621 | HB1 | 30.766 | −8.714 | 8.653 | H | PRO |
| | | 622 | HB2 | 30.306 | −7.645 | 7.294 | H | PRO |
| | | 623 | CG | 28.699 | −8.266 | 8.563 | C | PRO |
| | | 624 | CD1 | 28.316 | −9.038 | 9.673 | C | PRO |
| | | 625 | HD1 | 29.045 | −9.277 | 10.432 | H | PRO |
| | | 626 | CE1 | 27.009 | −9.527 | 9.789 | C | PRO |
| | | 627 | HE1 | 26.731 | −10.125 | 10.644 | H | PRO |
| | | 628 | CZ | 26.065 | −9.245 | 8.793 | C | PRO |
| | | 629 | HZ | 25.056 | −9.622 | 8.879 | H | PRO |
| | | 630 | CD2 | 27.741 | −7.986 | 7.572 | C | PRO |
| | | 631 | HD2 | 28.023 | −7.411 | 6.703 | H | PRO |
| | | 632 | CE2 | 26.432 | −8.473 | 7.684 | C | PRO |
| | | 633 | HE2 | 25.705 | −8.254 | 6.916 | H | PRO |
| | | 634 | C | 29.867 | −5.372 | 8.813 | C | PRO |
| | | 635 | O | 28.987 | −4.897 | 9.529 | O | PRO |
| 41 | HIS | 636 | N | 30.239 | −4.81 | 7.646 | N | PRO |
| | | 637 | HN | 30.962 | −5.208 | 7.087 | H | PRO |
| | | 638 | CA | 29.632 | −3.624 | 7.101 | C | PRO |
| | | 639 | HA | 28.757 | −3.352 | 7.672 | H | PRO |
| | | 640 | CB | 30.61 | −2.428 | 7.023 | C | PRO |
| | | 641 | HB1 | 30.065 | −1.503 | 6.74 | H | PRO |
| | | 642 | HB2 | 31.383 | −2.625 | 6.248 | H | PRO |
| | | 643 | ND1 | 30.797 | −1.72 | 9.466 | N | PRO |
| | | 644 | HD1 | 29.859 | −1.393 | 9.58 | H | PRO |
| | | 645 | CG | 31.355 | −2.206 | 8.305 | C | PRO |
| | | 646 | CE1 | 31.769 | −1.753 | 10.411 | C | PRO |
| | | 647 | HE1 | 31.601 | −1.451 | 11.445 | H | PRO |
| | | 648 | NE2 | 32.909 | −2.221 | 9.939 | N | PRO |
| | | 649 | CD2 | 32.646 | −2.506 | 8.611 | C | PRO |
| | | 650 | HD2 | 33.42 | −2.929 | 7.982 | H | PRO |
| | | 651 | C | 29.213 | −3.987 | 5.708 | C | PRO |
| | | 652 | O | 29.955 | −4.656 | 4.989 | O | PRO |
| 42 | LEU | 653 | N | 28.005 | −3.555 | 5.298 | N | PRO |
| | | 654 | HN | 27.418 | −3.01 | 5.891 | H | PRO |
| | | 655 | CA | 27.513 | −3.773 | 3.959 | C | PRO |
| | | 656 | HA | 28.248 | −4.288 | 3.359 | H | PRO |
| | | 657 | CB | 26.164 | −4.526 | 3.894 | C | PRO |
| | | 658 | HB1 | 25.806 | −4.55 | 2.843 | H | PRO |
| | | 659 | HB2 | 25.411 | −3.97 | 4.492 | H | PRO |
| | | 660 | CG | 26.214 | −5.983 | 4.406 | C | PRO |
| | | 661 | HG | 26.57 | −5.963 | 5.459 | H | PRO |
| | | 662 | CD1 | 24.801 | −6.587 | 4.409 | C | PRO |
| | | 663 | HD11 | 24.389 | −6.609 | 3.377 | H | PRO |
| | | 664 | HD12 | 24.122 | −5.972 | 5.037 | H | PRO |
| | | 665 | HD13 | 24.819 | −7.622 | 4.811 | H | PRO |
| | | 666 | CD2 | 27.18 | −6.873 | 3.601 | C | PRO |
| | | 667 | HD21 | 28.224 | −6.502 | 3.689 | H | PRO |
| | | 668 | HD22 | 26.894 | −6.883 | 2.528 | H | PRO |
| | | 669 | HD23 | 27.153 | −7.915 | 3.986 | H | PRO |
| | | 670 | C | 27.316 | −2.415 | 3.364 | C | PRO |
| | | 671 | O | 26.95 | −1.466 | 4.058 | O | PRO |
| 43 | GLY | 672 | N | 27.595 | −2.299 | 2.051 | N | PRO |
| | | 673 | HN | 27.919 | −3.073 | 1.514 | H | PRO |
| | | 674 | CA | 27.443 | −1.07 | 1.321 | C | PRO |
| | | 675 | HA1 | 28.41 | −0.83 | 0.906 | H | PRO |
| | | 676 | HA2 | 27.041 | −0.294 | 1.956 | H | PRO |
| | | 677 | C | 26.503 | −1.291 | 0.186 | C | PRO |
| | | 678 | O | 26.465 | −2.369 | −0.407 | O | PRO |
| 44 | GLY | 679 | N | 25.733 | −0.237 | −0.151 | N | PRO |
| | | 680 | HN | 25.794 | 0.613 | 0.368 | H | PRO |
| | | 681 | CA | 24.827 | −0.235 | −1.273 | C | PRO |
| | | 682 | HA1 | 23.948 | 0.322 | −0.983 | H | PRO |
| | | 683 | HA2 | 24.61 | −1.25 | −1.572 | H | PRO |
| | | 684 | C | 25.472 | 0.5 | −2.389 | C | PRO |
| | | 685 | O | 25.937 | 1.619 | −2.199 | O | PRO |
| 45 | PHE | 686 | N | 25.502 | −0.108 | −3.589 | N | PRO |
| | | 687 | HN | 25.092 | −1.007 | −3.723 | H | PRO |
| | | 688 | CA | 26.185 | 0.47 | −4.721 | C | PRO |
| | | 689 | HA | 26.559 | 1.451 | −4.47 | H | PRO |
| | | 690 | CB | 27.365 | −0.386 | −5.24 | C | PRO |
| | | 691 | HB1 | 27.735 | −0.005 | −6.216 | H | PRO |
| | | 692 | HB2 | 27.066 | −1.449 | −5.362 | H | PRO |
| | | 693 | CG | 28.503 | −0.32 | −4.262 | C | PRO |
| | | 694 | CD1 | 28.589 | −1.223 | −3.188 | C | PRO |
| | | 695 | HD1 | 27.83 | −1.981 | −3.062 | H | PRO |
| | | 696 | CE1 | 29.645 | −1.139 | −2.271 | C | PRO |
| | | 697 | HE1 | 29.698 | −1.837 | −1.448 | H | PRO |
| | | 698 | CZ | 30.627 | −0.152 | −2.424 | C | PRO |
| | | 699 | HZ | 31.443 | −0.082 | −1.72 | H | PRO |
| | | 700 | CD2 | 29.49 | 0.669 | −4.402 | C | PRO |
| | | 701 | HD2 | 29.432 | 1.375 | −5.217 | H | PRO |
| | | 702 | CE2 | 30.548 | 0.755 | −3.489 | C | PRO |
| | | 703 | HE2 | 31.299 | 1.522 | −3.603 | H | PRO |
| | | 704 | C | 25.189 | 0.651 | −5.825 | C | PRO |
| | | 705 | O | 24.481 | −0.277 | −6.221 | O | PRO |
| 46 | TYR | 706 | N | 25.119 | 1.893 | −6.342 | N | PRO |
| | | 707 | HN | 25.66 | 2.643 | −5.97 | H | PRO |
| | | 708 | CA | 24.356 | 2.206 | −7.517 | C | PRO |
| | | 709 | HA | 24.382 | 1.356 | −8.183 | H | PRO |
| | | 710 | CB | 22.901 | 2.618 | −7.175 | C | PRO |
| | | 711 | HB1 | 22.873 | 3.604 | −6.664 | H | PRO |
| | | 712 | HB2 | 22.463 | 1.86 | −6.49 | H | PRO |
| | | 713 | CG | 22.033 | 2.658 | −8.402 | C | PRO |
| | | 714 | CD1 | 21.504 | 3.874 | −8.866 | C | PRO |
| | | 715 | HD1 | 21.722 | 4.79 | −8.337 | H | PRO |
| | | 716 | CE1 | 20.7 | 3.908 | −10.012 | C | PRO |
| | | 717 | HE1 | 20.3 | 4.849 | −10.362 | H | PRO |
| | | 718 | CZ | 20.416 | 2.724 | −10.705 | C | PRO |
| | | 719 | OH | 19.612 | 2.766 | −11.864 | O | PRO |
| | | 720 | HH | 19.558 | 1.875 | −12.219 | H | PRO |
| | | 721 | CD2 | 21.739 | 1.475 | −9.101 | C | PRO |
| | | 722 | HD2 | 22.135 | 0.532 | −8.753 | H | PRO |
| | | 723 | CE2 | 20.935 | 1.506 | −10.248 | C | PRO |
| | | 724 | HE2 | 20.722 | 0.587 | −10.774 | H | PRO |
| | | 725 | C | 25.107 | 3.341 | −8.158 | C | PRO |
| | | 726 | O | 25.533 | 4.273 | −7.478 | O | PRO |
| 47 | GLY | 727 | N | 25.329 | 3.263 | −9.49 | N | PRO |
| | | 728 | HN | 25.01 | 2.48 | −10.018 | H | PRO |
| | | 729 | CA | 26.075 | 4.268 | −10.223 | C | PRO |
| | | 730 | HA1 | 25.787 | 5.246 | −9.868 | H | PRO |
| | | 731 | HA2 | 25.856 | 4.121 | −11.271 | H | PRO |
| | | 732 | C | 27.563 | 4.12 | −10.046 | C | PRO |
| | | 733 | O | 28.327 | 4.998 | −10.442 | O | PRO |
| 48 | GLY | 734 | N | 28.009 | 3.007 | −9.413 | N | PRO |
| | | 735 | HN | 27.359 | 2.305 | −9.135 | H | PRO |
| | | 736 | CA | 29.4 | 2.764 | −9.082 | C | PRO |
| | | 737 | HA1 | 30.019 | 3.127 | −9.89 | H | PRO |
| | | 738 | HA2 | 29.505 | 1.701 | −8.922 | H | PRO |
| | | 739 | C | 29.828 | 3.461 | −7.817 | C | PRO |
| | | 740 | O | 31.009 | 3.463 | −7.482 | O | PRO |
| 49 | LYS | 741 | N | 28.871 | 4.073 | −7.086 | N | PRO |
| | | 742 | HN | 27.915 | 4.036 | −7.365 | H | PRO |
| | | 743 | CA | 29.139 | 4.881 | −5.921 | C | PRO |
| | | 744 | HA | 30.202 | 4.94 | −5.737 | H | PRO |
| | | 745 | CB | 28.554 | 6.308 | −6.089 | C | PRO |
| | | 746 | HB1 | 27.456 | 6.234 | −6.245 | H | PRO |
| | | 747 | HB2 | 28.985 | 6.75 | −7.012 | H | PRO |
| | | 748 | CG | 28.832 | 7.272 | −4.92 | C | PRO |
| | | 749 | HG1 | 29.931 | 7.409 | −4.829 | H | PRO |
| | | 750 | HG2 | 28.46 | 6.83 | −3.971 | H | PRO |
| | | 751 | CD | 28.139 | 8.632 | −5.1 | C | PRO |
| | | 752 | HD1 | 27.044 | 8.453 | −5.157 | H | PRO |
| | | 753 | HD2 | 28.453 | 9.077 | −6.068 | H | PRO |
| | | 754 | CE | 28.402 | 9.63 | −3.963 | C | PRO |
| | | 755 | HE1 | 28.115 | 9.197 | −2.98 | H | PRO |
| | | 756 | HE2 | 27.816 | 10.557 | −4.138 | H | PRO |
| | | 757 | NZ | 29.83 | 10.008 | −3.889 | N | PRO |
| | | 758 | HZ1 | 30.212 | 10.125 | −4.849 | H | PRO |
| | | 759 | HZ2 | 30.357 | 9.271 | −3.378 | H | PRO |
| | | 760 | HZ3 | 29.931 | 10.903 | −3.37 | H | PRO |
| | | 761 | C | 28.474 | 4.225 | −4.742 | C | PRO |
| | | 762 | O | 27.363 | 3.708 | −4.858 | O | PRO |
| 50 | LEU | 763 | N | 29.156 | 4.229 | −3.571 | N | PRO |
| | | 764 | HN | 30.098 | 4.554 | −3.525 | H | PRO |
| | | 765 | CA | 28.587 | 3.855 | −2.295 | C | PRO |
| | | 766 | HA | 28.141 | 2.879 | −2.413 | H | PRO |
| | | 767 | CB | 29.683 | 3.8 | −1.206 | C | PRO |
| | | 768 | HB1 | 30.151 | 4.804 | −1.112 | H | PRO |
| | | 769 | HB2 | 30.478 | 3.104 | −1.549 | H | PRO |
| | | 770 | CG | 29.222 | 3.336 | 0.191 | C | PRO |
| | | 771 | HG | 28.412 | 4.008 | 0.546 | H | PRO |
| | | 772 | CD1 | 28.675 | 1.906 | 0.172 | C | PRO |
| | | 773 | HD11 | 29.448 | 1.198 | −0.196 | H | PRO |
| | | 774 | HD12 | 27.778 | 1.828 | −0.478 | H | PRO |
| | | 775 | HD13 | 28.385 | 1.612 | 1.203 | H | PRO |
| | | 776 | CD2 | 30.38 | 3.443 | 1.194 | C | PRO |
| | | 777 | HD21 | 30.766 | 4.484 | 1.233 | H | PRO |
| | | 778 | HD22 | 31.206 | 2.769 | 0.881 | H | PRO |
| | | 779 | HD23 | 30.043 | 3.15 | 2.211 | H | PRO |
| | | 780 | C | 27.517 | 4.845 | −1.873 | C | PRO |
| | | 781 | O | 27.811 | 5.997 | −1.553 | O | PRO |
| 51 | ILE | 782 | N | 26.238 | 4.406 | −1.893 | N | PRO |
| | | 783 | HN | 26.036 | 3.463 | −2.144 | H | PRO |
| | | 784 | CA | 25.094 | 5.272 | −1.694 | C | PRO |
| | | 785 | HA | 25.436 | 6.258 | −1.416 | H | PRO |
| | | 786 | CB | 24.254 | 5.426 | −2.966 | C | PRO |
| | | 787 | HB | 23.337 | 6.01 | −2.739 | H | PRO |
| | | 788 | CG2 | 25.086 | 6.272 | −3.955 | C | PRO |
| | | 789 | HG21 | 25.984 | 5.711 | −4.29 | H | PRO |
| | | 790 | HG22 | 25.424 | 7.217 | −3.478 | H | PRO |
| | | 791 | HG23 | 24.481 | 6.522 | −4.853 | H | PRO |
| | | 792 | CG1 | 23.827 | 4.103 | −3.66 | C | PRO |
| | | 793 | HG11 | 24.717 | 3.458 | −3.823 | H | PRO |
| | | 794 | HG12 | 23.45 | 4.383 | −4.667 | H | PRO |
| | | 795 | CD1 | 22.722 | 3.3 | −2.97 | C | PRO |
| | | 796 | HD1 | 21.861 | 3.956 | −2.719 | H | PRO |
| | | 797 | HD2 | 23.097 | 2.825 | −2.038 | H | PRO |
| | | 798 | HD3 | 22.362 | 2.493 | −3.644 | H | PRO |
| | | 799 | C | 24.239 | 4.808 | −0.54 | C | PRO |
| | | 800 | O | 23.206 | 5.412 | −0.26 | O | PRO |
| 52 | SER | 801 | N | 24.649 | 3.752 | 0.192 | N | PRO |
| | | 802 | HN | 25.492 | 3.261 | −0.013 | H | PRO |
| | | 803 | CA | 23.92 | 3.308 | 1.362 | C | PRO |
| | | 804 | HA | 23.625 | 4.177 | 1.932 | H | PRO |
| | | 805 | CB | 22.682 | 2.431 | 1.023 | C | PRO |
| | | 806 | HB1 | 23.008 | 1.481 | 0.548 | H | PRO |
| | | 807 | HB2 | 22.042 | 2.976 | 0.296 | H | PRO |
| | | 808 | OG | 21.887 | 2.123 | 2.165 | O | PRO |
| | | 809 | HG1 | 21.291 | 2.862 | 2.308 | H | PRO |
| | | 810 | C | 24.9 | 2.531 | 2.191 | C | PRO |
| | | 811 | O | 25.879 | 2.008 | 1.663 | O | PRO |
| 53 | VAL | 812 | N | 24.667 | 2.454 | 3.517 | N | PRO |
| | | 813 | HN | 23.865 | 2.884 | 3.924 | H | PRO |
| | | 814 | CA | 25.547 | 1.77 | 4.43 | C | PRO |
| | | 815 | HA | 25.974 | 0.921 | 3.917 | H | PRO |
| | | 816 | CB | 26.682 | 2.662 | 4.941 | C | PRO |
| | | 817 | HB | 27.202 | 3.074 | 4.05 | H | PRO |
| | | 818 | CG1 | 26.156 | 3.853 | 5.772 | C | PRO |
| | | 819 | HG11 | 25.421 | 4.447 | 5.188 | H | PRO |
| | | 820 | HG12 | 27.002 | 4.515 | 6.054 | H | PRO |
| | | 821 | HG13 | 25.663 | 3.504 | 6.704 | H | PRO |
| | | 822 | CG2 | 27.708 | 1.824 | 5.726 | C | PRO |
| | | 823 | HG21 | 27.98 | 0.916 | 5.146 | H | PRO |
| | | 824 | HG22 | 27.292 | 1.495 | 6.702 | H | PRO |
| | | 825 | HG23 | 28.623 | 2.425 | 5.912 | H | PRO |
| | | 826 | C | 24.709 | 1.236 | 5.565 | C | PRO |
| | | 827 | O | 23.713 | 1.84 | 5.959 | O | PRO |
| 54 | ALA | 828 | N | 25.1 | 0.064 | 6.107 | N | PRO |
| | | 829 | HN | 25.863 | −0.452 | 5.725 | H | PRO |
| | | 830 | CA | 24.531 | −0.483 | 7.307 | C | PRO |
| | | 831 | HA | 24.263 | 0.32 | 7.977 | H | PRO |
| | | 832 | CB | 23.316 | −1.389 | 7.033 | C | PRO |
| | | 833 | HB1 | 23.592 | −2.215 | 6.343 | H | PRO |
| | | 834 | HB2 | 22.507 | −0.797 | 6.554 | H | PRO |
| | | 835 | HB3 | 22.922 | −1.821 | 7.977 | H | PRO |
| | | 836 | C | 25.637 | −1.281 | 7.942 | C | PRO |
| | | 837 | O | 26.483 | −1.843 | 7.245 | O | PRO |
| 55 | SER | 838 | N | 25.675 | −1.312 | 9.291 | N | PRO |
| | | 839 | HN | 24.978 | −0.868 | 9.848 | H | PRO |
| | | 840 | CA | 26.744 | −1.934 | 10.042 | C | PRO |
| | | 841 | HA | 27.379 | −2.508 | 9.382 | H | PRO |
| | | 842 | CB | 27.604 | −0.91 | 10.808 | C | PRO |
| | | 843 | HB1 | 28.373 | −1.429 | 11.418 | H | PRO |
| | | 844 | HB2 | 26.973 | −0.294 | 11.484 | H | PRO |
| | | 845 | OG | 28.27 | −0.056 | 9.884 | O | PRO |
| | | 846 | HG1 | 27.599 | 0.509 | 9.496 | H | PRO |
| | | 847 | C | 26.135 | −2.89 | 11.025 | C | PRO |
| | | 848 | O | 25.047 | −2.661 | 11.547 | O | PRO |
| 56 | PHE | 849 | N | 26.825 | −4.021 | 11.268 | N | PRO |
| | | 850 | HN | 27.716 | −4.187 | 10.852 | H | PRO |
| | | 851 | CA | 26.257 | −5.157 | 11.954 | C | PRO |
| | | 852 | HA | 25.417 | −4.853 | 12.56 | H | PRO |
| | | 853 | CB | 25.868 | −6.307 | 10.982 | C | PRO |
| | | 854 | HB1 | 25.352 | −7.127 | 11.526 | H | PRO |
| | | 855 | HB2 | 26.775 | −6.722 | 10.492 | H | PRO |
| | | 856 | CG | 24.964 | −5.806 | 9.888 | C | PRO |
| | | 857 | CD1 | 25.508 | −5.295 | 8.694 | C | PRO |
| | | 858 | HD1 | 26.579 | −5.287 | 8.555 | H | PRO |
| | | 859 | CE1 | 24.682 | −4.753 | 7.705 | C | PRO |
| | | 860 | HE1 | 25.115 | −4.337 | 6.808 | H | PRO |
| | | 861 | CZ | 23.294 | −4.744 | 7.883 | C | PRO |
| | | 862 | HZ | 22.652 | −4.344 | 7.113 | H | PRO |
| | | 863 | CD2 | 23.569 | −5.812 | 10.046 | C | PRO |
| | | 864 | HD2 | 23.134 | −6.207 | 10.952 | H | PRO |
| | | 865 | CE2 | 22.739 | −5.293 | 9.044 | C | PRO |
| | | 866 | HE2 | 21.666 | −5.312 | 9.167 | H | PRO |
| | | 867 | C | 27.347 | −5.679 | 12.844 | C | PRO |
| | | 868 | O | 28.516 | −5.676 | 12.464 | O | PRO |
| 57 | HIS | 869 | N | 27.004 | −6.122 | 14.069 | N | PRO |
| | | 870 | HN | 26.067 | −6.095 | 14.408 | H | PRO |
| | | 871 | CA | 27.995 | −6.654 | 14.976 | C | PRO |
| | | 872 | HA | 28.677 | −7.279 | 14.419 | H | PRO |
| | | 873 | CB | 28.776 | −5.561 | 15.746 | C | PRO |
| | | 874 | HB1 | 29.249 | −4.876 | 15.011 | H | PRO |
| | | 875 | HB2 | 29.596 | −6.038 | 16.325 | H | PRO |
| | | 876 | ND1 | 26.909 | −3.915 | 16.343 | N | PRO |
| | | 877 | HD1 | 26.523 | −3.83 | 15.424 | H | PRO |
| | | 878 | CG | 27.959 | −4.733 | 16.705 | C | PRO |
| | | 879 | CE1 | 26.437 | −3.356 | 17.482 | C | PRO |
| | | 880 | HE1 | 25.582 | −2.679 | 17.5 | H | PRO |
| | | 881 | NE2 | 27.103 | −3.753 | 18.548 | N | PRO |
| | | 882 | CD2 | 28.061 | −4.624 | 18.058 | C | PRO |
| | | 883 | HD2 | 28.749 | −5.11 | 18.739 | H | PRO |
| | | 884 | C | 27.295 | −7.528 | 15.966 | C | PRO |
| | | 885 | O | 26.113 | −7.334 | 16.232 | O | PRO |
| 58 | GLN | 886 | N | 28.007 | −8.531 | 16.529 | N | PRO |
| | | 887 | HN | 28.964 | −8.691 | 16.296 | H | PRO |
| | | 888 | CA | 27.472 | −9.389 | 17.564 | C | PRO |
| | | 889 | HA | 26.495 | −9.717 | 17.241 | H | PRO |
| | | 890 | CB | 28.354 | −10.64 | 17.774 | C | PRO |
| | | 891 | HB1 | 29.281 | −10.354 | 18.316 | H | PRO |
| | | 892 | HB2 | 28.654 | −11.009 | 16.77 | H | PRO |
| | | 893 | CG | 27.637 | −11.796 | 18.494 | C | PRO |
| | | 894 | HG1 | 26.764 | −12.133 | 17.895 | H | PRO |
| | | 895 | HG2 | 27.266 | −11.474 | 19.491 | H | PRO |
| | | 896 | CD | 28.596 | −12.972 | 18.665 | C | PRO |
| | | 897 | OE1 | 28.572 | −13.939 | 17.895 | O | PRO |
| | | 898 | NE2 | 29.482 | −12.878 | 19.695 | N | PRO |
| | | 899 | HE21 | 29.447 | −12.082 | 20.3 | H | PRO |
| | | 900 | HE22 | 30.121 | −13.626 | 19.873 | H | PRO |
| | | 901 | C | 27.32 | −8.612 | 18.854 | C | PRO |
| | | 902 | O | 28.165 | −7.782 | 19.184 | O | PRO |
| 59 | ALA | 903 | N | 26.232 | −8.856 | 19.611 | N | PRO |
| | | 904 | HN | 25.568 | −9.555 | 19.358 | H | PRO |
| | | 905 | CA | 25.892 | −8.012 | 20.727 | C | PRO |
| | | 906 | HA | 26.746 | −7.935 | 21.383 | H | PRO |
| | | 907 | CB | 25.43 | −6.606 | 20.285 | C | PRO |
| | | 908 | HB1 | 24.57 | −6.686 | 19.586 | H | PRO |
| | | 909 | HB2 | 26.254 | −6.081 | 19.756 | H | PRO |
| | | 910 | HB3 | 25.13 | −5.986 | 21.156 | H | PRO |
| | | 911 | C | 24.766 | −8.653 | 21.491 | C | PRO |
| | | 912 | O | 23.6 | −8.285 | 21.354 | O | PRO |
| 60 | GLU | 913 | N | 25.102 | −9.649 | 22.34 | N | PRO |
| | | 914 | HN | 26.02 | −10.037 | 22.352 | H | PRO |
| | | 915 | CA | 24.206 | −10.228 | 23.316 | C | PRO |
| | | 916 | HA | 23.312 | −10.55 | 22.802 | H | PRO |
| | | 917 | CB | 24.821 | −11.455 | 24.034 | C | PRO |
| | | 918 | HB1 | 24.172 | −11.736 | 24.891 | H | PRO |
| | | 919 | HB2 | 25.815 | −11.178 | 24.444 | H | PRO |
| | | 920 | CG | 24.964 | −12.725 | 23.154 | C | PRO |
| | | 921 | HG1 | 23.963 | −13.034 | 22.783 | H | PRO |
| | | 922 | HG2 | 25.364 | −13.549 | 23.784 | H | PRO |
| | | 923 | CD | 25.9 | −12.556 | 21.956 | C | PRO |
| | | 924 | OE1 | 26.971 | −11.907 | 22.108 | O | PRO |
| | | 925 | OE2 | 25.554 | −13.086 | 20.865 | O | PRO |
| | | 926 | C | 23.792 | −9.178 | 24.324 | C | PRO |
| | | 927 | O | 24.623 | −8.435 | 24.844 | O | PRO |
| 61 | HIS | 928 | N | 22.47 | −9.074 | 24.584 | N | PRO |
| | | 929 | HN | 21.813 | −9.709 | 24.185 | H | PRO |
| | | 930 | CA | 21.893 | −7.922 | 25.236 | C | PRO |
| | | 931 | HA | 22.665 | −7.198 | 25.455 | H | PRO |
| | | 932 | CB | 20.826 | −7.258 | 24.335 | C | PRO |
| | | 933 | HB1 | 19.922 | −7.902 | 24.293 | H | PRO |
| | | 934 | HB2 | 21.23 | −7.2 | 23.302 | H | PRO |
| | | 935 | ND1 | 19.208 | −5.3 | 24.514 | N | PRO |
| | | 936 | HD1 | 18.437 | −5.739 | 24.052 | H | PRO |
| | | 937 | CG | 20.443 | −5.864 | 24.751 | C | PRO |
| | | 938 | CE1 | 19.267 | −4.021 | 24.959 | C | PRO |
| | | 939 | HE1 | 18.422 | −3.334 | 24.911 | H | PRO |
| | | 940 | NE2 | 20.455 | −3.722 | 25.45 | N | PRO |
| | | 941 | CD2 | 21.196 | −4.88 | 25.315 | C | PRO |
| | | 942 | HD2 | 22.231 | −4.892 | 25.632 | H | PRO |
| | | 943 | C | 21.246 | −8.333 | 26.528 | C | PRO |
| | | 944 | O | 20.829 | −9.476 | 26.697 | O | PRO |
| 62 | SER | 945 | N | 21.158 | −7.389 | 27.49 | N | PRO |
| | | 946 | HN | 21.534 | −6.475 | 27.355 | H | PRO |
| | | 947 | CA | 20.485 | −7.555 | 28.762 | C | PRO |
| | | 948 | HA | 20.867 | −8.452 | 29.226 | H | PRO |
| | | 949 | CB | 20.762 | −6.359 | 29.711 | C | PRO |
| | | 950 | HB1 | 21.831 | −6.381 | 30.013 | H | PRO |
| | | 951 | HB2 | 20.141 | −6.435 | 30.63 | H | PRO |
| | | 952 | OG | 20.52 | −5.111 | 29.066 | O | PRO |
| | | 953 | HG1 | 20.303 | −4.475 | 29.752 | H | PRO |
| | | 954 | C | 18.987 | −7.729 | 28.613 | C | PRO |
| | | 955 | O | 18.377 | −8.525 | 29.323 | O | PRO |
| 63 | GLU | 956 | N | 18.365 | −6.976 | 27.677 | N | PRO |
| | | 957 | HN | 18.889 | −6.341 | 27.114 | H | PRO |
| | | 958 | CA | 16.927 | −6.866 | 27.571 | C | PRO |
| | | 959 | HA | 16.474 | −7.093 | 28.525 | H | PRO |
| | | 960 | CB | 16.525 | −5.43 | 27.153 | C | PRO |
| | | 961 | HB1 | 15.418 | −5.354 | 27.114 | H | PRO |
| | | 962 | HB2 | 16.919 | −5.217 | 26.136 | H | PRO |
| | | 963 | CG | 17.045 | −4.35 | 28.121 | C | PRO |
| | | 964 | HG1 | 18.155 | −4.369 | 28.176 | H | PRO |
| | | 965 | HG2 | 16.636 | −4.532 | 29.137 | H | PRO |
| | | 966 | CD | 16.617 | −2.967 | 27.645 | C | PRO |
| | | 967 | OE1 | 15.752 | −2.351 | 28.325 | O | PRO |
| | | 968 | OE2 | 17.147 | −2.501 | 26.604 | O | PRO |
| | | 969 | C | 16.361 | −7.814 | 26.538 | C | PRO |
| | | 970 | O | 15.164 | −7.784 | 26.258 | O | PRO |
| 64 | LEU | 971 | N | 17.21 | −8.681 | 25.944 | N | PRO |
| | | 972 | HN | 18.159 | −8.748 | 26.24 | H | PRO |
| | | 973 | CA | 16.82 | −9.571 | 24.873 | C | PRO |
| | | 974 | HA | 15.753 | −9.733 | 24.9 | H | PRO |
| | | 975 | CB | 17.254 | −9.067 | 23.472 | C | PRO |
| | | 976 | HB1 | 16.949 | −9.798 | 22.693 | H | PRO |
| | | 977 | HB2 | 18.363 | −9.006 | 23.447 | H | PRO |
| | | 978 | CG | 16.691 | −7.68 | 23.083 | C | PRO |
| | | 979 | HG | 16.878 | −6.981 | 23.925 | H | PRO |
| | | 980 | CD1 | 17.437 | −7.121 | 21.865 | C | PRO |
| | | 981 | HD11 | 17.293 | −7.791 | 20.991 | H | PRO |
| | | 982 | HD12 | 18.526 | −7.048 | 22.074 | H | PRO |
| | | 983 | HD13 | 17.057 | −6.11 | 21.605 | H | PRO |
| | | 984 | CD2 | 15.175 | −7.701 | 22.823 | C | PRO |
| | | 985 | HD21 | 14.627 | −8.063 | 23.72 | H | PRO |
| | | 986 | HD22 | 14.932 | −8.367 | 21.967 | H | PRO |
| | | 987 | HD23 | 14.813 | −6.678 | 22.586 | H | PRO |
| | | 988 | C | 17.491 | −10.891 | 25.143 | C | PRO |
| | | 989 | O | 18.334 | −10.999 | 26.033 | O | PRO |
| 65 | GLN | 990 | N | 17.105 | −11.948 | 24.395 | N | PRO |
| | | 991 | HN | 16.436 | −11.858 | 23.661 | H | PRO |
| | | 992 | CA | 17.637 | −13.272 | 24.613 | C | PRO |
| | | 993 | HA | 18.648 | −13.179 | 24.979 | H | PRO |
| | | 994 | CB | 16.792 | −14.101 | 25.62 | C | PRO |
| | | 995 | HB1 | 16.192 | −14.882 | 25.107 | H | PRO |
| | | 996 | HB2 | 16.067 | −13.41 | 26.1 | H | PRO |
| | | 997 | CG | 17.617 | −14.737 | 26.762 | C | PRO |
| | | 998 | HG1 | 16.926 | −15.236 | 27.473 | H | PRO |
| | | 999 | HG2 | 18.166 | −13.944 | 27.314 | H | PRO |
| | | 1000 | CD | 18.593 | −15.792 | 26.232 | C | PRO |
| | | 1001 | OE1 | 18.188 | −16.888 | 25.834 | O | PRO |
| | | 1002 | NE2 | 19.912 | −15.449 | 26.21 | N | PRO |
| | | 1003 | HE21 | 20.586 | −16.12 | 25.903 | H | PRO |
| | | 1004 | HE22 | 20.194 | −14.534 | 26.5 | H | PRO |
| | | 1005 | C | 17.713 | −13.97 | 23.281 | C | PRO |
| | | 1006 | O | 16.913 | −13.719 | 22.381 | O | PRO |
| 66 | GLY | 1007 | N | 18.712 | −14.858 | 23.132 | N | PRO |
| | | 1008 | HN | 19.333 | −15.057 | 23.887 | H | PRO |
| | | 1009 | CA | 19.041 | −15.508 | 21.892 | C | PRO |
| | | 1010 | HA1 | 19.004 | −14.789 | 21.087 | H | PRO |
| | | 1011 | HA2 | 18.389 | −16.361 | 21.771 | H | PRO |
| | | 1012 | C | 20.45 | −15.983 | 22.042 | C | PRO |
| | | 1013 | O | 21.252 | −15.352 | 22.729 | O | PRO |
| 67 | LYS | 1014 | N | 20.776 | −17.139 | 21.424 | N | PRO |
| | | 1015 | HN | 20.106 | −17.614 | 20.859 | H | PRO |
| | | 1016 | CA | 22.081 | −17.763 | 21.494 | C | PRO |
| | | 1017 | HA | 22.355 | −17.806 | 22.537 | H | PRO |
| | | 1018 | CB | 22.013 | −19.215 | 20.952 | C | PRO |
| | | 1019 | HB1 | 21.756 | −19.203 | 19.871 | H | PRO |
| | | 1020 | HB2 | 21.17 | −19.708 | 21.481 | H | PRO |
| | | 1021 | CG | 23.271 | −20.069 | 21.211 | C | PRO |
| | | 1022 | HG1 | 22.972 | −21.109 | 21.463 | H | PRO |
| | | 1023 | HG2 | 23.777 | −19.654 | 22.108 | H | PRO |
| | | 1024 | CD | 24.278 | −20.119 | 20.044 | C | PRO |
| | | 1025 | HD1 | 25.296 | −20.187 | 20.482 | H | PRO |
| | | 1026 | HD2 | 24.228 | −19.168 | 19.472 | H | PRO |
| | | 1027 | CE | 24.112 | −21.318 | 19.094 | C | PRO |
| | | 1028 | HE1 | 24.232 | −22.272 | 19.652 | H | PRO |
| | | 1029 | HE2 | 24.876 | −21.273 | 18.288 | H | PRO |
| | | 1030 | NZ | 22.778 | −21.333 | 18.451 | N | PRO |
| | | 1031 | HZ1 | 22.639 | −20.443 | 17.932 | H | PRO |
| | | 1032 | HZ2 | 22.046 | −21.411 | 19.185 | H | PRO |
| | | 1033 | HZ3 | 22.708 | −22.134 | 17.791 | H | PRO |
| | | 1034 | C | 23.137 | −16.937 | 20.785 | C | PRO |
| | | 1035 | O | 24.237 | −16.751 | 21.304 | O | PRO |
| 68 | LYS | 1036 | N | 22.8 | −16.386 | 19.596 | N | PRO |
| | | 1037 | HN | 21.929 | −16.607 | 19.164 | H | PRO |
| | | 1038 | CA | 23.597 | −15.367 | 18.952 | C | PRO |
| | | 1039 | HA | 24.347 | −15.003 | 19.638 | H | PRO |
| | | 1040 | CB | 24.297 | −15.802 | 17.645 | C | PRO |
| | | 1041 | HB1 | 24.819 | −14.916 | 17.224 | H | PRO |
| | | 1042 | HB2 | 23.551 | −16.151 | 16.899 | H | PRO |
| | | 1043 | CG | 25.34 | −16.902 | 17.869 | C | PRO |
| | | 1044 | HG1 | 24.851 | −17.899 | 17.837 | H | PRO |
| | | 1045 | HG2 | 25.741 | −16.766 | 18.896 | H | PRO |
| | | 1046 | CD | 26.504 | −16.85 | 16.867 | C | PRO |
| | | 1047 | HD1 | 26.714 | −15.792 | 16.6 | H | PRO |
| | | 1048 | HD2 | 26.199 | −17.372 | 15.935 | H | PRO |
| | | 1049 | CE | 27.795 | −17.473 | 17.412 | C | PRO |
| | | 1050 | HE1 | 28.573 | −17.501 | 16.619 | H | PRO |
| | | 1051 | HE2 | 27.609 | −18.505 | 17.781 | H | PRO |
| | | 1052 | NZ | 28.332 | −16.666 | 18.537 | N | PRO |
| | | 1053 | HZ1 | 27.642 | −16.61 | 19.313 | H | PRO |
| | | 1054 | HZ2 | 28.503 | −15.695 | 18.204 | H | PRO |
| | | 1055 | HZ3 | 29.221 | −17.079 | 18.883 | H | PRO |
| | | 1056 | C | 22.694 | −14.215 | 18.629 | C | PRO |
| | | 1057 | O | 21.69 | −14.364 | 17.934 | O | PRO |
| 69 | GLN | 1058 | N | 23.052 | −13.021 | 19.141 | N | PRO |
| | | 1059 | HN | 23.883 | −12.923 | 19.684 | H | PRO |
| | | 1060 | CA | 22.269 | −11.822 | 18.982 | C | PRO |
| | | 1061 | HA | 21.396 | −12.007 | 18.373 | H | PRO |
| | | 1062 | CB | 21.852 | −11.228 | 20.347 | C | PRO |
| | | 1063 | HB1 | 21.272 | −10.293 | 20.193 | H | PRO |
| | | 1064 | HB2 | 22.78 | −10.969 | 20.9 | H | PRO |
| | | 1065 | CG | 21.025 | −12.206 | 21.208 | C | PRO |
| | | 1066 | HG1 | 21.552 | −13.182 | 21.265 | H | PRO |
| | | 1067 | HG2 | 20.03 | −12.378 | 20.744 | H | PRO |
| | | 1068 | CD | 20.824 | −11.655 | 22.625 | C | PRO |
| | | 1069 | OE1 | 20.447 | −10.497 | 22.823 | O | PRO |
| | | 1070 | NE2 | 21.099 | −12.519 | 23.642 | N | PRO |
| | | 1071 | HE21 | 20.974 | −12.231 | 24.592 | H | PRO |
| | | 1072 | HE22 | 21.376 | −13.456 | 23.43 | H | PRO |
| | | 1073 | C | 23.16 | −10.838 | 18.282 | C | PRO |
| | | 1074 | O | 24.327 | −10.695 | 18.636 | O | PRO |
| 70 | TYR | 1075 | N | 22.634 | −10.153 | 17.248 | N | PRO |
| | | 1076 | HN | 21.694 | −10.296 | 16.947 | H | PRO |
| | | 1077 | CA | 23.387 | −9.188 | 16.482 | C | PRO |
| | | 1078 | HA | 24.357 | −9.024 | 16.927 | H | PRO |
| | | 1079 | CB | 23.542 | −9.58 | 14.989 | C | PRO |
| | | 1080 | HB1 | 23.81 | −8.695 | 14.373 | H | PRO |
| | | 1081 | HB2 | 22.594 | −10.01 | 14.6 | H | PRO |
| | | 1082 | CG | 24.641 | −10.599 | 14.829 | C | PRO |
| | | 1083 | CD1 | 24.446 | −11.952 | 15.164 | C | PRO |
| | | 1084 | HD1 | 23.488 | −12.277 | 15.542 | H | PRO |
| | | 1085 | CH1 | 25.48 | −12.883 | 14.999 | C | PRO |
| | | 1086 | HE1 | 25.317 | −13.921 | 15.251 | H | PRO |
| | | 1087 | CZ | 26.728 | −12.469 | 14.519 | C | PRO |
| | | 1088 | OH | 27.774 | −13.406 | 14.379 | O | PRO |
| | | 1089 | HH | 28.604 | −12.937 | 14.485 | H | PRO |
| | | 1090 | CD2 | 25.891 | −10.204 | 14.322 | C | PRO |
| | | 1091 | HD2 | 26.051 | −9.173 | 14.041 | H | PRO |
| | | 1092 | CE2 | 26.936 | −11.126 | 14.183 | C | PRO |
| | | 1093 | HE2 | 27.895 | −10.793 | 13.814 | H | PRO |
| | | 1094 | C | 22.644 | −7.888 | 16.571 | C | PRO |
| | | 1095 | O | 21.42 | −7.868 | 16.65 | O | PRO |
| 71 | GLN | 1096 | N | 23.385 | −6.76 | 16.574 | N | PRO |
| | | 1097 | HN | 24.378 | −6.806 | 16.502 | H | PRO |
| | | 1098 | CA | 22.823 | −5.437 | 16.687 | C | PRO |
| | | 1099 | HA | 21.747 | −5.509 | 16.748 | H | PRO |
| | | 1100 | CB | 23.306 | −4.687 | 17.95 | C | PRO |
| | | 1101 | HB1 | 24.417 | −4.652 | 17.943 | H | PRO |
| | | 1102 | HB2 | 23.003 | −5.293 | 18.83 | H | PRO |
| | | 1103 | CG | 22.727 | −3.267 | 18.117 | C | PRO |
| | | 1104 | HG1 | 21.619 | −3.323 | 18.174 | H | PRO |
| | | 1105 | HG2 | 23.01 | −2.628 | 17.253 | H | PRO |
| | | 1106 | CD | 23.26 | −2.61 | 19.391 | C | PRO |
| | | 1107 | OE1 | 24.019 | −3.202 | 20.166 | O | PRO |
| | | 1108 | NE2 | 22.851 | −1.329 | 19.607 | N | PRO |
| | | 1109 | HE21 | 23.157 | −0.852 | 20.431 | H | PRO |
| | | 1110 | HE22 | 22.26 | −0.87 | 18.943 | H | PRO |
| | | 1111 | C | 23.168 | −4.65 | 15.448 | C | PRO |
| | | 1112 | O | 24.278 | −4.712 | 14.919 | O | PRO |
| 72 | LEU | 1113 | N | 22.163 | −3.898 | 14.96 | N | PRO |
| | | 1114 | HN | 21.279 | −3.917 | 15.422 | H | PRO |
| | | 1115 | CA | 22.184 | −3.08 | 13.779 | C | PRO |
| | | 1116 | HA | 22.918 | −3.472 | 13.091 | H | PRO |
| | | 1117 | CB | 20.768 | −3.109 | 13.159 | C | PRO |
| | | 1118 | HB1 | 20.032 | −2.752 | 13.911 | H | PRO |
| | | 1119 | HB2 | 20.519 | −4.173 | 12.959 | H | PRO |
| | | 1120 | CG | 20.554 | −2.332 | 11.849 | C | PRO |
| | | 1121 | HG | 20.59 | −1.242 | 12.065 | H | PRO |
| | | 1122 | CD1 | 21.63 | −2.654 | 10.808 | C | PRO |
| | | 1123 | HD11 | 21.75 | −3.757 | 10.756 | H | PRO |
| | | 1124 | HD12 | 22.606 | −2.199 | 11.081 | H | PRO |
| | | 1125 | HD13 | 21.337 | −2.276 | 9.805 | H | PRO |
| | | 1126 | CD2 | 19.163 | −2.662 | 11.283 | C | PRO |
| | | 1127 | HD21 | 18.37 | −2.367 | 12.003 | H | PRO |
| | | 1128 | HD22 | 19.074 | −3.751 | 11.084 | H | PRO |
| | | 1129 | HD23 | 19.005 | −2.119 | 10.327 | H | PRO |
| | | 1130 | C | 22.549 | −1.666 | 14.161 | C | PRO |
| | | 1131 | O | 21.937 | −1.07 | 15.046 | O | PRO |
| 73 | ARG | 1132 | N | 23.582 | −1.104 | 13.499 | N | PRO |
| | | 1133 | HN | 24.046 | −1.597 | 12.768 | H | PRO |
| | | 1134 | CA | 24.128 | 0.2 | 13.792 | C | PRO |
| | | 1135 | HA | 23.434 | 0.785 | 14.378 | H | PRO |
| | | 1136 | CB | 25.521 | 0.109 | 14.479 | C | PRO |
| | | 1137 | HB1 | 26.036 | 1.093 | 14.452 | H | PRO |
| | | 1138 | HB2 | 26.145 | −0.603 | 13.898 | H | PRO |
| | | 1139 | CG | 25.495 | −0.373 | 15.946 | C | PRO |
| | | 1140 | HG1 | 26.532 | −0.638 | 16.243 | H | PRO |
| | | 1141 | HG2 | 24.887 | −1.301 | 16.007 | H | PRO |
| | | 1142 | CD | 24.945 | 0.645 | 16.959 | C | PRO |
| | | 1143 | HD1 | 24.915 | 0.219 | 17.984 | H | PRO |
| | | 1144 | HD2 | 23.914 | 0.948 | 16.676 | H | PRO |
| | | 1145 | NE | 25.798 | 1.882 | 16.936 | N | PRO |
| | | 1146 | HE | 25.569 | 2.596 | 16.274 | H | PRO |
| | | 1147 | CZ | 26.857 | 2.119 | 17.756 | C | PRO |
| | | 1148 | NH1 | 27.258 | 1.236 | 18.694 | N | PRO |
| | | 1149 | HH11 | 26.746 | 0.398 | 18.882 | H | PRO |
| | | 1150 | HH12 | 28.021 | 1.463 | 19.3 | H | PRO |
| | | 1151 | NH2 | 27.532 | 3.282 | 17.621 | N | PRO |
| | | 1152 | HH21 | 28.305 | 3.514 | 18.211 | H | PRO |
| | | 1153 | HH22 | 27.195 | 3.942 | 16.949 | H | PRO |
| | | 1154 | C | 24.338 | 0.904 | 12.478 | C | PRO |
| | | 1155 | O | 24.398 | 0.275 | 11.423 | O | PRO |
| 74 | GLY | 1156 | N | 24.483 | 2.249 | 12.531 | N | PRO |
| | | 1157 | HN | 24.388 | 2.72 | 13.405 | H | PRO |
| | | 1158 | CA | 25.019 | 3.045 | 11.446 | C | PRO |
| | | 1159 | HA1 | 26.011 | 2.668 | 11.245 | H | PRO |
| | | 1160 | HA2 | 25.04 | 4.066 | 11.797 | H | PRO |
| | | 1161 | C | 24.267 | 3.036 | 10.141 | C | PRO |
| | | 1162 | O | 24.893 | 3.185 | 9.094 | O | PRO |
| 75 | VAL | 1163 | N | 22.926 | 2.853 | 10.143 | N | PRO |
| | | 1164 | HN | 22.408 | 2.741 | 10.988 | H | PRO |
| | | 1165 | CA | 22.18 | 2.689 | 8.908 | C | PRO |
| | | 1166 | HA | 22.806 | 2.145 | 8.216 | H | PRO |
| | | 1167 | CB | 20.899 | 1.883 | 9.064 | C | PRO |
| | | 1168 | HB | 20.14 | 2.463 | 9.631 | H | PRO |
| | | 1169 | CG1 | 20.326 | 1.527 | 7.673 | C | PRO |
| | | 1170 | HG11 | 19.999 | 2.436 | 7.125 | H | PRO |
| | | 1171 | HG12 | 19.448 | 0.855 | 7.784 | H | PRO |
| | | 1172 | HG13 | 21.091 | 1.001 | 7.063 | H | PRO |
| | | 1173 | CG2 | 21.211 | 0.603 | 9.857 | C | PRO |
| | | 1174 | HG21 | 21.493 | 0.831 | 10.907 | H | PRO |
| | | 1175 | HG22 | 22.039 | 0.039 | 9.378 | H | PRO |
| | | 1176 | HG23 | 20.308 | −0.044 | 9.881 | H | PRO |
| | | 1177 | C | 21.85 | 4.031 | 8.299 | C | PRO |
| | | 1178 | O | 21.236 | 4.887 | 8.935 | O | PRO |
| 76 | ALA | 1179 | N | 22.259 | 4.24 | 7.03 | N | PRO |
| | | 1180 | HN | 22.752 | 3.541 | 6.518 | H | PRO |
| | | 1181 | CA | 22.007 | 5.475 | 6.338 | C | PRO |
| | | 1182 | HA | 21.038 | 5.861 | 6.62 | H | PRO |
| | | 1183 | CB | 23.094 | 6.526 | 6.613 | C | PRO |
| | | 1184 | HB1 | 24.103 | 6.111 | 6.402 | H | PRO |
| | | 1185 | HB2 | 23.057 | 6.829 | 7.682 | H | PRO |
| | | 1186 | HB3 | 22.938 | 7.431 | 5.988 | H | PRO |
| | | 1187 | C | 21.989 | 5.205 | 4.862 | C | PRO |
| | | 1188 | O | 22.64 | 4.284 | 4.378 | O | PRO |
| 77 | THR | 1189 | N | 21.234 | 6.035 | 4.114 | N | PRO |
| | | 1190 | HN | 20.685 | 6.748 | 4.542 | H | PRO |
| | | 1191 | CA | 21.187 | 6.012 | 2.671 | C | PRO |
| | | 1192 | HA | 22.012 | 5.43 | 2.286 | H | PRO |
| | | 1193 | CB | 19.882 | 5.469 | 2.103 | C | PRO |
| | | 1194 | HB | 19.021 | 6.003 | 2.56 | H | PRO |
| | | 1195 | OG1 | 19.767 | 4.085 | 2.411 | O | PRO |
| | | 1196 | HG1 | 18.86 | 3.845 | 2.207 | H | PRO |
| | | 1197 | CG2 | 19.811 | 5.599 | 0.571 | C | PRO |
| | | 1198 | HG21 | 20.67 | 5.082 | 0.092 | H | PRO |
| | | 1199 | HG22 | 19.811 | 6.664 | 0.254 | H | PRO |
| | | 1200 | HG23 | 18.873 | 5.137 | 0.195 | H | PRO |
| | | 1201 | C | 21.412 | 7.44 | 2.265 | C | PRO |
| | | 1202 | O | 20.878 | 8.367 | 2.873 | O | PRO |
| 78 | LEU | 1203 | N | 22.264 | 7.644 | 1.236 | N | PRO |
| | | 1204 | HN | 22.661 | 6.865 | 0.756 | H | PRO |
| | | 1205 | CA | 22.657 | 8.93 | 0.71 | C | PRO |
| | | 1206 | HA | 23.092 | 9.478 | 1.533 | H | PRO |
| | | 1207 | CB | 23.716 | 8.756 | −0.406 | C | PRO |
| | | 1208 | HB1 | 23.259 | 8.181 | −1.241 | H | PRO |
| | | 1209 | HB2 | 24.543 | 8.134 | −0.002 | H | PRO |
| | | 1210 | CG | 24.335 | 10.05 | −0.982 | C | PRO |
| | | 1211 | HG | 23.516 | 10.668 | −1.411 | H | PRO |
| | | 1212 | CD1 | 25.062 | 10.898 | 0.074 | C | PRO |
| | | 1213 | HD11 | 25.925 | 10.338 | 0.492 | H | PRO |
| | | 1214 | HD12 | 24.387 | 11.192 | 0.906 | H | PRO |
| | | 1215 | HD13 | 25.45 | 11.825 | −0.4 | H | PRO |
| | | 1216 | CD2 | 25.285 | 9.709 | −2.138 | C | PRO |
| | | 1217 | HD21 | 24.746 | 9.126 | −2.916 | H | PRO |
| | | 1218 | HD22 | 26.14 | 9.104 | −1.767 | H | PRO |
| | | 1219 | HD23 | 25.675 | 10.637 | −2.608 | H | PRO |
| | | 1220 | C | 21.481 | 9.723 | 0.188 | C | PRO |
| | | 1221 | O | 20.521 | 9.176 | −0.352 | O | PRO |
| 79 | GLU | 1222 | N | 21.539 | 11.061 | 0.365 | N | PRO |
| | | 1223 | HN | 22.327 | 11.481 | 0.808 | H | PRO |
| | | 1224 | CA | 20.54 | 12.001 | −0.078 | C | PRO |
| | | 1225 | HA | 19.606 | 11.713 | 0.381 | H | PRO |
| | | 1226 | CB | 20.901 | 13.432 | 0.387 | C | PRO |
| | | 1227 | HB1 | 20.063 | 14.12 | 0.145 | H | PRO |
| | | 1228 | HB2 | 21.805 | 13.783 | −0.156 | H | PRO |
| | | 1229 | CG | 21.186 | 13.486 | 1.905 | C | PRO |
| | | 1230 | HG1 | 22.074 | 12.869 | 2.161 | H | PRO |
| | | 1231 | HG2 | 20.307 | 13.077 | 2.448 | H | PRO |
| | | 1232 | CD | 21.454 | 14.904 | 2.39 | C | PRO |
| | | 1233 | OE1 | 22.43 | 15.535 | 1.906 | O | PRO |
| | | 1234 | OE2 | 20.702 | 15.368 | 3.291 | O | PRO |
| | | 1235 | C | 20.375 | 11.959 | −1.585 | C | PRO |
| | | 1236 | O | 21.352 | 11.982 | −2.334 | O | PRO |
| 80 | GLY | 1237 | N | 19.107 | 11.866 | −2.047 | N | PRO |
| | | 1238 | HN | 18.338 | 11.888 | −1.412 | H | PRO |
| | | 1239 | CA | 18.76 | 11.707 | −3.447 | C | PRO |
| | | 1240 | HA1 | 19.447 | 12.293 | −4.041 | H | PRO |
| | | 1241 | HA2 | 17.738 | 12.037 | −3.554 | H | PRO |
| | | 1242 | C | 18.825 | 10.281 | −3.936 | C | PRO |
| | | 1243 | O | 18.566 | 10.025 | −5.109 | O | PRO |
| 81 | TYR | 1244 | N | 19.151 | 9.312 | −3.05 | N | PRO |
| | | 1245 | HN | 19.439 | 9.541 | −2.123 | H | PRO |
| | | 1246 | CA | 19.151 | 7.894 | −3.365 | C | PRO |
| | | 1247 | HA | 18.775 | 7.729 | −4.364 | H | PRO |
| | | 1248 | CB | 20.554 | 7.248 | −3.218 | C | PRO |
| | | 1249 | HB1 | 20.498 | 6.142 | −3.307 | H | PRO |
| | | 1250 | HB2 | 21.003 | 7.499 | −2.233 | H | PRO |
| | | 1251 | CG | 21.464 | 7.743 | −4.308 | C | PRO |
| | | 1252 | CD1 | 21.589 | 7.022 | −5.509 | C | PRO |
| | | 1253 | HD1 | 21.03 | 6.108 | −5.647 | H | PRO |
| | | 1254 | CE1 | 22.437 | 7.476 | −6.528 | C | PRO |
| | | 1255 | HE1 | 22.531 | 6.914 | −7.445 | H | PRO |
| | | 1256 | CZ | 23.165 | 8.66 | −6.356 | C | PRO |
| | | 1257 | OH | 24.013 | 9.116 | −7.386 | O | PRO |
| | | 1258 | HH | 24.374 | 9.964 | −7.118 | H | PRO |
| | | 1259 | CD2 | 22.2 | 8.929 | −4.147 | C | PRO |
| | | 1260 | HD2 | 22.11 | 9.494 | −3.232 | H | PRO |
| | | 1261 | CE2 | 23.044 | 9.39 | −5.167 | C | PRO |
| | | 1262 | HE2 | 23.599 | 10.305 | −5.025 | H | PRO |
| | | 1263 | C | 18.203 | 7.197 | −2.418 | C | PRO |
| | | 1264 | O | 18.208 | 5.973 | −2.304 | O | PRO |
| 82 | ARG | 1265 | N | 17.35 | 7.973 | −1.716 | N | PRO |
| | | 1266 | HN | 17.34 | 8.96 | −1.86 | H | PRO |
| | | 1267 | CA | 16.401 | 7.473 | −0.751 | C | PRO |
| | | 1268 | HA | 16.803 | 6.58 | −0.295 | H | PRO |
| | | 1269 | CB | 16.129 | 8.496 | 0.371 | C | PRO |
| | | 1270 | HB1 | 15.317 | 8.125 | 1.032 | H | PRO |
| | | 1271 | HB2 | 15.795 | 9.458 | −0.073 | H | PRO |
| | | 1272 | CG | 17.373 | 8.732 | 1.241 | C | PRO |
| | | 1273 | HG1 | 18.199 | 9.092 | 0.592 | H | PRO |
| | | 1274 | HG2 | 17.682 | 7.759 | 1.679 | H | PRO |
| | | 1275 | CD | 17.144 | 9.744 | 2.365 | C | PRO |
| | | 1276 | HD1 | 16.366 | 9.386 | 3.073 | H | PRO |
| | | 1277 | HD2 | 16.845 | 10.726 | 1.94 | H | PRO |
| | | 1278 | NE | 18.442 | 9.881 | 3.095 | N | PRO |
| | | 1279 | HE | 19.143 | 9.178 | 2.976 | H | PRO |
| | | 1280 | CZ | 18.777 | 10.948 | 3.859 | C | PRO |
| | | 1281 | NH1 | 17.926 | 11.968 | 4.099 | N | PRO |
| | | 1282 | HH11 | 16.979 | 11.933 | 3.78 | H | PRO |
| | | 1283 | HH12 | 18.253 | 12.762 | 4.611 | H | PRO |
| | | 1284 | NH2 | 20.019 | 11.001 | 4.394 | N | PRO |
| | | 1285 | HH21 | 20.647 | 10.239 | 4.237 | H | PRO |
| | | 1286 | HH22 | 20.271 | 11.816 | 4.915 | H | PRO |
| | | 1287 | C | 15.1 | 7.106 | −1.419 | C | PRO |
| | | 1288 | O | 14.755 | 7.634 | −2.477 | O | PRO |
| 83 | GLU | 1289 | N | 14.375 | 6.147 | −0.798 | N | PRO |
| | | 1290 | HN | 14.705 | 5.77 | 0.064 | H | PRO |
| | | 1291 | CA | 13.117 | 5.584 | −1.253 | C | PRO |
| | | 1292 | HA | 12.861 | 4.857 | −0.497 | H | PRO |
| | | 1293 | CB | 11.938 | 6.594 | −1.346 | C | PRO |
| | | 1294 | HB1 | 11.033 | 6.081 | −1.735 | H | PRO |
| | | 1295 | HB2 | 12.203 | 7.394 | −2.07 | H | PRO |
| | | 1296 | CG | 11.557 | 7.248 | −0.003 | C | PRO |
| | | 1297 | HG1 | 10.734 | 7.976 | −0.166 | H | PRO |
| | | 1298 | HG2 | 12.43 | 7.793 | 0.416 | H | PRO |
| | | 1299 | CD | 11.083 | 6.199 | 0.995 | C | PRO |
| | | 1300 | OE1 | 11.69 | 6.118 | 2.094 | O | PRO |
| | | 1301 | OE2 | 10.102 | 5.465 | 0.683 | O | PRO |
| | | 1302 | C | 13.247 | 4.806 | −2.545 | C | PRO |
| | | 1303 | O | 12.314 | 4.738 | −3.345 | O | PRO |
| 84 | GLN | 1304 | N | 14.413 | 4.16 | −2.751 | N | PRO |
| | | 1305 | HN | 15.155 | 4.223 | −2.088 | H | PRO |
| | | 1306 | CA | 14.709 | 3.37 | −3.926 | C | PRO |
| | | 1307 | HA | 13.881 | 3.396 | −4.619 | H | PRO |
| | | 1308 | CB | 16.007 | 3.85 | −4.63 | C | PRO |
| | | 1309 | HB1 | 16.141 | 3.282 | −5.576 | H | PRO |
| | | 1310 | HB2 | 16.88 | 3.631 | −3.98 | H | PRO |
| | | 1311 | CG | 16.052 | 5.361 | −4.948 | C | PRO |
| | | 1312 | HG1 | 16.988 | 5.59 | −5.501 | H | PRO |
| | | 1313 | HG2 | 16.066 | 5.937 | −3.999 | H | PRO |
| | | 1314 | CD | 14.864 | 5.792 | −5.813 | C | PRO |
| | | 1315 | OE1 | 14.629 | 5.241 | −6.894 | O | PRO |
| | | 1316 | NE2 | 14.1 | 6.811 | −5.327 | N | PRO |
| | | 1317 | HE21 | 13.314 | 7.122 | −5.862 | H | PRO |
| | | 1318 | HE22 | 14.3 | 7.199 | −4.428 | H | PRO |
| | | 1319 | C | 14.923 | 1.938 | −3.497 | C | PRO |
| | | 1320 | O | 15.326 | 1.1 | −4.304 | O | PRO |
| 85 | LYS | 1321 | N | 14.664 | 1.642 | −2.199 | N | PRO |
| | | 1322 | HN | 14.327 | 2.355 | −1.588 | H | PRO |
| | | 1323 | CA | 14.837 | 0.356 | −1.561 | C | PRO |
| | | 1324 | HA | 14.505 | 0.498 | −0.544 | H | PRO |
| | | 1325 | CB | 13.983 | −0.784 | −2.174 | C | PRO |
| | | 1326 | HB1 | 14.177 | −1.728 | −1.621 | H | PRO |
| | | 1327 | HB2 | 14.273 | −0.939 | −3.235 | H | PRO |
| | | 1328 | CG | 12.467 | −0.538 | −2.097 | C | PRO |
| | | 1329 | HG1 | 11.952 | −1.366 | −2.63 | H | PRO |
| | | 1330 | HG2 | 12.211 | 0.411 | −2.615 | H | PRO |
| | | 1331 | CD | 11.967 | −0.502 | −0.646 | C | PRO |
| | | 1332 | HD1 | 12.385 | 0.39 | −0.133 | H | PRO |
| | | 1333 | HD2 | 12.358 | −1.398 | −0.118 | H | PRO |
| | | 1334 | CE | 10.447 | −0.494 | −0.484 | C | PRO |
| | | 1335 | HE1 | 10.218 | −0.467 | 0.603 | H | PRO |
| | | 1336 | HE2 | 9.979 | −1.394 | −0.937 | H | PRO |
| | | 1337 | NZ | 9.844 | 0.705 | −1.1 | N | PRO |
| | | 1338 | HZ1 | 9.759 | 0.578 | −2.128 | H | PRO |
| | | 1339 | HZ2 | 10.431 | 1.538 | −0.892 | H | PRO |
| | | 1340 | HZ3 | 8.903 | 0.862 | −0.684 | H | PRO |
| | | 1341 | C | 16.288 | −0.037 | −1.436 | C | PRO |
| | | 1342 | O | 16.626 | −1.222 | −1.437 | O | PRO |
| 86 | ALA | 1343 | N | 17.189 | 0.963 | −1.277 | N | PRO |
| | | 1344 | HN | 16.907 | 1.919 | −1.292 | H | PRO |
| | | 1345 | CA | 18.593 | 0.723 | −1.048 | C | PRO |
| | | 1346 | HA | 18.931 | −0.014 | −1.762 | H | PRO |
| | | 1347 | CB | 19.428 | 2.007 | −1.216 | C | PRO |
| | | 1348 | HB1 | 19.142 | 2.772 | −0.462 | H | PRO |
| | | 1349 | HB2 | 19.262 | 2.438 | −2.226 | H | PRO |
| | | 1350 | HB3 | 20.512 | 1.79 | −1.104 | H | PRO |
| | | 1351 | C | 18.827 | 0.174 | 0.333 | C | PRO |
| | | 1352 | O | 19.46 | −0.867 | 0.482 | O | PRO |
| 87 | GLY | 1353 | N | 18.249 | 0.822 | 1.374 | N | PRO |
| | | 1354 | HN | 17.723 | 1.657 | 1.233 | H | PRO |
| | | 1355 | CA | 18.458 | 0.447 | 2.758 | C | PRO |
| | | 1356 | HA1 | 17.977 | 1.2 | 3.365 | H | PRO |
| | | 1357 | HA2 | 19.521 | 0.377 | 2.934 | H | PRO |
| | | 1358 | C | 17.836 | −0.877 | 3.086 | C | PRO |
| | | 1359 | O | 18.407 | −1.662 | 3.839 | O | PRO |
| 88 | SER | 1360 | N | 16.66 | −1.174 | 2.485 | N | PRO |
| | | 1361 | HN | 16.182 | −0.473 | 1.962 | H | PRO |
| | | 1362 | CA | 15.978 | −2.452 | 2.587 | C | PRO |
| | | 1363 | HA | 15.788 | −2.647 | 3.632 | H | PRO |
| | | 1364 | CB | 14.644 | −2.494 | 1.807 | C | PRO |
| | | 1365 | HB1 | 14.219 | −3.52 | 1.815 | H | PRO |
| | | 1366 | HB2 | 14.799 | −2.182 | 0.752 | H | PRO |
| | | 1367 | OG | 13.688 | −1.636 | 2.413 | O | PRO |
| | | 1368 | HG1 | 13.999 | −0.74 | 2.267 | H | PRO |
| | | 1369 | C | 16.816 | −3.581 | 2.052 | C | PRO |
| | | 1370 | O | 16.834 | −4.664 | 2.631 | O | PRO |
| 89 | SER | 1371 | N | 17.55 | −3.345 | 0.938 | N | PRO |
| | | 1372 | HN | 17.544 | −2.447 | 0.505 | H | PRO |
| | | 1373 | CA | 18.399 | −4.348 | 0.326 | C | PRO |
| | | 1374 | HA | 17.785 | −5.213 | 0.123 | H | PRO |
| | | 1375 | CB | 19.052 | −3.878 | −1.002 | C | PRO |
| | | 1376 | HB1 | 19.653 | −4.707 | −1.434 | H | PRO |
| | | 1377 | HB2 | 19.725 | −3.011 | −0.828 | H | PRO |
| | | 1378 | OG | 18.071 | −3.516 | −1.968 | O | PRO |
| | | 1379 | HG1 | 17.76 | −2.639 | −1.732 | H | PRO |
| | | 1380 | C | 19.514 | −4.786 | 1.25 | C | PRO |
| | | 1381 | O | 19.752 | −5.981 | 1.391 | O | PRO |
| 90 | LEU | 1382 | N | 20.201 | −3.832 | 1.927 | N | PRO |
| | | 1383 | HN | 19.98 | −2.866 | 1.818 | H | PRO |
| | | 1384 | CA | 21.319 | −4.128 | 2.807 | C | PRO |
| | | 1385 | HA | 22.033 | −4.727 | 2.262 | H | PRO |
| | | 1386 | CB | 22.024 | −2.855 | 3.346 | C | PRO |
| | | 1387 | HB1 | 22.626 | −3.119 | 4.242 | H | PRO |
| | | 1388 | HB2 | 21.261 | −2.116 | 3.67 | H | PRO |
| | | 1389 | CG | 23.012 | −2.18 | 2.373 | C | PRO |
| | | 1390 | HG | 23.721 | −2.954 | 2.006 | H | PRO |
| | | 1391 | CD1 | 22.339 | −1.529 | 1.164 | C | PRO |
| | | 1392 | HD11 | 21.63 | −0.747 | 1.512 | H | PRO |
| | | 1393 | HD12 | 21.797 | −2.272 | 0.54 | H | PRO |
| | | 1394 | HD13 | 23.105 | −1.036 | 0.528 | H | PRO |
| | | 1395 | CD2 | 23.825 | −1.121 | 3.125 | C | PRO |
| | | 1396 | HD21 | 24.352 | −1.581 | 3.988 | H | PRO |
| | | 1397 | HD22 | 23.154 | −0.32 | 3.504 | H | PRO |
| | | 1398 | HD23 | 24.58 | −0.656 | 2.455 | H | PRO |
| | | 1399 | C | 20.91 | −4.909 | 4.029 | C | PRO |
| | | 1400 | O | 21.552 | −5.893 | 4.385 | O | PRO |
| 91 | VAL | 1401 | N | 19.829 | −4.478 | 4.711 | N | PRO |
| | | 1402 | HN | 19.307 | −3.684 | 4.408 | H | PRO |
| | | 1403 | CA | 19.415 | −5.06 | 5.971 | C | PRO |
| | | 1404 | HA | 20.309 | −5.185 | 6.564 | H | PRO |
| | | 1405 | CB | 18.503 | −4.149 | 6.78 | C | PRO |
| | | 1406 | HB | 18.271 | −4.623 | 7.757 | H | PRO |
| | | 1407 | CG1 | 19.263 | −2.839 | 7.088 | C | PRO |
| | | 1408 | HG11 | 19.49 | −2.278 | 6.156 | H | PRO |
| | | 1409 | HG12 | 20.217 | −3.056 | 7.616 | H | PRO |
| | | 1410 | HG13 | 18.643 | −2.185 | 7.738 | H | PRO |
| | | 1411 | CG2 | 17.18 | −3.885 | 6.039 | C | PRO |
| | | 1412 | HG21 | 16.533 | −4.788 | 6.044 | H | PRO |
| | | 1413 | HG22 | 17.364 | −3.582 | 4.986 | H | PRO |
| | | 1414 | HG23 | 16.638 | −3.054 | 6.539 | H | PRO |
| | | 1415 | C | 18.838 | −6.45 | 5.803 | C | PRO |
| | | 1416 | O | 19.073 | −7.316 | 6.641 | O | PRO |
| 92 | LYS | 1417 | N | 18.09 | −6.709 | 4.703 | N | PRO |
| | | 1418 | HN | 17.875 | −5.989 | 4.049 | H | PRO |
| | | 1419 | CA | 17.575 | −8.024 | 4.374 | C | PRO |
| | | 1420 | HA | 17.157 | −8.448 | 5.275 | H | PRO |
| | | 1421 | CB | 16.446 | −7.957 | 3.326 | C | PRO |
| | | 1422 | HB1 | 16.144 | −8.983 | 3.026 | H | PRO |
| | | 1423 | HB2 | 16.812 | −7.427 | 2.421 | H | PRO |
| | | 1424 | CG | 15.207 | −7.232 | 3.876 | C | PRO |
| | | 1425 | HG1 | 15.505 | −6.219 | 4.221 | H | PRO |
| | | 1426 | HG2 | 14.818 | −7.787 | 4.757 | H | PRO |
| | | 1427 | CD | 14.095 | −7.08 | 2.832 | C | PRO |
| | | 1428 | HD1 | 13.686 | −8.083 | 2.583 | H | PRO |
| | | 1429 | HD2 | 14.562 | −6.659 | 1.916 | H | PRO |
| | | 1430 | CE | 12.977 | −6.142 | 3.296 | C | PRO |
| | | 1431 | HE1 | 13.404 | −5.141 | 3.518 | H | PRO |
| | | 1432 | HE2 | 12.474 | −6.538 | 4.204 | H | PRO |
| | | 1433 | NZ | 11.954 | −5.96 | 2.241 | N | PRO |
| | | 1434 | HZ1 | 12.427 | −5.715 | 1.348 | H | PRO |
| | | 1435 | HZ2 | 11.307 | −5.198 | 2.527 | H | PRO |
| | | 1436 | HZ3 | 11.422 | −6.845 | 2.118 | H | PRO |
| | | 1437 | C | 18.666 | −8.964 | 3.91 | C | PRO |
| | | 1438 | O | 18.659 | −10.14 | 4.262 | O | PRO |
| 93 | HIS | 1439 | N | 19.664 | −8.455 | 3.146 | N | PRO |
| | | 1440 | HN | 19.623 | −7.511 | 2.828 | H | PRO |
| | | 1441 | CA | 20.856 | −9.189 | 2.751 | C | PRO |
| | | 1442 | HA | 20.542 | −10.082 | 2.231 | H | PRO |
| | | 1443 | CB | 21.736 | −8.331 | 1.809 | C | PRO |
| | | 1444 | HB1 | 21.993 | −7.376 | 2.315 | H | PRO |
| | | 1445 | HB2 | 21.122 | −8.073 | 0.919 | H | PRO |
| | | 1446 | ND1 | 24.191 | −9.02 | 1.993 | N | PRO |
| | | 1447 | HD1 | 24.327 | −8.737 | 2.942 | H | PRO |
| | | 1448 | CG | 23.006 | −8.954 | 1.29 | C | PRO |
| | | 1449 | CE1 | 25.116 | −9.546 | 1.155 | C | PRO |
| | | 1450 | HE1 | 26.155 | −9.706 | 1.446 | H | PRO |
| | | 1451 | NE2 | 24.613 | −9.826 | −0.031 | N | PRO |
| | | 1452 | CD2 | 23.283 | −9.455 | 0.057 | C | PRO |
| | | 1453 | HD2 | 22.633 | −9.57 | −0.802 | H | PRO |
| | | 1454 | C | 21.662 | −9.609 | 3.959 | C | PRO |
| | | 1455 | O | 22.188 | −10.716 | 4.023 | O | PRO |
| 94 | ALA | 1456 | N | 21.752 | −8.732 | 4.98 | N | PRO |
| | | 1457 | HN | 21.378 | −7.811 | 4.897 | H | PRO |
| | | 1458 | CA | 22.351 | −9.054 | 6.25 | C | PRO |
| | | 1459 | HA | 23.35 | −9.414 | 6.054 | H | PRO |
| | | 1460 | CB | 22.451 | −7.831 | 7.156 | C | PRO |
| | | 1461 | HB1 | 21.447 | −7.429 | 7.41 | H | PRO |
| | | 1462 | HB2 | 23.019 | −7.034 | 6.631 | H | PRO |
| | | 1463 | HB3 | 22.988 | −8.073 | 8.098 | H | PRO |
| | | 1464 | C | 21.613 | −10.135 | 6.995 | C | PRO |
| | | 1465 | O | 22.254 | −10.998 | 7.58 | O | PRO |
| 95 | GLU | 1466 | N | 20.257 | −10.155 | 6.97 | N | PRO |
| | | 1467 | HN | 19.74 | −9.429 | 6.523 | H | PRO |
| | | 1468 | CA | 19.467 | −11.224 | 7.561 | C | PRO |
| | | 1469 | HA | 19.739 | −11.289 | 8.604 | H | PRO |
| | | 1470 | CB | 17.938 | −11.006 | 7.471 | C | PRO |
| | | 1471 | HB1 | 17.419 | −11.928 | 7.812 | H | PRO |
| | | 1472 | HB2 | 17.648 | −10.825 | 6.414 | H | PRO |
| | | 1473 | CG | 17.434 | −9.849 | 8.351 | C | PRO |
| | | 1474 | HG1 | 17.929 | −8.9 | 8.054 | H | PRO |
| | | 1475 | HG2 | 17.674 | −10.054 | 9.416 | H | PRO |
| | | 1476 | CD | 15.924 | −9.704 | 8.206 | C | PRO |
| | | 1477 | OE1 | 15.209 | −10.709 | 8.46 | O | PRO |
| | | 1478 | OE2 | 15.453 | −8.592 | 7.843 | O | PRO |
| | | 1479 | C | 19.773 | −12.563 | 6.932 | C | PRO |
| | | 1480 | O | 19.846 | −13.564 | 7.635 | O | PRO |
| 96 | GLU | 1481 | N | 19.997 | −12.609 | 5.598 | N | PRO |
| | | 1482 | HN | 19.896 | −11.788 | 5.041 | H | PRO |
| | | 1483 | CA | 20.415 | −13.8 | 4.879 | C | PRO |
| | | 1484 | HA | 19.686 | −14.574 | 5.072 | H | PRO |
| | | 1485 | CB | 20.481 | −13.539 | 3.351 | C | PRO |
| | | 1486 | HB1 | 20.855 | −14.442 | 2.824 | H | PRO |
| | | 1487 | HB2 | 21.209 | −12.72 | 3.167 | H | PRO |
| | | 1488 | CG | 19.115 | −13.15 | 2.737 | C | PRO |
| | | 1489 | HG1 | 18.583 | −12.441 | 3.406 | H | PRO |
| | | 1490 | HG2 | 18.482 | −14.057 | 2.629 | H | PRO |
| | | 1491 | CD | 19.24 | −12.467 | 1.371 | C | PRO |
| | | 1492 | OE1 | 20.377 | −12.303 | 0.855 | O | PRO |
| | | 1493 | OE2 | 18.175 | −12.063 | 0.83 | O | PRO |
| | | 1494 | C | 21.767 | −14.305 | 5.361 | C | PRO |
| | | 1495 | O | 21.94 | −15.503 | 5.581 | O | PRO |
| 97 | ILE | 1496 | N | 22.745 | −13.389 | 5.585 | N | PRO |
| | | 1497 | HN | 22.582 | −12.428 | 5.373 | H | PRO |
| | | 1498 | CA | 24.051 | −13.696 | 6.154 | C | PRO |
| | | 1499 | HA | 24.478 | −14.501 | 5.575 | H | PRO |
| | | 1500 | CB | 25.004 | −12.494 | 6.091 | C | PRO |
| | | 1501 | HB | 24.51 | −11.616 | 6.561 | H | PRO |
| | | 1502 | CG2 | 26.31 | −12.776 | 6.876 | C | PRO |
| | | 1503 | HG21 | 26.786 | −13.709 | 6.505 | H | PRO |
| | | 1504 | HG22 | 26.117 | −12.887 | 7.965 | H | PRO |
| | | 1505 | HG23 | 27.032 | −11.94 | 6.765 | H | PRO |
| | | 1506 | CG1 | 25.292 | −12.137 | 4.61 | C | PRO |
| | | 1507 | HG11 | 24.33 | −11.987 | 4.075 | H | PRO |
| | | 1508 | HG12 | 25.803 | −12.996 | 4.125 | H | PRO |
| | | 1509 | CD1 | 26.137 | −10.87 | 4.421 | C | PRO |
| | | 1510 | HD1 | 27.144 | −10.983 | 4.877 | H | PRO |
| | | 1511 | HD2 | 25.632 | −9.997 | 4.885 | H | PRO |
| | | 1512 | HD3 | 26.277 | −10.664 | 3.338 | H | PRO |
| | | 1513 | C | 23.929 | −14.191 | 7.584 | C | PRO |
| | | 1514 | O | 24.548 | −15.181 | 7.962 | O | PRO |
| 98 | LEU | 1515 | N | 23.106 | −13.526 | 8.42 | N | PRO |
| | | 1516 | HN | 22.629 | −12.708 | 8.108 | H | PRO |
| | | 1517 | CA | 22.9 | −13.883 | 9.807 | C | PRO |
| | | 1518 | HA | 23.875 | −13.994 | 10.257 | H | PRO |
| | | 1519 | CB | 22.123 | −12.786 | 10.564 | C | PRO |
| | | 1520 | HB1 | 21.861 | −13.145 | 11.582 | H | PRO |
| | | 1521 | HB2 | 21.175 | −12.58 | 10.023 | H | PRO |
| | | 1522 | CG | 22.896 | −11.458 | 10.735 | C | PRO |
| | | 1523 | HG | 23.176 | −11.075 | 9.73 | H | PRO |
| | | 1524 | CD1 | 21.999 | −10.394 | 11.39 | C | PRO |
| | | 1525 | HD11 | 21.685 | −10.724 | 12.404 | H | PRO |
| | | 1526 | HD12 | 21.09 | −10.224 | 10.774 | H | PRO |
| | | 1527 | HD13 | 22.549 | −9.433 | 11.482 | H | PRO |
| | | 1528 | CD2 | 24.207 | −11.631 | 11.521 | C | PRO |
| | | 1529 | HD21 | 24.941 | −12.234 | 10.944 | H | PRO |
| | | 1530 | HD22 | 24.006 | −12.14 | 12.487 | H | PRO |
| | | 1531 | HD23 | 24.661 | −10.638 | 11.726 | H | PRO |
| | | 1532 | C | 22.214 | −15.223 | 9.971 | C | PRO |
| | | 1533 | O | 22.603 | −16.006 | 10.831 | O | PRO |
| 99 | ARG | 1534 | N | 21.211 | −15.539 | 9.12 | N | PRO |
| | | 1535 | HN | 20.874 | −14.862 | 8.47 | H | PRO |
| | | 1536 | CA | 20.561 | −16.836 | 9.043 | C | PRO |
| | | 1537 | HA | 20.202 | −17.085 | 10.03 | H | PRO |
| | | 1538 | CB | 19.361 | −16.816 | 8.068 | C | PRO |
| | | 1539 | HB1 | 19.047 | −17.853 | 7.82 | H | PRO |
| | | 1540 | HB2 | 19.67 | −16.316 | 7.125 | H | PRO |
| | | 1541 | CG | 18.133 | −16.096 | 8.656 | C | PRO |
| | | 1542 | HG1 | 18.442 | −15.109 | 9.062 | H | PRO |
| | | 1543 | HG2 | 17.751 | −16.703 | 9.504 | H | PRO |
| | | 1544 | CD | 17.021 | −15.874 | 7.623 | C | PRO |
| | | 1545 | HD1 | 16.73 | −16.837 | 7.152 | H | PRO |
| | | 1546 | HD2 | 17.371 | −15.168 | 6.84 | H | PRO |
| | | 1547 | NE | 15.823 | −15.332 | 8.349 | N | PRO |
| | | 1548 | HE | 15.6 | −15.735 | 9.237 | H | PRO |
| | | 1549 | CZ | 15.031 | −14.323 | 7.907 | C | PRO |
| | | 1550 | NH1 | 15.295 | −13.646 | 6.768 | N | PRO |
| | | 1551 | HH11 | 16.106 | −13.872 | 6.228 | H | PRO |
| | | 1552 | HH12 | 14.695 | −12.886 | 6.519 | H | PRO |
| | | 1553 | NH2 | 13.936 | −13.975 | 8.625 | N | PRO |
| | | 1554 | HH21 | 13.653 | −14.526 | 9.41 | H | PRO |
| | | 1555 | HH22 | 13.377 | −13.208 | 8.31 | H | PRO |
| | | 1556 | C | 21.519 | −17.932 | 8.624 | C | PRO |
| | | 1557 | O | 21.453 | −19.042 | 9.146 | O | PRO |
| 100 | LYS | 1558 | N | 22.458 | −17.633 | 7.694 | N | PRO |
| | | 1559 | HN | 22.454 | −16.74 | 7.251 | H | PRO |
| | | 1560 | CA | 23.527 | −18.523 | 7.275 | C | PRO |
| | | 1561 | HA | 23.067 | −19.441 | 6.94 | H | PRO |
| | | 1562 | CB | 24.33 | −17.91 | 6.1 | C | PRO |
| | | 1563 | HB1 | 24.852 | −16.989 | 6.439 | H | PRO |
| | | 1564 | HB2 | 23.609 | −17.608 | 5.311 | H | PRO |
| | | 1565 | CG | 25.366 | −18.852 | 5.467 | C | PRO |
| | | 1566 | HG1 | 24.836 | −19.766 | 5.125 | H | PRO |
| | | 1567 | HG2 | 26.114 | −19.147 | 6.233 | H | PRO |
| | | 1568 | CD | 26.101 | −18.208 | 4.28 | C | PRO |
| | | 1569 | HD1 | 26.585 | −17.273 | 4.634 | H | PRO |
| | | 1570 | HD2 | 25.355 | −17.923 | 3.507 | H | PRO |
| | | 1571 | CE | 27.182 | −19.095 | 3.641 | C | PRO |
| | | 1572 | HE1 | 27.982 | −19.327 | 4.377 | H | PRO |
| | | 1573 | HE2 | 27.63 | −18.575 | 2.767 | H | PRO |
| | | 1574 | NZ | 26.619 | −20.38 | 3.163 | N | PRO |
| | | 1575 | HZ1 | 27.334 | −20.896 | 2.611 | H | PRO |
| | | 1576 | HZ2 | 25.785 | −20.194 | 2.569 | H | PRO |
| | | 1577 | HZ3 | 26.34 | −20.957 | 3.982 | H | PRO |
| | | 1578 | C | 24.468 | −18.866 | 8.419 | C | PRO |
| | | 1579 | O | 24.924 | −20.001 | 8.54 | O | PRO |
| 101 | ARG | 1580 | N | 24.742 | −17.882 | 9.309 | N | PRO |
| | | 1581 | HN | 24.393 | −16.961 | 9.153 | H | PRO |
| | | 1582 | CA | 25.523 | −18.052 | 10.519 | C | PRO |
| | | 1583 | HA | 26.376 | −18.676 | 10.295 | H | PRO |
| | | 1584 | CB | 26.014 | −16.693 | 11.079 | C | PRO |
| | | 1585 | HB1 | 26.514 | −16.854 | 12.058 | H | PRO |
| | | 1586 | HB2 | 25.135 | −16.037 | 11.254 | H | PRO |
| | | 1587 | CG | 27.008 | −15.946 | 10.17 | C | PRO |
| | | 1588 | HG1 | 26.561 | −15.803 | 9.163 | H | PRO |
| | | 1589 | HG2 | 27.922 | −16.567 | 10.049 | H | PRO |
| | | 1590 | CD | 27.386 | −14.579 | 10.755 | C | PRO |
| | | 1591 | HD1 | 27.868 | −14.721 | 11.746 | H | PRO |
| | | 1592 | HD2 | 26.481 | −13.944 | 10.867 | H | PRO |
| | | 1593 | NE | 28.343 | −13.882 | 9.832 | N | PRO |
| | | 1594 | HE | 28.532 | −14.271 | 8.93 | H | PRO |
| | | 1595 | CZ | 29.035 | −12.772 | 10.2 | C | PRO |
| | | 1596 | NH1 | 28.864 | −12.219 | 11.422 | N | PRO |
| | | 1597 | HH11 | 28.211 | −12.63 | 12.059 | H | PRO |
| | | 1598 | HH12 | 29.401 | −11.419 | 11.686 | H | PRO |
| | | 1599 | NH2 | 29.916 | −12.211 | 9.341 | N | PRO |
| | | 1600 | HH21 | 29.992 | −12.546 | 8.402 | H | PRO |
| | | 1601 | HH22 | 30.506 | −11.459 | 9.634 | H | PRO |
| | | 1602 | C | 24.72 | −18.732 | 11.617 | C | PRO |
| | | 1603 | O | 25.282 | −19.167 | 12.621 | O | PRO |
| 102 | GLY | 1604 | N | 23.381 | −18.844 | 11.449 | N | PRO |
| | | 1605 | HN | 22.952 | −18.498 | 10.618 | H | PRO |
| | | 1606 | CA | 22.491 | −19.51 | 12.38 | C | PRO |
| | | 1607 | HA1 | 23.013 | −20.345 | 12.823 | H | PRO |
| | | 1608 | HA2 | 21.625 | −19.815 | 11.812 | H | PRO |
| | | 1609 | C | 22.011 | −18.619 | 13.489 | C | PRO |
| | | 1610 | O | 21.45 | −19.106 | 14.469 | O | PRO |
| 103 | ALA | 1611 | N | 22.236 | −17.291 | 13.381 | N | PRO |
| | | 1612 | HN | 22.642 | −16.919 | 12.549 | N | PRO |
| | | 1613 | CA | 21.885 | −16.313 | 14.386 | C | PRO |
| | | 1614 | HA | 22.344 | −16.65 | 15.304 | H | PRO |
| | | 1615 | CB | 22.442 | −14.918 | 14.06 | C | PRO |
| | | 1616 | HB1 | 21.977 | −14.528 | 13.13 | H | PRO |
| | | 1617 | HB2 | 23.541 | −14.976 | 13.905 | H | PRO |
| | | 1618 | HB3 | 22.242 | −14.204 | 14.888 | H | PRO |
| | | 1619 | C | 20.399 | −16.198 | 14.647 | C | PRO |
| | | 1620 | O | 19.568 | −16.346 | 13.751 | O | PRO |
| 104 | ASP | 1621 | N | 20.05 | −15.953 | 15.93 | N | PRO |
| | | 1622 | HN | 20.741 | −15.852 | 16.642 | H | PRO |
| | | 1623 | CA | 18.698 | −15.877 | 16.428 | C | PRO |
| | | 1624 | HA | 18.151 | −16.729 | 16.054 | H | PRO |
| | | 1625 | CB | 18.662 | −15.862 | 17.98 | C | PRO |
| | | 1626 | HB1 | 17.613 | −15.76 | 18.331 | H | PRO |
| | | 1627 | HB2 | 19.246 | −15.002 | 18.372 | H | PRO |
| | | 1628 | CG | 19.245 | −17.14 | 18.57 | C | PRO |
| | | 1629 | OD1 | 18.495 | −17.851 | 19.29 | O | PRO |
| | | 1630 | OD2 | 20.458 | −17.407 | 18.358 | O | PRO |
| | | 1631 | C | 17.985 | −14.629 | 15.974 | C | PRO |
| | | 1632 | O | 16.822 | −14.684 | 15.577 | O | PRO |
| 105 | MET | 1633 | N | 18.66 | −13.46 | 16.041 | N | PRO |
| | | 1634 | HN | 19.623 | −13.417 | 16.297 | H | PRO |
| | | 1635 | CA | 17.957 | −12.207 | 15.921 | C | PRO |
| | | 1636 | HA | 17.209 | −12.299 | 15.148 | H | PRO |
| | | 1637 | CB | 17.255 | −11.804 | 17.246 | C | PRO |
| | | 1638 | HB1 | 16.58 | −12.637 | 17.537 | H | PRO |
| | | 1639 | HB2 | 16.619 | −10.912 | 17.063 | H | PRO |
| | | 1640 | CG | 18.207 | −11.512 | 18.424 | C | PRO |
| | | 1641 | HG1 | 18.725 | −10.548 | 18.234 | H | PRO |
| | | 1642 | HG2 | 18.988 | −12.302 | 18.445 | H | PRO |
| | | 1643 | SD | 17.4 | −11.464 | 20.054 | S | PRO |
| | | 1644 | CE | 16.128 | −10.22 | 19.706 | C | PRO |
| | | 1645 | HE1 | 15.563 | −9.971 | 20.63 | H | PRO |
| | | 1646 | HE2 | 15.39 | −10.585 | 18.96 | H | PRO |
| | | 1647 | HE3 | 16.584 | −9.285 | 19.318 | H | PRO |
| | | 1648 | C | 18.878 | −11.097 | 15.503 | C | PRO |
| | | 1649 | O | 20.097 | −11.167 | 15.66 | O | PRO |
| 106 | ILE | 1650 | N | 18.257 | −10.027 | 14.962 | N | PRO |
| | | 1651 | HN | 17.268 | −10.031 | 14.836 | H | PRO |
| | | 1652 | CA | 18.882 | −8.768 | 14.646 | C | PRO |
| | | 1653 | HA | 19.885 | −8.767 | 15.045 | H | PRO |
| | | 1654 | CB | 18.946 | −8.479 | 13.14 | C | PRO |
| | | 1655 | HB | 19.565 | −9.283 | 12.689 | H | PRO |
| | | 1656 | CG2 | 17.551 | −8.575 | 12.482 | C | PRO |
| | | 1657 | HG21 | 16.882 | −7.771 | 12.857 | H | PRO |
| | | 1658 | HG22 | 17.075 | −9.558 | 12.687 | H | PRO |
| | | 1659 | HG23 | 17.642 | −8.457 | 11.381 | H | PRO |
| | | 1660 | CG1 | 19.628 | −7.133 | 12.786 | C | PRO |
| | | 1661 | HG11 | 19.046 | −6.292 | 13.221 | H | PRO |
| | | 1662 | HG12 | 19.593 | −7.018 | 11.681 | H | PRO |
| | | 1663 | CD1 | 21.089 | −7.028 | 13.23 | C | PRO |
| | | 1664 | HD1 | 21.663 | −7.923 | 12.908 | H | PRO |
| | | 1665 | HD2 | 21.155 | −6.941 | 14.336 | H | PRO |
| | | 1666 | HD3 | 21.567 | −6.129 | 12.786 | H | PRO |
| | | 1667 | C | 18.063 | −7.755 | 15.412 | C | PRO |
| | | 1668 | O | 16.835 | −7.802 | 15.41 | O | PRO |
| 107 | TRP | 1669 | N | 18.718 | −6.836 | 16.149 | N | PRO |
| | | 1670 | HN | 19.714 | −6.82 | 16.2 | H | PRO |
| | | 1671 | CA | 18.018 | −5.878 | 16.972 | C | PRO |
| | | 1672 | HA | 16.997 | −5.788 | 16.629 | H | PRO |
| | | 1673 | CB | 17.985 | −6.259 | 18.476 | C | PRO |
| | | 1674 | HB1 | 17.457 | −7.233 | 18.555 | H | PRO |
| | | 1675 | HB2 | 17.375 | −5.513 | 19.028 | H | PRO |
| | | 1676 | CG | 19.317 | −6.413 | 19.2 | C | PRO |
| | | 1677 | CD1 | 20.102 | −7.526 | 19.337 | C | PRO |
| | | 1678 | HD1 | 19.917 | −8.471 | 18.848 | H | PRO |
| | | 1679 | NE1 | 21.156 | −7.266 | 20.184 | N | PRO |
| | | 1680 | HE1 | 21.874 | −7.873 | 20.444 | H | PRO |
| | | 1681 | CE2 | 21.043 | −5.974 | 20.642 | C | PRO |
| | | 1682 | CD2 | 19.905 | −5.401 | 20.037 | C | PRO |
| | | 1683 | CE3 | 19.524 | −4.094 | 20.323 | C | PRO |
| | | 1684 | HE3 | 18.659 | −3.634 | 19.869 | H | PRO |
| | | 1685 | CZ3 | 20.284 | −3.376 | 21.257 | C | PRO |
| | | 1686 | HZ3 | 19.995 | −2.369 | 21.519 | H | PRO |
| | | 1687 | CZ2 | 21.817 | −5.251 | 21.542 | C | PRO |
| | | 1688 | HZ2 | 22.69 | −5.679 | 22.011 | H | PRO |
| | | 1689 | CH2 | 21.411 | −3.948 | 21.864 | C | PRO |
| | | 1690 | HH2 | 21.978 | −3.373 | 22.581 | H | PRO |
| | | 1691 | C | 18.632 | −4.531 | 16.752 | C | PRO |
| | | 1692 | O | 19.704 | −4.415 | 16.169 | O | PRO |
| 108 | CYS | 1693 | N | 17.924 | −3.457 | 17.151 | N | PRO |
| | | 1694 | HN | 17.06 | −3.543 | 17.641 | H | PRO |
| | | 1695 | CA | 18.331 | −2.126 | 16.789 | C | PRO |
| | | 1696 | HA | 19.408 | −2.078 | 16.719 | H | PRO |
| | | 1697 | CB | 17.669 | −1.729 | 15.438 | C | PRO |
| | | 1698 | HB1 | 16.576 | −1.597 | 15.586 | H | PRO |
| | | 1699 | HB2 | 17.788 | −2.588 | 14.744 | H | PRO |
| | | 1700 | SG | 18.385 | −0.259 | 14.644 | S | PRO |
| | | 1701 | HG1 | 19.6 | −0.768 | 14.5 | H | PRO |
| | | 1702 | C | 17.886 | −1.181 | 17.872 | C | PRO |
| | | 1703 | O | 16.783 | −1.304 | 18.394 | O | PRO |
| 109 | ASN | 1704 | N | 18.727 | −0.175 | 18.203 | N | PRO |
| | | 1705 | HN | 19.656 | −0.134 | 17.845 | H | PRO |
| | | 1706 | CA | 18.316 | 0.984 | 18.965 | C | PRO |
| | | 1707 | HA | 17.404 | 0.782 | 19.508 | H | PRO |
| | | 1708 | CB | 19.405 | 1.529 | 19.922 | C | PRO |
| | | 1709 | HB1 | 19.082 | 2.497 | 20.36 | H | PRO |
| | | 1710 | HB2 | 20.358 | 1.683 | 19.372 | H | PRO |
| | | 1711 | CG | 19.62 | 0.549 | 21.074 | C | PRO |
| | | 1712 | OD1 | 18.688 | −0.135 | 21.505 | O | PRO |
| | | 1713 | ND2 | 20.874 | 0.507 | 21.602 | N | PRO |
| | | 1714 | HD21 | 21.03 | −0.069 | 22.404 | H | PRO |
| | | 1715 | HD22 | 21.601 | 1.071 | 21.21 | H | PRO |
| | | 1716 | C | 18.031 | 2.042 | 17.938 | C | PRO |
| | | 1717 | O | 18.853 | 2.913 | 17.667 | O | PRO |
| 110 | ALA | 1718 | N | 16.841 | 1.952 | 17.314 | N | PRO |
| | | 1719 | HN | 16.196 | 1.238 | 17.576 | H | PRO |
| | | 1720 | CA | 16.361 | 2.865 | 16.31 | C | PRO |
| | | 1721 | HA | 17.124 | 2.951 | 15.55 | H | PRO |
| | | 1722 | CB | 15.061 | 2.345 | 15.678 | C | PRO |
| | | 1723 | HB1 | 14.26 | 2.262 | 16.443 | H | PRO |
| | | 1724 | HB2 | 15.231 | 1.336 | 15.246 | H | PRO |
| | | 1725 | HB3 | 14.708 | 3.02 | 14.869 | H | PRO |
| | | 1726 | C | 16.085 | 4.231 | 16.88 | C | PRO |
| | | 1727 | O | 15.731 | 4.363 | 18.047 | O | PRO |
| 111 | ARG | 1728 | N | 16.217 | 5.297 | 16.058 | N | PRO |
| | | 1729 | HN | 16.591 | 5.192 | 15.14 | H | PRO |
| | | 1730 | CA | 15.633 | 6.593 | 16.35 | C | PRO |
| | | 1731 | HA | 15.963 | 6.902 | 17.331 | H | PRO |
| | | 1732 | CB | 16.011 | 7.68 | 15.316 | C | PRO |
| | | 1733 | HB1 | 15.519 | 8.639 | 15.585 | H | PRO |
| | | 1734 | HB2 | 15.644 | 7.382 | 14.311 | H | PRO |
| | | 1735 | CG | 17.52 | 7.93 | 15.23 | C | PRO |
| | | 1736 | HG1 | 18.034 | 6.977 | 14.981 | H | PRO |
| | | 1737 | HG2 | 17.876 | 8.262 | 16.229 | H | PRO |
| | | 1738 | CD | 17.909 | 8.99 | 14.196 | C | PRO |
| | | 1739 | HD1 | 17.47 | 9.973 | 14.468 | H | PRO |
| | | 1740 | HD2 | 17.58 | 8.695 | 13.177 | H | PRO |
| | | 1741 | NE | 19.403 | 9.078 | 14.215 | N | PRO |
| | | 1742 | HE | 19.904 | 8.376 | 14.722 | H | PRO |
| | | 1743 | CZ | 20.103 | 10.145 | 13.756 | C | PRO |
| | | 1744 | NH1 | 19.506 | 11.16 | 13.095 | N | PRO |
| | | 1745 | HH11 | 18.543 | 11.096 | 12.834 | H | PRO |
| | | 1746 | HH12 | 20.05 | 11.967 | 12.863 | H | PRO |
| | | 1747 | NH2 | 21.438 | 10.2 | 13.969 | N | PRO |
| | | 1748 | HH21 | 21.897 | 9.451 | 14.446 | H | PRO |
| | | 1749 | HH22 | 21.974 | 10.981 | 13.648 | H | PRO |
| | | 1750 | C | 14.125 | 6.48 | 16.351 | C | PRO |
| | | 1751 | O | 13.571 | 5.66 | 15.625 | O | PRO |
| 112 | THR | 1752 | N | 13.408 | 7.312 | 17.142 | N | PRO |
| | | 1753 | HN | 13.838 | 7.955 | 17.771 | H | PRO |
| | | 1754 | CA | 11.95 | 7.311 | 17.123 | C | PRO |
| | | 1755 | HA | 11.615 | 6.284 | 17.126 | H | PRO |
| | | 1756 | CB | 11.314 | 8.007 | 18.32 | C | PRO |
| | | 1757 | HB | 10.209 | 8.006 | 18.213 | H | PRO |
| | | 1758 | OG1 | 11.76 | 9.354 | 18.45 | O | PRO |
| | | 1759 | HG1 | 11.216 | 9.736 | 19.142 | H | PRO |
| | | 1760 | CG2 | 11.675 | 7.232 | 19.602 | C | PRO |
| | | 1761 | HG21 | 12.772 | 7.238 | 19.779 | H | PRO |
| | | 1762 | HG22 | 11.336 | 6.177 | 19.517 | H | PRO |
| | | 1763 | HG23 | 11.176 | 7.687 | 20.485 | H | PRO |
| | | 1764 | C | 11.419 | 7.956 | 15.858 | C | PRO |
| | | 1765 | O | 10.275 | 7.733 | 15.47 | O | PRO |
| 113 | SER | 1766 | N | 12.27 | 8.747 | 15.167 | N | PRO |
| | | 1767 | HN | 13.183 | 8.935 | 15.519 | H | PRO |
| | | 1768 | CA | 11.978 | 9.36 | 13.891 | C | PRO |
| | | 1769 | HA | 10.947 | 9.68 | 13.885 | H | PRO |
| | | 1770 | CB | 12.894 | 10.593 | 13.659 | C | PRO |
| | | 1771 | HB1 | 12.695 | 11.338 | 14.459 | H | PRO |
| | | 1772 | HB2 | 12.668 | 11.068 | 12.68 | H | PRO |
| | | 1773 | OG | 14.277 | 10.24 | 13.708 | O | PRO |
| | | 1774 | HG1 | 14.769 | 11.04 | 13.907 | H | PRO |
| | | 1775 | C | 12.178 | 8.385 | 12.75 | C | PRO |
| | | 1776 | O | 11.71 | 8.625 | 11.639 | O | PRO |
| 114 | ALA | 1777 | N | 12.886 | 7.26 | 13.001 | N | PRO |
| | | 1778 | HN | 13.23 | 7.074 | 13.918 | H | PRO |
| | | 1779 | CA | 13.237 | 6.282 | 11.996 | C | PRO |
| | | 1780 | HA | 12.77 | 6.529 | 11.054 | H | PRO |
| | | 1781 | CB | 14.765 | 6.204 | 11.8 | C | PRO |
| | | 1782 | HB1 | 15.269 | 5.861 | 12.729 | H | PRO |
| | | 1783 | HB2 | 15.163 | 7.208 | 11.537 | H | PRO |
| | | 1784 | HB3 | 15.024 | 5.506 | 10.976 | H | PRO |
| | | 1785 | C | 12.738 | 4.919 | 12.408 | C | PRO |
| | | 1786 | O | 13.178 | 3.903 | 11.875 | O | PRO |
| 115 | SER | 1787 | N | 11.793 | 4.843 | 13.374 | N | PRO |
| | | 1788 | HN | 11.416 | 5.658 | 13.806 | H | PRO |
| | | 1789 | CA | 11.291 | 3.576 | 13.869 | C | PRO |
| | | 1790 | HA | 12.126 | 2.899 | 13.965 | H | PRO |
| | | 1791 | CB | 10.635 | 3.701 | 15.269 | C | PRO |
| | | 1792 | HB1 | 11.404 | 4.054 | 15.988 | H | PRO |
| | | 1793 | HB2 | 10.274 | 2.708 | 15.615 | H | PRO |
| | | 1794 | OG | 9.55 | 4.626 | 15.286 | O | PRO |
| | | 1795 | HG1 | 9.316 | 4.751 | 16.209 | H | PRO |
| | | 1796 | C | 10.318 | 2.955 | 12.896 | C | PRO |
| | | 1797 | O | 10.139 | 1.74 | 12.884 | O | PRO |
| 116 | GLY | 1798 | N | 9.709 | 3.779 | 12.01 | N | PRO |
| | | 1799 | HN | 9.863 | 4.764 | 12.036 | H | PRO |
| | | 1800 | CA | 8.777 | 3.324 | 11.006 | C | PRO |
| | | 1801 | HA1 | 8.26 | 4.199 | 10.639 | H | PRO |
| | | 1802 | HA2 | 8.111 | 2.602 | 11.455 | H | PRO |
| | | 1803 | C | 9.461 | 2.679 | 9.838 | C | PRO |
| | | 1804 | O | 8.838 | 1.89 | 9.135 | O | PRO |
| 117 | TYR | 1805 | N | 10.774 | 2.953 | 9.632 | N | PRO |
| | | 1806 | HN | 11.235 | 3.616 | 10.217 | H | PRO |
| | | 1807 | CA | 11.619 | 2.298 | 8.646 | C | PRO |
| | | 1808 | HA | 11.137 | 2.364 | 7.682 | H | PRO |
| | | 1809 | CB | 13.01 | 3.009 | 8.597 | C | PRO |
| | | 1810 | HB1 | 13.354 | 3.172 | 9.641 | H | PRO |
| | | 1811 | HB2 | 12.901 | 4.006 | 8.121 | H | PRO |
| | | 1812 | CG | 14.117 | 2.278 | 7.87 | C | PRO |
| | | 1813 | CD1 | 13.978 | 1.796 | 6.556 | C | PRO |
| | | 1814 | HD1 | 13.06 | 1.967 | 6.013 | H | PRO |
| | | 1815 | CE1 | 15.014 | 1.071 | 5.946 | C | PRO |
| | | 1816 | HE1 | 14.882 | 0.693 | 4.943 | H | PRO |
| | | 1817 | CZ | 16.207 | 0.842 | 6.646 | C | PRO |
| | | 1818 | OH | 17.25 | 0.082 | 6.086 | O | PRO |
| | | 1819 | HH | 16.94 | −0.285 | 5.254 | H | PRO |
| | | 1820 | CD2 | 15.329 | 2.056 | 8.545 | C | PRO |
| | | 1821 | HD2 | 15.456 | 2.431 | 9.55 | H | PRO |
| | | 1822 | CE2 | 16.369 | 1.349 | 7.937 | C | PRO |
| | | 1823 | HE2 | 17.287 | 1.174 | 8.478 | H | PRO |
| | | 1824 | C | 11.761 | 0.832 | 8.995 | C | PRO |
| | | 1825 | O | 11.611 | −0.044 | 8.147 | O | PRO |
| 118 | TYR | 1826 | N | 11.999 | 0.54 | 10.29 | N | PRO |
| | | 1827 | HN | 12.091 | 1.269 | 10.964 | H | PRO |
| | | 1828 | CA | 12.212 | −0.801 | 10.782 | C | PRO |
| | | 1829 | HA | 12.791 | −1.35 | 10.054 | H | PRO |
| | | 1830 | CB | 12.959 | −0.779 | 12.131 | C | PRO |
| | | 1831 | HB1 | 13.086 | −1.804 | 12.541 | H | PRO |
| | | 1832 | HB2 | 12.409 | −0.152 | 12.865 | H | PRO |
| | | 1833 | CG | 14.333 | −0.216 | 11.931 | C | PRO |
| | | 1834 | CD1 | 15.269 | −0.92 | 11.157 | C | PRO |
| | | 1835 | HD1 | 15.007 | −1.877 | 10.729 | H | PRO |
| | | 1836 | CE1 | 16.535 | −0.382 | 10.915 | C | PRO |
| | | 1837 | HE1 | 17.227 | −0.911 | 10.276 | H | PRO |
| | | 1838 | CZ | 16.896 | 0.845 | 11.48 | C | PRO |
| | | 1839 | OH | 18.182 | 1.366 | 11.234 | O | PRO |
| | | 1840 | HH | 18.276 | 2.173 | 11.745 | H | PRO |
| | | 1841 | CD2 | 14.695 | 1.024 | 12.478 | C | PRO |
| | | 1842 | HD2 | 13.976 | 1.577 | 13.064 | H | PRO |
| | | 1843 | CE2 | 15.976 | 1.552 | 12.266 | C | PRO |
| | | 1844 | HE2 | 16.236 | 2.508 | 12.695 | H | PRO |
| | | 1845 | C | 10.912 | −1.544 | 10.941 | C | PRO |
| | | 1846 | O | 10.888 | −2.769 | 11.017 | O | PRO |
| 119 | ARG | 1847 | N | 9.779 | −0.817 | 10.92 | N | PRO |
| | | 1848 | HN | 9.819 | 0.177 | 10.848 | H | PRO |
| | | 1849 | CA | 8.462 | −1.383 | 11.077 | C | PRO |
| | | 1850 | HA | 8.527 | −2.287 | 11.664 | H | PRO |
| | | 1851 | CB | 7.592 | −0.354 | 11.823 | C | PRO |
| | | 1852 | HB1 | 7.282 | 0.464 | 11.138 | H | PRO |
| | | 1853 | HB2 | 8.236 | 0.108 | 12.601 | H | PRO |
| | | 1854 | CG | 6.374 | −0.916 | 12.569 | C | PRO |
| | | 1855 | HG1 | 6.72 | −1.748 | 13.219 | H | PRO |
| | | 1856 | HG2 | 5.631 | −1.319 | 11.848 | H | PRO |
| | | 1857 | CD | 5.735 | 0.176 | 13.436 | C | PRO |
| | | 1858 | HD1 | 5.228 | 0.938 | 12.806 | H | PRO |
| | | 1859 | HD2 | 6.526 | 0.661 | 14.048 | H | PRO |
| | | 1860 | NE | 4.735 | −0.449 | 14.355 | N | PRO |
| | | 1861 | HE | 4.512 | −1.419 | 14.257 | H | PRO |
| | | 1862 | CZ | 4.213 | 0.203 | 15.424 | C | PRO |
| | | 1863 | NH1 | 4.539 | 1.481 | 15.716 | N | PRO |
| | | 1864 | HH11 | 5.298 | 1.927 | 15.241 | H | PRO |
| | | 1865 | HH12 | 4.217 | 1.855 | 16.586 | H | PRO |
| | | 1866 | NH2 | 3.371 | −0.456 | 16.253 | N | PRO |
| | | 1867 | HH21 | 3.227 | −1.436 | 16.11 | H | PRO |
| | | 1868 | HH22 | 3.081 | 0.001 | 17.094 | H | PRO |
| | | 1869 | C | 7.884 | −1.721 | 9.714 | C | PRO |
| | | 1870 | O | 6.82 | −2.328 | 9.605 | O | PRO |
| 120 | LYS | 1871 | N | 8.637 | −1.404 | 8.631 | N | PRO |
| | | 1872 | HN | 9.458 | −0.847 | 8.732 | H | PRO |
| | | 1873 | CA | 8.373 | −1.872 | 7.285 | C | PRO |
| | | 1874 | HA | 7.343 | −2.179 | 7.185 | H | PRO |
| | | 1875 | CB | 8.718 | −0.784 | 6.239 | C | PRO |
| | | 1876 | HB1 | 8.62 | −1.19 | 5.209 | H | PRO |
| | | 1877 | HB2 | 9.777 | −0.481 | 6.379 | H | PRO |
| | | 1878 | CG | 7.859 | 0.488 | 6.332 | C | PRO |
| | | 1879 | HG1 | 8.377 | 1.292 | 5.767 | H | PRO |
| | | 1880 | HG2 | 7.786 | 0.812 | 7.391 | H | PRO |
| | | 1881 | CD | 6.44 | 0.334 | 5.775 | C | PRO |
| | | 1882 | HD1 | 5.914 | −0.441 | 6.373 | H | PRO |
| | | 1883 | HD2 | 6.495 | −0.047 | 4.732 | H | PRO |
| | | 1884 | CE | 5.61 | 1.629 | 5.812 | C | PRO |
| | | 1885 | HE1 | 5.586 | 2.053 | 6.839 | H | PRO |
| | | 1886 | HE2 | 4.569 | 1.424 | 5.481 | H | PRO |
| | | 1887 | NZ | 6.17 | 2.659 | 4.904 | N | PRO |
| | | 1888 | HZ1 | 7.006 | 2.277 | 4.418 | H | PRO |
| | | 1889 | HZ2 | 6.447 | 3.515 | 5.426 | H | PRO |
| | | 1890 | HZ3 | 5.468 | 2.913 | 4.18 | H | PRO |
| | | 1891 | C | 9.267 | −3.063 | 7 | C | PRO |
| | | 1892 | O | 9.146 | −3.709 | 5.959 | O | PRO |
| 121 | LEU | 1893 | N | 10.174 | −3.398 | 7.948 | N | PRO |
| | | 1894 | HN | 10.257 | −2.851 | 8.778 | H | PRO |
| | | 1895 | CA | 11.112 | −4.494 | 7.838 | C | PRO |
| | | 1896 | HA | 11.073 | −4.928 | 6.85 | H | PRO |
| | | 1897 | CB | 12.55 | −4.012 | 8.145 | C | PRO |
| | | 1898 | HB1 | 13.251 | −4.873 | 8.148 | H | PRO |
| | | 1899 | HB2 | 12.565 | −3.56 | 9.16 | H | PRO |
| | | 1900 | CG | 13.089 | −2.959 | 7.15 | C | PRO |
| | | 1901 | HG | 12.331 | −2.154 | 7.038 | H | PRO |
| | | 1902 | CD1 | 14.366 | −2.3 | 7.696 | C | PRO |
| | | 1903 | HD11 | 15.1 | −3.076 | 8 | H | PRO |
| | | 1904 | HD12 | 14.138 | −1.666 | 8.58 | H | PRO |
| | | 1905 | HD13 | 14.824 | −1.65 | 6.92 | H | PRO |
| | | 1906 | CD2 | 13.34 | −3.549 | 5.754 | C | PRO |
| | | 1907 | HD21 | 12.393 | −3.93 | 5.316 | H | PRO |
| | | 1908 | HD22 | 14.071 | −4.383 | 5.813 | H | PRO |
| | | 1909 | HD23 | 13.745 | −2.768 | 5.076 | H | PRO |
| | | 1910 | C | 10.746 | −5.576 | 8.831 | C | PRO |
| | | 1911 | O | 11.393 | −6.622 | 8.889 | O | PRO |
| 122 | GLY | 1912 | N | 9.658 | −5.367 | 9.61 | N | PRO |
| | | 1913 | HN | 9.148 | −4.513 | 9.536 | H | PRO |
| | | 1914 | CA | 9.105 | −6.367 | 10.498 | C | PRO |
| | | 1915 | HA1 | 9.193 | −7.336 | 10.029 | H | PRO |
| | | 1916 | HA2 | 8.074 | −6.089 | 10.661 | H | PRO |
| | | 1917 | C | 9.761 | −6.435 | 11.849 | C | PRO |
| | | 1918 | O | 9.635 | −7.447 | 12.535 | O | PRO |
| 123 | PHE | 1919 | N | 10.474 | −5.367 | 12.277 | N | PRO |
| | | 1920 | HN | 10.593 | −4.56 | 11.704 | H | PRO |
| | | 1921 | CA | 10.98 | −5.246 | 13.631 | C | PRO |
| | | 1922 | HA | 11.292 | −6.218 | 13.985 | H | PRO |
| | | 1923 | CB | 12.137 | −4.219 | 13.811 | C | PRO |
| | | 1924 | HB1 | 12.327 | −4.067 | 14.895 | H | PRO |
| | | 1925 | HB2 | 11.83 | −3.245 | 13.375 | H | PRO |
| | | 1926 | CG | 13.456 | −4.606 | 13.183 | C | PRO |
| | | 1927 | CD1 | 13.607 | −4.742 | 11.791 | C | PRO |
| | | 1928 | HD1 | 12.756 | −4.61 | 11.138 | H | PRO |
| | | 1929 | CE1 | 14.855 | −5.031 | 11.227 | C | PRO |
| | | 1930 | HE1 | 14.957 | −5.132 | 10.157 | H | PRO |
| | | 1931 | CZ | 15.977 | −5.18 | 12.051 | C | PRO |
| | | 1932 | HZ | 16.939 | −5.404 | 11.615 | H | PRO |
| | | 1933 | CD2 | 14.598 | −4.748 | 13.995 | C | PRO |
| | | 1934 | HD2 | 14.513 | −4.623 | 15.065 | H | PRO |
| | | 1935 | CE2 | 15.848 | −5.039 | 13.437 | C | PRO |
| | | 1936 | HE2 | 16.713 | −5.151 | 14.074 | H | PRO |
| | | 1937 | C | 9.851 | −4.748 | 14.51 | C | PRO |
| | | 1938 | O | 8.984 | −4.001 | 14.055 | O | PRO |
| 124 | SER | 1939 | N | 9.849 | −5.15 | 15.8 | N | PRO |
| | | 1940 | HN | 10.557 | −5.765 | 16.139 | H | PRO |
| | | 1941 | CA | 8.842 | −4.758 | 16.765 | C | PRO |
| | | 1942 | HA | 8.128 | −4.089 | 16.306 | H | PRO |
| | | 1943 | CB | 8.081 | −5.949 | 17.396 | C | PRO |
| | | 1944 | HB1 | 7.344 | −5.58 | 18.141 | H | PRO |
| | | 1945 | HB2 | 8.789 | −6.633 | 17.911 | H | PRO |
| | | 1946 | OG | 7.382 | −6.68 | 16.392 | O | PRO |
| | | 1947 | HG1 | 6.526 | −6.898 | 16.767 | H | PRO |
| | | 1948 | C | 9.515 | −4.014 | 17.88 | C | PRO |
| | | 1949 | O | 10.59 | −4.396 | 18.333 | O | PRO |
| 125 | GLU | 1950 | N | 8.882 | −2.906 | 18.327 | N | PRO |
| | | 1951 | HN | 8.014 | −2.624 | 17.926 | H | PRO |
| | | 1952 | CA | 9.317 | −2.063 | 19.423 | C | PRO |
| | | 1953 | HA | 10.349 | −1.803 | 19.241 | H | PRO |
| | | 1954 | CB | 8.48 | −0.763 | 19.514 | C | PRO |
| | | 1955 | HB1 | 8.865 | −0.117 | 20.331 | H | PRO |
| | | 1956 | HB2 | 7.437 | −1.045 | 19.771 | H | PRO |
| | | 1957 | CG | 8.478 | 0.05 | 18.2 | C | PRO |
| | | 1958 | HG1 | 8.315 | −0.622 | 17.331 | H | PRO |
| | | 1959 | HG2 | 9.453 | 0.567 | 18.067 | H | PRO |
| | | 1960 | CD | 7.342 | 1.065 | 18.178 | C | PRO |
| | | 1961 | OE1 | 7.614 | 2.272 | 17.942 | O | PRO |
| | | 1962 | OE2 | 6.17 | 0.628 | 18.348 | O | PRO |
| | | 1963 | C | 9.245 | −2.774 | 20.757 | C | PRO |
| | | 1964 | O | 8.419 | −3.664 | 20.955 | O | PRO |
| 126 | GLN | 1965 | N | 10.121 | −2.382 | 21.706 | N | PRO |
| | | 1966 | HN | 10.819 | −1.695 | 21.52 | H | PRO |
| | | 1967 | CA | 10.137 | −2.947 | 23.031 | C | PRO |
| | | 1968 | HA | 9.153 | −3.319 | 23.277 | H | PRO |
| | | 1969 | CB | 11.186 | −4.08 | 23.171 | C | PRO |
| | | 1970 | HB1 | 12.203 | −3.662 | 23.017 | H | PRO |
| | | 1971 | HB2 | 11.003 | −4.808 | 22.353 | H | PRO |
| | | 1972 | CG | 11.12 | −4.832 | 24.517 | C | PRO |
| | | 1973 | HG1 | 10.118 | −5.297 | 24.63 | H | PRO |
| | | 1974 | HG2 | 11.278 | −4.128 | 25.361 | H | PRO |
| | | 1975 | CD | 12.171 | −5.945 | 24.574 | C | PRO |
| | | 1976 | OE1 | 12.074 | −6.949 | 23.862 | O | PRO |
| | | 1977 | NE2 | 13.186 | −5.766 | 25.465 | N | PRO |
| | | 1978 | HE21 | 13.865 | −6.489 | 25.587 | H | PRO |
| | | 1979 | HE22 | 13.244 | −4.918 | 25.992 | H | PRO |
| | | 1980 | C | 10.488 | −1.853 | 24.006 | C | PRO |
| | | 1981 | O | 11.37 | −1.035 | 23.747 | O | PRO |
| 127 | GLY | 1982 | N | 9.802 | −1.85 | 25.177 | N | PRO |
| | | 1983 | HN | 9.064 | −2.506 | 25.318 | H | PRO |
| | | 1984 | CA | 10.09 | −1.005 | 26.319 | C | PRO |
| | | 1985 | HA1 | 11.103 | −1.219 | 26.624 | H | PRO |
| | | 1986 | HA2 | 9.368 | −1.264 | 27.08 | H | PRO |
| | | 1987 | C | 9.983 | 0.478 | 26.093 | C | PRO |
| | | 1988 | O | 9.479 | 0.954 | 25.078 | O | PRO |
| 128 | GLU | 1989 | N | 10.436 | 1.244 | 27.107 | N | PRO |
| | | 1990 | HN | 10.806 | 0.825 | 27.932 | H | PRO |
| | | 1991 | CA | 10.427 | 2.688 | 27.125 | C | PRO |
| | | 1992 | HA | 9.421 | 2.99 | 26.874 | H | PRO |
| | | 1993 | CB | 10.782 | 3.269 | 28.518 | C | PRO |
| | | 1994 | HB1 | 10.692 | 4.376 | 28.486 | H | PRO |
| | | 1995 | HB2 | 11.838 | 3.026 | 28.761 | H | PRO |
| | | 1996 | CG | 9.894 | 2.753 | 29.672 | C | PRO |
| | | 1997 | HG1 | 10.154 | 3.296 | 30.606 | H | PRO |
| | | 1998 | HG2 | 10.068 | 1.669 | 29.838 | H | PRO |
| | | 1999 | CD | 8.415 | 2.983 | 29.378 | C | PRO |
| | | 2000 | OE1 | 8.026 | 4.156 | 29.13 | O | PRO |
| | | 2001 | OE2 | 7.649 | 1.983 | 29.388 | O | PRO |
| | | 2002 | C | 11.351 | 3.319 | 26.11 | C | PRO |
| | | 2003 | O | 12.376 | 2.754 | 25.728 | O | PRO |
| 129 | VAL | 2004 | N | 10.983 | 4.543 | 25.666 | N | PRO |
| | | 2005 | HN | 10.113 | 4.929 | 25.961 | H | PRO |
| | | 2006 | CA | 11.824 | 5.466 | 24.928 | C | PRO |
| | | 2007 | HA | 12.228 | 4.937 | 24.077 | H | PRO |
| | | 2008 | CB | 11.016 | 6.672 | 24.44 | C | PRO |
| | | 2009 | HB | 10.586 | 7.199 | 25.319 | H | PRO |
| | | 2010 | CG1 | 11.873 | 7.681 | 23.643 | C | PRO |
| | | 2011 | HG11 | 12.338 | 7.186 | 22.763 | H | PRO |
| | | 2012 | HG12 | 12.672 | 8.127 | 24.273 | H | PRO |
| | | 2013 | HG13 | 11.232 | 8.511 | 23.278 | H | PRO |
| | | 2014 | CG2 | 9.85 | 6.167 | 23.563 | C | PRO |
| | | 2015 | HG21 | 9.153 | 5.521 | 24.141 | H | PRO |
| | | 2016 | HG22 | 10.24 | 5.587 | 22.7 | H | PRO |
| | | 2017 | HG23 | 9.27 | 7.03 | 23.173 | H | PRO |
| | | 2018 | C | 12.98 | 5.91 | 25.813 | C | PRO |
| | | 2019 | O | 12.819 | 6.074 | 27.022 | O | PRO |
| 130 | PHE | 2020 | N | 14.18 | 6.107 | 25.227 | N | PRO |
| | | 2021 | HN | 14.308 | 5.964 | 24.248 | H | PRO |
| | | 2022 | CA | 15.348 | 6.559 | 25.95 | C | PRO |
| | | 2023 | HA | 15.032 | 7.129 | 26.811 | H | PRO |
| | | 2024 | C13 | 16.3 | 5.417 | 26.421 | C | PRO |
| | | 2025 | HB1 | 15.821 | 4.892 | 27.275 | H | PRO |
| | | 2026 | HB2 | 17.269 | 5.829 | 26.775 | H | PRO |
| | | 2027 | CG | 16.57 | 4.383 | 25.35 | C | PRO |
| | | 2028 | CD1 | 15.757 | 3.239 | 25.256 | C | PRO |
| | | 2029 | HD1 | 14.943 | 3.105 | 25.953 | H | PRO |
| | | 2030 | CE1 | 15.983 | 2.281 | 24.263 | C | PRO |
| | | 2031 | HE1 | 15.348 | 1.409 | 24.203 | H | PRO |
| | | 2032 | CZ | 17.032 | 2.45 | 23.353 | C | PRO |
| | | 2033 | HZ | 17.201 | 1.71 | 22.584 | H | PRO |
| | | 2034 | CD2 | 17.634 | 4.533 | 24.442 | C | PRO |
| | | 2035 | HD2 | 18.273 | 5.401 | 24.504 | H | PRO |
| | | 2036 | CE2 | 17.863 | 3.573 | 23.446 | C | PRO |
| | | 2037 | HE2 | 18.681 | 3.699 | 22.752 | H | PRO |
| | | 2038 | C | 16.069 | 7.511 | 25.037 | C | PRO |
| | | 2039 | O | 16.164 | 7.279 | 23.835 | O | PRO |
| 131 | ASP | 2040 | N | 16.573 | 8.637 | 25.583 | N | PRO |
| | | 2041 | HN | 16.521 | 8.821 | 26.562 | H | PRO |
| | | 2042 | CA | 17.094 | 9.712 | 24.771 | C | PRO |
| | | 2043 | HA | 16.803 | 9.579 | 23.74 | H | PRO |
| | | 2044 | CB | 16.621 | 11.115 | 25.237 | C | PRO |
| | | 2045 | HB1 | 17.146 | 11.903 | 24.656 | H | PRO |
| | | 2046 | HB2 | 16.852 | 11.258 | 26.315 | H | PRO |
| | | 2047 | CG | 15.12 | 11.319 | 25.03 | C | PRO |
| | | 2048 | OD1 | 14.408 | 10.37 | 24.61 | O | PRO |
| | | 2049 | OD2 | 14.659 | 12.467 | 25.276 | O | PRO |
| | | 2050 | C | 18.594 | 9.657 | 24.823 | C | PRO |
| | | 2051 | O | 19.191 | 9.571 | 25.895 | O | PRO |
| 132 | THR | 2052 | N | 19.233 | 9.698 | 23.634 | N | PRO |
| | | 2053 | HN | 18.73 | 9.784 | 22.778 | H | PRO |
| | | 2054 | CA | 20.671 | 9.613 | 23.507 | C | PRO |
| | | 2055 | HA | 21.133 | 9.492 | 24.476 | H | PRO |
| | | 2056 | CB | 21.126 | 8.457 | 22.621 | C | PRO |
| | | 2057 | HB | 20.72 | 8.568 | 21.593 | H | PRO |
| | | 2058 | OG1 | 20.646 | 7.224 | 23.141 | O | PRO |
| | | 2059 | HG1 | 20.919 | 6.548 | 22.515 | H | PRO |
| | | 2060 | CG2 | 22.665 | 8.388 | 22.57 | C | PRO |
| | | 2061 | HG21 | 23.081 | 8.287 | 23.596 | H | PRO |
| | | 2062 | HG22 | 23.092 | 9.302 | 22.104 | H | PRO |
| | | 2063 | HG23 | 22.994 | 7.515 | 21.968 | H | PRO |
| | | 2064 | C | 21.093 | 10.908 | 22.858 | C | PRO |
| | | 2065 | O | 20.798 | 11.087 | 21.678 | O | PRO |
| 133 | PRO | 2066 | N | 21.765 | 11.851 | 23.509 | N | PRO |
| | | 2067 | CD | 21.985 | 11.878 | 24.956 | C | PRO |
| | | 2068 | HD1 | 22.473 | 10.942 | 25.303 | H | PRO |
| | | 2069 | HD2 | 21.007 | 12.018 | 25.464 | H | PRO |
| | | 2070 | CA | 22.213 | 13.071 | 22.858 | C | PRO |
| | | 2071 | HA | 21.417 | 13.456 | 22.238 | H | PRO |
| | | 2072 | CB | 22.555 | 14.014 | 24.023 | C | PRO |
| | | 2073 | HB1 | 21.647 | 14.597 | 24.289 | H | PRO |
| | | 2074 | HB2 | 23.379 | 14.721 | 23.79 | H | PRO |
| | | 2075 | CG | 22.897 | 13.083 | 25.193 | C | PRO |
| | | 2076 | HG1 | 23.957 | 12.763 | 25.11 | H | PRO |
| | | 2077 | HG2 | 22.728 | 13.56 | 26.182 | H | PRO |
| | | 2078 | C | 23.434 | 12.761 | 22.005 | C | PRO |
| | | 2079 | O | 24.231 | 11.934 | 22.45 | O | PRO |
| 134 | PRO | 2080 | N | 23.641 | 13.311 | 20.813 | N | PRO |
| | | 2081 | CD | 24.966 | 13.17 | 20.2 | C | PRO |
| | | 2082 | HD1 | 25.057 | 12.172 | 19.72 | H | PRO |
| | | 2083 | HD2 | 25.757 | 13.294 | 20.97 | H | PRO |
| | | 2084 | CA | 22.911 | 14.445 | 20.258 | C | PRO |
| | | 2085 | HA | 22.29 | 14.953 | 20.981 | H | PRO |
| | | 2086 | CB | 24.043 | 15.316 | 19.696 | C | PRO |
| | | 2087 | HB1 | 24.497 | 15.893 | 20.529 | H | PRO |
| | | 2088 | HB2 | 23.711 | 16.028 | 18.911 | H | PRO |
| | | 2089 | CG | 25.067 | 14.302 | 19.178 | C | PRO |
| | | 2090 | HG1 | 24.762 | 13.933 | 18.175 | H | PRO |
| | | 2091 | HG2 | 26.089 | 14.734 | 19.118 | H | PRO |
| | | 2092 | C | 22.05 | 13.92 | 19.13 | C | PRO |
| | | 2093 | O | 21.752 | 14.667 | 18.2 | O | PRO |
| 135 | VAL | 2094 | N | 21.626 | 12.64 | 19.206 | N | PRO |
| | | 2095 | HN | 21.843 | 12.078 | 20 | H | PRO |
| | | 2096 | CA | 20.931 | 11.945 | 18.141 | C | PRO |
| | | 2097 | HA | 20.883 | 12.579 | 17.268 | H | PRO |
| | | 2098 | CB | 21.65 | 10.663 | 17.733 | C | PRO |
| | | 2099 | HB | 21.025 | 10.093 | 17.012 | H | PRO |
| | | 2100 | CG1 | 22.958 | 11.049 | 17.008 | C | PRO |
| | | 2101 | HG11 | 23.645 | 11.592 | 17.691 | H | PRO |
| | | 2102 | HG12 | 22.741 | 11.697 | 16.131 | H | PRO |
| | | 2103 | HG13 | 23.474 | 10.134 | 16.649 | H | PRO |
| | | 2104 | CG2 | 21.946 | 9.769 | 18.954 | C | PRO |
| | | 2105 | HG21 | 21.022 | 9.564 | 19.536 | H | PRO |
| | | 2106 | HG22 | 22.689 | 10.245 | 19.628 | H | PRO |
| | | 2107 | HG23 | 22.366 | 8.801 | 18.608 | H | PRO |
| | | 2108 | C | 19.494 | 11.678 | 18.551 | C | PRO |
| | | 2109 | O | 18.783 | 10.89 | 17.924 | O | PRO |
| 136 | GLY | 2110 | N | 19.026 | 12.397 | 19.599 | N | PRO |
| | | 2111 | HN | 19.66 | 12.983 | 20.098 | H | PRO |
| | | 2112 | CA | 17.648 | 12.451 | 20.045 | C | PRO |
| | | 2113 | HA1 | 17.057 | 12.711 | 19.179 | H | PRO |
| | | 2114 | HA2 | 17.619 | 13.207 | 20.816 | H | PRO |
| | | 2115 | C | 17.088 | 11.177 | 20.638 | C | PRO |
| | | 2116 | O | 17.817 | 10.223 | 20.912 | O | PRO |
| 137 | PRO | 2117 | N | 15.778 | 11.16 | 20.886 | N | PRO |
| | | 2118 | CD | 14.938 | 12.359 | 20.826 | C | PRO |
| | | 2119 | HD1 | 14.753 | 12.605 | 19.758 | H | PRO |
| | | 2120 | HD2 | 15.406 | 13.221 | 21.348 | H | PRO |
| | | 2121 | CA | 15.004 | 9.994 | 21.295 | C | PRO |
| | | 2122 | HA | 15.298 | 9.764 | 22.308 | H | PRO |
| | | 2123 | CB | 13.548 | 10.465 | 21.229 | C | PRO |
| | | 2124 | HB1 | 12.902 | 9.93 | 21.957 | H | PRO |
| | | 2125 | HB2 | 13.146 | 10.336 | 20.201 | H | PRO |
| | | 2126 | CG | 13.633 | 11.963 | 21.515 | C | PRO |
| | | 2127 | HG1 | 12.754 | 12.524 | 21.131 | H | PRO |
| | | 2128 | HG2 | 13.728 | 12.129 | 22.61 | H | PRO |
| | | 2129 | C | 15.223 | 8.74 | 20.477 | C | PRO |
| | | 2130 | O | 15.199 | 8.8 | 19.246 | O | PRO |
| 138 | HIS | G | N | 15.437 | 7.598 | 21.159 | N | PRO |
| | | 2132 | HN | 15.46 | 7.59 | 22.155 | H | PRO |
| | | 2133 | CA | 15.645 | 6.308 | 20.553 | C | PRO |
| | | 2134 | HA | 15.354 | 6.342 | 19.513 | H | PRO |
| | | 2135 | CB | 17.108 | 5.812 | 20.683 | C | PRO |
| | | 2136 | HB1 | 17.163 | 4.719 | 20.488 | H | PRO |
| | | 2137 | HB2 | 17.472 | 5.993 | 21.717 | H | PRO |
| | | 2138 | CD2 | 18.656 | 5.911 | 18.618 | C | PRO |
| | | 2139 | HD2 | 18.608 | 4.901 | 18.232 | H | PRO |
| | | 2140 | CG | 18.047 | 6.453 | 19.698 | C | PRO |
| | | 2141 | NE2 | 19.351 | 6.915 | 18.003 | N | PRO |
| | | 2142 | HE2 | 19.865 | 6.86 | 17.147 | H | PRO |
| | | 2143 | ND1 | 18.384 | 7.779 | 19.718 | N | PRO |
| | | 2144 | HD1 | 18.06 | 8.478 | 20.355 | H | PRO |
| | | 2145 | CE1 | 19.167 | 8.033 | 18.682 | C | PRO |
| | | 2146 | HE1 | 19.568 | 8.991 | 18.424 | H | PRO |
| | | 2147 | C | 14.733 | 5.323 | 21.236 | C | PRO |
| | | 2148 | O | 14.193 | 5.582 | 22.309 | O | PRO |
| 139 | ILE | 2149 | N | 14.53 | 4.156 | 20.596 | N | PRO |
| | | 2150 | HN | 14.968 | 3.968 | 19.72 | H | PRO |
| | | 2151 | CA | 13.626 | 3.133 | 21.055 | C | PRO |
| | | 2152 | HA | 13.573 | 3.178 | 22.133 | H | PRO |
| | | 2153 | CB | 12.222 | 3.289 | 20.45 | C | PRO |
| | | 2154 | HB | 11.833 | 4.271 | 20.796 | H | PRO |
| | | 2155 | CG2 | 12.276 | 3.351 | 18.904 | C | PRO |
| | | 2156 | HG21 | 12.647 | 2.393 | 18.48 | H | PRO |
| | | 2157 | HG22 | 12.932 | 4.176 | 18.551 | H | PRO |
| | | 2158 | HG23 | 11.256 | 3.53 | 18.502 | H | PRO |
| | | 2159 | CG1 | 11.208 | 2.221 | 20.926 | C | PRO |
| | | 2160 | HG11 | 11.546 | 1.213 | 20.602 | H | PRO |
| | | 2161 | HG12 | 10.24 | 2.415 | 20.415 | H | PRO |
| | | 2162 | CD1 | 10.959 | 2.217 | 22.436 | C | PRO |
| | | 2163 | HD1 | 10.617 | 3.216 | 22.781 | H | PRO |
| | | 2164 | HD2 | 11.877 | 1.944 | 22.999 | H | PRO |
| | | 2165 | HD3 | 10.173 | 1.473 | 22.687 | H | PRO |
| | | 2166 | C | 14.262 | 1.817 | 20.682 | C | PRO |
| | | 2167 | O | 14.827 | 1.667 | 19.6 | O | PRO |
| 140 | LEU | 2168 | N | 14.217 | 0.823 | 21.601 | N | PRO |
| | | 2169 | HN | 13.783 | 0.958 | 22.488 | H | PRO |
| | | 2170 | CA | 14.677 | −0.522 | 21.339 | C | PRO |
| | | 2171 | HA | 15.633 | −0.457 | 20.84 | H | PRO |
| | | 2172 | CB | 14.829 | −1.347 | 22.642 | C | PRO |
| | | 2173 | HB1 | 13.843 | −1.396 | 23.151 | H | PRO |
| | | 2174 | HB2 | 15.521 | −0.803 | 23.32 | H | PRO |
| | | 2175 | CG | 15.363 | −2.79 | 22.473 | C | PRO |
| | | 2176 | HG | 14.704 | −3.33 | 21.761 | H | PRO |
| | | 2177 | CD1 | 16.794 | −2.82 | 21.917 | C | PRO |
| | | 2178 | HD11 | 17.481 | −2.27 | 22.595 | H | PRO |
| | | 2179 | HD12 | 16.844 | −2.353 | 20.91 | H | PRO |
| | | 2180 | HD13 | 17.149 | −3.869 | 21.835 | H | PRO |
| | | 2181 | CD2 | 15.301 | −3.551 | 23.804 | C | PRO |
| | | 2182 | HD21 | 14.264 | −3.542 | 24.204 | H | PRO |
| | | 2183 | HD22 | 15.967 | −3.068 | 24.55 | H | PRO |
| | | 2184 | HD23 | 15.625 | −4.605 | 23.67 | H | PRO |
| | | 2185 | C | 13.695 | −1.214 | 20.428 | C | PRO |
| | | 2186 | O | 12.485 | −1.139 | 20.633 | O | PRO |
| 141 | MET | 2187 | N | 14.202 | −1.909 | 19.393 | N | PRO |
| | | 2188 | HN | 15.18 | −1.917 | 19.201 | H | PRO |
| | | 2189 | CA | 13.393 | −2.678 | 18.486 | C | PRO |
| | | 2190 | HA | 12.449 | −2.916 | 18.953 | H | PRO |
| | | 2191 | CB | 13.164 | −1.976 | 17.123 | C | PRO |
| | | 2192 | HB1 | 12.539 | −2.635 | 16.484 | H | PRO |
| | | 2193 | HB2 | 14.141 | −1.824 | 16.617 | H | PRO |
| | | 2194 | CG | 12.467 | −0.606 | 17.245 | C | PRO |
| | | 2195 | HG1 | 13.129 | 0.064 | 17.835 | H | PRO |
| | | 2196 | HG2 | 11.532 | −0.733 | 17.83 | H | PRO |
| | | 2197 | SD | 12.084 | 0.194 | 15.659 | S | PRO |
| | | 2198 | CE | 10.708 | −0.874 | 15.141 | C | PRO |
| | | 2199 | HE1 | 10.322 | −0.569 | 14.145 | H | PRO |
| | | 2200 | HE2 | 9.862 | −0.819 | 15.86 | H | PRO |
| | | 2201 | HE3 | 11.022 | −1.938 | 15.072 | H | PRO |
| | | 2202 | C | 14.136 | −3.962 | 18.246 | C | PRO |
| | | 2203 | O | 15.353 | −4.027 | 18.417 | O | PRO |
| 142 | TYR | 2204 | N | 13.413 | −5.028 | 17.847 | N | PRO |
| | | 2205 | HN | 12.422 | −4.981 | 17.752 | H | PRO |
| | | 2206 | CA | 14.012 | −6.325 | 17.643 | C | PRO |
| | | 2207 | HA | 15.029 | −6.188 | 17.308 | H | PRO |
| | | 2208 | CB | 14.023 | −7.217 | 18.925 | C | PRO |
| | | 2209 | HB1 | 14.539 | −6.675 | 19.746 | H | PRO |
| | | 2210 | HB2 | 14.588 | −8.151 | 18.719 | H | PRO |
| | | 2211 | CG | 12.64 | −7.599 | 19.407 | C | PRO |
| | | 2212 | CD1 | 11.897 | −6.744 | 20.237 | C | PRO |
| | | 2213 | HD1 | 12.329 | −5.811 | 20.568 | H | PRO |
| | | 2214 | CE1 | 10.584 | −7.071 | 20.604 | C | PRO |
| | | 2215 | HE1 | 10.011 | −6.398 | 21.224 | H | PRO |
| | | 2216 | CZ | 10.006 | −8.264 | 20.154 | C | PRO |
| | | 2217 | OH | 8.68 | −8.579 | 20.516 | O | PRO |
| | | 2218 | HH | 8.459 | −9.427 | 20.123 | H | PRO |
| | | 2219 | CD2 | 12.052 | −8.803 | 18.973 | C | PRO |
| | | 2220 | HD2 | 12.604 | −9.462 | 18.32 | H | PRO |
| | | 2221 | CE2 | 10.742 | −9.132 | 19.338 | C | PRO |
| | | 2222 | HE2 | 10.301 | −10.05 | 18.976 | H | PRO |
| | | 2223 | C | 13.279 | −7.024 | 16.53 | C | PRO |
| | | 2224 | O | 12.081 | −6.825 | 16.343 | O | PRO |
| 143 | LYS | 2225 | N | 13.996 | −7.874 | 15.771 | N | PRO |
| | | 2226 | HN | 14.987 | −7.95 | 15.853 | H | PRO |
| | | 2227 | CA | 13.394 | −8.778 | 14.829 | C | PRO |
| | | 2228 | HA | 12.339 | −8.888 | 15.034 | H | PRO |
| | | 2229 | CB | 13.611 | −8.332 | 13.367 | C | PRO |
| | | 2230 | HB1 | 14.699 | −8.291 | 13.147 | H | PRO |
| | | 2231 | HB2 | 13.218 | −7.298 | 13.257 | H | PRO |
| | | 2232 | CG | 12.908 | −9.213 | 12.326 | C | PRO |
| | | 2233 | HG1 | 11.811 | −9.142 | 12.486 | H | PRO |
| | | 2234 | HG2 | 13.207 | −10.274 | 12.464 | H | PRO |
| | | 2235 | CD | 13.266 | −8.798 | 10.893 | C | PRO |
| | | 2236 | HD1 | 14.365 | −8.903 | 10.77 | H | PRO |
| | | 2237 | HD2 | 13.018 | −7.726 | 10.74 | H | PRO |
| | | 2238 | CE | 12.587 | −9.656 | 9.821 | C | PRO |
| | | 2239 | HE1 | 12.752 | −10.737 | 10.02 | H | PRO |
| | | 2240 | HE2 | 12.995 | −9.409 | 8.818 | H | PRO |
| | | 2241 | NZ | 11.134 | −9.399 | 9.785 | N | PRO |
| | | 2242 | HZ1 | 10.994 | −8.407 | 9.507 | H | PRO |
| | | 2243 | HZ2 | 10.733 | −9.569 | 10.729 | H | PRO |
| | | 2244 | HZ3 | 10.69 | −10.031 | 9.088 | H | PRO |
| | | 2245 | C | 14.069 | −10.106 | 15.028 | C | PRO |
| | | 2246 | O | 15.285 | −10.225 | 14.892 | O | PRO |
| 144 | ARG | 2247 | N | 13.286 | −11.156 | 15.348 | N | PRO |
| | | 2248 | HN | 12.305 | −11.052 | 15.492 | H | PRO |
| | | 2249 | CA | 13.799 | −12.499 | 15.459 | C | PRO |
| | | 2250 | HA | 14.837 | −12.456 | 15.756 | H | PRO |
| | | 2251 | CB | 13.038 | −13.319 | 16.523 | C | PRO |
| | | 2252 | HB1 | 12.012 | −13.557 | 16.17 | H | PRO |
| | | 2253 | HB2 | 12.935 | −12.666 | 17.415 | H | PRO |
| | | 2254 | CG | 13.77 | −14.607 | 16.949 | C | PRO |
| | | 2255 | HG1 | 14.834 | −14.363 | 17.157 | H | PRO |
| | | 2256 | HG2 | 13.751 | −15.325 | 16.102 | H | PRO |
| | | 2257 | CD | 13.178 | −15.293 | 18.19 | C | PRO |
| | | 2258 | HD1 | 13.707 | −16.245 | 18.412 | H | PRO |
| | | 2259 | HD2 | 12.102 | −15.517 | 18.028 | H | PRO |
| | | 2260 | NE | 13.295 | −14.352 | 19.359 | N | PRO |
| | | 2261 | HE | 12.572 | −13.674 | 19.495 | H | PRO |
| | | 2262 | CZ | 14.391 | −14.244 | 20.152 | C | PRO |
| | | 2263 | NH1 | 15.427 | −15.109 | 20.093 | N | PRO |
| | | 2264 | HH11 | 15.376 | −15.936 | 19.534 | H | PRO |
| | | 2265 | HH12 | 16.188 | −14.982 | 20.729 | H | PRO |
| | | 2266 | NH2 | 14.463 | −13.235 | 21.052 | N | PRO |
| | | 2267 | HH21 | 13.749 | −12.536 | 21.103 | H | PRO |
| | | 2268 | HH22 | 15.284 | −13.173 | 21.62 | H | PRO |
| | | 2269 | C | 13.711 | −13.126 | 14.09 | C | PRO |
| | | 2270 | O | 12.676 | −13.058 | 13.429 | O | PRO |
| 145 | ILE | 2271 | N | 14.838 | −13.699 | 13.611 | N | PRO |
| | | 2272 | HN | 15.643 | −13.812 | 14.188 | H | PRO |
| | | 2273 | CA | 15.013 | −14.07 | 12.221 | C | PRO |
| | | 2274 | HA | 14.156 | −13.746 | 11.649 | H | PRO |
| | | 2275 | CB | 16.249 | −13.433 | 11.591 | C | PRO |
| | | 2276 | HB | 16.341 | −13.77 | 10.536 | H | PRO |
| | | 2277 | CG2 | 16.008 | −11.91 | 11.553 | C | PRO |
| | | 2278 | HG21 | 15.957 | −11.491 | 12.581 | H | PRO |
| | | 2279 | HG22 | 15.055 | −11.682 | 11.029 | H | PRO |
| | | 2280 | HG23 | 16.827 | −11.398 | 11.005 | H | PRO |
| | | 2281 | CG1 | 17.557 | −13.82 | 12.324 | C | PRO |
| | | 2282 | HG11 | 17.652 | −14.926 | 12.351 | H | PRO |
| | | 2283 | HG12 | 17.505 | −13.462 | 13.374 | H | PRO |
| | | 2284 | CD1 | 18.825 | −13.25 | 11.684 | C | PRO |
| | | 2285 | HD1 | 18.834 | −12.14 | 11.733 | H | PRO |
| | | 2286 | HD2 | 18.901 | −13.561 | 10.62 | H | PRO |
| | | 2287 | HD3 | 19.717 | −13.63 | 12.226 | H | PRO |
| | | 2288 | C | 15.075 | −15.568 | 12.088 | C | PRO |
| | | 2289 | O | 15.411 | −16.089 | 11.025 | O | PRO |
| 146 | THR | 2290 | N | 14.704 | −16.298 | 13.161 | N | PRO |
| | | 2291 | HN | 14.424 | −15.854 | 14.008 | H | PRO |
| | | 2292 | CA | 14.607 | −17.742 | 13.169 | C | PRO |
| | | 2293 | HA | 15.551 | −18.142 | 12.829 | H | PRO |
| | | 2294 | CB | 14.304 | −18.312 | 14.548 | C | PRO |
| | | 2295 | HB | 14.147 | −19.409 | 14.478 | H | PRO |
| | | 2296 | OG1 | 13.134 | −17.713 | 15.097 | O | PRO |
| | | 2297 | HG1 | 12.485 | −17.79 | 14.394 | H | PRO |
| | | 2298 | CG2 | 15.493 | −18.045 | 15.49 | C | PRO |
| | | 2299 | HG21 | 15.631 | −16.956 | 15.657 | H | PRO |
| | | 2300 | HG22 | 16.43 | −18.46 | 15.06 | H | PRO |
| | | 2301 | HG23 | 15.315 | −18.527 | 16.475 | H | PRO |
| | | 2302 | C | 13.5 | −18.208 | 12.201 | C | PRO |
| | | 2303 | OT1 | 13.822 | −18.993 | 11.271 | O | PRO |
| | | 2304 | OT2 | 12.33 | −17.769 | 12.37 | O | PRO |
| 150 | ACO | 2305 | N1A | 11.04 | 6.933 | 8.529 | N | LIG |
| | | 2306 | C2A | 10.012 | 6.106 | 8.286 | C | LIG |
| | | 2307 | H2 | 9.191 | 6.171 | 9 | H | LIG |
| | | 2308 | N3A | 9.839 | 5.208 | 7.303 | N | LIG |
| | | 2309 | C4A | 10.901 | 5.205 | 6.484 | C | LIG |
| | | 2310 | C5A | 12.029 | 6.007 | 6.613 | C | LIG |
| | | 2311 | C6A | 12.112 | 6.918 | 7.679 | C | LIG |
| | | 2312 | N6A | 13.221 | 7.701 | 7.816 | N | LIG |
| | | 2313 | H61 | 13.345 | 8.288 | 8.616 | H | LIG |
| | | 2314 | H62 | 14.01 | 7.566 | 7.217 | H | LIG |
| | | 2315 | N7A | 12.913 | 5.735 | 5.605 | N | LIG |
| | | 2316 | C8A | 12.347 | 4.799 | 4.894 | C | LIG |
| | | 2317 | H8 | 12.787 | 4.35 | 4.003 | H | LIG |
| | | 2318 | N9A | 11.115 | 4.421 | 5.372 | N | LIG |
| | | 2319 | C1B | 10.228 | 3.387 | 4.822 | C | LIG |
| | | 2320 | H1′ | 9.262 | 3.36 | 5.371 | H | LIG |
| | | 2321 | C4B | 10.637 | 1.327 | 3.764 | C | LIG |
| | | 2322 | H4′ | 10.152 | 0.358 | 4.012 | H | LIG |
| | | 2323 | O4B | 10.778 | 2.095 | 4.99 | O | LIG |
| | | 2324 | C2B | 9.989 | 3.58 | 3.353 | C | LIG |
| | | 2325 | H2′ | 10.909 | 3.927 | 2.836 | H | LIG |
| | | 2326 | O2B | 8.939 | 4.489 | 3.107 | O | LIG |
| | | 2327 | HO2′ | 9.044 | 4.803 | 2.206 | H | LIG |
| | | 2328 | C3B | 9.678 | 2.159 | 2.896 | C | LIG |
| | | 2329 | H3′ | 9.995 | 2.05 | 1.836 | H | LIG |
| | | 2330 | O3B | 8.301 | 1.788 | 3.097 | O | LIG |
| | | 2331 | P3B | 7.578 | 0.856 | 2.044 | P | LIG |
| | | 2332 | O7A | 8.285 | −0.438 | 1.953 | O | LIG |
| | | 2333 | O8A | 6.125 | 0.84 | 2.331 | O | LIG |
| | | 2334 | O9A | 7.74 | 1.578 | 0.646 | O | LIG |
| | | 2335 | C5B | 12.006 | 1.038 | 3.113 | C | LIG |
| | | 2336 | H5′1 | 12.588 | 0.402 | 3.814 | H | LIG |
| | | 2337 | H5′2 | 11.822 | 0.448 | 2.189 | H | LIG |
| | | 2338 | O5B | 12.75 | 2.23 | 2.821 | O | LIG |
| | | 2339 | P1A | 14.052 | 2.162 | 1.929 | P | LIG |
| | | 2340 | O1A | 13.744 | 2.724 | 0.594 | O | LIG |
| | | 2341 | O2A | 14.623 | 0.798 | 1.972 | O | LIG |
| | | 2342 | O3A | 15.017 | 3.134 | 2.713 | O | LIG |
| | | 2343 | P2A | 16.203 | 4.103 | 2.326 | P | LIG |
| | | 2344 | O4A | 15.647 | 5.389 | 1.856 | O | LIG |
| | | 2345 | O5A | 17.148 | 3.413 | 1.417 | O | LIG |
| | | 2346 | O6A | 16.94 | 4.307 | 3.699 | O | LIG |
| | | 2347 | CBP | 16.997 | 5.431 | 5.856 | C | LIG |
| | | 2348 | CCP | 16.215 | 4.525 | 4.915 | C | LIG |
| | | 2349 | H121 | 15.206 | 4.956 | 4.742 | H | LIG |
| | | 2350 | H122 | 16.08 | 3.545 | 5.42 | H | LIG |
| | | 2351 | CDP | 18.361 | 4.743 | 6.085 | C | LIG |
| | | 2352 | H131 | 18.203 | 3.693 | 6.412 | H | LIG |
| | | 2353 | H132 | 18.965 | 4.731 | 5.152 | H | LIG |
| | | 2354 | H133 | 18.935 | 5.265 | 6.88 | H | LIG |
| | | 2355 | CEP | 16.184 | 5.489 | 7.167 | C | LIG |
| | | 2356 | H141 | 16.068 | 4.464 | 7.58 | H | LIG |
| | | 2357 | H142 | 16.703 | 6.105 | 7.933 | H | LIG |
| | | 2358 | H143 | 15.17 | 5.906 | 6.991 | H | LIG |
| | | 2359 | CAP | 17.132 | 6.806 | 5.168 | C | LIG |
| | | 2360 | H10 | 17.505 | 6.637 | 4.135 | H | LIG |
| | | 2361 | OAP | 15.873 | 7.484 | 5.095 | O | LIG |
| | | 2362 | HO10 | 15.359 | 7.051 | 4.41 | H | LIG |
| | | 2363 | C9P | 18.166 | 7.681 | 5.841 | C | LIG |
| | | 2364 | O9P | 19.288 | 7.813 | 5.353 | O | LIG |
| | | 2365 | N8P | 17.811 | 8.32 | 6.973 | N | LIG |
| | | 2366 | HN8 | 16.897 | 8.209 | 7.354 | H | LIG |
| | | 2367 | C7P | 18.691 | 9.239 | 7.637 | C | LIG |
| | | 2368 | H71 | 19.317 | 9.777 | 6.893 | H | LIG |
| | | 2369 | H72 | 18.08 | 10.014 | 8.147 | H | LIG |
| | | 2370 | C6P | 19.622 | 8.562 | 8.652 | C | LIG |
| | | 2371 | HC1 | 20.17 | 7.782 | 8.144 | H | LIG |
| | | 2372 | HC2 | 20.26 | 9.323 | 9.075 | H | LIG |
| | | 2373 | C5P | 18.837 | 7.957 | 9.766 | C | LIG |
| | | 2374 | O5P | 17.969 | 8.6 | 10.352 | O | LIG |
| | | 2375 | N4P | 19.158 | 6.699 | 10.115 | N | LIG |
| | | 2376 | H4 | 19.842 | 6.168 | 9.621 | H | LIG |
| | | 2377 | C3P | 18.619 | 6.086 | 11.293 | C | LIG |
| | | 2378 | H31 | 17.543 | 5.86 | 11.137 | H | LIG |
| | | 2379 | H32 | 18.707 | 6.798 | 12.142 | H | LIG |
| | | 2380 | C2P | 19.38 | 4.799 | 11.634 | C | LIG |
| | | 2381 | H151 | 20.429 | 5.053 | 11.617 | H | LIG |
| | | 2382 | H152 | 19.144 | 4.071 | 10.872 | H | LIG |
| | | 2383 | S1P | 18.97 | 4.122 | 13.276 | S | LIG |
| | | 2384 | C | 20.392 | 3.069 | 13.565 | C | LIG |
| | | 2385 | O | 21.208 | 2.802 | 12.685 | O | LIG |
| | | 2386 | CH3 | 20.565 | 2.543 | 14.95 | C | LIG |
| | | 2387 | HB21 | 19.631 | 2.069 | 15.32 | H | LIG |
| | | 2388 | HB22 | 21.379 | 1.787 | 14.966 | H | LIG |
| | | 2389 | HB23 | 20.831 | 3.364 | 15.65 | H | LIG |
| 151 | GLF | 2390 | C | 25.252 | 4.742 | 15.152 | C | LIG |
| | | 2391 | OC2 | 26.209 | 4.893 | 15.957 | O | LIG |
| | | 2392 | OC1 | 24.621 | 3.656 | 15.041 | O | LIG |
| | | 2393 | C1 | 24.896 | 5.976 | 14.321 | C | LIG |
| | | 2394 | H11 | 25.719 | 6.155 | 13.597 | H | LIG |
| | | 2395 | H12 | 24.838 | 6.855 | 14.999 | H | LIG |
| | | 2396 | N | 23.617 | 5.914 | 13.542 | N | LIG |
| | | 2397 | HN1 | 23.675 | 5.136 | 12.854 | H | LIG |
| | | 2398 | HN2 | 23.522 | 6.82 | 13.039 | H | LIG |
| | | 2399 | C2 | 22.354 | 5.74 | 14.334 | C | LIG |
| | | 2400 | H21 | 21.515 | 5.771 | 13.606 | H | LIG |
| | | 2401 | H22 | 22.382 | 4.732 | 14.801 | H | LIG |
| | | 2402 | P | 22.04 | 7.038 | 15.677 | P | LIG |
| | | 2403 | OP2 | 22.736 | 8.214 | 15.118 | O | LIG |
| | | 2404 | OP1 | 20.566 | 7.091 | 15.65 | O | LIG |
| | | 2405 | OP3 | 22.639 | 6.379 | 16.858 | O | LIG |
| 161 | HOH | 2406 | OH2 | 16.789 | 3.914 | −1.162 | O | WAT |
| | | 2407 | H1 | 16.956 | 3.817 | −0.225 | H | WAT |
| | | 2408 | H2 | 17.392 | 4.603 | −1.443 | H | WAT |
| 162 | HOH | 2409 | OH2 | 34.813 | −5.787 | 4.916 | O | WAT |
| | | 2410 | H1 | 34.179 | −5.073 | 4.852 | H | WAT |
| | | 2411 | H2 | 35.636 | −5.406 | 4.607 | H | WAT |
| 163 | HOH | 2412 | OH2 | 27.594 | 2.743 | 8.984 | O | WAT |
| | | 2413 | H1 | 27.825 | 2.907 | 8.07 | H | WAT |
| | | 2414 | H2 | 26.653 | 2.912 | 9.028 | H | WAT |
| 164 | HOH | 2415 | OH2 | 41.145 | −1.022 | 8.832 | O | WAT |
| | | 2416 | H1 | 41.929 | −0.856 | 9.355 | H | WAT |
| | | 2417 | H2 | 40.797 | −0.152 | 8.639 | H | WAT |
| 165 | HOH | 2418 | OH2 | 35.185 | −1.464 | 0.428 | O | WAT |
| | | 2419 | H1 | 35.215 | −0.747 | 1.061 | H | WAT |
| | | 2420 | H2 | 35.701 | −1.151 | −0.315 | H | WAT |
| 166 | HOH | 2421 | OH2 | 29.313 | −1.078 | 14.048 | O | WAT |
| | | 2422 | H1 | 29.299 | −0.841 | 14.976 | H | WAT |
| | | 2423 | H2 | 30.138 | −0.719 | 13.721 | H | WAT |
| 167 | HOH | 2424 | OH2 | 29.387 | 5.58 | 3.736 | O | WAT |
| | | 2425 | H1 | 30.274 | 5.237 | 3.625 | H | WAT |
| | | 2426 | H2 | 29.108 | 5.817 | 2.852 | H | WAT |
| 168 | HOH | 2427 | OH2 | 35.502 | 6.703 | 3.149 | O | WAT |
| | | 2428 | H1 | 36.066 | 6.2 | 2.561 | H | WAT |
| | | 2429 | H2 | 35.557 | 6.243 | 3.987 | H | WAT |
| 170 | HOH | 2430 | OH2 | 35.225 | −3.907 | 10.358 | O | WAT |
| | | 2431 | H1 | 35.714 | −3.542 | 11.095 | H | WAT |
| | | 2432 | H2 | 34.453 | −3.346 | 10.281 | H | WAT |
| 171 | HOH | 2433 | OH2 | 11.214 | 3.019 | −0.149 | O | WAT |
| | | 2434 | H1 | 12.123 | 2.934 | 0.137 | H | WAT |
| | | 2435 | H2 | 10.93 | 3.864 | 0.201 | H | WAT |
| 172 | HOH | 2436 | OH2 | 21.489 | 0.176 | 17.393 | O | WAT |
| | | 2437 | H1 | 21.656 | −0.22 | 16.538 | H | WAT |
| | | 2438 | H2 | 21.616 | 1.115 | 17.251 | H | WAT |
| 173 | HOH | 2439 | OH2 | 14.012 | 7.121 | 2.955 | O | WAT |
| | | 2440 | H1 | 14.588 | 6.5 | 2.51 | H | WAT |
| | | 2441 | H2 | 13.127 | 6.825 | 2.74 | H | WAT |
| 174 | HOH | 2442 | OH2 | 35.5 | −6.442 | 14.3 | O | WAT |
| | | 2443 | H1 | 35.111 | −5.576 | 14.183 | H | WAT |
| | | 2444 | H2 | 34.934 | −6.876 | 14.938 | H | WAT |
| 175 | HOH | 2445 | OH2 | 38.131 | −5.29 | 6.33 | O | WAT |
| | | 2446 | H1 | 37.567 | −4.861 | 6.973 | H | WAT |
| | | 2447 | H2 | 37.767 | −5.034 | 5.483 | H | WAT |
| 176 | HOH | 2448 | OH2 | 29.977 | −3.271 | 12.378 | O | WAT |
| | | 2449 | H1 | 29.474 | −4.071 | 12.528 | H | WAT |
| | | 2450 | H2 | 29.678 | −2.668 | 13.058 | H | WAT |
| 177 | HOH | 2451 | OH2 | 13.274 | 0.129 | 25.376 | O | WAT |
| | | 2452 | H1 | 13.012 | 1.033 | 25.549 | H | WAT |
| | | 2453 | H2 | 12.601 | −0.207 | 24.784 | H | WAT |
| 178 | HOH | 2454 | OH2 | 32.356 | 4.138 | −3.187 | O | WAT |
| | | 2455 | H1 | 32.696 | 3.857 | −2.337 | H | WAT |
| | | 2456 | H2 | 33.115 | 4.111 | −3.77 | H | WAT |
| 179 | HOH | 2457 | OH2 | 37.973 | 0.268 | 1.793 | O | WAT |
| | | 2458 | H1 | 38.198 | 0.956 | 1.166 | H | WAT |
| | | 2459 | H2 | 37.045 | 0.409 | 1.98 | H | WAT |
| 180 | HOH | 2460 | OH2 | 19.387 | 17.718 | 2.993 | O | WAT |
| | | 2461 | H1 | 19.908 | 18.42 | 3.382 | H | WAT |
| | | 2462 | H2 | 19.943 | 16.941 | 3.059 | H | WAT |
| 181 | HOH | 2463 | OH2 | 40.36 | 4.068 | 9.526 | O | WAT |
| | | 2464 | H1 | 40.772 | 3.531 | 8.849 | H | WAT |
| | | 2465 | H2 | 39.784 | 3.465 | 9.994 | H | WAT |
| 182 | HOH | 2466 | OH2 | 30.958 | 9.003 | −1.334 | O | WAT |
| | | 2467 | H1 | 31.543 | 9.737 | −1.146 | H | WAT |
| | | 2468 | H2 | 30.498 | 8.846 | −0.51 | H | WAT |
| 183 | HOH | 2469 | OH2 | 27.913 | −10.85 | 0.383 | O | WAT |
| | | 2470 | H1 | 28.517 | −11.373 | 0.91 | H | WAT |
| | | 2471 | H2 | 27.247 | −11.473 | 0.093 | H | WAT |
| 184 | HOH | 2472 | OH2 | 10.44 | −10.788 | 16.002 | O | WAT |
| | | 2473 | H1 | 10.083 | −9.938 | 15.745 | H | WAT |
| | | 2474 | H2 | 9.725 | −11.406 | 15.849 | H | WAT |
| 185 | HOH | 2475 | OH2 | 9.653 | 6.669 | 11.482 | O | WAT |
| | | 2476 | H1 | 9.111 | 7.125 | 12.126 | H | WAT |
| | | 2477 | H2 | 10.307 | 7.316 | 11.217 | H | WAT |
| 186 | HOH | 2478 | OH2 | 30.887 | −15.014 | 16.886 | O | WAT |
| | | 2479 | H1 | 30.885 | −14.996 | 15.929 | H | WAT |
| | | 2480 | H2 | 30.126 | −14.49 | 17.137 | H | WAT |
| 187 | HOH | 2481 | OH2 | 28.724 | 6.574 | 1.126 | O | WAT |
| | | 2482 | H1 | 28.454 | 6.402 | 0.224 | H | WAT |
| | | 2483 | H2 | 29.034 | 7.48 | 1.116 | H | WAT |
| 188 | HOH | 2484 | OH2 | 22.139 | 14.64 | 15.436 | O | WAT |
| | | 2485 | H1 | 22.754 | 15.366 | 15.331 | H | WAT |
| | | 2486 | H2 | 21.923 | 14.642 | 16.369 | H | WAT |
| 189 | HOH | 2487 | OH2 | 21.376 | 13.362 | 13.046 | O | WAT |
| | | 2488 | H1 | 22.262 | 13.079 | 12.82 | H | WAT |
| | | 2489 | H2 | 21.485 | 13.872 | 13.849 | H | WAT |
| 190 | HOH | 2490 | OH2 | 24.158 | −7.787 | −4.312 | O | WAT |
| | | 2491 | H1 | 23.427 | −8.301 | −3.97 | H | WAT |
| | | 2492 | H2 | 24.875 | −7.953 | −3.7 | H | WAT |
| 191 | HOH | 2493 | OH2 | 8.366 | 3.947 | −0.811 | O | WAT |
| | | 2494 | H1 | 8.188 | 3.188 | −0.255 | H | WAT |
| | | 2495 | H2 | 8.973 | 4.482 | −0.299 | H | WAT |
| 192 | HOH | 2496 | OH2 | 35.873 | 1.702 | 13.193 | O | WAT |
| | | 2497 | H1 | 35.977 | 2.346 | 12.493 | H | WAT |
| | | 2498 | H2 | 34.927 | 1.573 | 13.262 | H | WAT |
| 193 | HOH | 2499 | OH2 | 32.296 | −10.503 | 9.719 | O | WAT |
| | | 2500 | H1 | 32.359 | −9.633 | 10.115 | H | WAT |
| | | 2501 | H2 | 33.13 | −10.923 | 9.93 | H | WAT |
| 194 | HOH | 2502 | OH2 | 9.275 | −8.442 | 15.099 | O | WAT |
| | | 2503 | H1 | 8.674 | −7.836 | 15.533 | H | WAT |
| | | 2504 | H2 | 9.367 | −8.095 | 14.212 | H | WAT |
| 195 | HOH | 2505 | OH2 | 36.644 | −2.72 | 12.56 | O | WAT |
| | | 2506 | H1 | 37.271 | −3.419 | 12.747 | H | WAT |
| | | 2507 | H2 | 35.905 | −2.898 | 13.142 | H | WAT |
| 196 | HOH | 2508 | OH2 | 16.946 | −6.561 | 8.787 | O | WAT |
| | | 2509 | H1 | 16.317 | −7.229 | 8.515 | H | WAT |
| | | 2510 | H2 | 17.739 | −6.761 | 8.29 | H | WAT |
| 198 | HOH | 2511 | OH2 | 5.863 | −2.048 | 17.748 | O | WAT |
| | | 2512 | H1 | 5.979 | −1.104 | 17.86 | H | WAT |
| | | 2513 | H2 | 5.573 | −2.357 | 18.606 | H | WAT |
| 199 | HOH | 2514 | OH2 | 36.67 | 4.794 | 1.343 | O | WAT |
| | | 2515 | H1 | 36.263 | 3.929 | 1.301 | H | WAT |
| | | 2516 | H2 | 37.56 | 4.657 | 1.018 | H | WAT |
| 200 | HOH | 2517 | OH2 | 25.141 | 0.637 | −10.805 | O | WAT |
| | | 2518 | H1 | 24.679 | 0.083 | −11.434 | H | WAT |
| | | 2519 | H2 | 25.917 | 0.131 | −10.564 | H | WAT |
| 201 | HOH | 2520 | OH2 | 33.622 | 3.411 | −0.745 | O | WAT |
| | | 2521 | H1 | 34.245 | 2.902 | −0.227 | H | WAT |
| | | 2522 | H2 | 33.596 | 4.267 | −0.317 | H | WAT |
| 202 | HOH | 2523 | OH2 | 12.494 | −18.125 | 9.085 | O | WAT |
| | | 2524 | H1 | 12.902 | −18.372 | 9.915 | H | WAT |
| | | 2525 | H2 | 11.684 | −18.634 | 9.055 | H | WAT |
| 203 | HOH | 2526 | OH2 | 8.429 | 5.585 | 26.917 | O | WAT |
| | | 2527 | H1 | 8.271 | 5.027 | 27.679 | H | WAT |
| | | 2528 | H2 | 8.068 | 6.436 | 27.164 | H | WAT |
| 204 | HOH | 2529 | OH2 | 22.038 | 16.076 | 5.555 | O | WAT |
| | | 2530 | H1 | 22.912 | 16.342 | 5.267 | H | WAT |
| | | 2531 | H2 | 21.561 | 15.901 | 4.744 | H | WAT |
| 205 | HOH | 2532 | OH2 | 29.107 | −10.468 | 21.404 | O | WAT |
| | | 2533 | H1 | 28.744 | −9.598 | 21.236 | H | WAT |
| | | 2534 | H2 | 28.364 | −10.98 | 21.724 | H | WAT |
| 206 | HOH | 2535 | OH2 | 16.028 | 10.706 | 17.387 | O | WAT |
| | | 2536 | H1 | 16.974 | 10.783 | 17.51 | H | WAT |
| | | 2537 | H2 | 15.76 | 10.023 | 18.001 | H | WAT |
| 207 | HOH | 2538 | OH2 | 20.235 | −10.597 | −1.245 | O | WAT |
| | | 2539 | H1 | 20.407 | −11.153 | −0.486 | H | WAT |
| | | 2540 | H2 | 19.446 | −10.968 | −1.64 | H | WAT |
| 208 | HOH | 2541 | OH2 | 15.83 | −17.772 | 19.095 | O | WAT |
| | | 2542 | H1 | 16.784 | −17.825 | 19.155 | H | WAT |
| | | 2543 | H2 | 15.535 | −18.681 | 19.142 | H | WAT |
| 209 | HOH | 2544 | OH2 | 24.926 | −1.602 | 22.21 | O | WAT |
| | | 2545 | H1 | 24.624 | −1.786 | 23.1 | H | WAT |
| | | 2546 | H2 | 24.537 | −2.294 | 21.676 | H | WAT |
| 213 | HOH | 2547 | OH2 | 36.509 | −3.612 | 7.913 | O | WAT |
| | | 2548 | H1 | 37.195 | −2.981 | 8.131 | H | WAT |
| | | 2549 | H2 | 36.094 | −3.815 | 8.751 | H | WAT |
| 214 | HOH | 2550 | OH2 | 27.276 | 12.664 | −2.33 | O | WAT |
| | | 2551 | H1 | 28.165 | 12.841 | −2.637 | H | WAT |
| | | 2552 | H2 | 27.368 | 11.906 | −1.753 | H | WAT |
| 215 | HOH | 2553 | OH2 | 20.572 | −12.563 | 26.669 | O | WAT |
| | | 2554 | H1 | 19.876 | −11.911 | 26.585 | H | WAT |
| | | 2555 | H2 | 21.143 | −12.217 | 27.355 | H | WAT |
| 216 | HOH | 2556 | OH2 | 5.907 | 1.819 | 9.427 | O | WAT |
| | | 2557 | H1 | 5.47 | 1.003 | 9.183 | H | WAT |
| | | 2558 | H2 | 6.825 | 1.679 | 9.193 | H | WAT |
| 217 | HOH | 2559 | OH2 | 22.149 | −11.115 | 28.513 | O | WAT |
| | | 2560 | H1 | 22.908 | −10.711 | 28.933 | H | WAT |
| | | 2561 | H2 | 21.76 | −10.412 | 27.993 | H | WAT |
| 218 | HOH | 2562 | OH2 | 22.19 | −18.948 | 17.096 | O | WAT |
| | | 2563 | H1 | 21.904 | −18.961 | 16.183 | H | WAT |
| | | 2564 | H2 | 21.562 | −18.374 | 17.535 | H | WAT |
| 220 | HOH | 2565 | OH2 | 15.824 | 12.223 | 14.985 | O | WAT |
| | | 2566 | H1 | 15.984 | 13.14 | 15.21 | H | WAT |
| | | 2567 | H2 | 15.907 | 11.754 | 15.815 | H | WAT |
| 222 | HOH | 2568 | OH2 | 11.877 | 11.24 | 24.623 | O | WAT |
| | | 2569 | H1 | 12.041 | 12.076 | 25.058 | H | WAT |
| | | 2570 | H2 | 12.738 | 10.826 | 24.567 | H | WAT |
| 223 | HOH | 2571 | OH2 | 29.323 | 1.475 | 20.596 | O | WAT |
| | | 2572 | H1 | 29.119 | 1.446 | 21.53 | H | WAT |
| | | 2573 | H2 | 30.12 | 2.003 | 20.54 | H | WAT |
| 224 | HOH | 2574 | OH2 | 12.211 | −15.668 | 10.49 | O | WAT |
| | | 2575 | H1 | 12.204 | −16.332 | 9.801 | H | WAT |
| | | 2576 | H2 | 12.216 | −16.168 | 11.306 | H | WAT |
| 225 | HOH | 2577 | OH2 | 30.474 | 12.507 | −2.571 | O | WAT |
| | | 2578 | H1 | 30.887 | 13.334 | −2.82 | H | WAT |
| | | 2579 | H2 | 31.008 | 12.18 | −1.848 | H | WAT |
| 226 | HOH | 2580 | OH2 | 12.651 | −11.066 | 21.257 | O | WAT |
| | | 2581 | H1 | 12.576 | −10.668 | 22.124 | H | WAT |
| | | 2582 | H2 | 12.248 | −10.431 | 20.665 | H | WAT |
| 227 | HOH | 2583 | OH2 | 30.53 | 11.546 | 14.482 | O | WAT |
| | | 2584 | H1 | 31.422 | 11.892 | 14.453 | H | WAT |
| | | 2585 | H2 | 30.003 | 12.205 | 14.031 | H | WAT |
| 228 | HOH | 2586 | OH2 | 13.098 | −7.784 | 6.79 | O | WAT |
| | | 2587 | H1 | 12.679 | −7.305 | 7.506 | H | WAT |
| | | 2588 | H2 | 13.95 | −8.043 | 7.141 | H | WAT |
| 229 | HOH | 2589 | OH2 | 24.411 | 13.654 | −2.2 | O | WAT |
| | | 2590 | H1 | 25.174 | 13.219 | −2.582 | H | WAT |
| | | 2591 | H2 | 23.66 | 13.196 | −2.578 | H | WAT |
| 230 | HOH | 2592 | OH2 | 27.344 | −0.918 | −9.896 | O | WAT |
| | | 2593 | H1 | 27.577 | −0.908 | −8.968 | H | WAT |
| | | 2594 | H2 | 26.92 | −1.767 | −10.027 | H | WAT |
| 231 | HOH | 2595 | OH2 | 4.691 | −6.714 | 17.197 | O | WAT |
| | | 2596 | H1 | 4.136 | −7.233 | 17.778 | H | WAT |
| | | 2597 | H2 | 4.1 | −6.067 | 16.814 | H | WAT |
| 233 | HOH | 2598 | OH2 | 10.234 | −3.891 | 3.369 | O | WAT |
| | | 2599 | H1 | 9.878 | −3.868 | 4.257 | H | WAT |
| | | 2600 | H2 | 9.928 | −3.077 | 2.967 | H | WAT |
| 235 | HOH | 2601 | OH2 | 18.09 | −0.61 | 24.997 | O | WAT |
| | | 2602 | H1 | 17.432 | 0.083 | 25.055 | H | WAT |
| | | 2603 | H2 | 17.778 | −1.288 | 25.597 | H | WAT |
| 236 | HOH | 2604 | OH2 | 34.845 | −16.879 | 23.334 | O | WAT |
| | | 2605 | H1 | 35.397 | −17.66 | 23.364 | H | WAT |
| | | 2606 | H2 | 34.385 | −16.878 | 24.173 | H | WAT |
| 237 | HOH | 2607 | OH2 | 19.469 | −3.358 | 31.026 | O | WAT |
| | | 2608 | H1 | 19.665 | −3.088 | 31.924 | H | WAT |
| | | 2609 | H2 | 18.661 | −2.894 | 30.808 | H | WAT |
| 238 | HOH | 2610 | OH2 | 17.672 | −9.485 | 0.279 | O | WAT |
| | | 2611 | H1 | 17.717 | −10.408 | 0.529 | H | WAT |
| | | 2612 | H2 | 18.559 | −9.271 | −0.011 | H | WAT |
| 239 | HOH | 2613 | OH2 | 4.023 | −0.501 | 3.536 | O | WAT |
| | | 2614 | H1 | 4.785 | −0.099 | 3.119 | H | WAT |
| | | 2615 | H2 | 3.359 | −0.53 | 2.848 | H | WAT |
| 240 | HOH | 2616 | OH2 | 7.895 | 6.955 | 1.249 | O | WAT |
| | | 2617 | H1 | 8.704 | 6.526 | 0.971 | H | WAT |
| | | 2618 | H2 | 8.181 | 7.631 | 1.864 | H | WAT |
| 241 | HOH | 2619 | OH2 | 17.113 | −1.831 | 30.619 | O | WAT |
| | | 2620 | H1 | 16.611 | −2.062 | 29.838 | H | WAT |
| | | 2621 | H2 | 16.916 | −0.905 | 30.766 | H | WAT |
| 243 | HOH | 2622 | OH2 | 33.448 | −3.901 | 20.921 | O | WAT |
| | | 2623 | H1 | 32.839 | −3.445 | 21.502 | H | WAT |
| | | 2624 | H2 | 33.673 | −3.253 | 20.254 | H | WAT |
| 244 | HOH | 2625 | OH2 | 13.364 | −11.417 | 6.742 | O | WAT |
| | | 2626 | H1 | 14.008 | −11.1 | 7.375 | H | WAT |
| | | 2627 | H2 | 13.129 | −10.643 | 6.231 | H | WAT |
| 246 | HOH | 2628 | OH2 | 30.851 | 10.449 | −6.52 | O | WAT |
| | | 2629 | H1 | 31.62 | 10.837 | −6.938 | H | WAT |
| | | 2630 | H2 | 30.252 | 10.266 | −7.244 | H | WAT |
| 247 | HOH | 2631 | OH2 | 25.761 | −12.445 | −0.491 | O | WAT |
| | | 2632 | H1 | 25.201 | −13.022 | −1.01 | H | WAT |
| | | 2633 | H2 | 25.258 | −11.635 | −0.406 | H | WAT |
| 248 | HOH | 2634 | OH2 | 23.679 | −3.419 | −10.747 | O | WAT |
| | | 2635 | H1 | 23.697 | −4.222 | −11.267 | H | WAT |
| | | 2636 | H2 | 24.566 | −3.34 | −10.396 | H | WAT |
| 249 | HOH | 2637 | OH2 | 26.39 | −1.205 | 19.797 | O | WAT |
| | | 2638 | H1 | 26.604 | −2.108 | 19.56 | H | WAT |
| | | 2639 | H2 | 25.98 | −1.274 | 20.659 | H | WAT |
| 250 | HOH | 2640 | OH2 | 32.833 | 6.779 | 22.434 | O | WAT |
| | | 2641 | H1 | 32.885 | 6.806 | 23.39 | H | WAT |
| | | 2642 | H2 | 32.796 | 5.846 | 22.222 | H | WAT |
| 251 | HOH | 2643 | OH2 | 29.42 | 12.89 | 1.075 | O | WAT |
| | | 2644 | H1 | 29.832 | 13.576 | 1.601 | H | WAT |
| | | 2645 | H2 | 29.868 | 12.085 | 1.336 | H | WAT |
| 252 | HOH | 2646 | OH2 | 4.484 | −3.2 | 15.593 | O | WAT |
| | | 2647 | H1 | 5.059 | −3.804 | 15.124 | H | WAT |
| | | 2648 | H2 | 5.017 | −2.878 | 16.32 | H | WAT |
| 253 | HOH | 2649 | OH2 | 11.543 | −12.897 | 10.873 | O | WAT |
| | | 2650 | H1 | 11.824 | −12.847 | 11.787 | H | WAT |
| | | 2651 | H2 | 11.589 | −13.828 | 10.659 | H | WAT |
| 254 | HOH | 2652 | OH2 | 30.78 | 12.827 | 3.834 | O | WAT |
| | | 2653 | H1 | 31.029 | 12.217 | 3.139 | H | WAT |
| | | 2654 | H2 | 30.282 | 12.293 | 4.454 | H | WAT |
| 255 | HOH | 2655 | OH2 | 27.82 | −9.343 | −2.076 | O | WAT |
| | | 2656 | H1 | 27.122 | −8.689 | −2.035 | H | WAT |
| | | 2657 | H2 | 27.771 | −9.799 | −1.236 | H | WAT |
| 256 | HOH | 2658 | OH2 | 13.566 | −2.454 | 26.667 | O | WAT |
| | | 2659 | H1 | 13.58 | −1.597 | 26.241 | H | WAT |
| | | 2660 | H2 | 14.318 | −2.439 | 27.26 | H | WAT |
| 257 | HOH | 2661 | OH2 | 9.966 | 3.116 | −3.122 | O | WAT |
| | | 2662 | H1 | 10.727 | 3.689 | −3.217 | H | WAT |
| | | 2663 | H2 | 9.442 | 3.525 | −2.432 | H | WAT |
| 259 | HOH | 2664 | OH2 | 28.422 | 10.794 | −0.582 | O | WAT |
| | | 2665 | H1 | 28.755 | 10.094 | −0.02 | H | WAT |
| | | 2666 | H2 | 28.809 | 11.593 | −0.226 | H | WAT |
| 260 | HOH | 2667 | OH2 | 38.343 | −1.619 | 8.383 | O | WAT |
| | | 2668 | H1 | 38.389 | −0.917 | 7.734 | H | WAT |
| | | 2669 | H2 | 39.214 | −1.636 | 8.781 | H | WAT |
| 261 | HOH | 2670 | OH2 | 14.286 | 9.859 | 5.376 | O | WAT |
| | | 2671 | H1 | 14.827 | 9.083 | 5.228 | H | WAT |
| | | 2672 | H2 | 13.46 | 9.515 | 5.716 | H | WAT |
| 262 | HOH | 2673 | OH2 | 24.09 | 16.742 | 3.594 | O | WAT |
| | | 2674 | H1 | 24.217 | 17.66 | 3.355 | H | WAT |
| | | 2675 | H2 | 23.403 | 16.433 | 3.003 | H | WAT |
| 263 | HOH | 2676 | OH2 | 26.762 | −15.545 | 20.796 | O | WAT |
| | | 2677 | H1 | 26.508 | −14.622 | 20.802 | H | WAT |
| | | 2678 | H2 | 25.997 | −16.006 | 21.141 | H | WAT |
| 264 | HOH | 2679 | OH2 | 40.63 | −1.688 | 6.004 | O | WAT |
| | | 2680 | H1 | 39.952 | −1.017 | 5.93 | H | WAT |
| | | 2681 | H2 | 40.975 | −1.585 | 6.891 | H | WAT |
| 265 | HOH | 2682 | OH2 | 34.286 | −6.379 | 9.266 | O | WAT |
| | | 2683 | H1 | 33.812 | −6.8 | 9.984 | H | WAT |
| | | 2684 | H2 | 34.674 | −5.598 | 9.661 | H | WAT |
| 266 | HOH | 2685 | OH2 | 20.357 | 10.328 | −7.235 | O | WAT |
| | | 2686 | H1 | 19.667 | 10.243 | −6.577 | H | WAT |
| | | 2687 | H2 | 20.966 | 9.616 | −7.038 | H | WAT |
| 267 | HOH | 2688 | OH2 | 18.227 | −18.104 | 12.003 | O | WAT |
| | | 2689 | H1 | 18.586 | −17.382 | 12.519 | H | WAT |
| | | 2690 | H2 | 18.667 | −18.884 | 12.342 | H | WAT |
| 268 | HOH | 2691 | OH2 | 20.034 | 13.755 | 5.419 | O | WAT |
| | | 2692 | H1 | 20.36 | 14.263 | 6.161 | H | WAT |
| | | 2693 | H2 | 20.218 | 14.301 | 4.655 | H | WAT |
| 270 | HOH | 2694 | OH2 | 36.706 | 1.849 | 15.875 | O | WAT |
| | | 2695 | H1 | 35.89 | 1.555 | 16.279 | H | WAT |
| | | 2696 | H2 | 36.542 | 1.793 | 14.934 | H | WAT |
| 271 | HOH | 2697 | OH2 | 15.08 | 11.962 | 3.717 | O | WAT |
| | | 2698 | H1 | 14.304 | 12.467 | 3.475 | H | WAT |
| | | 2699 | H2 | 14.753 | 11.293 | 4.319 | H | WAT |
| 272 | HOH | 2700 | OH2 | 17.001 | 10.878 | 11.588 | O | WAT |
| | | 2701 | H1 | 17.272 | 10.11 | 11.084 | H | WAT |
| | | 2702 | H2 | 16.52 | 11.419 | 10.961 | H | WAT |
| 273 | HOH | 2703 | OH2 | 35.89 | −10.565 | 14.418 | O | WAT |
| | | 2704 | H1 | 36.608 | −11.168 | 14.229 | H | WAT |
| | | 2705 | H2 | 36.27 | −9.694 | 14.301 | H | WAT |
| 274 | HOH | 2706 | OH2 | 15.973 | 0.784 | −6.976 | O | WAT |
| | | 2707 | H1 | 15.769 | 0.794 | −6.041 | H | WAT |
| | | 2708 | H2 | 15.352 | 1.402 | −7.364 | H | WAT |
| 275 | HOH | 2709 | OH2 | 30.397 | −12.223 | 6.475 | O | WAT |
| | | 2710 | H1 | 31.064 | −11.544 | 6.577 | H | WAT |
| | | 2711 | H2 | 30.068 | −12.103 | 5.584 | H | WAT |
| 276 | HOH | 2712 | OH2 | 7.415 | 5.162 | 5.572 | O | WAT |
| | | 2713 | H1 | 7.869 | 5.21 | 4.731 | H | WAT |
| | | 2714 | H2 | 8.103 | 5.288 | 6.225 | H | WAT |
| 277 | HOH | 2715 | OH2 | 8.492 | −6.434 | 6.394 | O | WAT |
| | | 2716 | H1 | 8.677 | −5.515 | 6.197 | H | WAT |
| | | 2717 | H2 | 7.624 | −6.589 | 6.022 | H | WAT |
| 278 | HOH | 2718 | OH2 | 32.214 | −9.982 | 6.865 | O | WAT |
| | | 2719 | H1 | 32.276 | −10.185 | 7.799 | H | WAT |
| | | 2720 | H2 | 32.784 | −9.222 | 6.747 | H | WAT |
| 279 | HOH | 2721 | OH2 | 24.674 | −7.171 | 27.31 | O | WAT |
| | | 2722 | H1 | 24.695 | −7.618 | 26.464 | H | WAT |
| | | 2723 | H2 | 25.589 | −6.951 | 27.488 | H | WAT |
| 280 | HOH | 2724 | OH2 | 13.514 | −5.774 | −0.219 | O | WAT |
| | | 2725 | H1 | 13.99 | −5.102 | −0.708 | H | WAT |
| | | 2726 | H2 | 14.069 | −6.551 | −0.277 | H | WAT |
| 281 | HOH | 2727 | OH2 | 5.265 | 3.108 | 28.822 | O | WAT |
| | | 2728 | H1 | 6.008 | 2.546 | 29.039 | H | WAT |
| | | 2729 | H2 | 5.635 | 3.99 | 28.776 | H | WAT |
| 282 | HOH | 2730 | OH2 | 20.729 | −1.117 | 24.118 | O | WAT |
| | | 2731 | H1 | 20.809 | −1.955 | 24.574 | H | WAT |
| | | 2732 | H2 | 19.85 | −0.807 | 24.336 | H | WAT |
| 283 | HOH | 2733 | OH2 | 32.867 | −6.26 | −1.588 | O | WAT |
| | | 2734 | H1 | 33.772 | −5.994 | −1.747 | H | WAT |
| | | 2735 | H2 | 32.565 | −5.679 | −0.889 | H | WAT |
| 286 | HOH | 2736 | OH2 | 12.396 | −9.672 | 23.659 | O | WAT |
| | | 2737 | H1 | 12.293 | −9.897 | 24.583 | H | WAT |
| | | 2738 | H2 | 12.283 | −8.722 | 23.629 | H | WAT |
| 287 | HOH | 2739 | OH2 | 15.414 | 14.441 | 23.64 | O | WAT |
| | | 2740 | H1 | 15.151 | 13.702 | 24.19 | H | WAT |
| | | 2741 | H2 | 15.024 | 15.205 | 24.063 | H | WAT |
| 288 | HOH | 2742 | OH2 | 21.332 | −3.295 | −8.937 | O | WAT |
| | | 2743 | H1 | 20.802 | −3.825 | −9.532 | H | WAT |
| | | 2744 | H2 | 22.179 | −3.217 | −9.376 | H | WAT |
| 290 | HOH | 2745 | OH2 | 23.028 | 3.792 | 17.286 | O | WAT |
| | | 2746 | H1 | 22.806 | 4.719 | 17.202 | H | WAT |
| | | 2747 | H2 | 23.592 | 3.611 | 16.534 | H | WAT |
| 291 | HOH | 2748 | OH2 | 3.662 | 1.475 | 18.547 | O | WAT |
| | | 2749 | H1 | 3.553 | 2.001 | 19.339 | H | WAT |
| | | 2750 | H2 | 4.574 | 1.187 | 18.574 | H | WAT |
| 292 | HOH | 2751 | OH2 | 35.309 | 6.968 | 18.526 | O | WAT |
| | | 2752 | H1 | 35.691 | 7.531 | 17.853 | H | WAT |
| | | 2753 | H2 | 34.977 | 6.211 | 18.045 | H | WAT |
| 293 | HOH | 2754 | OH2 | 18.022 | −0.568 | −8.26 | O | WAT |
| | | 2755 | H1 | 17.919 | −0.32 | −9.179 | H | WAT |
| | | 2756 | H2 | 17.313 | −0.11 | −7.808 | H | WAT |
| 294 | HOH | 2757 | OH2 | 29.154 | −14.743 | 7.181 | O | WAT |
| | | 2758 | H1 | 28.807 | −15.219 | 6.427 | H | WAT |
| | | 2759 | H2 | 29.622 | −14 | 6.801 | H | WAT |
| 298 | HOH | 2760 | OH2 | 13.982 | 9.368 | 10.184 | O | WAT |
| | | 2761 | H1 | 13.134 | 9.162 | 10.578 | H | WAT |
| | | 2762 | H2 | 14.527 | 9.646 | 10.92 | H | WAT |
| 299 | HOH | 2763 | OH2 | 38.701 | 1.924 | 10.338 | O | WAT |
| | | 2764 | H1 | 39.116 | 1.566 | 9.553 | H | WAT |
| | | 2765 | H2 | 38.543 | 1.162 | 10.895 | H | WAT |
| 302 | HOH | 2766 | OH2 | 13.316 | 11.756 | 17.617 | O | WAT |
| | | 2767 | H1 | 14.205 | 11.431 | 17.473 | H | WAT |
| | | 2768 | H2 | 12.797 | 10.97 | 17.786 | H | WAT |
| 305 | HOH | 2769 | OH2 | 29.722 | 4.371 | 19.089 | O | WAT |
| | | 2770 | H1 | 29.998 | 5.273 | 18.923 | H | WAT |
| | | 2771 | H2 | 30.412 | 4.007 | 19.643 | H | WAT |
| 307 | HOH | 2772 | OH2 | 38.262 | 15.916 | 14.472 | O | WAT |
| | | 2773 | H1 | 38.127 | 15.012 | 14.758 | H | WAT |
| | | 2774 | H2 | 39.124 | 15.909 | 14.056 | H | WAT |
| 308 | HOH | 2775 | OH2 | 15.275 | −4.041 | −1.509 | O | WAT |
| | | 2776 | H1 | 15.322 | −3.1 | −1.68 | H | WAT |
| | | 2777 | H2 | 16.172 | −4.352 | −1.633 | H | WAT |
| 310 | HOH | 2778 | OH2 | 17.834 | −3.694 | −4.856 | O | WAT |
| | | 2779 | H1 | 17.104 | −4.306 | −4.949 | H | WAT |
| | | 2780 | H2 | 18.039 | −3.706 | −3.921 | H | WAT |
| 317 | HOH | 2781 | OH2 | 30.546 | 7.047 | 18.386 | O | WAT |
| | | 2782 | H1 | 31.299 | 7.227 | 18.949 | H | WAT |
| | | 2783 | H2 | 30.744 | 7.503 | 17.568 | H | WAT |
| 318 | HOH | 2784 | OH2 | 24.337 | −19.839 | 1.508 | O | WAT |
| | | 2785 | H1 | 23.58 | −20.394 | 1.323 | H | WAT |
| | | 2786 | H2 | 24.142 | −19.012 | 1.066 | H | WAT |
| 319 | HOH | 2787 | OH2 | 6.391 | −4.832 | 14.4 | O | WAT |
| | | 2788 | H1 | 6.625 | −5.582 | 14.947 | H | WAT |
| | | 2789 | H2 | 7.231 | −4.443 | 14.155 | H | WAT |
| 323 | HOH | 2790 | OH2 | 32.141 | 11.459 | −0.46 | O | WAT |
| | | 2791 | H1 | 33.009 | 11.832 | −0.304 | H | WAT |
| | | 2792 | H2 | 31.8 | 11.271 | 0.414 | H | WAT |
| 324 | HOH | 2793 | OH2 | 14.138 | 2.731 | −7.857 | O | WAT |
| | | 2794 | H1 | 14.323 | 3.615 | −7.538 | H | WAT |
| | | 2795 | H2 | 13.393 | 2.84 | −8.448 | H | WAT |
| 327 | HOH | 2796 | OH2 | 8.39 | 8.079 | 13.51 | O | WAT |
| | | 2797 | H1 | 8.961 | 7.902 | 14.257 | H | WAT |
| | | 2798 | H2 | 7.798 | 8.766 | 13.817 | H | WAT |
| 329 | HOH | 2799 | OH2 | 18.034 | 14.982 | 2.873 | O | WAT |
| | | 2800 | H1 | 17.743 | 15.891 | 2.799 | H | WAT |
| | | 2801 | H2 | 18.983 | 15.042 | 2.976 | H | WAT |
| 335 | HOH | 2802 | OH2 | 18.689 | −18.557 | 23.705 | O | WAT |
| | | 2803 | H1 | 18.065 | −19.273 | 23.829 | H | WAT |
| | | 2804 | H2 | 18.537 | −17.975 | 24.449 | H | WAT |
| 342 | HOH | 2805 | OH2 | 9.267 | 0.134 | −3.877 | O | WAT |
| | | 2806 | H1 | 9.227 | 1.004 | −4.272 | H | WAT |
| | | 2807 | H2 | 9.104 | −0.468 | −4.604 | H | WAT |
| 343 | HOH | 2808 | OH2 | 26.082 | −22.116 | 5.38 | O | WAT |
| | | 2809 | H1 | 25.762 | −21.794 | 6.223 | H | WAT |
| | | 2810 | H2 | 25.967 | −23.066 | 5.427 | H | WAT |
| 348 | HOH | 2811 | OH2 | 4.751 | −0.625 | 8.681 | O | WAT |
| | | 2812 | H1 | 5.281 | −1.39 | 8.904 | H | WAT |
| | | 2813 | H2 | 4.273 | −0.885 | 7.894 | H | WAT |
| 349 | HOH | 2814 | OH2 | 4.737 | 3.206 | 2.458 | O | WAT |
| | | 2815 | H1 | 5.066 | 3.841 | 1.822 | H | WAT |
| | | 2816 | H2 | 5.099 | 2.368 | 2.17 | H | WAT |
| 350 | HOH | 2817 | OH2 | 29.932 | −2.467 | −9.956 | O | WAT |
| | | 2818 | H1 | 29.466 | −3.249 | −10.253 | H | WAT |
| | | 2819 | H2 | 29.356 | −1.74 | −10.192 | H | WAT |
| 351 | HOH | 2820 | OH2 | 29.255 | 7.611 | −10.151 | O | WAT |
| | | 2821 | H1 | 29.81 | 7.658 | −10.93 | H | WAT |
| | | 2822 | H2 | 28.899 | 6.723 | −10.164 | H | WAT |
| 370 | HOH | 2823 | OH2 | 29.874 | 18.074 | 10.832 | O | WAT |
| | | 2824 | H1 | 30.504 | 17.364 | 10.706 | H | WAT |
| | | 2825 | H2 | 29.455 | 17.876 | 11.67 | H | WAT |
| 378 | HOH | 2826 | OH2 | 16.249 | −18.947 | 10.048 | O | WAT |
| | | 2827 | H1 | 16.886 | −18.61 | 10.679 | H | WAT |
| | | 2828 | H2 | 15.426 | −18.983 | 10.536 | H | WAT |
| 400 | HOH | 2829 | OH2 | 28.347 | −4.792 | −10.343 | O | WAT |
| | | 2830 | H1 | 28.084 | −5.29 | −11.117 | H | WAT |
| | | 2831 | H2 | 27.556 | −4.322 | −10.08 | H | WAT |
| 414 | HOH | 2832 | OH2 | 23.03 | 2.229 | 19.657 | O | WAT |
| | | 2833 | H1 | 23.911 | 2.388 | 19.998 | H | WAT |
| | | 2834 | H2 | 22.982 | 2.756 | 18.86 | H | WAT |
| 416 | HOH | 2835 | OH2 | 7.12 | 2.84 | 15.324 | O | WAT |
| | | 2836 | H1 | 7.281 | 2.64 | 16.246 | H | WAT |
| | | 2837 | H2 | 7.827 | 3.438 | 15.082 | H | WAT |
| 417 | HOH | 2838 | OH2 | 23.036 | 6.369 | 19.424 | O | WAT |
| | | 2839 | H1 | 22.813 | 6.455 | 18.497 | H | WAT |
| | | 2840 | H2 | 22.63 | 5.546 | 19.694 | H | WAT |
| 418 | HOH | 2841 | OH2 | 8.789 | 4.663 | 18.008 | O | WAT |
| | | 2842 | H1 | 8.396 | 3.79 | 18.028 | H | WAT |
| | | 2843 | H2 | 8.396 | 5.12 | 18.752 | H | WAT |

*The data are derived from a homology modeling structure based on PDB:2JDD (GLYAT variant R7 + AcCoA + 3PG complex). The initial glyphosate structure is manually docked into the active site according to its similarity with 3PG. The initial R11 GLYAT structure was created by mutation from 2JDD and the stereo-chemical conflict was eliminated from local side-chain rotamer refinement. The structural model underwent a series of energy minimizations with CHARMm, on newly added hydrogen (CONJ, 500 cycles), on hydrogen and glyphosate (500 cycles), on non-backbone atoms (200 cycles), and on whole system (200 cycles). The minimized model further underwent a molecular dynamics simulation (~20,000 cylces) at 300 K. and subsequent energy minimization (500 cycles).
^{a}ResI: The residue ids in the structure
^{b}ResN: The residue names; the common amino acid residue with three letter representation; GLF representing Glyphosate; ACO representing Acetyl Co-enzyme A; and HOH representing water.
^{c}AtomI: The atom ids in structure.
^{d}AtomN: The atom name.
^{e}X, Y, Z: The atom coordinates of X, Y, and Z axes in Angstroms.
^{f}ElemN: The corresponding element symbol for each atom.
^{g}SegN: The segment names in the complex, Pro representing peptide, LIG representing the bound ligands, and WAT representing surrounding waters.

1. A method for evaluating the potential of a polypeptide to associate with glyphosate with a higher binding affinity when compared to a native glyphosate N-acetyltransferase (GLYAT) polypeptide or higher binding specificity for glyphosate when compared to a native GLYAT polypeptide, or a combination thereof, said method comprising:
(a) providing a three-dimensional molecular structure of at least a substrate binding cavity of a glyphosate N-acetyltransferase (GLYAT) polypeptide, wherein said GLYAT polypeptide is bound to glyphosate and an acetyl donor, wherein the three-dimensional molecular structure of said substrate binding cavity comprises:
(i) at least the atomic coordinates of Table 1 or Table 2; or
(ii) a structural variant of the substrate binding cavity of part (i), wherein said structural variant comprises a root mean square deviation from the back-bone atoms of the amino acids of Table 1 or Table 2 of not more than 2 Å;
(b) providing one or more three-dimensional molecular structures of one or more candidate polypeptides bound to glyphosate and an acetyl donor; wherein steps (a) and (b) can be performed in any order; and
(c) determining if the three-dimensional molecular structure of the candidate polypeptide comprises the substrate binding cavity of part a(i) or a(ii) to evaluate the potential of the candidate polypeptide to associate with glyphosate with a higher binding affinity or higher binding specificity or both when compared to a native GLYAT polypeptide.

2. The method of claim 1, wherein said substrate binding cavity comprises the atomic coordinates of Table 1 and Table 3 or a structural variant of the substrate binding cavity, wherein said structural variant comprises a root mean square deviation from the back-bone atoms of the amino acids of Table 1 and Table 3 of not more than 2 Å.

3. The method of claim 1, wherein said substrate binding cavity comprises the atomic coordinates of Table 2 and Table 4 or a structural variant of the substrate binding cavity, wherein said structural variant comprises a root mean square deviation from the back-bone atoms of the amino acids of Table 2 and Table 4 of not more than 2 Å.

4. The method of claim 1, wherein said substrate binding cavity comprises the atomic coordinates of Table 1 and Table 5; Table 3 and Table 5; Table 1, Table 3, and Table 5, or a structural variant of the substrate binding cavity, wherein said structural variant comprises a root mean square deviation from the back-bone atoms of the amino acids of Table 1 and Table 5; Table 3 and Table 5; or Table 1, Table 3, and Table 5 of not more than 2 Å.

5. The method of claim 1, wherein said substrate binding cavity comprises the atomic coordinates of Table 2 and Table 6; Table 4 and Table 6; Table 2, Table 4, and Table 6, or a structural variant of the substrate binding cavity, wherein said structural variant comprises a root mean square deviation from the back-bone atoms of the amino acids of Table 2 and Table 6; Table 4 and Table 6; or Table 2, Table 4, and Table 6 of not more than 2 Å.

6. The method of claim 1, wherein said acetyl donor comprises acetyl coA.

7. The method of claim 1, wherein said candidate polypeptide comprises a GLYAT polypeptide.

8. The method of claim 1, further comprising altering the primary structure of the candidate polypeptide to maximize a similarity between the three-dimensional molecular structure of part a(i) or a(ii) and the three-dimensional molecular structure of the candidate polypeptide.

9. The method of claim 1, wherein said method further comprises producing said candidate polypeptide.

10. The method of claim 9, wherein said method further comprises assaying the affinity, specificity, or both of said candidate polypeptide for glyphosate.

11. A method for evaluating the potential of a candidate polypeptide to have N-acetyltransferase activity with a higher catalytic rate (k_{cat}) for a substrate when compared to a native GLYAT polypeptide, said method comprising:
(a) providing a three-dimensional molecular structure of at least a GNAT wedge joining region of a GLYAT polypeptide, wherein the GLYAT polypeptide is bound to glyphosate and an acetyl donor, wherein the GNAT wedge joining region comprises:
(i) at least the atomic coordinates of Table 7 or Table 8; or
(ii) a structural variant of the GNAT wedge joining region of part (i), wherein said structural variant comprises a root mean square deviation from the back-bone atoms of the amino acids of Table 7 or Table 8 of not more than 2 Å, wherein said GLYAT polypeptide is bound to glyphosate and an acetyl donor;
(b) providing one or more three-dimensional molecular structures of one or more candidate polypeptides bound to a substrate and an acetyl donor, wherein said candidate polypeptide is an N-acetyltransferase comprising a GNAT wedge; wherein steps (a) and (b) can be performed in any order; and
(c) determining if the three-dimensional molecular structure of the candidate polypeptide comprises the GNAT wedge joining region of part (i) or (ii) to evaluate the potential of the candidate polypeptide to have N-acetyltransferase activity with a higher catalytic rate (k_{cat}) for a substrate when compared to a native GLYAT polypeptide.

12. The method of claim 11, wherein said GNAT wedge joining region comprises the atomic coordinates of Table 7 and Table 9 or a structural variant of the wedge joining region, wherein said structural variant comprises a root mean square deviation from the back-bone atoms of the amino acids of Table 7 and Table 9 of not more than 2 Å.

13. The method of claim 11, wherein said GNAT wedge joining region comprises the atomic coordinates of Table 8 and Table 10 or a structural variant of the wedge joining region, wherein said structural variant comprises a root mean square deviation from the back-bone atoms of the amino acids of Table 8 and Table 10 of not more than 2 Å.

14. The method of claim 11, wherein said method further comprises producing said candidate polypeptide.

15. The method of claim 14, wherein said method further comprises assaying the catalytic rate of said candidate polypeptide for said substrate.

16. The method of claim 11, wherein said substrate comprises glyphosate.

17. The method of claim 16, wherein said three-dimensional molecular structure of a GLYAT polypeptide further comprises a substrate binding domain, wherein the substrate binding domain comprises:
(i) at least the atomic coordinates of Table 1 or Table 2; or
(ii) a structural variant of the substrate binding cavity of part (i), wherein said structural variant comprises a root mean square deviation from the back-bone atoms of the amino acids of Table 1 or Table 2 of not more than 2 Å; and
wherein said method further comprises determining if the three-dimensional molecular structure of the candidate polypeptide comprises the substrate binding cavity of (i) or (ii) to evaluate the potential of the candidate polypeptide to have N-acetyltransferase activity with a higher catalytic rate (k_{cat}) for glyphosate when compared to a native GLYAT polypeptide.

18. The method of claim 17, wherein said substrate binding cavity comprises the atomic coordinates of Table 1 and Table 3 or a structural variant of the substrate binding cavity, wherein said structural variant comprises a root mean square deviation from the back-bone atoms of the amino acids of Table 1 and Table 3 of not more than 2 Å.

19. The method of claim 17, wherein said substrate binding cavity comprises the atomic coordinates of Table 2 and Table 4 or a structural variant of the substrate binding cavity, wherein said structural variant comprises a root mean square deviation from the back-bone atoms of the amino acids of Table 2 and Table 4 of not more than 2 Å.

20. The method of claim 17, wherein said substrate binding cavity comprises the atomic coordinates of Table 1 and Table 5; Table 3 and Table 5; Table 1, Table 3, and Table 5, or a structural variant of the substrate binding cavity, wherein said structural variant comprises a root mean square deviation from the back-bone atoms of the amino acids of Table 1 and Table 5; Table 3 and Table 5; or Table 1, Table 3, and Table 5 of not more than 2 Å.

21. The method of claim 17, wherein said substrate binding cavity comprises the atomic coordinates of Table 2 and Table 6; Table 4 and Table 6; Table 2, Table 4, and Table 6, or a structural variant of the substrate binding cavity, wherein said structural variant comprises a root mean square deviation from the back-bone atoms of the amino acids of Table 2 and Table 6; Table 4 and Table 6; or Table 2, Table 4, and Table 6 of not more than 2 Å.

22. The method of claim 11, wherein said acetyl donor comprises acetyl coA.

23. The method of claim 11, wherein said candidate polypeptide comprises a GLYAT polypeptide.

24. The method of claim 11, further comprising altering a primary structure of the candidate polypeptide to maximize a similarity between the three-dimensional molecular structure of the GNAT wedge joining region of the GLYAT polypeptide and the three-dimensional molecular structure of the candidate polypeptide.

25. A computer-readable storage medium encoded with the atomic coordinates of a glyphosate N-acetyltransferase (GLYAT) polypeptide bound to glyphosate and acetyl coenzyme A, said atomic coordinates comprising:
(a) a three-dimensional representation of at least a substrate binding cavity comprising at least the atomic coordinates of Table 1 or Table 2; or
(b) a variant of the three-dimensional representation of part (a), wherein said variant comprises a root mean square deviation from the back-bone atoms of the amino acids of Table 1 or Table 2 of not more than 2 Å.

26. The computer-readable storage medium of claim 25, wherein said substrate binding cavity comprises the atomic coordinates of Table 1 and Table 3 or a structural variant of the substrate binding cavity, wherein said structural variant comprises a root mean square deviation from the back-bone atoms of the amino acids of Table 1 and Table 3 of not more than 2 Å.

27. The computer-readable storage medium of claim 25, wherein said substrate binding cavity comprises the atomic coordinates of Table 2 and Table 4 or a structural variant of the substrate binding cavity, wherein said structural variant comprises a root mean square deviation from the back-bone atoms of the amino acids of Table 2 and Table 4 of not more than 2 Å.

28. The computer-readable storage medium of claim 25, wherein said substrate binding cavity comprises the atomic coordinates of Table 1 and Table 5; Table 3 and Table 5; Table 1, Table 3, and Table 5, or a structural variant of the substrate binding cavity, wherein said structural variant comprises a root mean square deviation from the back-bone atoms of the amino acids of Table 1 and Table 5; Table 3 and Table 5; or Table 1, Table 3, and Table 5 of not more than 2 Å.

29. The computer-readable storage medium of claim 25, wherein said substrate binding cavity comprises the atomic coordinates of Table 2 and Table 6; Table 4 and Table 6; Table 2, Table 4, and Table 6, or a structural variant of the substrate binding cavity, wherein said structural variant comprises a root mean square deviation from the back-bone atoms of the amino acids of Table 2 and Table 6; Table 4 and Table 6; or Table 2, Table 4, and Table 6 of not more than 2 Å.

30. The computer-readable storage medium of claim 25, wherein said atomic coordinates of a glyphosate N-acetyltransferase (GLYAT) polypeptide bound to glyphosate and an acetyl donor comprise the atomic coordinates of Table 18 or Table 19.

31. A computer-readable storage medium encoded with the atomic coordinates of a glyphosate N-acetyltransferase (GLYAT) polypeptide bound to glyphosate and an acetyl donor, said atomic coordinates comprising:
(a) a three-dimensional representation of at least a wedge joining region comprising at least the atomic coordinates of Table 7 or Table 8; or
(b) a variant of the three-dimensional representation of part (a), wherein said variant comprises a root mean square deviation from the back-bone atoms of the amino acids of Table 7 or Table 8 of not more than 2 Å.

32. The computer-readable storage medium of claim 31, wherein said GNAT wedge joining region comprises the atomic coordinates of Table 7 and Table 9 or a structural variant of the wedge joining region, wherein said structural variant comprises a root mean square deviation from the back-bone atoms of the amino acids of Table 7 and Table 9 of not more than 2 Å.

33. The computer-readable storage medium of claim 31, wherein said GNAT wedge joining region comprises the atomic coordinates of Table 8 and Table 10 or a structural variant of the wedge joining region, wherein said structural variant comprises a root mean square deviation from the back-bone atoms of the amino acids of Table 8 and Table 10 of not more than 2 Å.

34. A recombinant GNAT polypeptide having an array of amino acid side chains which together comprise a glyphosate acetyltransferase active site, said active site being composed of:
(i) at least the atomic coordinates of Table 1 or Table 2; or
(ii) a structural variant of the substrate binding cavity of part (i), wherein said structural variant comprises a root mean square deviation from the back-bone atoms of the amino acids of Table 1 or Table 2 of not more than 2 Å,
wherein said GNAT polypeptide has less than about 60% sequence identity to the native GLYAT sequence as set forth in SEQ ID NO:3.

35. A recombinant GNAT polypeptide having an array of amino acid side chains which together comprise a glyphosate acetyltransferase active site, said active site being composed of:
(i) at least the atomic coordinates of Table 7 or Table 8; or
(ii) a structural variant of the GNAT wedge joining region of part (i), wherein said structural variant comprises a root mean square deviation from the back-bone atoms of the amino acids of Table 7 or Table 8 of not more than 2 Å, wherein said GLYAT polypeptide is bound to glyphosate and an acetyl donor,
wherein said GNAT polypeptide has less than about 60% sequence identity to the native GLYAT sequence as set forth in SEQ ID NO:3.
